SEQUENCE LISTING (207 sequences)

SEQ ID NO: 1 - protein, 426 aa, Artificial Sequence
  Description of Artificial Sequence Activin Polypeptide
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 2 - protein, 407 aa, Artificial Sequence
  Description of Artificial Sequence Activin Polypeptide
MDGLPGRALGAACLLLLAAGWLGPEAWGSPTPPPTPAAPPPPPPPGSPGGSQDTCTSCGGFRRPEELGRVDGDFLEAVKRHILSRLQMRGRPNITHAVPKAAMVTALRKLHAGKVREDGRVEIPHLDGHASPGADGQERVSEIISFAETDGLASSRVRLYFFISNEGNQNLFVVQASLWLYLKLLPYVLEKGSRRKVRVKVYFQEQGHGDRWNMVEKRVDLKRSGWHTFPLTEAIQALFERGERRLNLDVQCDSCQELAVVPVFVDPGEESHRPFVVVQARLGDSRHRIRKRGLECDGRTNLCCRQQFFIDFRLIGWNDWIIAPTGYYGNYCEGSCPAYLAGVPGSASSFHTAVVNQYRMRGLNPGTVNSCCIPTKLSTMSMLYFDDEYNIVKRDVPNMIVEECGCA

SEQ ID NO: 3 - protein, 513 aa, Artificial Sequence
  Description of Artificial Sequence Activin Polypeptide
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 4 - protein, 405 aa, Artificial Sequence
  Description of Artificial Sequence Activin Polypeptide
MCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 5 - protein, 513 aa, Artificial Sequence; identical to SEQ ID NO: 3
  Description of Artificial Sequence Activin Polypeptide

SEQ ID NO: 6 - protein, 512 aa, Artificial Sequence
  Description of Artificial Sequence Activin Polypeptide
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 7 - protein, 509 aa, Artificial Sequence
  Description of Artificial Sequence Activin Polypeptide
MVDGVMILPVLIMIALPSPSMEDEKPKVNPKLYMCVCEGLSCGNEDHCEGQQCFSSLSINDGFHVYQKGCFQVYEQGKMTCKTPPSPGQAVECCQGDWCNRNITAQLPTKGKSFPGTQNFHLEVGLIILSVVFAVCLLACLLGVALRKFKRRNQERLNPRDVEYGTIEGLITTNVGDSTLADLLDHSCTSGSGSGLPFLVQRTVARQITLLECVGKGRYGEVWRGSWQGENVAVKIFSSRDEKSWFRETELYNTVMLRHENILGFIASDMTSRHSSTQLWLITHYHEMGSLYDYLQLTTLDTVSCLRIVLSIASGLAHLHIEIFGTQGKPAIAHRDLKSKNILVKKNGQCCIADLGLAVMHSQSTNQLDVGNNPRVGTKRYMAPEVLDETIQVDCFDSYKRVDIWAFGLVLWEVARRMVSNGIVEDYKPPFYDVVPNDPSFEDMRKVVCVDQQRPNIPNRWFSDPTLTSLAKLMKECWYQNPSARLTALRIKKTLTKIDNSLDKLKTDC

SEQ ID NO: 8 - protein, 505 aa, Artificial Sequence
  Description of Artificial Sequence Activin Polypeptide
MAESAGASSFFPLVVLLLAGSGGSGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVELVGIIAGPVFLLFLIIIIVFLVINYHQRVYHNRQRLDMEDPSCEMCLSKDKTLQDLVYDLSTSGSGSGLPLFVQRTVARTIVLQEIIGKGRFGEVWRGRWRGGDVAVKIFSSREERSWFREAEIYQTVMLRHENILGFIAADNKDNGTWTQLWLVSDYHEHGSLFDYLNRYTVTIEGMIKLALSAASGLAHLHMEIVGTQGKPGIAHRDLKSKNILVKKNGMCAIADLGLAVRHDAVTDTIDIAPNQRVGTKRYMAPEVLDETINMKHFDSFKCADIYALGLVYWEIARRCNSGGVHEEYQLPYYDLVPSDPSIEEMRKVVCDQKLRPNIPNWWQSYEALRVMGKMMRECWYANGAARLTALRIKKTLSQLSVQEDVKI

SEQ ID NO: 9 - protein, 453 aa, Artificial Sequence
  Description of Artificial Sequence Activin Polypeptide
MVSIFNLDGMEHHVRTCIPKVELVPAGKPFYCLSSEDLRNTHCCYTDYCNRIDLRVPSGHLKEPEHPSMWGPVELVGIIAGPVFLLFLIIIIVFLVINYHQRVYHNRQRLDMEDPSCEMCLSKDKTLQDLVYDLSTSGSGSGLPLFVQRTVARTIVLQEIIGKGRFGEVWRGRWRGGDVAVKIFSSREERSWFREAEIYQTVMLRHENILGFIAADNKDNGTWTQLWLVSDYHEHGSLFDYLNRYTVTIEGMIKLALSAASGLAHLHMEIVGTQGKPGIAHRDLKSKNILVKKNGMCAIADLGLAVRHDAVTDTIDIAPNQRVGTKRYMAPEVLDETINMKHFDSFKCADIYALGLVYWEIARRCNSGGVHEEYQLPYYDLVPSDPSIEEMRKVVCDQKLRPNIPNWWQSYEALRVMGKMMRECWYANGAARLTALRIKKTLSQLSVQEDVKI

SEQ ID NO: 10 - protein, 546 aa, Artificial Sequence
  Description of Artificial Sequence Activin Polypeptide
MAESAGASSFFPLVVLLLAGSGGSGPRGVQALLCACTSCLQANYTCETDGACMVSIFNLDGMEHHVRTCIPKVELVPAGKPFYCLSSEDLRNTHCCYTDYCNRIDLRVPSGHLKEPEHPSMWGPVELVGIIAGPVFLLFLIIIIVFLVINYHQRVYHNRQRLDMEDPSCEMCLSKDKTLQDLVYDLSTSGSGSGLPLFVQRTVARTIVLQEIIGKGRFGEVWRGRWRGGDVAVKIFSSREERSWFREAEIYQTVMLRHENILGFIAADNKADCSFLTLPWEVVMVSAAPKLRSLRLQYKGGRGRARFLFPLNNGTWTQLWLVSDYHEHGSLFDYLNRYTVTIEGMIKLALSAASGLAHLHMEIVGTQGKPGIAHRDLKSKNILVKKNGMCAIADLGLAVRHDAVTDTIDIAPNQRVGTKRYMAPEVLDETINMKHFDSFKCADIYALGLVYWEIARRCNSGGVHEEYQLPYYDLVPSDPSIEEMRKVVCDQKLRPNIPNWWQSYEALRVMGKMMRECWYANGAARLTALRIKKTLSQLSVQEDVKI

SEQ ID NO: 11 - DNA, 2175 bp, Artificial Sequence
  Description of Artificial Sequence Antisense nucleic acid
agtacagtataaaacttcacagtgccaataccatgaagaggagctcagacagctcttaccacatgatacaagagccggctggtggaagagtggggaccagaaagagaatttgctgaagaggagaaggaaaaaaaaaacaccaaaaaaaaaaataaaaaaatccacacacacaaaaaaacctgcgcgtgaggggggaggaaaagcagggccttttaaaaaggcaatcacaacaacttttgctgccaggatgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcggggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatagagttgcccagcccagggggaaagggagcaagagttgtccagagaagacagtggcaaaatgaagaaatttttaaggtttctgagttaaccagaaaaatagaaattaaaaacaaaacaaaaaaaaaaacaaaaaaaaacaaaagtaaattaaaaacaaaacctgatgaaacagatgaaggaagatgtggaaaaaatccttagccagggctcagagatgaagcagtgaaagagacaggaattgggagggaaagggagaatggtgtaccctttatttcttctgaaatcacactgatgacatcagttgtttaaacggggtattgtcctttccccccttgaggttcccttgtgagccttgaatcaaccaatctagtctgcagtagtgtggactagaacaacccaaatagcatctagaaagccatgagtttgaaagggcccatcacaggcactttcctacccaattacccaggtcataaggtatgtctgtgtgacacttatctctgtgtatatcagcatacacacacacacacacacacacacacacacacacaggcatttccacacattacatatatacacatactggtaaaagaacaatcgtgtgcaggtggtcacacttcctttttctgtaccacttttgcaacaaaacaa

SEQ ID NO: 12 - DNA, 3218 bp, Artificial Sequence
  Description of Artificial Sequence Antisense nucleic acid
actcggctcgcctcgcggcgggcgccctcgtcgccagcggcgcaccatggacgggctgcccggtcgggcgctgggggccgcctgccttctgctgctggcggccggctggctggggcctgaggcctggggctcacccacgcccccgccgacgcctgccgcgccgccgccacccccgccacccggatccccgggtggctcgcaggacacctgtacgtcgtgcggcggcttccggcggccagaggagctcggccgagtggacggcgacttcctggaggcggtgaagcggcacatcttgagccgcctgcagatgcggggccggcccaacatcacgcacgccgtgcctaaggccgccatggtcacggccctgcgcaagctgcacgcgggcaaggtgcgcgaggacggccgcgtggagatcccgcacctcgacggccacgccagcccgggcgccgacggccaggagcgcgtttccgaaatcatcagcttcgccgagacagatggcctcgcctcctcccgggtccgcctatacttcttcatctccaacgaaggcaaccagaacctgtttgtggtccaggccagcctgtggctttacctgaaactcctgccctacgtcctggagaagggcagccggcggaaggtgcgggtcaaagtgtacttccaggagcagggccacggtgacaggtggaacatggtggagaagagggtggacctcaagcgcagcggctggcataccttcccactcacggaggccatccaggccttgtttgagcggggcgagcggcgactcaacctagacgtgcagtgtgacagctgccaggagctggccgtggtgccggtgttcgtggacccaggcgaagagtcgcaccggccctttgtggtggtgcaggctcggctgggcgacagcaggcaccgcattcgcaagcgaggcctggagtgcgatggccggaccaacctctgttgcaggcaacagttcttcattgacttccgcctcatcggctggaacgactggatcatagcacccaccggctactacgggaactactgtgagggcagctgcccagcctacctggcaggggtccccggctctgcctcctccttccacacggctgtggtgaaccagtaccgcatgcggggtctgaaccccggcacggtgaactcctgctgcattcccaccaagctgagcaccatgtccatgctgtacttcgatgatgagtacaacatcgtcaagcgggacgtgcccaacatgattgtggaggagtgcggctgcgcctgacagtgcaaggcaggggcacggtggtggggcacggagggcagtcccgggtgggcttcttccagcccccgcgggaacgggggtacacggtgggctgagtacagtcattctgttgggctgtggagatagtgccagggtgcggcctgagatatttttctacagcttcatagagcaaccagtcaaaaccagagcgagaaccctcaactgacatgaaatactttaaaatgcacacgtagccacgcacagccagacgcatcctgccacccacacagcagcctccaggataccagcaaatggatgcggtgacaaatggcagcttagctacaaatgcctgtcagtcggagagaatggggtgagcagccaccattcccaccagctggcccggccactctgaattgcgccttccgagcacacataaaagcacaaagacagagacgcagagagagagagagagccacggagaggaaaagcagatgcaggggtggggagcgcagctcggcggaggctgcgtgtgccccgtggcttttaccaggcctgctctgcctggctcgatgtctgcttcttccccagcctgggatccttcgtgcttcaaggcctggggagcctgtccttccatgcccttgtcgagggaaagagacccagaaaggacacaacccgtcagagacctgggagcaggggcaatgaccgtttgactgtttgtggcttgggcctctgacatgacttatgtgtgtgtgtgtttttggggtggggagggagggagagaagagggggctaaatttgatgctttaactgatctccaacagttgacaggtcatccttgccagttgtataactgaaaaaggacttttctaccaggtatgaccttttaagtgaaaatctgaattgttctaaatggaaagaaaaaaagttgcaatctgtgcccttcattggggacattcctctaggactggtttggggacgggtgggaatgacccctaggcaaggggatgagaccgcaggaggaaatggcggggaggaggcattcttgaactgctgaggatggggggtgtcccctcagcggaggccaagggaggggagcagcctagttggtcttggagagatggggaaggctttcagctgatttgcagaagttgcccatgtgggccccagccatcagggctggccgtggacgtggcccctgcccactcacctgcccgcctgcccgcccgcccgcatagcacttgcagacctgcctgaacgcacatgacatagcacttgccgatctgcgtgtgtccagaagtggcccttggccgagcgccgaactcgctcgccctctagatgtccaagtgccacgtgaactatgcaatttaaagggttgacccacactagacgaaactggactcgtacgactctttttatattttttatacttgaaatgaaatcctttgcttcttttttaagcgaatgattgcttttaatgtttgcactgatttagttgcatgattagtcagaaactgccatttgaaaaaaagttatttttatagcagcaaaaaaaaaaaaaaaagaatacagttaaatgtattatacataattttggaaccaaagaggccaacagatcagttttaattttattagacggtgaggccatctgagatgaggtggacgttctgagcagtcccttgagtggcctgccaacgtttcagggtatgaatggattttgtttattcggtttgatgtgtcttttccatccttacacacccagaaggtagagtaaaaatgactatgatagaatgcaggtgtgtatccttaaatcctcatctttatgtttatttaataaagctccccttagattctgtttcataataatttaaaaccaaacaattttcccatagacttgctgttaaagtattgtacgtttgtgtacagtttaagaaaataaaagattgagtgccacgggaaaaaaaaaaaaaaa

SEQ ID NO: 13 - DNA, 5374 bp, Artificial Sequence
  Description of Artificial Sequence Antisense nucleic acid
aggagaccgaaaacgcggccgagcccggagcccggagctggagccagagcctggaccagaacttggccgccgcctgcaccgccgccgccgctgccgcccgccgccccttccccgcgccgcagccgcctcgccgccaccgccgcgagctcggccgccagtggtcctcggactttaggtgtctgggttgaaggaggtttgtctccgaggaagacccagggaactggatatctagcgagaacttcctccggattccccggcgcctcgggaaaatgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtctatgatggttgcgccatctgtgcacactaagaaatgggactctgaactggagctgctaagctaaagaaactgcttacagtttattttctgtgtaaaatgagtaggatgtctcttggaaatgttaagaaagaagaccctttgttgaaaaatgttgctctgggagacttactgcattgccgacagcacagatgtgaaggacatgagactaagagaaaccttgcaaactctataaagaaacttttgaaaaagtgtacatgaagaatgtagccctctccaaatcaaggatcttttggacctggctaatggagtgtttgaaaactgacatcagatttcttaatgtctgtcagaagacactaattccttaaatgaactactgctattttttttaaatcaaaaacttttcatttcagattttaaaaagggtaacttgtttttattgcatttgctgttgtttctataaatgactattgtaatgccaatatgacacagcttgtgaatgtttagtgtgctgctgttctgtgtacataaagtcatcaaagtggggtacagtaaagaggcttccaagcattactttaacctccctcaacaaggtatacctcagttccacggttgctaaattataaaattgaaaacactaacaaaatttgaataataaatcgatccatgttttgtaacaaattcactgtgttatttaaggaaaaaaaggtaagctatgcttagtgccaacaataagtggccattcgtaaagcagtgttttagcatttcttgtgctggcttgtaatgtagggaaaaaaagtgctgttttttgaaaagatggtgtcatttcccccttcttcccatgttttaaagccccatcttatatccagttcccaaaatttgcatacttacctaagtattttttttaggtgtgctgtgtttggggaatatttgaaaatttaaagcatgatttaaaattttttaaagtgagctgtgacactggaaagctcttcattttatcttttaaaatagagttttttctatttatatatgtaaaattgtagtgtatttcttttcaccaaacagtgtgtgggacattctttatcactgttttaggatcacctcaggaagtgtcgttacccagaattccccactgtctgctatgagacttgtaactttatcactatacttctgcttggtgccatcttgtcagagtaatatttgatgtctgtgatatgtaaagaattatcctaggataaagatattaaactttaagcagatttcagatgttactgctttaaaacaaatcagggataacaaattaaacgtataacttaaaatatgcaatgacatttagaggtaaccaatgttgatataggtagcatagcctagcctcctccccaaaattgcttttacaactaacactgatactaatttaggatagttcatgccttatccttgctaagaaaatggaattgatggtaggcaggtgctaaagtgcttttcaaaacaatattacgttagaatacaattggattcttcctcaaatttatacaggccaaaaagtaaaacattaattttctgaatttccagattaccaatcaattaatcaacaaatagccagtattatgctgtgtatttctgtcaggtcattttaaaatccatgttaattttataaaagaattttttacatgtcactgtcaggagctcactgtgaatgtgttgtcttcaaatggttatttaaccacacagtacactacattttacatatatgtacgtaatctctgggaatagtaaattaattatgttatttataaacaatacataggtcaacagactttaagcagggaggaaaagaagagtaatagcgtctgtgtgctgcagaccattcagaactgtcacgtgtgtccccatggtctcattcattgtattcctagcaattcccttttcaatgttgagttcacctctttatttcacaaagtacttggtctctcaatttcttgatctggttttgcttccatttaaaaactaatcaagaagggaaaatattgagaatgtgcatacaagaaaatcattaatttcctgaagatgaatttctacctgttgtgaacatttaactttctttttaaaagttaaacaaaaataaacaagggatattatgatgaatgtttggcttatgtgagtactagagataaaatttttaaacccagttattcacaatataaaatgttttcaagttagaaaaaatttttagaaatcctgggtattgtatttaactgtagctaaccaattttaaaacttgtattcttttgagaactattattaatagaaaaactttttataagcagtaaaataagaatgttccagtgactacctgtccttatacctagtcttgttaaaactttcttttgcagggtatttagtgtttggtttacagtcagtgcagagtgggcaagttaacagaaagtttgagctagagatactggaaaaaaaaaagatcaaagaatgagaaaaatggtgatccattttggggcaaactgagaccccccaaataactctttcctcatgtgtatggtgctcctcatgactcgtcttgtattttgcctttctgatacccatcagaactgctgctgctctaacttatactctttaccttgcccagatctccgcgtaaggaatgctttatgatcaacttgccataggactgatggattaaccagtgttcggctttatttgaagtctatgccctgcacagctcttgtatgtattttagatgctagaagtttttttagcatgtgatgtgtgattcttgtttgaattctaggtaccttgtgaattccagaaaaagagactgtgcttcacgattgttagtcccatgaacttgcactatctatctttcatggtgatgttttgaaaatacaatcaggaaaaaacccaacacctttggaatttaaaatagaatcatatcatgaaatttaaaaagaatctcttctgttgcatttcctcacccctaagtaacagctacatttaagtaaaatgcaggtggtaggggaaaaaaaaccatggcgagatggtggtttagtggaataaactgattactggtttttttgtttttttttttttttttaaagaaagaagcttcatcacagatactttccagtttctcttttatacttttttgaaagattactttttaggaacatttggtatgatatgcataaaattatttatccatttatgggcaaaatgatacaagtagcatcttgattgaacatcatttacctcagatattcaaccagcagtacgttttttatgcagtctcaacccatatcccatttgttacctctcagaatattggtaagcagttattttcgctttactctgtatttcttgtgttttgggcacaggttattgtactactgtcaaatcgtacttgctattttttctgcaagtatttaacagaaagcttaaaatccccataaaaccccaccttggataagtgattgttaaatattgtacaaataaaatgtatgctatccccattccatccccaagttaaataaaaaaatgaatacggtatgaaaaaaaaaaa

SEQ ID NO: 14 - DNA, 5263 bp, Artificial Sequence
  Description of Artificial Sequence Antisense nucleic acid
tgagcgtttttttttttttttttttttttttttggtctgggcttccgaatatgttttatgacggttgattttacaccaggaggtttgtctccgaggaagacccagggaactggatatctagcgagaacttcctccggattccccggcgcctcgggaaaatgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtctatgatggttgcgccatctgtgcacactaagaaatgggactctgaactggagctgctaagctaaagaaactgcttacagtttattttctgtgtaaaatgagtaggatgtctcttggaaatgttaagaaagaagaccctttgttgaaaaatgttgctctgggagacttactgcattgccgacagcacagatgtgaaggacatgagactaagagaaaccttgcaaactctataaagaaacttttgaaaaagtgtacatgaagaatgtagccctctccaaatcaaggatcttttggacctggctaatggagtgtttgaaaactgacatcagatttcttaatgtctgtcagaagacactaattccttaaatgaactactgctattttttttaaatcaaaaacttttcatttcagattttaaaaagggtaacttgtttttattgcatttgctgttgtttctataaatgactattgtaatgccaatatgacacagcttgtgaatgtttagtgtgctgctgttctgtgtacataaagtcatcaaagtggggtacagtaaagaggcttccaagcattactttaacctccctcaacaaggtatacctcagttccacggttgctaaattataaaattgaaaacactaacaaaatttgaataataaatcgatccatgttttgtaacaaattcactgtgttatttaaggaaaaaaaggtaagctatgcttagtgccaacaataagtggccattcgtaaagcagtgttttagcatttcttgtgctggcttgtaatgtagggaaaaaaagtgctgttttttgaaaagatggtgtcatttcccccttcttcccatgttttaaagccccatcttatatccagttcccaaaatttgcatacttacctaagtattttttttaggtgtgctgtgtttggggaatatttgaaaatttaaagcatgatttaaaattttttaaagtgagctgtgacactggaaagctcttcattttatcttttaaaatagagttttttctatttatatatgtaaaattgtagtgtatttcttttcaccaaacagtgtgtgggacattctttatcactgttttaggatcacctcaggaagtgtcgttacccagaattccccactgtctgctatgagacttgtaactttatcactatacttctgcttggtgccatcttgtcagagtaatatttgatgtctgtgatatgtaaagaattatcctaggataaagatattaaactttaagcagatttcagatgttactgctttaaaacaaatcagggataacaaattaaacgtataacttaaaatatgcaatgacatttagaggtaaccaatgttgatataggtagcatagcctagcctcctccccaaaattgcttttacaactaacactgatactaatttaggatagttcatgccttatccttgctaagaaaatggaattgatggtaggcaggtgctaaagtgcttttcaaaacaatattacgttagaatacaattggattcttcctcaaatttatacaggccaaaaagtaaaacattaattttctgaatttccagattaccaatcaattaatcaacaaatagccagtattatgctgtgtatttctgtcaggtcattttaaaatccatgttaattttataaaagaattttttacatgtcactgtcaggagctcactgtgaatgtgttgtcttcaaatggttatttaaccacacagtacactacattttacatatatgtacgtaatctctgggaatagtaaattaattatgttatttataaacaatacataggtcaacagactttaagcagggaggaaaagaagagtaatagcgtctgtgtgctgcagaccattcagaactgtcacgtgtgtccccatggtctcattcattgtattcctagcaattcccttttcaatgttgagttcacctctttatttcacaaagtacttggtctctcaatttcttgatctggttttgcttccatttaaaaactaatcaagaagggaaaatattgagaatgtgcatacaagaaaatcattaatttcctgaagatgaatttctacctgttgtgaacatttaactttctttttaaaagttaaacaaaaataaacaagggatattatgatgaatgtttggcttatgtgagtactagagataaaatttttaaacccagttattcacaatataaaatgttttcaagttagaaaaaatttttagaaatcctgggtattgtatttaactgtagctaaccaattttaaaacttgtattcttttgagaactattattaatagaaaaactttttataagcagtaaaataagaatgttccagtgactacctgtccttatacctagtcttgttaaaactttcttttgcagggtatttagtgtttggtttacagtcagtgcagagtgggcaagttaacagaaagtttgagctagagatactggaaaaaaaaaagatcaaagaatgagaaaaatggtgatccattttggggcaaactgagaccccccaaataactctttcctcatgtgtatggtgctcctcatgactcgtcttgtattttgcctttctgatacccatcagaactgctgctgctctaacttatactctttaccttgcccagatctccgcgtaaggaatgctttatgatcaacttgccataggactgatggattaaccagtgttcggctttatttgaagtctatgccctgcacagctcttgtatgtattttagatgctagaagtttttttagcatgtgatgtgtgattcttgtttgaattctaggtaccttgtgaattccagaaaaagagactgtgcttcacgattgttagtcccatgaacttgcactatctatctttcatggtgatgttttgaaaatacaatcaggaaaaaacccaacacctttggaatttaaaatagaatcatatcatgaaatttaaaaagaatctcttctgttgcatttcctcacccctaagtaacagctacatttaagtaaaatgcaggtggtaggggaaaaaaaaccatggcgagatggtggtttagtggaataaactgattactggtttttttgtttttttttttttttttaaagaaagaagcttcatcacagatactttccagtttctcttttatacttttttgaaagattactttttaggaacatttggtatgatatgcataaaattatttatccatttatgggcaaaatgatacaagtagcatcttgattgaacatcatttacctcagatattcaaccagcagtacgttttttatgcagtctcaacccatatcccatttgttacctctcagaatattggtaagcagttattttcgctttactctgtatttcttgtgttttgggcacaggttattgtactactgtcaaatcgtacttgctattttttctgcaagtatttaacagaaagcttaaaatccccataaaaccccaccttggataagtgattgttaaatattgtacaaataaaatgtatgctatccccattccatccccaagttaaataaaaaaatgaatacggtatgaaaaaaaaaaa

SEQ ID NO: 15 - DNA, 5177 bp, Artificial Sequence
  Description of Artificial Sequence Antisense nucleic acid
aggagaccgaaaacgcggccgagcccggagcccggagctggagccagagcctggaccagaacttggccgccgcctgcaccgccgccgccgctgccgcccgccgccccttccccgcgccgcagccgcctcgccgccaccgccgcgagctcggccgccagtggtcctcggactttaggtgtctgggttgaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtctatgatggttgcgccatctgtgcacactaagaaatgggactctgaactggagctgctaagctaaagaaactgcttacagtttattttctgtgtaaaatgagtaggatgtctcttggaaatgttaagaaagaagaccctttgttgaaaaatgttgctctgggagacttactgcattgccgacagcacagatgtgaaggacatgagactaagagaaaccttgcaaactctataaagaaacttttgaaaaagtgtacatgaagaatgtagccctctccaaatcaaggatcttttggacctggctaatggagtgtttgaaaactgacatcagatttcttaatgtctgtcagaagacactaattccttaaatgaactactgctattttttttaaatcaaaaacttttcatttcagattttaaaaagggtaacttgtttttattgcatttgctgttgtttctataaatgactattgtaatgccaatatgacacagcttgtgaatgtttagtgtgctgctgttctgtgtacataaagtcatcaaagtggggtacagtaaagaggcttccaagcattactttaacctccctcaacaaggtatacctcagttccacggttgctaaattataaaattgaaaacactaacaaaatttgaataataaatcgatccatgttttgtaacaaattcactgtgttatttaaggaaaaaaaggtaagctatgcttagtgccaacaataagtggccattcgtaaagcagtgttttagcatttcttgtgctggcttgtaatgtagggaaaaaaagtgctgttttttgaaaagatggtgtcatttcccccttcttcccatgttttaaagccccatcttatatccagttcccaaaatttgcatacttacctaagtattttttttaggtgtgctgtgtttggggaatatttgaaaatttaaagcatgatttaaaattttttaaagtgagctgtgacactggaaagctcttcattttatcttttaaaatagagttttttctatttatatatgtaaaattgtagtgtatttcttttcaccaaacagtgtgtgggacattctttatcactgttttaggatcacctcaggaagtgtcgttacccagaattccccactgtctgctatgagacttgtaactttatcactatacttctgcttggtgccatcttgtcagagtaatatttgatgtctgtgatatgtaaagaattatcctaggataaagatattaaactttaagcagatttcagatgttactgctttaaaacaaatcagggataacaaattaaacgtataacttaaaatatgcaatgacatttagaggtaaccaatgttgatataggtagcatagcctagcctcctccccaaaattgcttttacaactaacactgatactaatttaggatagttcatgccttatccttgctaagaaaatggaattgatggtaggcaggtgctaaagtgcttttcaaaacaatattacgttagaatacaattggattcttcctcaaatttatacaggccaaaaagtaaaacattaattttctgaatttccagattaccaatcaattaatcaacaaatagccagtattatgctgtgtatttctgtcaggtcattttaaaatccatgttaattttataaaagaattttttacatgtcactgtcaggagctcactgtgaatgtgttgtcttcaaatggttatttaaccacacagtacactacattttacatatatgtacgtaatctctgggaatagtaaattaattatgttatttataaacaatacataggtcaacagactttaagcagggaggaaaagaagagtaatagcgtctgtgtgctgcagaccattcagaactgtcacgtgtgtccccatggtctcattcattgtattcctagcaattcccttttcaatgttgagttcacctctttatttcacaaagtacttggtctctcaatttcttgatctggttttgcttccatttaaaaactaatcaagaagggaaaatattgagaatgtgcatacaagaaaatcattaatttcctgaagatgaatttctacctgttgtgaacatttaactttctttttaaaagttaaacaaaaataaacaagggatattatgatgaatgtttggcttatgtgagtactagagataaaatttttaaacccagttattcacaatataaaatgttttcaagttagaaaaaatttttagaaatcctgggtattgtatttaactgtagctaaccaattttaaaacttgtattcttttgagaactattattaatagaaaaactttttataagcagtaaaataagaatgttccagtgactacctgtccttatacctagtcttgttaaaactttcttttgcagggtatttagtgtttggtttacagtcagtgcagagtgggcaagttaacagaaagtttgagctagagatactggaaaaaaaaaagatcaaagaatgagaaaaatggtgatccattttggggcaaactgagaccccccaaataactctttcctcatgtgtatggtgctcctcatgactcgtcttgtattttgcctttctgatacccatcagaactgctgctgctctaacttatactctttaccttgcccagatctccgcgtaaggaatgctttatgatcaacttgccataggactgatggattaaccagtgttcggctttatttgaagtctatgccctgcacagctcttgtatgtattttagatgctagaagtttttttagcatgtgatgtgtgattcttgtttgaattctaggtaccttgtgaattccagaaaaagagactgtgcttcacgattgttagtcccatgaacttgcactatctatctttcatggtgatgttttgaaaatacaatcaggaaaaaacccaacacctttggaatttaaaatagaatcatatcatgaaatttaaaaagaatctcttctgttgcatttcctcacccctaagtaacagctacatttaagtaaaatgcaggtggtaggggaaaaaaaaccatggcgagatggtggtttagtggaataaactgattactggtttttttgtttttttttttttttttaaagaaagaagcttcatcacagatactttccagtttctcttttatacttttttgaaagattactttttaggaacatttggtatgatatgcataaaattatttatccatttatgggcaaaatgatacaagtagcatcttgattgaacatcatttacctcagatattcaaccagcagtacgttttttatgcagtctcaacccatatcccatttgttacctctcagaatattggtaagcagttattttcgctttactctgtatttcttgtgttttgggcacaggttattgtactactgtcaaatcgtacttgctattttttctgcaagtatttaacagaaagcttaaaatccccataaaaccccaccttggataagtgattgttaaatattgtacaaataaaatgtatgctatccccattccatccccaagttaaataaaaaaatgaatacggtatgaaaaaaaaaaa

SEQ ID NO: 16 - DNA, 11373 bp, Artificial Sequence
  Description of Artificial Sequence Antisense nucleic acid
gtgcgcggggcggcgccgcggaacatgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatctaagcccaggacatgagtgtctgtccagactcagtggatctgaagaaaaaaggaaaaaaagttgtgttttgttttggaaatcccataaaaccaacaaacacataaaatgcagctgctattttaccttgactttttattattattattataattattataattattattattaatattattttttggattggatcagtttttaccagcatattgctctactgtatcacaaacagcggacacgtcagcaggcgttgaggtgctgagctgtggatgcagaaccagcgccatgctgaagagcctcagccacctcctgtcctttgggattcgtttttcccgctttctctttgtttgtcgtctcagaatctgtgacacaaagaaacccatctcctgtcttaggaaacctaatgctgcaaactctacctagaggaacctttgaagactgttacataagaacataccttcctcagaagaggagtttcctctgccctctgcccttctcccctgcctccctccctcccctccttttattttgttttagtgagcttaagaaacagcagatgtgtctttcacggatctaacgggtgttgtcctgatcgagaaaaaaactgggatgagaatggtttggactggagttggaaggggaggacggtactgggggtagggtttggaacagagctacactggactcgggcacattcggagcagcatcctttagtatggaggctacttctcaggtaaccaggaattgaggggaaggaccttgtggaggccgagcattaacagcaagagcggggtttggagaaagtctgagattgggtgcagccctgacttacctgctggccctgaccagtttcttttcactaacttggccttgggcataggatgaaacattttttctgccctaattttaaaactaggtgagggtagaatcatcacaggttaggaatacattcttcataagacacgatgctgtaaatacccttaatggacgaaaagttgaaatacttttgtttcctcttggagcagttcagggaaatgcccacaggggattgtcctgcacagatagggcaagaggatttcctgggtggagtctgccaaggcctgcctcgctggggaccccagagtcctgcacctctggttccgccccaggtggtgacattactgtccccgttctgtggctcgtggacaagactttctccagaccccttaaagtggtacatattctaaaaaactgtttttctattatgccataaccttgctctagtcagtgaatgttcctaatgctgctgtttcaacatttgaattctttttaatttatgaaacatgctaaattttttttttcaaacaaaacacacacatccacatatacacatgcttcgctatgtggcttccaaggtttaaattttgaaaagtaaaagaattaaaacttcacgaccacagatcacctcaaaccagaaatacctcagaattttctacttatgtaaggtttattatatattttgttagttgtgttgtcttgtagtaagtatattttaatgtaagttggcttttgtgacaaggaagtttaaaagaaatagagaaaaagaaaaaagtttgcatcttctagggagtgctaccatttttgtttgataacgcccccttgtaaataattgtcatcaactgtaggttggctgtctgggccaagtctgggcatttatcagtcttgtttgtgaaggcttttccttctggtttctttagatcattttatttaaaaacagtgcatctcttcatcgtgagggtaggcaaggcgggggccgtggggagaggttgacctgggtgagaactgaagaggccgcctcctcttgggttgtttggagcttcacatgtaattcacatgtaacatgtaacttgatcggtcagtgttcagaatgacaagtaaccccgcttaaacttggtagaaggatggcccttagacctgaatggggtgattttacttgggatttaacttcttcagcaaattaacagcaacgttggaagagatctgtggcgcctctgtgaagcacaccgtgactcaggccagtcttttagtgcagcgtgtctgggagtgaagggttttgcccttgctggtcttggagtccacagtgtgaggggcactgcacatgcctgggcatctacctagtgtgctatgttcagtgtctggggcttactgccccggggtcctttcctctgggtgttggggcacagggtgctatgggaggcccatttgcttccctctcggagctcagtttttgcttcatgggtcaaaatgtgggctggccaagtggttacaggaacagggtttcggtaagctatgttgtctttttttttttttttttttttttttttaatggtttgattttgtgctgtggtattttttttcccttagaataatttttaatggcaaaacaggccttacagcagttgcttttctttaccatttatttctttaagaagctttaaaatatttattgaaaagtgccatatctaatttctttagctttcgcctcaggcagtgcaggcatctttacttttcatcctcagaagaaacaaacgactaacaaatgtagcaaatttactgcaggaatagttaggtcatgatactacctgaacactaaaccccagcctctttgtttggttttagttcctctgggtggtttttcttttgtgtgctggcttgattcttgtgagaagttttgacctggccaagggagggttgagccatggttctggtgtgggactttgcggtcaagacacagtacagacaggtcaggcctgcgtgccttttctctgggtggcctccccgttaggcccaccgtacgctcagccactatagtgtccctgtggggccttgccatcagattgtgtgtcaggagatggtacctttttggtgtggctggggaggagtgtggtccatgccagttctttgggcttcaggccactcttcccctcatgctgtggtgtaaagtgcacccatcaggtggtatatctggttctgatggcaagaagaaggtgggggatctccttatagggcatgggtctaggagcacagatgggccttttgccccgggtaaatgcttgtctgtttgctgtcatgtgttctttgaggagtgagccatctcgagccctgctttgaatttactgggtcatagagcctctgcctgtgctcttttccataatgacttcatgtgacatgcacttttggtgggctcagataattggtttctttttgtttttgacctcaggctctgtggcagactggggaaaatggggcctggcatcattttccctgtcaatgggaggggctgttccatgcagggtgggaggggaccaagttagcagagagtagccaaggatccttgcttcttcctttctagtgtgctgtcatccaagcaggctcctggctgtagggatgggccttggggaagaatcttctttgaaagcatctatgataactgagaagtcatccctagttggagaaatccagtaatgagcagaaggaggaagcaagtgaggacagaggccattgtattacagtgtcacgcagagggccctcaatgatggggcattggggaaggctgtagacatagtcatcagaacatcctggcctggcataagctgggttttctcctgggaccattggtcctcagcaggagttctttgcatgagttgctcaggggcaagggctgcaagtgggctgtgcttaggagaaagtgacacctggcagtgagggaagatggtgagcattattagcctttgttgtccagcatggccttcttgtcctgtctgctctggagaggagcctgtgggaccagtcctgcctggggagggcatacccacacgtgccagctgattctgactctgaatacatcatgtccggacttgggggtgtttctgcagaaaaaggaggttgtttttcagccttgaacatcttcaggaggatagagactcttgctcacatattcttagcaaagggaagggtctctcatctccaggccacagagatagttcttccattgccctaagaggctaggctaaccctcttgacataacttagacagcaaagcacttcatcctgtagttgggctctgtcacctttctcttcagttggccacattctcgtttcctccatcctgctatgctttgtgtgctcgggctgtgtgtggggtttttccctggtggaaggaagcccagctgtgtattgaatgtccttcatgtgttgtgtgtggctcagaaagcctgtcacttggcccctgtgctctgagccgtgagggtggggaggtggctgttccattaaagtgggagtattggatggccctcttgaaactagaattttgccttttttagtatgcagtataaagtttccagcatctattggtaacacaaagatttgctggtttttaaaataatacagtaagcataagtatgtaagtttttagaattggtactagaagttggacagctagttattctcgagaactttatttcactagaaaaatatactaattggaaagcagtttccaggagttaactcagtttaattttcagtctcagttattttagcctgttgagtttttgatggcacacctttggagagatggccacgcctgattcccatttcaggggcatcagaccatacctttttaagaagctccgtgaatctagtcatctacccttcatcctgggcgaacagccaaaaagagaaggggacaaggtgtctttttctccttctcactggggtgacatgaattcttttagttaatggctgtttgcaaattctaaactaatgaaatacttagcagctaacatgttcaatctagtaatgatgagtttaaatctcaattgacagtaatgttttagataaacaggcccagtaattcagttgatgaactgtatatcttctcagtctagatttgtaaatgtttaatgaattcagggttataagcatagttctttaagtaagattccagatagttgatttgcaaccagcagtctacctatgaatgtatcccaaacctttagaagattggaaaagatttttgaaataatgatttagttttgtaggaaaaacacccccttgaaaattaattcggttgacccagtaacattttttaaaacaattggtggctccaaaaggcctgccaacaaagaaaagtccaaattatctagtgggacattttgaatgttttatgtttattttgggtccactgtaaactttggttcaaaaaagaatttgaatttaaagaatttaccattatttaaattattaccaagtttttacattttcatgatggtattttccaggtatgaatgaaacatgactttttgattgtggtacttcctgtatcccctgtagtgccaaaaccagtgatactttatttgctcctatggcagctcatagaggtaaccgaagtgatttttcctcagtaattgaaacacatattctctaaatgccaatgtgtggtgatgggccctgcactgccttcatttctctagggcagtgtctttggattgtctagggcctaggtaattctgagaactactgtaaaccaaccacagggcactaaagcaatgtacacaccactctttgtgtgtatggaaggggttatataaacctgggctatgctggacatctacagaagagtattacattcacttgcaaagtttacatttttgagctcacagttatgaaaaatatgacccacaagtttttcaggcaggtgaggatgggtcttcttgcaaatgcatgagttctgtcttgagtcctgggaacttctctgttggttgagtgtgggctcattccctgactctcctaatcatgtttgcgtcagaatgttagcattgtaaataaaagaataggttgtataatagatacacaacacttgaaactttactttaaaaaaatcgatagttctacatatatatttagttatatcacttgacagatttcttctacacagtgtggagattgttttataccacagattatttttataaagttagtgaatttgaatgattttgtaatcagagctaatgagctttacctttcaagagaaacgtacactggagcatgagtggtgtggaacttttacttagtgtttatatggattcttgtgatacactggcagactggagtcaatttgcgggtcttttttggccaaaactccacttgtggttgtgtaggacagtgatattcagctcagcttcttgtggattgggaggagagagggcctgcaatgtgttttacattggtgcttcctcctgagatttctgttgaacaaagggttctgaggtcaaaaattagtttgtaagcctttgccataggacatagtcatgtgagagtgtttgggggaacagaaattgtataggggtgcctattggggtgggatgggactcgaataagattcaggtacaaaaactttgaaatgagaatctggtggtttgagtaatccaccagactgaattatctaagatcacattatccaggttggggggcagaattacccagttaagtaattgttcagaaaagtggggagggtggcatgtggatgcagtgatccaattaaatggagagctgccaggcacattttgtcctctctggtcagtgagaatggttgggttggctcgctgcttcaatctgtggaatcagccaggagcccagtgaggaagctcagaaccccagtaacagcagagcatctttcagatagctccagagttttcctgcttttctgaggaagctcagcatcactgccacaatacggaaagtggtcttcattttagcctatttatttttaggcagagagtggatggttatttgtgtgggacttttggtggcgatatataatgaataattaagttaatttctggtatgcataatggccagtcctgaggcccagctgaagacctgtcccccagaccctgcccgctggcttcaggctgctgcttctagacagaggtgcactggacgggatagttttatcaagagaatccctaatgtgtcattttaaaccagctgtgctttttattcattctggttgagcgtataggtttacactttaccctttttatacttggaataaatttagttccagcagatctagtagcactccagaaaccaaccccatctgttccccataaaaagaacattttctctgctctccagccacgtgtcttggaatgtaattctgttgtgcctttgtttttatcactctcttcgccccaaaagcaactgctgtaagcttttttctacttgtcttttctagtccccaacctctacctttttcctttttcccagccctaatttctggatgcacttctgtgatccaggtattttaagaaccagttacctcagacctcatgttgaacagtgtcgccatctgggtcctcttgatactgcagacttttaacgtacacatgcaggaaccctgctgagcgtgggcacttgttttaaagcaaaactcttcccaaggactgaagaaagggcttctggcaagctcgtcatggcattgtggtgggatgggtctagagtgtcatctgaatggtgcttcctgtgttcctctttgaattctgccattttcagtattcttgtgtgtctgaataggcaaagcgatttaattggctggtcttgcacgcaaattagttccaaagataagctctttgtaacacatttccagtcgctaatgctcaaatgtagaacattcctttaaatggcaggataaaaaacccactatccaccatagtgcattttgggaagatgtctgtagcatatgttgctgtgaaattaggccttgtgggatatggctgtttgtcattttgatgtattttaaataaatatatatattttttaaagagccttttttaccagttcaaaaagtttaattaaccagcagtcaccgcatctgaatttttgtctctggggcatagatggcagaccaagattaaaagtggtaactcagctatacgagcatgggctaccttcctgggctctcctgcagtcctgtagacctgctgttccgcagaccatgggacacaaggtcagtgtgttcccagtgagggtcccaagtcagtcatcttaagtgtttgttctctgccccattcagtggactgttgacttcagtccctgcaagtgctttagcccgagtggggttttctcagagcactgccacgagttaagtgtgtgtttagccaaataatttctccgtaagggaaaaatgcagtcacccaaattttaccaacaatgacagagatgagagtagaaaagattaggcaacatctgagttttaacttgaaaagtgtccaagtcatcatgaaaggccgactgggagcaagtgattattagagattcttcaggagacctcatctgaaaatgttaagactgccagtgagggaaggaattgttaaaatgccagcggcttttttttcctctttttttctgtaattctgtaaaaatgcagagaaagttgagtggtacttcagaattgagggagagggttaccgcagagtagaaatatatttctagatttcagttccacaccacaaatccacaacaatgccatttttcaactgtacaaaaatctgcttatgaactggacatgatcttaatggtagtgtcaaaggccaagtttttcacctgttaatatttttccacatttgtccttgaatctgaataactttatacagtactgtaaatttaacttacatcgagtttgttgtcaattcttatgaaaagagctttctgcatgtaacacatacggttaaagaacacagcaaaggacaaaatttgcaggaacagttttggaaccaacagaaaatgtcaccttttatttgccatcttatatatatctatcagttttaccagctacttctaaatttgtacattatttgtaagggaaagaaggaaaaccctaagacttgtctaacttagtggagaatgtgtgtgttgggcttaggatggatagctaagtcttattgagctgtgttacctaacttgtatataaaaattgtaattaaaagtttgggttcacctgtttctcacagtttaaaatgatgagtaattgcaaactctggaaatgtgactagtatatgatttaaggctgtagaagcaaggaagctctttcaagtgctaaaactaaagacttctagtttttggctcaaataagtactgtttgtataccaggatatgtgagatgtaaatgtagtaggtcacttttcacccttgtagctataaaataaaaattttgtagaacagaaatagcttgtactactgaattaacaaaagttatactaaagtatcatgtttaaaaaaaatatatatatatatacagagttaagcttgttgctgttaccctgtctggatttgaaaagtgtgctgatttatatatatatattacacacacacacacacacacacacacacacacacacacacacacacacacacacacacacacacacacacacatacacctaaaatggcctaaagcagacatccatgtaattacagttgcaaaatgaaaacattttggaaagaacattgtatcatagttcattcatttgcagtggatctttgttcctttttactgtggtaattttagaaatgagtgtcaagtttgaaattagatctgctaagttggggttttgctgcttgaactctgcactgggtcctcaaataaaccgatgtgaatgtagttttttccccctgtgtgaagaagcagttacaccccaacaataggaggaaaaatctagaactatttcaagttttatctttttgtatatgaaaataaaataataataaaacaa

SEQ ID NO: 17 - DNA, 3062 bp, Artificial Sequence
  Description of Artificial Sequence Antisense nucleic acid
gaagagatgtgggcctctggggccgctggattcagtaacttccgtcgggttctagactggctcggctctgtccagtttgtgccagatagtctcccaccccctccccacccctcctttcccctggagatttgaacgctgcttgcatgggagaaaagctacttagagaagaaaacgttccacttagtaacagaagaaaagtcttggttaaaaagttgtcatgaatttggcttttggagagaggcagcaagcctggagcattggtaagcgtcacactgccaaagtgagagctgctggagaactcataatcccaggaacgcctcttctactctccgagtaccccagtgaccagagtgagagaagctctgaacgagggcacgcggcttgaaggactgtgggcagatgtgaccaagagcctgcattaagttgtacaatggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagccgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggagaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgttgacattttcatagtgtcaagaaggaagatttgacgttgttgtcattgtccagctgggacctaatgctggcctgactggttgtcagaatggaatccatctgtctccctccccaaatggctgctttgacaaggcagacgtcgtacccagccatgtgttggggagacatcaaaaccaccctaacctcgctcgatgactgtgaactgggcatttcacgaactgttcacactgcagagactaatgttggacagacactgttgcaaaggtagggactggaggaacacagagaaatcctaaaagagatctgggcattaagtcagtggctttgcatagctttcacaagtctcctagacactccccacgggaaactcaaggaggtggtgaatttttaatcagcaatattgcctgtgcttctcttctttattgcactaggaattctttgcattccttacttgcactgttactcttaattttaaagacccaacttgccaaaatgttggctgcgtactccactggtctgtctttggataataggaattcaatttggcaaaacaaaatgtaatgtcagactttgctgcattttacacatgtgctgatgtttacaatgatgccgaacattaggaattgtttatacacaactttgcaaattatttattacttgtgcacttagtagtttttacaaaactgctttgtgcatatgttaaagcttatttttatgtggtcttatgattttattacagaaatgtttttaacactatactctaaaatggacattttcttttattatcagttaaaatcacattttaagtgcttcacatttgtatgtgtgtagactgtaactttttttcagttcatatgcagaacgtatttagccattacccacgtgacaccaccgaatatattactgatttagaagcaaagatttcagtagaattttagtcctgaacgctacggggaaaatgcattttcttcagaattatccattacgtgcatttaaactctgccagaaaaaaataactattttgttttaatctactttttgtatttagtagttatttgtataaattaaataaactgttttcaagtcaaaaaaaaaaaaaaaaaaaa

SEQ ID NO: 18 - DNA, 2881 bp, Artificial Sequence
  Description of Artificial Sequence Antisense nucleic acid
ctgcagcgcccggctgcctcgcactccgcctcccccggctcagcccccggccgcggcgggacccgagcctggagcattggtaagcgtcacactgccaaagtgagagctgctggagaactcataatcccaggaacgcctcttctactctccgagtaccccagtgaccagagtgagagaagctctgaacgagggcacgcggcttgaaggactgtgggcagatgtgaccaagagcctgcattaagttgtacaatggtagatggagtgatgattcttcctgtgcttatcatgattgctctcccctcccctagtatggaagatgagaagcccaaggtcaaccccaaactctacatgtgtgtgtgtgaaggtctctcctgcggtaatgaggaccactgtgaaggccagcagtgcttttcctcactgagcatcaacgatggcttccacgtctaccagaaaggctgcttccaggtttatgagcagggaaagatgacctgtaagaccccgccgtcccctggccaagccgtggagtgctgccaaggggactggtgtaacaggaacatcacggcccagctgcccactaaaggaaaatccttccctggaacacagaatttccacttggaggttggcctcattattctctctgtagtgttcgcagtatgtcttttagcctgcctgctgggagttgctctccgaaaatttaaaaggcgcaaccaagaacgcctcaatccccgagacgtggagtatggcactatcgaagggctcatcaccaccaatgttggagacagcactttagcagatttattggatcattcgtgtacatcaggaagtggctctggtcttccttttctggtacaaagaacagtggctcgccagattacactgttggagtgtgtcgggaaaggcaggtatggtgaggtgtggaggggcagctggcaaggggagaatgttgccgtgaagatcttctcctcccgtgatgagaagtcatggttcagggaaacggaattgtacaacactgtgatgctgaggcatgaaaatatcttaggtttcattgcttcagacatgacatcaagacactccagtacccagctgtggttaattacacattatcatgaaatgggatcgttgtacgactatcttcagcttactactctggatacagttagctgccttcgaatagtgctgtccatagctagtggtcttgcacatttgcacatagagatatttgggacccaagggaaaccagccattgcccatcgagatttaaagagcaaaaatattctggttaagaagaatggacagtgttgcatagcagatttgggcctggcagtcatgcattcccagagcaccaatcagcttgatgtggggaacaatccccgtgtgggcaccaagcgctacatggcccccgaagttctagatgaaaccatccaggtggattgtttcgattcttataaaagggtcgatatttgggcctttggacttgttttgtgggaagtggccaggcggatggtgagcaatggtatagtggaggattacaagccaccgttctacgatgtggttcccaatgacccaagttttgaagatatgaggaaggtagtctgtgtggatcaacaaaggccaaacatacccaacagatggttctcagacccgacattaacctctctggccaagctaatgaaagaatgctggtatcaaaatccatccgcaagactcacagcactgcgtatcaaaaagactttgaccaaaattgataattccctcgacaaattgaaaactgactgttgacattttcatagtgtcaagaaggaagatttgacgttgttgtcattgtccagctgggacctaatgctggcctgactggttgtcagaatggaatccatctgtctccctccccaaatggctgctttgacaaggcagacgtcgtacccagccatgtgttggggagacatcaaaaccaccctaacctcgctcgatgactgtgaactgggcatttcacgaactgttcacactgcagagactaatgttggacagacactgttgcaaaggtagggactggaggaacacagagaaatcctaaaagagatctgggcattaagtcagtggctttgcatagctttcacaagtctcctagacactccccacgggaaactcaaggaggtggtgaatttttaatcagcaatattgcctgtgcttctcttctttattgcactaggaattctttgcattccttacttgcactgttactcttaattttaaagacccaacttgccaaaatgttggctgcgtactccactggtctgtctttggataataggaattcaatttggcaaaacaaaatgtaatgtcagactttgctgcattttacacatgtgctgatgtttacaatgatgccgaacattaggaattgtttatacacaactttgcaaattatttattacttgtgcacttagtagtttttacaaaactgctttgtgcatatgttaaagcttatttttatgtggtcttatgattttattacagaaatgtttttaacactatactctaaaatggacattttcttttattatcagttaaaatcacattttaagtgcttcacatttgtatgtgtgtagactgtaactttttttcagttcatatgcagaacgtatttagccattacccacgtgacaccaccgaatatattactgatttagaagcaaagatttcagtagaattttagtcctgaacgctacggggaaaatgcattttcttcagaattatccattacgtgcatttaaactctgccagaaaaaaataactattttgttttaatctactttttgtatttagtagttatttgtataaattaaataaactgttttcaagtcaaaaaaaaaaaaaaaaaaaa

SEQ ID NO: 19 - DNA, 4564 bp, Artificial Sequence
  Description of Artificial Sequence Antisense nucleic acid
ggggaggcgccgggggcgcgcgcgcgcgcgctgggcgctgctgggctgcggcggcggcggcggcggcggtggttactatggcggagtcggccggagcctcctccttcttcccccttgttgtcctcctgctcgccggcagcggcgggtccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatctaactgctccctctctccacacggagctcctggcagcgagaactacgcacagctgccgcgttgagcgtacgatggaggcctacctctcgtttctgcccagccctctgtggccaggagccctggcccgcaagagggacagagcccgggagagactcgctcactcccatgttgggtttgagacagacaccttttctatttacctcctaatggcatggagactctgagagcgaattgtgtggagaactcagtgccacacctcgaactggttgtagtgggaagtcccgcgaaacccggtgcatctggcacgtggccaggagccatgacaggggcgcttgggaggggccggaggaaccgaggtgttgccagtgctaagctgccctgagggtttccttcggggaccagcccacagcacaccaaggtggcccggaagaaccagaagtgcagcccctctcacaggcagctctgagccgcgctttcccctcctccctgggatggacgctgccgggagactgccagtggagacggaatctgccgctttgtctgtccagccgtgtgtgcatgtgccgaggtgcgtcccccgttgtgcctggttcgtgccatgcccttacacgtgcgtgtgagtgtgtgtgtgtgtctgtaggtgcgcacttacctgcttgagctttctgtgcatgtgcaggtcgggggtgtggtcgtcatgctgtccgtgcttgctggtgcctcttttcagtagtgagcagcatctagtttccctggtgcccttccctggaggtctctccctcccccagagcccctcatgccacagtggtactctgtgtctggcaggctactctgcccaccccagcatcagcacagctctcctcctccatctcagactgtggaaccaaagctggcccagttgtccatgacaaaagaggcttttgggccaaaatgtgagggtggtgggtgggatgggcagggaaggaatcctggtggaagtcttgggtgttagtgtcagccatgggaaatgagccagcccaagggcatcatcctcagcagcatcgaggaagggccgaggaatgtgaagccagatctcgggactcagattggaatgttacatctgtctttcatctcccagatcctggaaacagcagtgtatatttttggtggtggtgggtttggggtggggaagggaagggcgggcaaggagtggggagggagtctggggtgggagggaggcatctgcatgggtcttcttttactggactgtctgatcagggtggagggaaggtgagaggtttgcatccacttcaggagccctactgaagggaacagcctgagccgaacatgttatttaacctgagtatagtatttaacgaagcctagaagcacggctgtgggtggtgatttggtcagcatatcttaggtatataataactttgaagccataacttttaactggagtggtttgatttctttttttaattttattgggagggtttggattttaactttttttaatgttgttaaatattaagtttttgtaaaaggaaaaccatctctgtgattacctctcaatctatttgtttttaaagaaatccctaaaaaaaaaaattatccaattgaacgcacatagctcaatcacactggaaatgtttgtccttgcacctgagcctgttcccactcagcagtgagagttcctctttgccctgaggctcagtctctctcgtattttgtccccacccccaattccttgagtggtttttgctctagggccctttcttgcactgtccagctggttgtaccctctccaggcatttattcaacaaatgtgggtgaagtgcctgctgggtgccaggtgctgggaatacatctgtggacaagacatgcttgggtcctactcctggagcactgtaaaaagagctgattcaagtaagtagatgcctgttttgagaccagaaggtttcataattggttctacgacccttttgagcctagaattattgttcttatataagatcactgaagaaagaggaacccccacaaccccctccacaaagagaccaggggcgggtgatgagacctggggtttagaaccccaggtgagacctcaaatcactgcattcattctgagcccccttcctgtccccaggggaggtgtattgtgtatgtagccttagagcatctctgcctccaacccagcagttctctgccaaagcttgtggaggagggagagccctgtccctgccctcaggctccccagtgctcctggcccttctatttatttgactgattattgcttctttccttgcattaaaggagatcttcccctaacctttgggccaatttactggccactaatttcgtttaaataccattgtgtcattggggggaccgtctttacccctgctgacctcccacctatccgccctgcagcagaaccttggcggtttataggtaatgatggaacttagactcctcttcccagagtcacaagtagcctctgggatctgccaacacacgtccactcccaagccactagcccactccccagttggcccttctgcccttaccccacacacagtccaactcttccacctctggggaagatggagcaggtctttgggaagctcccacacccacctctgccactcttaacactaagtgagagttggggagaaactgaagccgtgtttttggccccccgaggctaaccctgatccatagtgctacctgcacctctggattctggattcacagaccaagtccaagcccgttcttacgtcgccataaaggcccccgaacggcattctcggtacttctgtttgtttttgtacattttattagaaaggactgtaaaatagccacttagacactttacctcttcagtatgcaaatgtaaataaattgtaatataggaaatcttttgttttaatataagaatgagcctgtccaatttctgctgtacattattaaaagttttattcacagag

SEQ ID NO: 20 - DNA, 4516 bp, Artificial Sequence
  Description of Artificial Sequence Antisense nucleic acid
aatgcttctcctaagcacctcgtgtgtgttcttcggcctcactgctctgtggcttaggtatctgtgctgtggggtttgagaaacatggtgaaggtgtatgaacagagcttgacatttgtgctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatctaactgctccctctctccacacggagctcctggcagcgagaactacgcacagctgccgcgttgagcgtacgatggaggcctacctctcgtttctgcccagccctctgtggccaggagccctggcccgcaagagggacagagcccgggagagactcgctcactcccatgttgggtttgagacagacaccttttctatttacctcctaatggcatggagactctgagagcgaattgtgtggagaactcagtgccacacctcgaactggttgtagtgggaagtcccgcgaaacccggtgcatctggcacgtggccaggagccatgacaggggcgcttgggaggggccggaggaaccgaggtgttgccagtgctaagctgccctgagggtttccttcggggaccagcccacagcacaccaaggtggcccggaagaaccagaagtgcagcccctctcacaggcagctctgagccgcgctttcccctcctccctgggatggacgctgccgggagactgccagtggagacggaatctgccgctttgtctgtccagccgtgtgtgcatgtgccgaggtgcgtcccccgttgtgcctggttcgtgccatgcccttacacgtgcgtgtgagtgtgtgtgtgtgtctgtaggtgcgcacttacctgcttgagctttctgtgcatgtgcaggtcgggggtgtggtcgtcatgctgtccgtgcttgctggtgcctcttttcagtagtgagcagcatctagtttccctggtgcccttccctggaggtctctccctcccccagagcccctcatgccacagtggtactctgtgtctggcaggctactctgcccaccccagcatcagcacagctctcctcctccatctcagactgtggaaccaaagctggcccagttgtccatgacaaaagaggcttttgggccaaaatgtgagggtggtgggtgggatgggcagggaaggaatcctggtggaagtcttgggtgttagtgtcagccatgggaaatgagccagcccaagggcatcatcctcagcagcatcgaggaagggccgaggaatgtgaagccagatctcgggactcagattggaatgttacatctgtctttcatctcccagatcctggaaacagcagtgtatatttttggtggtggtgggtttggggtggggaagggaagggcgggcaaggagtggggagggagtctggggtgggagggaggcatctgcatgggtcttcttttactggactgtctgatcagggtggagggaaggtgagaggtttgcatccacttcaggagccctactgaagggaacagcctgagccgaacatgttatttaacctgagtatagtatttaacgaagcctagaagcacggctgtgggtggtgatttggtcagcatatcttaggtatataataactttgaagccataacttttaactggagtggtttgatttctttttttaattttattgggagggtttggattttaactttttttaatgttgttaaatattaagtttttgtaaaaggaaaaccatctctgtgattacctctcaatctatttgtttttaaagaaatccctaaaaaaaaaaattatccaattgaacgcacatagctcaatcacactggaaatgtttgtccttgcacctgagcctgttcccactcagcagtgagagttcctctttgccctgaggctcagtctctctcgtattttgtccccacccccaattccttgagtggtttttgctctagggccctttcttgcactgtccagctggttgtaccctctccaggcatttattcaacaaatgtgggtgaagtgcctgctgggtgccaggtgctgggaatacatctgtggacaagacatgcttgggtcctactcctggagcactgtaaaaagagctgattcaagtaagtagatgcctgttttgagaccagaaggtttcataattggttctacgacccttttgagcctagaattattgttcttatataagatcactgaagaaagaggaacccccacaaccccctccacaaagagaccaggggcgggtgatgagacctggggtttagaaccccaggtgagacctcaaatcactgcattcattctgagcccccttcctgtccccaggggaggtgtattgtgtatgtagccttagagcatctctgcctccaacccagcagttctctgccaaagcttgtggaggagggagagccctgtccctgccctcaggctccccagtgctcctggcccttctatttatttgactgattattgcttctttccttgcattaaaggagatcttcccctaacctttgggccaatttactggccactaatttcgtttaaataccattgtgtcattggggggaccgtctttacccctgctgacctcccacctatccgccctgcagcagaaccttggcggtttataggtaatgatggaacttagactcctcttcccagagtcacaagtagcctctgggatctgccaacacacgtccactcccaagccactagcccactccccagttggcccttctgcccttaccccacacacagtccaactcttccacctctggggaagatggagcaggtctttgggaagctcccacacccacctctgccactcttaacactaagtgagagttggggagaaactgaagccgtgtttttggccccccgaggctaaccctgatccatagtgctacctgcacctctggattctggattcacagaccaagtccaagcccgttcttacgtcgccataaaggcccccgaacggcattctcggtacttctgtttgtttttgtacattttattagaaaggactgtaaaatagccacttagacactttacctcttcagtatgcaaatgtaaataaattgtaatataggaaatcttttgttttaatataagaatgagcctgtccaatttctgctgtacattattaaaagttttattcacagag

SEQ ID NO: 21 - DNA, 4687 bp, Artificial Sequence
  Description of Artificial Sequence Antisense nucleic acid
ggggaggcgccgggggcgcgcgcgcgcgcgctgggcgctgctgggctgcggcggcggcggcggcggcggtggttactatggcggagtcggccggagcctcctccttcttcccccttgttgtcctcctgctcgccggcagcggcgggtccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagcagactgctcattcctcacattgccatgggaagttgtaatggtctctgctgcccccaagctgaggagccttagactccaatacaagggaggaaggggaagagcaagatttttattcccactgaataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatctaactgctccctctctccacacggagctcctggcagcgagaactacgcacagctgccgcgttgagcgtacgatggaggcctacctctcgtttctgcccagccctctgtggccaggagccctggcccgcaagagggacagagcccgggagagactcgctcactcccatgttgggtttgagacagacaccttttctatttacctcctaatggcatggagactctgagagcgaattgtgtggagaactcagtgccacacctcgaactggttgtagtgggaagtcccgcgaaacccggtgcatctggcacgtggccaggagccatgacaggggcgcttgggaggggccggaggaaccgaggtgttgccagtgctaagctgccctgagggtttccttcggggaccagcccacagcacaccaaggtggcccggaagaaccagaagtgcagcccctctcacaggcagctctgagccgcgctttcccctcctccctgggatggacgctgccgggagactgccagtggagacggaatctgccgctttgtctgtccagccgtgtgtgcatgtgccgaggtgcgtcccccgttgtgcctggttcgtgccatgcccttacacgtgcgtgtgagtgtgtgtgtgtgtctgtaggtgcgcacttacctgcttgagctttctgtgcatgtgcaggtcgggggtgtggtcgtcatgctgtccgtgcttgctggtgcctcttttcagtagtgagcagcatctagtttccctggtgcccttccctggaggtctctccctcccccagagcccctcatgccacagtggtactctgtgtctggcaggctactctgcccaccccagcatcagcacagctctcctcctccatctcagactgtggaaccaaagctggcccagttgtccatgacaaaagaggcttttgggccaaaatgtgagggtggtgggtgggatgggcagggaaggaatcctggtggaagtcttgggtgttagtgtcagccatgggaaatgagccagcccaagggcatcatcctcagcagcatcgaggaagggccgaggaatgtgaagccagatctcgggactcagattggaatgttacatctgtctttcatctcccagatcctggaaacagcagtgtatatttttggtggtggtgggtttggggtggggaagggaagggcgggcaaggagtggggagggagtctggggtgggagggaggcatctgcatgggtcttcttttactggactgtctgatcagggtggagggaaggtgagaggtttgcatccacttcaggagccctactgaagggaacagcctgagccgaacatgttatttaacctgagtatagtatttaacgaagcctagaagcacggctgtgggtggtgatttggtcagcatatcttaggtatataataactttgaagccataacttttaactggagtggtttgatttctttttttaattttattgggagggtttggattttaactttttttaatgttgttaaatattaagtttttgtaaaaggaaaaccatctctgtgattacctctcaatctatttgtttttaaagaaatccctaaaaaaaaaaattatccaattgaacgcacatagctcaatcacactggaaatgtttgtccttgcacctgagcctgttcccactcagcagtgagagttcctctttgccctgaggctcagtctctctcgtattttgtccccacccccaattccttgagtggtttttgctctagggccctttcttgcactgtccagctggttgtaccctctccaggcatttattcaacaaatgtgggtgaagtgcctgctgggtgccaggtgctgggaatacatctgtggacaagacatgcttgggtcctactcctggagcactgtaaaaagagctgattcaagtaagtagatgcctgttttgagaccagaaggtttcataattggttctacgacccttttgagcctagaattattgttcttatataagatcactgaagaaagaggaacccccacaaccccctccacaaagagaccaggggcgggtgatgagacctggggtttagaaccccaggtgagacctcaaatcactgcattcattctgagcccccttcctgtccccaggggaggtgtattgtgtatgtagccttagagcatctctgcctccaacccagcagttctctgccaaagcttgtggaggagggagagccctgtccctgccctcaggctccccagtgctcctggcccttctatttatttgactgattattgcttctttccttgcattaaaggagatcttcccctaacctttgggccaatttactggccactaatttcgtttaaataccattgtgtcattggggggaccgtctttacccctgctgacctcccacctatccgccctgcagcagaaccttggcggtttataggtaatgatggaacttagactcctcttcccagagtcacaagtagcctctgggatctgccaacacacgtccactcccaagccactagcccactccccagttggcccttctgcccttaccccacacacagtccaactcttccacctctggggaagatggagcaggtctttgggaagctcccacacccacctctgccactcttaacactaagtgagagttggggagaaactgaagccgtgtttttggccccccgaggctaaccctgatccatagtgctacctgcacctctggattctggattcacagaccaagtccaagcccgttcttacgtcgccataaaggcccccgaacggcattctcggtacttctgtttgtttttgtacattttattagaaaggactgtaaaatagccacttagacactttacctcttcagtatgcaaatgtaaataaattgtaatataggaaatcttttgttttaatataagaatgagcctgtccaatttctgctgtacattattaaaagttttattcacagag

SEQ ID NO: 22 - protein, 10 aa, Artificial Sequence
  CDR
GYTFTSSYIN

SEQ ID NO: 23 - protein, 10 aa, Artificial Sequence
  CDR
GYTFTSSYIN

SEQ ID NO: 24 - protein, 10 aa, Artificial Sequence
  CDR
GYTFTSSYIN

SEQ ID NO: 25 - protein, 10 aa, Artificial Sequence
  CDR
GYTFTSSYIN

SEQ ID NO: 26 - protein, 10 aa, Artificial Sequence
  CDR
GYTFTSSYIN

SEQ ID NO: 27 - protein, 10 aa, Artificial Sequence
  CDR
GYTFTSSYIN

SEQ ID NO: 28 - protein, 10 aa, Artificial Sequence
  CDR
GYTFTSSYIN

SEQ ID NO: 29 - protein, 10 aa, Artificial Sequence
  CDR
GYTFTSSYIN

SEQ ID NO: 30 - protein, 10 aa, Artificial Sequence
  CDR
GYTFTSSYIN

SEQ ID NO: 31 - protein, 10 aa, Artificial Sequence
  CDR
GYTFTSSYIN

SEQ ID NO: 32 - protein, 10 aa, Artificial Sequence
  CDR
GYTFTSSYIN

SEQ ID NO: 33 - protein, 10 aa, Artificial Sequence
  CDR
GYTFTSSYIN

SEQ ID NO: 34 - protein, 10 aa, Artificial Sequence
  CDR
GYTFTSSYIN

SEQ ID NO: 35 - protein, 10 aa, Artificial Sequence
  CDR
GYTFTSSYIN

SEQ ID NO: 36 - protein, 17 aa, Artificial Sequence
  CDR
TINPVSGNTSYAQKFQG

SEQ ID NO: 37 - protein, 17 aa, Artificial Sequence
  CDR
TINPVSGNTSYAQKFQG

SEQ ID NO: 38 - protein, 17 aa, Artificial Sequence
  CDR
TINPVSGNTSYAQKFQG

SEQ ID NO: 39 - protein, 17 aa, Artificial Sequence
  CDR
TINPVSGNTSYAQKFQG

SEQ ID NO: 40 - protein, 17 aa, Artificial Sequence
  CDR
MINAPIGTTRYAQKFQG

SEQ ID NO: 41 - protein, 17 aa, Artificial Sequence
  CDR
QINAASGMTRYAQKFQG

SEQ ID NO: 42 - protein, 17 aa, Artificial Sequence
  CDR
MINAPIGTTRYAQKFQG

SEQ ID NO: 43 - protein, 17 aa, Artificial Sequence
  CDR
TINPVSGNTRYAQKFQG

SEQ ID NO: 44 - protein, 17 aa, Artificial Sequence
  CDR
TINPVSGSTSYAQKFQG

SEQ ID NO: 45 - protein, 17 aa, Artificial Sequence
  CDR
QINAASGMTRYAQKFQG

SEQ ID NO: 46 - protein, 17 aa, Artificial Sequence
  CDR
NINAAAGITLYAQKFQG

SEQ ID NO: 47 - protein, 17 aa, Artificial Sequence
  CDR
TINPPTGGTYYAQKFQG

SEQ ID NO: 48 - protein, 17 aa, Artificial Sequence
  CDR
GINPPAGTTSYAQKFQG

SEQ ID NO: 49 - protein, 17 aa, Artificial Sequence
  CDR
NINPATGHADYAQKFQG

SEQ ID NO: 50 - protein, 6 aa, Artificial Sequence
  CDR
GGWFDY

SEQ ID NO: 51 - protein, 6 aa, Artificial Sequence
  CDR
GGWFDY

SEQ ID NO: 52 - protein, 6 aa, Artificial Sequence
  CDR
GGWFDY

SEQ ID NO: 53 - protein, 6 aa, Artificial Sequence
  CDR
GGWFDY

SEQ ID NO: 54 - protein, 6 aa, Artificial Sequence
  CDR
GGWFDY

SEQ ID NO: 55 - protein, 6 aa, Artificial Sequence
  CDR
GGWFDY

SEQ ID NO: 56 - protein, 6 aa, Artificial Sequence
  CDR
GGWFDY

SEQ ID NO: 57 - protein, 6 aa, Artificial Sequence
  CDR
GGWFDY

SEQ ID NO: 58 - protein, 6 aa, Artificial Sequence
  CDR
GGWFDY

SEQ ID NO: 59 - protein, 6 aa, Artificial Sequence
  CDR
GGWFDY

SEQ ID NO: 60 - protein, 6 aa, Artificial Sequence
  CDR
GGWFDY

SEQ ID NO: 61 - protein, 6 aa, Artificial Sequence
  CDR
GGWFDY

SEQ ID NO: 62 - protein, 6 aa, Artificial Sequence
  CDR
GGWFDY

SEQ ID NO: 63 - protein, 6 aa, Artificial Sequence
  CDR
GGWFDY

SEQ ID NO: 64 - protein, 14 aa, Artificial Sequence
  CDR
TGTSSDVGSYNYVN

SEQ ID NO: 65 - protein, 14 aa, Artificial Sequence
  CDR
TGTSSDVGSYNYVN

SEQ ID NO: 66 - protein, 14 aa, Artificial Sequence
  CDR
TGTSSDVGSYNYVN

SEQ ID NO: 67 - protein, 14 aa, Artificial Sequence
  CDR
TGTSSDVGSYNYVN

SEQ ID NO: 68 - protein, 14 aa, Artificial Sequence
  CDR
TGTSSDVGSYNYVN

SEQ ID NO: 69 - protein, 14 aa, Artificial Sequence
  CDR
TGTSSDVGSYNYVN

SEQ ID NO: 70 - protein, 14 aa, Artificial Sequence
  CDR
TGTSSDVGSYNYVN

SEQ ID NO: 71 - protein, 14 aa, Artificial Sequence
  CDR
TGTSSDVGSYNYVN

SEQ ID NO: 72 - protein, 14 aa, Artificial Sequence
  CDR
TGTSSDVGSYNYVN

SEQ ID NO: 73 - protein, 14 aa, Artificial Sequence
  CDR
TGTSSDVGSYNYVN

SEQ ID NO: 74 - protein, 14 aa, Artificial Sequence
  CDR
TGTSSDVGSYNYVN

SEQ ID NO: 75 - protein, 14 aa, Artificial Sequence
  CDR
TGTSSDVGSYNYVN

SEQ ID NO: 76 - protein, 14 aa, Artificial Sequence
  CDR
TGTSSDVGSYNYVN

SEQ ID NO: 77 - protein, 14 aa, Artificial Sequence
  CDR
TGTSSDVGSYNYVN

SEQ ID NO: 78 - protein, 11 aa, Artificial Sequence
  CDR
LMIYGVSKRPS

SEQ ID NO: 79 - protein, 11 aa, Artificial Sequence
  CDR
LMIYGVSKRPS

SEQ ID NO: 80 - protein, 11 aa, Artificial Sequence
  CDR
LMIYGVSKRPS

SEQ ID NO: 81 - protein, 11 aa, Artificial Sequence
  CDR
LMIYGVSKRPS

SEQ ID NO: 82 - protein, 11 aa, Artificial Sequence
  CDR
LMIYGVSKRPS

SEQ ID NO: 83 - protein, 11 aa, Artificial Sequence
  CDR
LMIYGVSKRPS

SEQ ID NO: 84 - protein, 11 aa, Artificial Sequence
  CDR
LMIYGVSKRPS

SEQ ID NO: 85 - protein, 11 aa, Artificial Sequence
  CDR
LMIYGVSKRPS

SEQ ID NO: 86 - protein, 11 aa, Artificial Sequence
  CDR
LMIYGVSKRPS

SEQ ID NO: 87 - protein, 11 aa, Artificial Sequence
  CDR
LMIYGVSKRPS

SEQ ID NO: 88 - protein, 11 aa, Artificial Sequence
  CDR
LMIYGVSKRPS

SEQ ID NO: 89 - protein, 11 aa, Artificial Sequence
  CDR
LMIYGVSKRPS

SEQ ID NO: 90 - protein, 11 aa, Artificial Sequence
  CDR
LMIYGVSKRPS

SEQ ID NO: 91 - protein, 11 aa, Artificial Sequence
  CDR
LMIYGVSKRPS

SEQ ID NO: 92 - protein, 9 aa, Artificial Sequence
  CDR
QAWTSKMAG

SEQ ID NO: 93 - protein, 9 aa, Artificial Sequence
  CDR
SSYTRMGHP

SEQ ID NO: 94 - protein, 10 aa, Artificial Sequence
  CDR
ATYGKGVTPP

SEQ ID NO: 95 - protein, 10 aa, Artificial Sequence
  CDR
GTFAGGSYYG

SEQ ID NO: 96 - protein, 9 aa, Artificial Sequence
  CDR
QAWTSKMAG

SEQ ID NO: 97 - protein, 9 aa, Artificial Sequence
  CDR
QAWTSKMAG

SEQ ID NO: 98 - protein, 10 aa, Artificial Sequence
  CDR
GTFAGGSYYG

SEQ ID NO: 99 - protein, 10 aa, Artificial Sequence
  CDR
GTFAGGSYYG

SEQ ID NO: 100 - protein, 10 aa, Artificial Sequence
  CDR
GTFAGGSYYG

SEQ ID NO: 101 - protein, 10 aa, Artificial Sequence
  CDR
GTFAGGSYYG

SEQ ID NO: 102 - protein, 10 aa, Artificial Sequence
  CDR
GTFAGGSYYG

SEQ ID NO: 103 - protein, 10 aa, Artificial Sequence
  CDR
GTFAGGSYYG

SEQ ID NO: 104 - protein, 10 aa, Artificial Sequence
  CDR
GTFAGGSYYG

SEQ ID NO: 105 - protein, 10 aa, Artificial Sequence
  CDR
GTFAGGSYYG

SEQ ID NO: 106 - protein, 112 aa, Artificial Sequence
  VL
DIALTQPASVSGSPGQSITISCTGTSSDVGSYNYVNWYQQHPGKAPKLMIYGVSKRPSGVSNRFSGSKSGNTASLTISGLQAEDEADYYCQAWTSKMAGVFGGGTKLTVLGQ

SEQ ID NO: 107 - protein, 112 aa, Artificial Sequence
  VL
DIALTQPASVSGSPGQSITISCTGTSSDVGSYNYVNWYQQHPGKAPKLMIYGVSKRPSGVSNRFSGSKSGNTASLTISGLQAEDEADYYCSSYTRMGHPVFGGGTKLTVLGQ

SEQ ID NO: 108 - protein, 113 aa, Artificial Sequence
  VL
DIALTQPASVSGSPGQSITISCTGTSSDVGSYNYVNWYQQHPGKAPKLMIYGVSKRPSGVSNRFSGSKSGNTASLTISGLQAEDEADYYCATYGKGVTPPVFGGGTKLTVLGQ

SEQ ID NO: 109 - protein, 113 aa, Artificial Sequence
  VL
DIALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQ

SEQ ID NO: 110 - protein, 112 aa, Artificial Sequence; identical to SEQ ID NO: 106
  VL

SEQ ID NO: 111 - protein, 112 aa, Artificial Sequence; identical to SEQ ID NO: 106
  VL

SEQ ID NO: 112 - protein, 113 aa, Artificial Sequence; identical to SEQ ID NO: 109
  VL

SEQ ID NO: 113 - protein, 113 aa, Artificial Sequence; identical to SEQ ID NO: 109
  VL

SEQ ID NO: 114 - protein, 113 aa, Artificial Sequence; identical to SEQ ID NO: 109
  VL

SEQ ID NO: 115 - protein, 113 aa, Artificial Sequence; identical to SEQ ID NO: 109
  VL

SEQ ID NO: 116 - protein, 113 aa, Artificial Sequence; identical to SEQ ID NO: 109
  VL

SEQ ID NO: 117 - protein, 113 aa, Artificial Sequence; identical to SEQ ID NO: 109
  VL

SEQ ID NO: 118 - protein, 113 aa, Artificial Sequence; identical to SEQ ID NO: 109
  VL

SEQ ID NO: 119 - protein, 113 aa, Artificial Sequence; identical to SEQ ID NO: 109
  VL

SEQ ID NO: 120 - protein, 115 aa, Artificial Sequence
  VH
QVQLVQSGAEVKKPGASVKVSCKASGYTFTSSYINWVRQAPGQGLEWMGTINPVSGNTSYAQKFQGRVTMTRDTSISTAYMELSSLRSEDTAVYYCARGGWFDYWGQGTLVTVSS

SEQ ID NO: 121 - protein, 115 aa, Artificial Sequence; identical to SEQ ID NO: 120
  VH

SEQ ID NO: 122 - protein, 115 aa, Artificial Sequence; identical to SEQ ID NO: 120
  VH

SEQ ID NO: 123 - protein, 115 aa, Artificial Sequence; identical to SEQ ID NO: 120
  VH

SEQ ID NO: 124 - protein, 115 aa, Artificial Sequence
  VH
QVQLVQSGAEVKKPGASVKVSCKASGYTFTSSYINWVRQAPGQGLEWMGMINAPIGTTRYAQKFQGRVTMTRDTSISTAYMELSSLRSEDTAVYYCARGGWFDYWGQGTLVTVSS

SEQ ID NO: 125 - protein, 115 aa, Artificial Sequence
  VH
QVQLVQSGAEVKKPGASVKVSCKASGYTFTSSYINWVRQAPGQGLEWMGQINAASGMTRYAQKFQGRVTMTRDTSISTAYMELSSLRSEDTAVYYCARGGWFDYWGQGTLVTVSS

SEQ ID NO: 126 - protein, 115 aa, Artificial Sequence; identical to SEQ ID NO: 124
  VH

SEQ ID NO: 127 - protein, 115 aa, Artificial Sequence
  VH
QVQLVQSGAEVKKPGASVKVSCKASGYTFTSSYINWVRQAPGQGLEWMGTINPVSGNTRYAQKFQGRVTMTRDTSISTAYMELSSLRSEDTAVYYCARGGWFDYWGQGTLVTVSS

SEQ ID NO: 128 - protein, 115 aa, Artificial Sequence
  VH
QVQLVQSGAEVKKPGASVKVSCKASGYTFTSSYINWVRQAPGQGLEWMGTINPVSGSTSYAQKFQGRVTMTRDTSISTAYMELSSLRSEDTAVYYCARGGWFDYWGQGTLVTVSS

SEQ ID NO: 129 - protein, 115 aa, Artificial Sequence; identical to SEQ ID NO: 125
  VH

SEQ ID NO: 130 - protein, 115 aa, Artificial Sequence
  VH
QVQLVQSGAEVKKPGASVKVSCKASGYTFTSSYINWVRQAPGQGLEWMGNINAAAGITLYAQKFQGRVTMTRDTSISTAYMELSSLRSEDTAVYYCARGGWFDYWGQGTLVTVSS

SEQ ID NO: 131 - protein, 115 aa, Artificial Sequence
  VH
QVQLVQSGAEVKKPGASVKVSCKASGYTFTSSYINWVRQAPGQGLEWMGTINPPTGGTYYAQKFQGRVTMTRDTSISTAYMELSSLRSEDTAVYYCARGGWFDYWGQGTLVTVSS

SEQ ID NO: 132 - protein, 115 aa, Artificial Sequence
  VH
QVQLVQSGAEVKKPGASVKVSCKASGYTFTSSYINWVRQAPGQGLEWMGGINPPAGTTSYAQKFQGRVTMTRDTSISTAYMELSSLRSEDTAVYYCARGGWFDYWGQGTLVTVSS

SEQ ID NO: 133 - protein, 115 aa, Artificial Sequence
  VH
QVQLVQSGAEVKKPGASVKVSCKASGYTFTSSYINWVRQAPGQGLEWMGNINPATGHADYAQKFQGRVTMTRDTSISTAYMELSSLRSEDTAVYYCARGGWFDYWGQGTLVTVSS

SEQ ID NO: 134 - DNA, 336 bp, Artificial Sequence
  VL
gatatcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgccaggcttggacttctaagatggctggtgtgtttggcggcggcacgaagttaaccgttcttggccag

SEQ ID NO: 135 - DNA, 336 bp, Artificial Sequence
  VL
gatatcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgctcttcttatactcgtatgggtcatcctgtgtttggcggcggcacgaagttaaccgttcttggccag

SEQ ID NO: 136 - DNA, 339 bp, Artificial Sequence
  VL
gatatcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgcgctacttatggtaagggtgttactcctcctgtgtttggcggcggcacgaagttaaccgttcttggccag

SEQ ID NO: 137 - DNA, 339 bp, Artificial Sequence
  VL
gatatcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgcggtacttttgctggtggttcttattatggtgtgtttggcggcggcacgaagttaaccgttcttggccag

SEQ ID NO: 138 - DNA, 336 bp, Artificial Sequence; identical to SEQ ID NO: 134
  VL

SEQ ID NO: 139 - DNA, 336 bp, Artificial Sequence; identical to SEQ ID NO: 134
  VL

SEQ ID NO: 140 - DNA, 339 bp, Artificial Sequence; identical to SEQ ID NO: 137
  VL

SEQ ID NO: 141 - DNA, 339 bp, Artificial Sequence; identical to SEQ ID NO: 137
  VL

SEQ ID NO: 142 - DNA, 339 bp, Artificial Sequence; identical to SEQ ID NO: 137
  VL

SEQ ID NO: 143 - DNA, 339 bp, Artificial Sequence; identical to SEQ ID NO: 137
  VL

SEQ ID NO: 144 - DNA, 339 bp, Artificial Sequence; identical to SEQ ID NO: 137
  VL

SEQ ID NO: 145 - DNA, 339 bp, Artificial Sequence
  VL
gatatcgcactgacccagccagcttcagtgagcggctcaccaggtcagagcattaccatctcgtgtactggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgcggtacttttgctggtggttcttattatggtgtgtttggcggcggcacgaagttaaccgttcttggccag

SEQ ID NO: 146 - DNA, 339 bp, Artificial Sequence; identical to SEQ ID NO: 137
  VL

SEQ ID NO: 147 - DNA, 339 bp, Artificial Sequence; identical to SEQ ID NO: 137
  VL

SEQ ID NO: 148 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcactatcaatccggtttctggcaatacgtcttacgcgcagaagtttcagggccgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 149 - DNA, 345 bp, Artificial Sequence; identical to SEQ ID NO: 148
  VH

SEQ ID NO: 150 - DNA, 345 bp, Artificial Sequence; identical to SEQ ID NO: 148
  VH

SEQ ID NO: 151 - DNA, 345 bp, Artificial Sequence; identical to SEQ ID NO: 148
  VH

SEQ ID NO: 152 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcatgattaatgctcctattggtactactcgttatgctcagaagtttcagggtcgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 153 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggccagattaatgctgcttctggtatgactcgttatgctcagaagtttcagggtcgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 154 - DNA, 345 bp, Artificial Sequence; identical to SEQ ID NO: 152
  VH

SEQ ID NO: 155 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcactatcaatccggtttctggcaatacgcgttacgcgcagaagtttcagggccgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 156 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcactatcaatccggtttctggctctacgtcttacgcgcagaagtttcagggccgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 157 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggccagattaatgctgcttctggtatgactcgttatgctcagaagtttcagggtcgggtcaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 158 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatgctgctgctggtattactctttatgctcagaagtttcagggtcgggtcaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 159 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcactattaatcctcctactggaggtacttattatgctcagaagtttcagggtcgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 160 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcggtattaatcctcctgctggtactacttcttatgctcagaagtttcagggtcgggtcaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 161 - DNA, 345 bp, Artificial Sequence
  VH
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatcctgctactggtcatgctgattatgctcagaagtttcagggtcgggtgaccatgacccgtgataccagcattagcaccgcgtatatggaactgagcagcctgcgtagcgaagatacggccgtgtattattgcgcgcgtggtggttggtttgattattggggccaaggcaccctggtgacggttagctca

SEQ ID NO: 162 - protein, 217 aa, Artificial Sequence
  light chain
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 163 - protein, 217 aa, Artificial Sequence; identical to SEQ ID NO: 162
  light chain

SEQ ID NO: 164 - protein, 217 aa, Artificial Sequence; identical to SEQ ID NO: 162
  light chain

SEQ ID NO: 165 - protein, 217 aa, Artificial Sequence; identical to SEQ ID NO: 162
  light chain

SEQ ID NO: 166 - protein, 217 aa, Artificial Sequence; identical to SEQ ID NO: 162
  light chain

SEQ ID NO: 167 - protein, 445 aa, Artificial Sequence
  heavy chain
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 445 aa, Artificial Sequence
  heavy chain
QVQLVQSGAEVKKPGASVKVSCKASGYTFTSSYINWVRQAPGQGLEWMGQINAASGMTRYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 445 aa, Artificial Sequence
  heavy chain
QVQLVQSGAEVKKPGASVKVSCKASGYTFTSSYINWVRQAPGQGLEWMGNINAAAGITLYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 445 aa, Artificial Sequence
  heavy chain
QVQLVQSGAEVKKPGASVKVSCKASGYTFTSSYINWVRQAPGQGLEWMGGINPPAGTTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 445 aa, Artificial Sequence
  heavy chain
QVQLVQSGAEVKKPGASVKVSCKASGYTFTSSYINWVRQAPGQGLEWMGNINPATGHADYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 217 aa, Artificial Sequence; identical to SEQ ID NO: 162
  light chain

SEQ ID NO: 173 - protein, 217 aa, Artificial Sequence; identical to SEQ ID NO: 162
  light chain

SEQ ID NO: 174 - protein, 217 aa, Artificial Sequence; identical to SEQ ID NO: 162
  light chain

SEQ ID NO: 175 - protein, 217 aa, Artificial Sequence; identical to SEQ ID NO: 162
  light chain

SEQ ID NO: 176 - protein, 217 aa, Artificial Sequence; identical to SEQ ID NO: 162
  light chain

SEQ ID NO: 177 - protein, 441 aa, Artificial Sequence
  heavy chain
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 178 - protein, 441 aa, Artificial Sequence
  heavy chain
QVQLVQSGAEVKKPGASVKVSCKASGYTFTSSYINWVRQAPGQGLEWMGQINAASGMTRYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 179 - protein, 441 aa, Artificial Sequence
  heavy chain
QVQLVQSGAEVKKPGASVKVSCKASGYTFTSSYINWVRQAPGQGLEWMGNINAAAGITLYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 180 - protein, 441 aa, Artificial Sequence
  heavy chain
QVQLVQSGAEVKKPGASVKVSCKASGYTFTSSYINWVRQAPGQGLEWMGGINPPAGTTSYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 181 - protein, 441 aa, Artificial Sequence
  heavy chain
QVQLVQSGAEVKKPGASVKVSCKASGYTFTSSYINWVRQAPGQGLEWMGNINPATGHADYAQKFQGRVTMTRDTSISTAYMELSRLRSDDTAVYYCARGGWFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 182 - DNA, 651 bp, Artificial Sequence
  light chain
cagagcgccctgacccagcccgccagcgtgtccggcagcccaggccagtctatcacaatcagctgcaccggcacctccagcgacgtgggcagctacaactacgtgaactggtatcagcagcaccccggcaaggcccccaagctgatgatctacggcgtgagcaagaggcccagcggcgtgtccaacaggttcagcggcagcaagagcggcaacaccgccagcctgacaatcagtgggctgcaggctgaggacgaggccgactactactgcggcacctttgccggcggatcatactacggcgtgttcggcggagggaccaagctgaccgtgctgggccagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 183 - DNA, 651 bp, Artificial Sequence; identical to SEQ ID NO: 182
  light chain

SEQ ID NO: 184 - DNA, 651 bp, Artificial Sequence
  light chain
cagagcgcactgacccagccagcttcagtgagcggctcaccaggtcagagcattaccatctcgtgtacgggtactagcagcgatgttggttcttataattatgtgaattggtaccagcagcatcccgggaaggcgccgaaacttatgatttatggtgtttctaagcgtccctcaggcgtgagcaaccgttttagcggatccaaaagcggcaacaccgcgagcctgaccattagcggcctgcaagcggaagacgaagcggattattattgcggtacttttgctggtggttcttattatggtgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 185 - DNA, 651 bp, Artificial Sequence; identical to SEQ ID NO: 184
  light chain

SEQ ID NO: 186 - DNA, 651 bp, Artificial Sequence; identical to SEQ ID NO: 184
  light chain

SEQ ID NO: 187 - DNA, 1335 bp, Artificial Sequence
  heavy chain
caggtgcagctggtgcagagcggagctgaggtgaagaagccaggcgccagcgtcaaggtgtcctgcaaggccagcggctacaccttcaccagcagctacatcaactgggtccgccaggctcctgggcagggactggagtggatgggcaccatcaaccccgtgtccggcagcaccagctacgcccagaagttccagggcagagtcaccatgaccagggacaccagcatcagcaccgcctacatggagctgtccaggctgagaagcgacgacaccgccgtgtactactgcgccaggggcggctggttcgactactggggccagggcaccctggtgaccgtgtcctcagctagcaccaagggccccagcgtgttccccctggcccccagcagcaagagcacctccggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagcccccgaagctgcaggcggcccttccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaggtctccaacaaggccctgcctgcccccatcgaaaagaccatcagcaaggccaagggccagccacgggagccccaggtgtacaccctgcccccttctcgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgtcacccggcaag

SEQ ID NO: 188 - DNA, 1335 bp, Artificial Sequence
  heavy chain
caggtgcagctggtgcagagcggagctgaggtgaagaagccaggcgccagcgtcaaggtgtcctgcaaggccagcggctacaccttcaccagcagctacatcaactgggtgcgccaggctccagggcagggactggagtggatgggccagatcaacgccgccagcggcatgaccagatacgcccagaagttccagggcagagtcacaatgaccagggacacctctatcagcaccgcctacatggagctgtccaggctgagaagcgacgacaccgccgtgtactactgcgccaggggcggctggttcgactactggggccagggcaccctggtgaccgtgtcctcagctagcaccaagggccccagcgtgttccccctggcccccagcagcaagagcacctccggcggcacagccgccctgggctgcctggtgaaggactacttccccgagcccgtgaccgtgtcctggaacagcggagccctgaccagcggcgtgcacaccttccccgccgtgctgcagagcagcggcctgtacagcctgtccagcgtggtgacagtgcccagcagcagcctgggcacccagacctacatctgcaacgtgaaccacaagcccagcaacaccaaggtggacaagagagtggagcccaagagctgcgacaagacccacacctgccccccctgcccagcccccgaagctgcaggcggcccttccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaggtctccaacaaggccctgcctgcccccatcgaaaagaccatcagcaaggccaagggccagccacgggagccccaggtgtacaccctgcccccttctcgggaggagatgaccaagaaccaggtgtccctgacctgtctggtgaagggcttctaccccagcgacatcgccgtggagtgggagagcaacggccagcccgagaacaactacaagaccacccccccagtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgtcacccggcaag

SEQ ID NO: 189 - DNA, 1335 bp, Artificial Sequence
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatgctgctgctggtattactctttatgctcagaagtttcagggtcgggtcaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 190 - DNA, 1335 bp, Artificial Sequence
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcggtattaatcctcctgctggtactacttcttatgctcagaagtttcagggtcgggtcaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 191 - DNA, 1335 bp, Artificial Sequence
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatcctgctactggtcatgctgattatgctcagaagtttcagggtcgggtgaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 192 - DNA, 651 bp, Artificial Sequence; identical to SEQ ID NO: 182
  light chain

SEQ ID NO: 193 - DNA, 651 bp, Artificial Sequence; identical to SEQ ID NO: 182
  light chain

SEQ ID NO: 194 - DNA, 651 bp, Artificial Sequence; identical to SEQ ID NO: 184
  light chain

SEQ ID NO: 195 - DNA, 651 bp, Artificial Sequence; identical to SEQ ID NO: 184
  light chain

SEQ ID NO: 196 - DNA, 651 bp, Artificial Sequence; identical to SEQ ID NO: 184
  light chain

SEQ ID NO: 197 - DNA, 1323 bp, Artificial Sequence
  heavy chain
caggtgcagctggtgcagagcggagctgaggtgaagaagccaggcgccagcgtcaaggtgtcctgcaaggccagcggctacaccttcaccagcagctacatcaactgggtccgccaggctcctgggcagggactggagtggatgggcaccatcaaccccgtgtccggcagcaccagctacgcccagaagttccagggcagagtcaccatgaccagggacaccagcatcagcaccgcctacatggagctgtccaggctgagaagcgacgacaccgccgtgtactactgcgccaggggcggctggttcgactactggggccagggcaccctggtgaccgtgtcctcagctagcaccaagggccccagcgtgttccccctggccccctgcagcagaagcaccagcgagagcacagccgccctgggctgcctggtgaaggactacttccccgagccagtgaccgtgtcctggaacagcggagccctgaccagcggcgtgcacaccttccccgccgtgctgcagagcagcggcctgtacagcctgtccagcgtggtgaccgtgcccagcagcaacttcggcacccagacctacacctgcaacgtggaccacaagcccagcaacaccaaggtggacaagaccgtggagaggaagtgctgcgtggagtgccccccctgcccagcccccccagtggccggaccctccgtgttcctgttcccccccaagcccaaggacaccctgatgatcagcaggacccccgaggtgacctgcgtggtggtggacgtgagccacgaggacccagaggtgcagttcaactggtacgtggacggcgtggaggtgcacaacgccaagaccaagcccagagaggaacagtttaacagcaccttcagggtggtgtccgtgctgaccgtggtgcaccaggactggctgaacggcaaagagtacaagtgcaaggtctccaacaagggcctgccagcccccatcgagaaaaccatcagcaagaccaagggccagccacgggagccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagcaggtggcagcagggcaacgtgttcagctgcagcgtgatgcacgaggccctgcacaaccactacacccagaagagcctgagcctgtcccccggcaag

SEQ ID NO: 198 - DNA, 1323 bp, Artificial Sequence
  heavy chain
caggtgcagctggtgcagagcggagctgaggtgaagaagccaggcgccagcgtcaaggtgtcctgcaaggccagcggctacaccttcaccagcagctacatcaactgggtgcgccaggctccagggcagggactggagtggatgggccagatcaacgccgccagcggcatgaccagatacgcccagaagttccagggcagagtcacaatgaccagggacacctctatcagcaccgcctacatggagctgtccaggctgagaagcgacgacaccgccgtgtactactgcgccaggggcggctggttcgactactggggccagggcaccctggtgaccgtgtcctcagctagcaccaagggccccagcgtgttccccctggccccctgcagcagaagcaccagcgagagcacagccgccctgggctgcctggtgaaggactacttccccgagccagtgaccgtgtcctggaacagcggagccctgaccagcggcgtgcacaccttccccgccgtgctgcagagcagcggcctgtacagcctgtccagcgtggtgaccgtgcccagcagcaacttcggcacccagacctacacctgcaacgtggaccacaagcccagcaacaccaaggtggacaagaccgtggagaggaagtgctgcgtggagtgccccccctgcccagcccccccagtggccggaccctccgtgttcctgttcccccccaagcccaaggacaccctgatgatcagcaggacccccgaggtgacctgcgtggtggtggacgtgagccacgaggacccagaggtgcagttcaactggtacgtggacggcgtggaggtgcacaacgccaagaccaagcccagagaggaacagtttaacagcaccttcagggtggtgtccgtgctgaccgtggtgcaccaggactggctgaacggcaaagagtacaagtgcaaggtctccaacaagggcctgccagcccccatcgagaaaaccatcagcaagaccaagggccagccacgggagccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagcaggtggcagcagggcaacgtgttcagctgcagcgtgatgcacgaggccctgcacaaccactacacccagaagagcctgagcctgtcccccggcaag

SEQ ID NO: 199 - DNA, 1323 bp, Artificial Sequence
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatgctgctgctggtattactctttatgctcagaagtttcagggtcgggtcaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcttccaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagttcaactggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 200 - DNA, 1323 bp, Artificial Sequence
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcggtattaatcctcctgctggtactacttcttatgctcagaagtttcagggtcgggtcaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcttccaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagttcaactggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 201 - DNA, 1323 bp, Artificial Sequence
  heavy chain
caggtgcaattggttcagagcggcgcggaagtgaaaaaaccgggcgcgagcgtgaaagtgagctgcaaagcctccggatatacctttacttcttcttatattaattgggtccgccaagcccctgggcagggtctcgagtggatgggcaatattaatcctgctactggtcatgctgattatgctcagaagtttcagggtcgggtgaccatgacccgtgataccagcattagcaccgcgtatatggaactgagccgcctgcgtagcgatgatacggccgtgtattattgcgcgcgtggtggttggtttgattattggggccaaggcaccctggtgacggttagctcagcttccaccaagggccccagcgtgttccccctggccccctgcagcagaagcaccagcgagagcacagccgccctgggctgcctggtgaaggactacttccccgagcccgtgaccgtgagctggaacagcggagccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagcggaagtgctgcgtggagtgccccccctgccctgcccctcctgtggccggaccctccgtgttcctgttcccccccaagcccaaggacaccctgatgatcagccggacccccgaggtgacctgcgtggtggtggacgtgagccacgaggaccccgaggtgcagttcaactggtacgtggacggcgtggaggtgcacaacgccaagaccaagccccgggaggaacagttcaacagcaccttccgggtggtgtccgtgctgaccgtggtgcaccaggactggctgaacggcaaagaatacaagtgcaaggtgtccaacaagggcctgcctgcccccatcgagaaaaccatcagcaagacaaagggccagcccagggaaccccaggtgtacaccctgccccccagccgggaggaaatgaccaagaaccaggtgtccctgacctgtctggtgaagggcttctaccccagcgacatcgccgtggagtgggagagcaacggccagcccgagaacaactacaagaccaccccccccatgctggacagcgacggcagcttcttcctgtacagcaagctgacagtggacaagagccggtggcagcagggcaacgtgttcagctgcagcgtgatgcacgaggccctgcacaaccactacacccagaagagcctgagcctgtcccccggcaaa

SEQ ID NO: 202 - DNA, 20 bp, Artificial Sequence
  Description of Artificial Sequence ACTB forward primer
accttctacaatgagctgcg

SEQ ID NO: 203 - DNA, 21 bp, Artificial Sequence
  Description of Artificial Sequence ACTB reverse primer
cctggatagcaacgtacatgg

SEQ ID NO: 204 - DNA, 18 bp, Artificial Sequence
  Description of Artificial Sequence LIF forward primer
atacgccacccatgtcac

SEQ ID NO: 205 - DNA, 21 bp, Artificial Sequence
  Description of Artificial Sequence LIF reverse primer
ccacatagcttgtccaggttg

SEQ ID NO: 206 - DNA, 21 bp, Artificial Sequence
  Description of Artificial Sequence PRDM1 forward primer
tgtggtattgtcgggactttg

SEQ ID NO: 207 - DNA, 23 bp, Artificial Sequence
  Description of Artificial Sequence PRDM1 reverse primer
ctttgggacattctttgggcctg

(19) United States
(12) Patent Application Publication
(10) Pub. No.: US 2018/0111991 A1
(43) Pub. Date: Apr. 26, 2018

(54) MODULATORS OF ACTIVIN AND METHODS FOR MODULATING IMMUNE RESPONSES AND T FOLLICULAR HELPER CELLS

(71) Applicants: LA JOLLA INSTITUTE FOR ALLERGY AND IMMUNOLOGY, La Jolla, CA (US); NOVARTIS AG, Basel (CH)

(72) Inventors: Andrew Todd MILLER, San Diego, CA (US); Shane CROTTY, San Diego, CA (US); Michela LOCCI, San Diego, CA (US)

(21) Appl. No.: 15/612,950

(22) Filed: Jun. 2, 2017

Related U.S. Application Data
(63) Continuation of application No. PCT/US2015/063827, filed on Dec. 3, 2015.
Application No. PCT/US2015/063827 is a continuation-in-part of application No. PCT/US2015/063500, filed on Dec. 2, 2015.
(60) Provisional application No. 62/086,700, filed on Dec. 2, 2014.

(51) Int. Cl. C07K 16/28; A61P 37/06

(52) U.S. Cl. CPC C07K 16/28; C07K 2317/76; A61P 37/06

(57) ABSTRACT

The invention provides methods and uses for stimulating, inducing, increasing or enhancing Tfh (follicular helper) cell differentiation, germinal center B cell development or an antibody response, and an immune response by administering activin or a subsequence thereof, or an activator of activin receptor, in an amount effective to stimulate, induce, increase or enhance Tfh (follicular helper) cell differentiation, germinal center B cell development or an antibody response, and an immune response, respectively. Also provided are peptides comprising or consisting of a subsequence of human activin sequence, in which the activin sequence or subsequence stimulates, induces, increases or enhances development of Tfh (follicular helper) cell differentiation, germinal center B cell development, an antibody response, an immune response and/or an immune response induced by a vaccine.

### RELATED APPLICATIONS

This patent application is a continuation application of International Application No. PCT/US2015/063827, filed Dec. 3, 2015 and claims the benefit of priority to International Application No. PCT/US2015/063500, filed Dec. 2, 2015 and U.S. patent application No. 62/086,700, filed Dec. 2, 2014, all of which applications are expressly incorporated herein by reference in their entirety.

This invention was made with government support under contract/grant number UM1-A1100663 awarded by NIH. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 2, 2017, is named LIAI0444667_ST25.txt and is 19.4 MB in size.

### FIELD OF THE INVENTION

### Background

Follicular helper T cells (Tfh) are the specialized providers of T cell help to B cells, and are essential for germinal centers, affinity maturation, and the development of most high affinity antibodies and memory B cells. Because of their important role regulating B cells and antibody responses, Tfh appear to be critical components of many protective immune responses against pathogens, as well as being positively associated with protective responses against multiple cancers. As such, there is strong interest in harnessing Tfh cell biology to enhance new vaccines. Tfh cell responses also are major components of a number of autoimmune diseases associated with autoantibody responses.

### BRIEF SUMMARY OF THE INVENTION

The invention is based at least in part on our finding that modulation of activin (activin A, activin AB or activin B), which is able to modulate various immune responses (e.g., stimulate induce, increase or enhance, or inhibit, decrease or reduce an immune response). For example activin can stimulate, induce, increase or enhance an immune response. Activin can also stimulate, induce, increase, or enhance Tfh (follicular helper) cell differentiation, germinal center B cell development or an antibody response. Therefore, provided are activators of activin A for use in methods to enhance Tfh and thereby improve vaccines against pathogens, or improve cancer immunotherapy. Additionally, we have found that inhibitors of activin can inhibit or reduce immune responses, such as inflammatory and autoimmune responses and inflammation. Thus provided are methods for blocking activin activity with inhibitors for treatments of autoimmune diseases that have a Tfh component, such as, for example, lupus, Sjogrens Syndrome, rheumatoid arthritis, and others.

In certain embodiments, the invention provides for a method for stimulating, inducing, increasing or enhancing Tfh (follicular helper) cell differentiation comprising contacting a cell with activin or a subsequence thereof or an activator of activin Receptor effective to stimulate, induce, increase or enhance Tfh (follicular helper) cell differentiation. In additional embodiments, the invention provides for a method for stimulating, inducing, increasing or enhancing Tfh (follicular helper) cell differentiation, germinal center B cell development or an antibody response, comprising administering activin or a subsequence thereof or an activator of activin receptor to a subject in amount effective to stimulate induce, increase or enhance Tfh (follicular helper) cell differentiation, germinal center B cell development or an antibody response in the subject. In further embodiments, the invention provides for a method for stimulating, inducing, increasing or enhancing an immune response, comprising administering activin or a subsequence thereof or an activator of activin receptor to a subject in an amount effective to stimulate, induce, increase or enhance an immune response in the subject. In still further embodiments, the invention provides for a method for stimulating or increasing an immune response induced by a vaccine, comprising administering activin or a subsequence thereof or an activator of activin receptor to a subject in an amount effective to stimulate, induce or increase an immune response induced by the vaccine in the subject. In particular aspects, the immune response comprises a cell-mediated or humoral immune response. In additional aspects, the immune response stimulated, induced or increased is against a bacterial, viral, fungal or parasite pathogen. In further aspects, the immune response stimulated, induced or increased is against cancer. In additional aspects, the vaccine comprises a bacterial, viral, fungal, parasite or cancer antigen. In additional aspects, the vaccine is administered prior to, during or following administration of an activator of activin receptor or activin or subsequence thereof. In further aspects, the subject is immunosuppressed or immunocompromised or has an immunodeficiency syndrome.

In additional embodiments, the invention provides a method for vaccinating a subject against a pathogen or cancer, comprising administering activin or a subsequence thereof or an activator of activin receptor and a pathogen or pathogen antigen or cancer antigen to a subject in an amount effective to vaccinate the subject against the pathogen or cancer. In particular aspects, the pathogen comprises a bacterial, viral, fungal or parasite pathogen. In additional aspects, the method provides the subject with protection against one or more physiological conditions, disorders, illness, diseases or symptoms caused by or associated with pathogen infection or pathology or cancer. In further aspects, the activator of activin or a subsequence thereof, or activin receptor is administered prior to, substantially contemporaneously with or following administration of the subject with the pathogen or pathogen antigen or cancer antigen. In additional aspects, the activator of activin or a subsequence thereof, or activin receptor is administered within 2-72 hours, 2-48 hours, 4-24 hours, 4-18 hours, or 6-12 hours of administration of the subject with the pathogen or pathogen antigen or cancer antigen.

In additional embodiments, the invention provides for a method for inhibiting, decreasing or reducing Tfh (follicular helper) cell differentiation, germinal center B cell development or an antibody response, comprising administering an inhibitor of activin or activin receptor to a subject in an amount effective to inhibit, decrease or reduce Tfh (follicular helper) cell differentiation, germinal center B cell development or an antibody response in the subject. In further aspects, the invention provides for a method for inhibiting or reducing an undesirable or aberrant immune response, comprising administering an inhibitor of activin or activin receptor to a subject in an amount effective to inhibit or reduce an undesirable or aberrant immune response in the subject. In further embodiments, the invention provides a method for inhibiting or reducing an undesirable or aberrant inflammatory response or inflammation, comprising administering an inhibitor of activin or activin receptor to a subject in an amount effective to inhibit or reduce an undesirable or aberrant inflammatory response or inflammation in the subject. In particular aspects, the inhibitor binds to activin or activin receptor. In further aspects, the inhibitor comprises Follistatin. In additional aspects, the inhibitor comprises an antibody or a fragment thereof that binds to activin or activin receptor, or an antisense nucleic acid sequence of activin or activin receptor. In further aspects, the antibody or fragment thereof comprises an Fab, Fab′, F(ab′)₂, Fv, Fd, single-chain Fv (scFv), disulfide-linked Fvs (sdFv), V, VH, trispecific (Fab₃), bispecific (Fab₂), diabody ((V_{L}-V_{H})₂ or (V_{H}-V_{L})₂), triabody (trivalent), tetrabody (tetravalent), minibody ((scFᵥ-C_{H}3)₂), bispecific single-chain Fv (Bis-scFv), IgGdeltaCH2, scFv-Fc or (scFv)₂-Fc fragment. In additional aspects, the antibody is human or humanized. In further aspects, the antibody comprises one or more of M244B, M248, LS Bio LS-C195902, R&D Systems Clone 132815, R&D Systems Clone 69403, R&D Systems AF338, Novus Biologicals MM0074-7L18, GeneTex 7L18, or Ray Biotech ACVR1B. In further aspects, the antisense binds to a nucleic acid encoding a mammalian activin or activin receptor. In further aspects, the antisense binds to a nucleic acid encoding a human activin or activin receptor. In additional aspects, the antisense binds to a nucleic acid encoding all or a portion of a human activin sequence. In further aspects, the antisense binds to a nucleic acid encoding all or a portion of a human activin receptor sequence. In additional aspects, the undesirable or aberrant immune response or undesirable or aberrant inflammatory response or inflammation is acute or chronic. In additional aspects, the undesirable or aberrant immune response or undesirable or aberrant inflammatory response or inflammation comprises an autoimmune disease. In further aspects, the autoimmune disease comprises: rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, Vitiligo, autoimmune hemolytic anemia, autoimmune thrombocytopenic purpura, pernicious anemia, Guillain-Barre syndrome, acute rheumatic fever, sympathetic ophthalmia, Goodpasture's syndrome, systemic necrotizing vasculitis, antiphospholipid syndrome or an allergy. In further aspects, the undesirable or aberrant immune response or undesirable or aberrant inflammatory response or inflammation comprises a cell mediated or humoral immune response. In additional aspects, production or one or more antibodies is reduced, decreased, inhibited or suppressed. In particular aspects, the subject is a mammal. In further aspects, the subject is a human. In additional aspects, the activin inhibitor binds to a mammalian activin or activin receptor. In additional aspects, the activin inhibitor binds to a human activin or activin receptor sequence. In further aspects, the activin comprises a dimer comprising two polypeptides each selected from inhibin-βA or inhibin-βB. In additional aspects, the activin is mammalian. In further aspects, the activin is human. In additional aspects, the activin comprises all or a portion of a human activin sequence. In further aspects, the activin receptor comprises one of ActRIIA or ActRIIB. In additional aspects, the activin receptor further comprises one of ALK2 or ALK4.

In additional embodiments of the present invention, the invention provides a peptide comprising or consisting of a subsequence of human activin sequence wherein the subsequence stimulates, induces, increases or enhances development of Tfh (follicular helper) cell differentiation, germinal center B cell development, an antibody response, an immune response or an immune response induced by a vaccine. In particular aspects, the polypeptide is isolated or purified. In additional aspects, the subsequence is from about 5 to 425 amino acids in length provided that the subsequence is at least one amino acid less in length than the full length activin sequence. In additional aspects, the subsequence is from 5 to 15, 20 to 25, 25 to 50, 50 to 100, 100 to 150, 150 to 200, 200 to 300 or 300 to 425 amino acids in length provided that the subsequence or portion is at least one amino acid less in length than a full length activin sequence.

In additional embodiments, the invention provides a pharmaceutical composition comprising the peptide of the above embodiments. In particular aspects, the composition is a vaccine. In additional aspects, the composition further comprises an adjuvant.

### BRIEF DESCRIPTION OF THE DRAWINGS

FIG. 1A-D: Inhibin beta A (INHBA) emerged from the screen as potent regulator of human Tfh differentiation. (A) Schematic of primary screen. Purified human naïve CD4 T cells from a leukapheresis donor were stimulated by anti-CD3/CD28 beads on 384 well plates on day 0. The GNF secretomics recombinant proteins were added at the beginning of the stimulation. Each secretomics protein was tested in duplicate. After 5 days of in vitro culture, cells were evaluated by automated FACS analysis for the expression of Tfh signature markers, including CXCR5 and PD-1. (B) Primary screen results. Relative enrichment of PD-1⁺CXCR5⁺ cell induction is reported as Z score for each recombinant protein. INHBA gene product is shown in red. (C) CXCR5 and PD-1 expression from repeat experiment in which INHBA gene product was tested again for its capacity to induce Tfh-like differentiation. (D) Confocal microscopy of INHBA expression in human tonsils. Tonsil sections were stained with anti-INHBA (red) anti-BCL6 (blue) and anti-CD3 (green) Abs. An overlay from one donor representative of six is shown on the left panel. Enlarged images on the right panels are representative INHBA expression on (I) germinal center, (II) T-B border and (III) T cell areas.

FIG. 2A-O: Activin A plays a synergic role with IL-12 and molds the human Tfh gene program. (A-B) Bead purified naïve CD4 T cells from multiple healthy subjects were stimulated by anti CD3/CD28 beads on 96 well plates, in the presence of commercial human recombinant activin A, with or without IL-12. PD-1 and CXCR5 expression was analyzed after 5 days by FACS. A representative donor is shown in (A). Data from 12 donors from 6 independent experiments are shown in (B). The dotted line shows the average basal induction of PD-1⁺CXCR5⁺ cells induced by beads from 13 donors. (C) The expression of PD-1 and CXCR5 was monitored by flow cytometry at 0, 1, 2, 3, 5, 7 and 10 days on live cells following in vitro differentiation. Data from one experiment representative of two are depicted (n=3). (D) Cells were cultured in vitro with different doses of activin A combined with a fixed amount of IL-12 for 5 days in the presence of anti-activin A mAb MOR8806 (block) or isotype control mAb (isotype). Graphs show mean and SEM from 3 independent experiments (n=8). Dotted lines indicate the average percentages of PD-1⁺CXCR5⁺ cells induced by IL-12 with isotype control mAb from 8 donors. (E) CCR7 MFI on day 5 in vitro differentiated cells. The graph shows mean and SEM of 8 donors from 4 independent experiments. * p<0.05, ** p<0.01 (F) BCL6 intranuclear expression was measured by FACS on day 5 of the in vitro culture. Data are cumulative of 3 experiments (n=7). * p<0.05 (G-I) Gene Set Enrichment Analysis (GSEA) showing enrichment of tonsil GC Tfh signature genes (done from previously publish tonsil gene expression data) in genes upregulated by activin A+IL-12 cultured cells in comparison to beads only (G), IL-12 (H) or activin A only (I) stimulated cells. (J) RNA-seq analysis on day 3 in vitro differentiated cells. The “Volcano” plots show the absolute gene expression data set (RPKM) from cells differentiated with activin A and IL-12 versus cells differentiated with beads only. Genes up and downregulated in cells differentiated with activin A and IL-12 versus beads only are represented in red and blue, respectively (red, activin A+IL-12 versus beads >2; blue, activin A+IL-12 versus beads <−2). (K) Heat-map showing absolute expression (RPKM) of selected Tfh signature genes on cells differentiated with beads only, IL-12, activin A or activin A+IL12. (L and N) Relative expression (2̂-ΔCt) of LIF and PRDM1 was determined by qPCR. (M and O) SLAMF1 and ITGβ7 expression by flow cytometry. In (L) to (0) all the data were obtained from day 3 in vitro differentiated cells and are from at least 3 or more independent experiments (n=9 or higher). Bars show mean and SEM. * p<0.05, ** p<0.01 and *** p<0.001.

FIG. 3A-E: CD4 T cells differentiated with activin A and IL-12 acquire functional signature molecules of Tfh cells. (A) Naïve CD4 T cells from 9 donors were stimulated by plate bound anti-CD3 mAb and hrICOS-L chimera for 5 days. CXCL13 released in the supernatant was determined by ELISA. Data are individual values, mean and SEM from 3 independent experiments (n=9). ** p<0.01 (B) Day 5 in vitro differentiated cells were stimulated with PMA/Ionomycin and the intracellular expression of CD40L and IL-21 was measured by FACS. One representative donor is depicted. The graph on the right shows individual data, mean and SEM from 3 independent experiments (n=7 donors). * p<0.05. (C) Day 3 differentiated cells were stimulated with PMA/Ionomycin to test intracellular expression of TNF-α and LT-α by FACS. One representative donor is shown. (D) Quantification of (C). * p<0.05, ** p<0.01 and *** p<0.001. (E) Day 3 in vitro differentiated CD4 T cells were cocultured with fresh autologous memory B cells in the presence of SEB for 7 days. B cells (CD19+ live cells) and plasmablast (CD19⁺CD20^{lo}CD38⁺ cells) were analyzed by flow cytometry. IgG released in the culture supernatants were measured by ELISA. In all graphs n=8. Data are cumulative from 4 experiments. Mean and SEM are shown. Statistical significance was evaluated by one-tailed Wilcoxon matched-pairs signed ranked test. * p<0.05 and ** p<0.01.

FIG. 4A-I: Activin A and TGFβ act independently from each other to drive in vitro Tfh differentiation. (A-B) RNAseq data from day 3 differentiated cells. (A) Absolute expression (RPKM) values in cells differentiated with TGFβ+IL-12 and activin A+IL-12. (B) “Volcano” plots showing genes differentially regulated in cells differentiated with activin A+IL-12 versus beads only condition. Genes upregulated in cells differentiated with TGFβ+IL-12 (TGFβ+IL-12 versus beads, fold change >2) are shown in red, while genes downregulated in TGFβ+IL-12 (TGFβ+IL-12 versus beads, FC<−2) are depicted in blue. (C) Day 5 in vitro differentiation with TGFβ, alone or with IL-12, in the presence of anti-activin A mAb or isotype control mAb (n=8, 3 independent experiments). (D-E) TGFβ was blocked by anti-TGFβ mAb in cells cultured in vitro with TGFβ and TGFβ+IL-12 (D) or activin A and activin A+IL-12 (E). (F) Representative PD-1 and CXCR5 expression after 5 days of in vitro culture. (G) Quantification of (F). Data are from 3 independent experiments (n=6). (H-I) FoxP3 induction in day 5 in vitro differentiated cells was determined by FACS. Data are cumulative from 4 experiments. In (D), (G) and (H), * p<0.05, ** p<0.01 and *** p<0.001.

FIG. 5A-C: IL-2 antagonizes activin A driven Tfh cell differentiation. (A-C) Day 5 in vitro differentiation with anti-IL-2 blocking mAbs, or isotype controls. PD-1 and CXCR5 expression were measured by FACS. Data are cumulative from 3 independent experiments (n=10). ** p<0.01

FIG. 6A-D: The role of activin A in Tfh differentiation is conserved for non-human primate CD4 T cells, but not mouse CD4 T cell. (A-B) Splenic naïve CD4 T cells from BALB/c or C57BL/6 mice were cultured in vitro for 5 days with IL-12, activin A, activin A and IL12 or medium only, in the presence of plate-bound anti-CD3 and anti-CD28 mAbs. PD-1 and CXCR5 expression from one representative mouse samples are shown in (A). Data from 4 or more independent experiments were combined in (B) as mean and SEM. (C-D) Day 5 in vitro differentiation of FACS sorted naïve CD4 T cells (live CD4⁺CD45RA⁺CD95⁻CD28⁺CCR7⁺ cells) from rhesus macaque PBMCs. Cells were activated by plate bound anti-CD3 and anti-CD28 mAbs in the presence of human recombinant proteins. (C) Representative PD-1 and CXCR5 expression. (D) is the quantification of (C). Data are from 5 independent experiments (n=7). * p<0.05.

FIG. 7A-G: Activin A activity is mediated by SMAD2/3 pathway. (A-B) Expression of phosphorylated-SMAD2/3 (P-SMAD) by human naïve CD4 T cells (CD4⁺C45RA⁺) was measured by flow cytometry following stimulation with activin A (red), activin A+SB 431542 (blue) and in unstimulated cells (grey). (A) P-SMAD expression on one representative donor at 1 hour following stimulation (B) Data are cumulative from 3 independent experiments (n=8). ** p<0.01. (C-D) Human naïve CD4 T cells were differentiated for 5 days in the presence of SB 431542 or vehicle (DMSO). (C) Representative PD-1 and CXCR5 expression following 5 days culture with vehicle or SB 431542 (50 μM) is shown. (D) Data are from 4 independent experiments (n=6 or more). * p<0.05 and ** p<0.01. (E) Naïve CD4 T cells were culture for 5 days with activin A+IL-12 in the presence of Galunisertib or vehicle (DMSO). The graph shows combined data from 3 independent experiments (n=6). * p<0.05(F-G) P-SMAD expression from tonsillar naïve CD4 T cells (CD4⁺CD45RO−), mTfh (CD4⁺CD45RO⁺PD-1^{int}CXCR5^{int}) and GC Tfh cells (CD4⁺CD45RO⁺PD-1^{hi}CXCR5^{hi}) following stimulation with activin A (red), activin A+Galunisertib (blue) and in unstimulated cells (grey). (A) P-SMAD expression on one representative donor at 1 hour following stimulation (B) Data are cumulative from 3 independent experiments (n=6).

FIG. 8A-D: Activin A promotes the differentiation of human Tfh cells. Human naïve CD4 T cells were stimulated with anti CD3/CD28 coated beads alone (or in combination with human recombinant IL-12, activin A or both cytokines. The induction of PD-1+CXCR5+ Tfh-like cells was measured by flow cytometry. A representative donor is shown. It was found that activin A alone was able to drive a massive PD-1 induction and some CXCR5 expression. Moreover, in combination with IL-12, activin A caused a dramatic increase in the coexpression of PD-1 and CXCR5, a feature of bona fide Tfh cells. B. Titration of recombinant activin A, with or without recombinant IL-12, and its effect on the induction of PD-1+CXCR5+ cells. Data are mean and SEM from 4 independent experiments. C. Induction of Bcl6 protein expression was measured by FACS intranuclear staining. The percentage of Bcl6 expressing cells from 7 donors is shown. D. The downregulation of CCR7 induced by activin A was measured by FACS as CCR7 Mean Fluorescence Intensity (MFI). The graph displays mean and SEM from 8 donors.

FIG. 9. Human naïve CD4 T cells were stimulated with anti CD3/CD28 coated beads and titration of recombinant activin A with or without 11-2, and its effect on the induction of PD1+CXCR5+ cells was measured by flow cytometry. It was found that activin A has a synergistic effect with IL-12.

FIG. 10. Titration of recombinant activin A, with or without recombinant IL-12, and its effect on the induction of CCR7+CXCR5+ cells was measured by flow cytometry. The effect of recombinant TGFβ with or without recombinant IL-12 was also measured. It was found that both activin A and TGFβ influence CCR7 downregulation.

FIG. 11. Titration of recombinant activin A, with or without recombinant IL-12, and its effect on the induction of BC16+CXCR5+ cells was measured by flow cytometry. The effect of recombinant TGFβ with or without recombinant IL-12 was also measured. No detectable induction of Bcl6 was found.

FIG. 12. Blocking of activin activity by various activin inhibitors including follistatin and anti-activin antibodies was tested. FACS analysis

FIG. 13. To determine if activin A was responsible for the TGFβ induced Tfh-like phenotype, human naïve CD4 T cells were stimulated with anti CD3/CD28 coated beads in combination with TGFβ, IL-12 and an anti-activin A blocking antibody. The induction of Tfh-like cells was measured by flow cytometry. (A) activin was found to be potently inhibited by blocking a blocking antibody. (B) It was found that the anti-activin A antibody alone did not affect TGFβ activity.

FIG. 14. To determine if TGFβ was responsible for the activin A induced Tfh-like phenotype, human naïve CD4 T cells were stimulated with anti CD3/CD28 coated beads and combinations of activin A, IL-12 and/or an anti-TGFβ blocking antibody. The induction of Tfh-like cells was measured by flow cytometry. It was found that the anti-TGFβ Ab neutralizes hrTGFβ at 1 ng/ml but does not significantly affect the activin A induced phenotype.

FIG. 15. The synergies between different interleukins and TGFβ or activin A were examined by measuring the induction of CXCR5+ cells or CXCR5+PD1+ cells by flow cytometry. It was found that: (1) IL-27 does not act in synergy with TGFβ or activin A, (2) IL-23 synergizes with TGFβ or activin A but less potently than IL-12 (3) IL-35 does not synergize with TGFβ or activin.

FIG. 16. The IL-21 production of Tfh cells in vitro was determined after stimulating cells with plate bound anti-CD3 and CD28 and either activin A, with or without IL-12, TGFβ with or without IL-12 or IL-12 alone.

FIG. 17. The CXCL13 production of Tfh cells in vitro was determined after simulating cells with plate bound anti-CD3 and recombinant ICOS ligand and either activin A, with or without IL-12, TGFβ with or without IL-12 or IL-12 alone.

FIG. 18A-B: Activin A is responsible for induction of the Tfh signature markers CXCR5 and PD-1. FIG. 18A shows flow cytometry analysis of bead purified naïve CD4 T cells from a representative healthy donor subject that were stimulated by dynabeads on 96 well plates, in the presence of commercial human recombinant activin A, with IL-12 (“activin A+IL-12”) or without IL-12 (“activin A”) as indicated at the top of each panel. PD-1 (y-axis) and CXCR5 (x-axis) expression were analyzed after 5 days by FACS. Results were similar for multiple healthy donors (data not shown). FIG. 18B shows the percentage of naïve CD4 T cells from 6 donors that were differentiated (PD1⁺CXCR5⁺ double positive cells) with activin A and IL-12, or IL-12 only (horizontal dotted line, average value), in the presence of anti-ACVR2 pan mAb MOR8806 (labelled as CDD861 in the figure), or isotype control mAb (Isotype). Data are cumulative of 2 experiments.

### DETAILED DESCRIPTION OF THE INVENTION

The invention is based at least in part on our finding that activin (e.g., activin A, activin AB or activin B), is able to modulate various immune responses (e.g., stimulate, induce, increase or enhance, or inhibit, decrease or reduce an immune response). Activin has been found to have roles in cell proliferation, differentiation, apoptosis, metabolism, homeostasis, immune response, wound repair and endocrine function. Activin is a dimer composed of two identical or very similar beta subunits expressed by the Inhibin beta gene. Activin can comprise two subunits of beta-A (activin A), two subunits of beta-B (activin B) or one subunit of beta-A and one subunit of beta-B (activin AB). Activin receptor comes in two types activin type 1 receptor and activin type 2 receptor. Activin binds to the type 2 receptor and initiates a cascade reaction that leads to the recruitment, phosphorylation and activation of type 1 receptor, which then phosphorylates SMAD2 and SMAD3.

For example, activin can stimulate, induce, increase or enhance an immune response. Activin can also stimulate, induce, increase or enhance Tfh (follicular helper) cell differentiation, germinal center B cell development or an antibody response. Activin can furthermore stimulate, induce, increase or enhance an immune response, for example, a response induced by a vaccine. Accordingly, activin and activators of activin receptor are useful for and the invention provides, among other things, methods for stimulating, inducing or increasing an immune response, such as an immune response induced by a vaccine. In respective embodiments, a method includes administering activin or an activator of activin receptor to a subject in an amount effective to stimulate, induce, increase or enhance an immune response in the subject, administering activin or an activator of activin receptor to a subject in an amount effective to stimulate, induce, increase or enhance Tfh (follicular helper) cell differentiation, germinal center B cell development and/or an antibody response, and administering activin or an activator of activin receptor to a subject in an amount effective to stimulate, induce, increase or enhance an immune response, such as a response induced by the vaccine in the subject.

Inhibitors of activin receptor can inhibit, decrease and/or reduce Tfh (follicular helper) cell differentiation, germinal center B cell development and/or an antibody response. Inhibitors of activin receptor can furthermore inhibit, decrease and/or reduce an undesirable or aberrant immune response. Activin receptor inhibitors can additionally inhibit, decrease and/or reduce an undesirable or aberrant inflammatory response or inflammation in the subject. Accordingly, activin receptor inhibitors are useful for and the invention provides, among other things, methods for inhibiting, decreasing and/or reducing such cell differentiation, germinal center B cell development, antibody response(s), undesirable and aberrant immune response(s), and undesirable and aberrant inflammatory response(s) and inflammation. In respective embodiments, a method includes administering an inhibitor of activin receptor to a subject in an amount effective to inhibit, decrease and/or reduce Tfh (follicular helper) cell differentiation, germinal center B cell development or an antibody response in the subject, administering an inhibitor of activin receptor to a subject in an amount effective to inhibit, decrease and/or reduce an undesirable or aberrant immune response in the subject, and administering an inhibitor of activin receptor to a subject in an amount effective to inhibit, decrease and/or reduce method for inhibiting or reducing an undesirable or aberrant inflammatory response or inflammation in the subject.

Inhibitors and activators of activin receptor “modulate” activin activity, function or expression. Modulate can mean any increase, stimulation, promotion, or enhancement, or decrease, reduction, inhibition, or prevention, in activin activity, function or expression. For example, modulating an immune response means that activity or function or an effect is that the immune response is detectably changed, e.g., an increase, stimulation, promotion, or enhancement, or decrease, reduction, inhibition, or prevention, of any immune function, such as inflammation, humoral or cell mediated immunity, activity, function or numbers of T and B cells, cytokine or chemokine production, antibody production, mitogen responsiveness, or symptoms thereof, which can be measured by a variety of methods disclosed herein or known to one of skill in the art.

Exemplary activators of activin receptor detectably induce, increase, promote, stimulate or enhance an activity, function or expression of activin or activin receptor. Thus, an activin receptor activator detectably induces, increases, promotes, stimulates or enhances one or more activin receptor activities or functions or activin or activin receptor expression, which can include, for example, modulation of an immune response, as set forth herein or otherwise one that one of skill in the art would know.

An activin receptor activator may act directly upon activin receptor. Such activators of activin receptor need not bind to activin receptor provided that they induce, increase, promote, stimulate or enhance one or more activin receptor activities or functions. For example, an activin receptor activator may indirectly interact with activin receptor, for example, by acting through an intermediary, for example, the activator binds to or modulates a molecule that in turn binds to or modulates activin receptor.

Exemplary inhibitors of activin receptor detectably reduce, decrease, inhibit, prevent or abrogate an activity or function of activin or activin receptor. Thus, an activin receptor inhibitor detectably reduces, decreases, inhibits, prevents or abrogates one or more activin receptor activities or functions. Activin receptor activities and functions can include, for example, binding of activin receptor to a ligand, activin receptor mediated signaling or expression, and/or an immune response, as set forth herein or otherwise one that one of skill in the art would know.

An activin receptor inhibitor may act directly upon activin receptor. Such inhibitors of activin receptor need not bind to activin receptor provided that they inhibit, reduce, suppress or in any way interfere with activin or activin receptor function or activity. For example, an activin receptor inhibitor may indirectly interact with activin receptor, for example, by acting through an intermediary, for example, the inhibitor binds to or modulates a molecule that in turn binds to or modulates activin or activin receptor.

Activin receptor activators and inhibitors include ligands that bind to activin receptor or a subsequence thereof (e.g., SEQ ID NO: 3-10). Non-limiting examples of ligands include polypeptides that bind to activin receptor, such as antibodies and activin. Additional exemplary activin receptor activators and inhibitors include antisense RNA and inhibitory nucleic acid against activin or activin receptor.

The term “bind,” or “binding,” means a physical interaction at the molecular level (directly or indirectly). Typically, binding is that which is specific or selective for activin receptor, i.e., is statistically significantly higher than the background or control binding for the assay. The term “specifically binds” refers to the ability to preferentially or selectively bind to activin receptor. Specific and selective binding can be distinguished from non-specific binding using assays known in the art (e g, immunoprecipitation, ELISA, flow cytometry, and Western blotting).

### Antibodies and Antigen Binding Fragments Thereof

As used herein, the terms “activin Receptor antibody,” “anti-activin Receptor” and “anti-activin Receptor antibody” refer to an antibody that specifically binds to activin Receptor. As used herein, the terms “activin antibody,” “anti-activin” and “anti-activin antibody” refer to an antibody that specifically binds to activin. Antibodies include monoclonal or polyclonal immunoglobulin molecules that belong to any class such as IgM, IgG, IgA, IgE, IgD, and any subclass thereof. Exemplary subclasses for IgG are IgG1, IgG2, IgG3 and IgG4.

A “monoclonal” antibody refers to an antibody that is based upon, obtained from or derived from a single clone, including any eukaryotic, prokaryotic, or phage clone. A “monoclonal” antibody is therefore defined structurally, and not the method by which it is produced.

Antibodies include full-length antibodies that include two heavy and two light chain sequences. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, 1-R3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g. effector cells) and the first component (Clq) of the classical complement system. Antibodies can have kappa or lambda light chain sequences, either full length as in naturally occurring antibodies, mixtures thereof (i.e., fusions of kappa and lambda chain sequences), and subsequences/fragments thereof. Naturally occurring antibody molecules contain two kappa or two lambda light chains.

Antibodies include antigen binding fragments of antibodies comprising subsequences of a full native antibody complex. The term “antigen-binding portion” of an antibody (or simply “antigen portion”), as used herein, refers to full length or one or more fragments of an antibody that retain the ability to specifically bind to an antigen (e.g. a portion of ActRII). It has been shown that the antigen-binding function of an antibody can be performed by fragments of a full-length antibody. Subsequences include all or a portion of a full-length antibody heavy or light chain, or a heavy or light chain variable region, which includes one or more CDRs of a heavy or light chain variable region sequence. In various aspects, a subsequence of a full length antibody heavy or light chain, or a heavy or light chain variable region, has a length from about 20-30, 30-50, 50-100, 100-150, 150-200, 200-250, 250-300, 300-400, or 400-500, amino acid residues. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., 1989 Nature 341:544-546), which consists of a V_{H} domain; and an isolated complementarity determining region (CDR).

Non-limiting representative antigen binding fragments of antibodies comprising antibody subsequences include but are not limited to Fab, Fab′, F (ab′)₂, Fv, Fd, single-chain Fv (scFv), disulfide-linked Fvs (sdFv), V_{L}, V_{H}, Camel Ig, V-NAR, VHH, trispecific (Fab₃), bispecific (Fab₂), diabody ((V_{L}—V_{H})₂ or (V_{H}—V_{L})₂), triabody (trivalent), tetrabody (tetravalent), minibody ((scFv-C_{H}3)₂), bispecific single-chain Fv (Bis-scFv), IgGdeltaCH2, scFv-Fc, (scFv)₂-Fc, affibody, aptamer, avimer or nanobody, or other antigen binding subsequences of an intact immunoglobulin. Antibodies include those that bind to more than one epitope (e.g., bispecific antibodies), or antibodies that can bind to one or more different antigens (e.g., bi- or multi-specific antibodies).

Antibodies include antibodies and antigen binding fragments of antibodies subsequences capable of binding to activin or activin receptor in solution or in solid phase, present on one or more cells in vitro, in primary cell isolates, passaged cells, cultured cells and immortalized cells, or in vivo. Specific non-limiting cell types that can express activin or activin receptor include pituitary, macrophages, osteoblasts, fibroblasts, testis, thyroid, prostate, lung, pancreatic islet cells, bronchial epithelial cells, smooth muscle cells, cardiac myocytes, placenta, uterus and ovary.

Antibodies include mammalian, primatized, humanized, fully human antibodies and chimeras. A mammalian antibody is an antibody which is produced by a mammal, transgenic or non-transgenic, or a non-mammalian organism engineered to produce a mammalian antibody, such as a non-mammalian cell (bacteria, yeast, insect cell), animal or plant.

The term “human” when used in reference to an antibody, means that the amino acid sequence of the antibody is fully human, i.e., human heavy and human light chain variable and human constant regions. Thus, all of the amino acids are human or exist in a human antibody. An antibody that is non-human may be made fully human by substituting the non-human amino acid residues with amino acid residues that exist in a human antibody. Amino acid residues present in human antibodies, CDR region maps and human antibody consensus residues are known in the art (see, e.g., Kabat, Sequences of Proteins of Immunological Interest, 4th Ed. US Department of Health and Human Services. Public Health Service (1987); Chothia and Lesk (1987). A consensus sequence of human VH subgroup III, based on a survey of 22 known human VH III sequences, and a consensus sequence of human VH kappa-chain subgroup I, based on a survey of 30 known human kappa I sequences is described in Padlan Mol. Immunol. 3:169 (1994); and Padlan Mol. Immunol. 28:489 (1991). Human antibodies therefore include antibodies in which one or more amino acid residues have been substituted with one or more amino acids present in any other human antibody.

The term “humanized” when used in reference to an antibody, means that the amino acid sequence of the antibody has non-human amino acid residues (e.g., mouse, rat, goat, rabbit, etc.) of one or more complementarity determining regions (CDRs) that specifically bind to the desired antigen in an acceptor human immunoglobulin molecule, and one or more human amino acid residues in the Fv framework region (FR), which are amino acid residues that flank the CDRs. Such antibodies typically have reduced immunogenicity and therefore a longer half-life in humans as compared to the non-human parent antibody from which one or more CDRs were obtained or are based upon.

Antibodies referred to as “primatized” are “humanized” except that the acceptor human immunoglobulin molecule and framework region amino acid residues may be any primate amino acid residue (e.g., ape, gibbon, gorilla, chimpanzees orangutan, macaque), in addition to any human residue. Human FR residues of the immunoglobulin can be replaced with corresponding non-human residues. Residues in the CDR or human framework regions can therefore be substituted with a corresponding residue from the non-human CDR or framework region donor antibody to alter, generally to improve, antigen affinity or specificity, for example. A humanized antibody may include residues, which are found neither in the human antibody nor in the donor CDR or framework sequences. For example, a FR substitution at a particular position that is not found in a human antibody or the donor non-human antibody may be predicted to improve binding affinity or specificity human antibody at that position. Antibody framework and CDR substitutions based upon molecular modeling are well known in the art, e.g., by modeling of the interactions of the CDR and framework residues to identify framework residues important for antigen binding and sequence comparison to identify unusual framework residues at particular positions (see, e.g., U.S. Pat. No. 5,585,089; and Riechmann et al., Nature 332:323 (1988)).

The term “chimeric” and grammatical variations thereof, when used in reference to an antibody, means that the amino acid sequence of the antibody contains one or more portions that are derived from, obtained or isolated from, or based upon two or more different species. For example, a portion of the antibody may be human (e.g., a constant region) and another portion of the antibody may be non-human (e.g., a murine heavy or murine light chain variable region). Thus, an example of a chimeric antibody is an antibody in which different portions of the antibody are of different species origins. Unlike a humanized or primatized antibody, a chimeric antibody can have the different species sequences in any region of the antibody.

Methods of producing polyclonal and monoclonal antibodies are known in the art. For example, activin, activin receptor or an immunogenic fragment thereof, optionally conjugated to a carrier such as keyhole limpet hemocyanin (KLH) or albumin (e.g., BSA), or mixed with an adjuvant such as Freund's complete or incomplete adjuvant, and used to immunize an animal. Using hybridoma technology, splenocytes from immunized animals that respond to activin or activin receptor can be isolated and fused with myeloma cells. Monoclonal antibodies produced by hybridomas can be screened for reactivity with activin, activin receptor or an immunogenic fragment thereof. Hybridoma, recombinant, and phage display methods are known in the art (see, for example, U.S. Pat. Nos. 4,902,614, 4,543,439, and 4,411,993; see, also Monoclonal Antibodies. Hybridomas: A New Dimension in Biological Analyses. Plenum Press, Kennett, McKearn, and Bechtol (eds.), 1980, and Harlow et al., Antibodies: A Laboratory Manual, Cold Spring Harbor Laboratory Press, 2nd ed. 1988).

Animals that may be immunized include primates, mice, rats, rabbits, goats, sheep, cattle, or guinea pigs. Initial and any optional subsequent immunization may be through intravenous, intraperitoneal, intramuscular, or subcutaneous routes. Additionally, to increase the immune response, antigen can be coupled to another protein such as ovalbumin or keyhole limpet hemocyanin (KLH), thyroglobulin and tetanus toxoid, or mixed with an adjuvant such as Freund's complete or incomplete adjuvant. Initial and any optional subsequent immunization may be through intraperitoneal, intramuscular, intraocular, or subcutaneous routes. Subsequent immunizations may be at the same or at different concentrations of antigen, and may be at regular or irregular intervals.

Animals include those genetically modified to include human gene loci, which can be used to produce human antibodies. Transgenic animals, such as human trans-chromosomic mice with one or more human immunoglobulin genes, are described, for example, in U.S. Pat. No. 5,939,598, WO 02/43478, and WO 02/092812. In brief, animals are immunized with activin, activin receptor or cells that express activin or activin receptor antigen. Using conventional hybridoma technology, splenocytes from immunized mice that are high responders to the antigen can be isolated and fused with myeloma cells. Antibodies that bind to activin or activin receptor can thereby be obtained.

Humanized antibodies can be produced using techniques known in the art including, for example, CDR-grafting (EP 239,400; WO91/09967; U.S. Pat. Nos. 5,225,539; 5,530,101; and 5,585,089), veneering or resurfacing (EP 592,106; EP 519,596; Padlan, Molecular Immunol. 28:489 (1991); Studnicka et al., Protein Engineering 7:805 (1994); Roguska. et al., Proc. Natl Acad. Sci. USA 9 1:969 (1994)), and chain shuffling (U.S. Pat. No. 5,565,332). Human consensus sequences (Padlan, Mol. Immunol. 31:169 (1994); and Padlan, Mol. Immunol. 28:489 (1991)) have previously used to produce humanized antibodies (Carter et al., Proc. Natl. Acad. Sci. USA 89:4285 (1992); and Presta et al., J. Immunol. 151:2623 (1993)). Additional methods for producing human polyclonal antibodies and human monoclonal antibodies are described (see, e.g., Kuroiwa et al., Nat. Biotechnol. 20:889 (2002); WO 98/24893; WO 92/01047; WO 96/34096; WO 96/33735; U.S. Pat. Nos. 5,413,923; 5,625,126; 5,633,425; 5,569,825; 5,661,016; 5,545,806; 5,814,318; 5,885,793; 5,916,771; and 5,939,598).

Methods for producing chimeric antibodies are known in the art (e.g., Morrison, Science 229: 1202 (1985); Oi et al., BioTechniques 4:214 (1986); Gillies et al., J. Immunol. Methods 125:191 (1989); and U.S. Pat. Nos. 5,807,715; 4,816,567; and 4,816,397). Chimeric antibodies in which a variable domain from an antibody of one species is substituted for the variable domain of another species are described, for example, in Munro, Nature 312:597 (1984); Neuberger et al., Nature 312:604 (1984); Sharon et al., Nature 309:364 (1984); Morrison et al., Proc. Natl Acad. Sci. USA 81:6851 (1984); Boulianne et al., Nature 312:643 (1984); Capon et al., Nature 337:525 (1989); and Traunecker et al., Nature 339:68 (1989).

Activin or activin receptor protein suitable for generating antibodies can be produced by any of a variety of standard protein purification or recombinant expression techniques. Forms of activin or activin receptor suitable for generating an immune response include activin or activin receptor subsequences, such as an immunogenic fragment. Additional forms of activin or activin receptor include activin or activin receptor expressing cells, activin or activin receptor containing preparations or extracts or fractions, partially purified activin or activin receptor. For example, an activin or activin receptor sequence can be produced by standard peptide synthesis techniques, such as solid-phase synthesis. A portion of the protein may contain an amino acid sequence such as a T7 tag or polyhistidine sequence to facilitate purification of expressed or synthesized protein. The protein may be expressed in a cell and purified. The protein may be expressed as a part of a larger protein (e.g., a fusion or chimera) by recombinant methods.

Suitable techniques that additionally may be employed in antibody generation methods include activin or activin receptor based affinity purification, non-denaturing gel purification, HPLC or RP-HPLC, size exclusion, purification on protein A column, or any combination of these techniques. Antibody isotype can be determined using an ELISA assay, for example, a human Ig can be identified using mouse Ig-absorbed anti-human Ig.

Non-limiting representative examples of antibodies that specifically bind to activin or activin receptor include antibodies denoted as M244B, M248, LS Bio LS-C195902, R&D Systems Clone 132815, R&D Systems Clone 69403, R&D Systems AF338, Novus Biologicals MM0074-7L18, GeneTex 7L18, or Ray Biotech ACVR1B.

In some embodiments, methods comprise use of antibodies comprising V_{H} amino acid sequences of isolated antibodies shown in SEQ ID NOs: 106-119 and V_{L} amino acid sequences of isolated antibodies shown in SEQ ID NOs: 120-133 respectively. Examples of preferred full length heavy chain amino acid sequences of antibodies of use in methods of the invention are shown in SEQ ID NOs: 167-171 and 177-181. Examples of preferred full length light chain amino acid sequences of antibodies of use in the methods of the invention are shown in SEQ ID NOs: 162-166 and 172-176 respectively. Other antibodies of use in the methods of the invention include amino acids that have been mutated by amino acid deletion, insertion or substitution, yet have at least 85, 90, 95, 96, 97, 98 or 99 percent identity in the CDR regions with the CDR regions depicted in the sequences described above. In some embodiments, methods comprise use of antibody compositions provided herein which include mutant amino acid sequences wherein no more than 1, 2, 3, 4 or 5 amino acids have been mutated by amino acid deletion, insertion or substitution in the CDR regions when compared with the CDR regions depicted in the sequence described above.

In some embodiments, methods comprise use of antibodies or antigen binding fragments comprising variable heavy chain parental nucleotide sequences shown in SEQ ID NOs: 148-161 and variable light chain parental nucleotide sequences shown in SEQ ID NOs: 134-147. In certain embodiments methods comprise use of full length light chain nucleotide sequences optimized for expression in a mammalian cell shown in SEQ ID NOs: 182-186 and 192-196, and full length heavy chain nucleotide sequences optimized for expression in a mammalian cell are shown in SEQ ID NOs: 187-191 and 197-201. Other embodiments provide for use of antibodies in methods of the invention including amino acids or nucleic acids that have been mutated, yet have at least 85 or more (e.g. 90, 95, 96, 97, 98, 99 or more) percent identity to the sequences described above. In some embodiments, it includes mutant amino acid sequences wherein no more than 1, 2, 3, 4 or 5 amino acids have been mutated by amino acid deletion, insertion or substitution in the variable regions when compared with the variable regions depicted in the sequence described above.

In some embodiments, the V_{H}, V_{L}, full length light chain, and full length heavy chain sequences (nucleotide sequences and amino acid sequences) can be “mixed and matched” to create other anti-ActRII binding molecules of the invention. ActRII binding of such “mixed and matched” antibodies can be tested using the binding assays described above and in the Examples (e.g. ELISAs), and others know in the art. When these chains are mixed and matched, a V_{H} sequence from a particular V_{H}/V_{L} pairing should be replaced with a structurally similar V_{H} sequence. Likewise a full length heavy chain sequence from a particular full length heavy chain/full length light chain pairing should be replaced with a structurally similar full length heavy chain sequence. Likewise, a V_{L} sequence from a particular V_{H}/V_{L} pairing should be replaced with a structurally similar V_{L} sequence. Likewise a full length light chain sequence from a particular full length heavy chain/full length light chain pairing should be replaced with a structurally similar full length light chain sequence. Accordingly, in one aspect, the invention provides methods using an isolated recombinant anti-ActRII antibody or antigen binding region thereof having: a heavy chain variable region comprising an amino acid sequence selected from the group consisting of SEQ ID NOs: 106-119; and a light chain variable region comprising an amino acid sequence selected from the group consisting of SEQ ID NOs: 120-133.

In another embodiment, the invention provides methods of use of an isolated recombinant anti-ActRII antibody or antigen binding fragment thereof having: a full length heavy chain comprising an amino acid sequence selected from the group consisting of SEQ ID NOs:106-119; and a full length light chain comprising an amino acid sequence selected from the group consisting of SEQ ID NOs:120-133.

In another embodiment, the invention provides methods of use of an isolated recombinant anti-ActRII antibody or antigen binding fragment thereof having a full length heavy chain encoded by a nucleotide sequence that has been optimized for expression in the cell of a mammalian selected from the group consisting of SEQ ID NOs:148-161, and a full length light chain encoded by a nucleotide sequence that has been optimized for expression in the cell of a mammalian selected from the group consisting of SEQ ID NOs:134-147.

In particular embodiments, provided methods comprise use of antibodies described herein, for example: the amino acid sequences of the V_{H} CDR1s of the antibodies are shown in SEQ ID NOs: 22-35; the amino acid sequences of the V_{H} CDR2s of the antibodies are shown in SEQ ID NOs: 36-49; the amino acid sequences of the V_{H} CDR3s of the antibodies are shown in SEQ ID NOs: 50-63; the amino acid sequences of the V_{L} CDR1s of the antibodies are shown in SEQ ID NOs: 64-77; the amino acid sequences of the V_{L} CDR2s of the antibodies are shown in SEQ ID NOs: 78-91; the amino acid sequences of the V_{L} CDR3s of the antibodies are shown in SEQ ID NOs: 92-105. CDR regions are delineated using the Kabat system (Kabat, E. A., et al., 1991 Sequences of Proteins of Immunological Interest, Fifth Edition, U.S. Department of Health and Human Services, NIH Publication No. 91-3242).

In one embodiment, provided methods comprise use of antibodies wherein an isolated recombinant anti-ActRII antibody, or antigen binding region thereof has: a heavy chain variable region CDR1 comprising an amino acid sequence selected from the group consisting of SEQ ID NOs: 22-35; a heavy chain variable region CDR2 comprising an amino acid sequence selected from the group consisting of SEQ ID NOs: 36-49; a heavy chain variable region CDR3 comprising an amino acid sequence selected from the group consisting of SEQ ID NOs: 50-63; a light chain variable region CDR1 comprising an amino acid sequence selected from the group consisting of SEQ ID NOs: 64-77; a light chain variable region CDR2 comprising an amino acid sequence selected from the group consisting of SEQ ID NOs: 78-91; and a light chain variable region CDR3 comprising an amino acid sequence selected from the group consisting of SEQ ID NOs: 92-105.

In one embodiment, provided methods comprise use of antibodies wherein the antibody comprises: a heavy chain variable region CDR1 of SEQ ID NO: 22; a heavy chain variable region CDR2 of SEQ ID NO: 36; a heavy chain variable region CDR3 of SEQ ID NO: 50; a light chain variable region CDR1 of SEQ ID NO: 64; a light chain variable region CDR2 of SEQ ID NO: 78; and a light chain variable region CDR3 of SEQ ID NO: 92.

In one embodiment, provided methods comprise use of antibodies wherein the antibody comprises: a heavy chain variable region CDR1 of SEQ ID NO: 23 a heavy chain variable region CDR2 of SEQ ID NO: 37; a heavy chain variable region CDR3 of SEQ ID NO: 51; a light chain variable region CDR1 of SEQ ID NO: 65; a light chain variable region CDR2 of SEQ ID NO: 79; and a light chain variable region CDR3 of SEQ ID NO: 93.

In one embodiment, provided methods comprise use of antibodies wherein the antibody comprises: a heavy chain variable region CDR1 of SEQ ID NO: 24; a heavy chain variable region CDR2 of SEQ ID NO: 38; a heavy chain variable region CDR3 of SEQ ID NO: 52; a light chain variable region CDR1 of SEQ ID NO: 66; a light chain variable region CDR2 of SEQ ID NO: 80; and a light chain variable region CDR3 of SEQ ID NO: 94.

In one embodiment, provided methods comprise use of antibodies wherein the antibody comprises: a heavy chain variable region CDR1 of SEQ ID NO: 25; a heavy chain variable region CDR2 of SEQ ID NO: 39; a heavy chain variable region CDR3 of SEQ ID NO: 53; a light chain variable region CDR1 of SEQ ID NO: 67; a light chain variable region CDR2 of SEQ ID NO: 81; and a light chain variable region CDR3 of SEQ ID NO: 95.

In one embodiment, provided methods comprise use of antibodies wherein, the antibody comprises: a heavy chain variable region CDR1 of SEQ ID NO: 26; a heavy chain variable region CDR2 of SEQ ID NO: 40; a heavy chain variable region CDR3 of SEQ ID NO: 54; a light chain variable region CDR1 of SEQ ID NO: 68; a light chain variable region CDR2 of SEQ ID NO: 82; and a light chain variable region CDR3 of SEQ ID NO: 96.

In one embodiment, provided methods comprise use of antibodies wherein the antibody comprises: a heavy chain variable region CDR1 of SEQ ID NO: 27; a heavy chain variable region CDR2 of SEQ ID NO: 41; a heavy chain variable region CDR3 of SEQ ID NO: 55; a light chain variable region CDR1 of SEQ ID NO: 69; a light chain variable region CDR2 of SEQ ID NO: 83; and a light chain variable region CDR3 of SEQ ID NO: 97.

In one embodiment, provided methods comprise use of antibodies wherein the antibody comprises: a heavy chain variable region CDR1 of SEQ ID NO: 28; a heavy chain variable region CDR2 of SEQ ID NO: 42; a heavy chain variable region CDR3 of SEQ ID NO: 56; a light chain variable region CDR1 of SEQ ID NO: 70; a light chain variable region CDR2 of SEQ ID NO: 84; and a light chain variable region CDR3 of SEQ ID NO: 98.

In one embodiment, provided methods comprise use of antibodies wherein the antibody comprises: a heavy chain variable region CDR1 of SEQ ID NO: 29; a heavy chain variable region CDR2 of SEQ ID NO: 43; a heavy chain variable region CDR3 of SEQ ID NO: 57; a light chain variable region CDR1 of SEQ ID NO: 71 a light chain variable region CDR2 of SEQ ID NO: 85; and a light chain variable region CDR3 of SEQ ID NO: 99.

In one embodiment, provided methods comprise use of antibodies wherein the antibody comprises: a heavy chain variable region CDR1 of SEQ ID NO: 30; a heavy chain variable region CDR2 of SEQ ID NO: 44; a heavy chain variable region CDR3 of SEQ ID NO: 58; a light chain variable region CDR1 of SEQ ID NO: 72; a light chain variable region CDR2 of SEQ ID NO: 86; and a light chain variable region CDR3 of SEQ ID NO: 100.

In one embodiment, provided methods comprise use of antibodies wherein the antibody comprises: a heavy chain variable region CDR1 of SEQ ID NO: 31; a heavy chain variable region CDR2 of SEQ ID NO: 45; a heavy chain variable region CDR3 of SEQ ID NO: 59; a light chain variable region CDR1 of SEQ ID NO: 73; a light chain variable region CDR2 of SEQ ID NO87; and a light chain variable region CDR3 of SEQ ID NO: 101.

In one embodiment, provided methods comprise use of antibodies wherein the antibody comprises: a heavy chain variable region CDR1 of SEQ ID NO: 32; a heavy chain variable region CDR2 of SEQ ID NO: 46; a heavy chain variable region CDR3 of SEQ ID NO: 60; a light chain variable region CDR1 of SEQ ID NO: 74; a light chain variable region CDR2 of SEQ ID NO: 88; and a light chain variable region CDR3 of SEQ ID NO: 102.

In one embodiment, provided methods comprise use of antibodies wherein the antibody comprises: a heavy chain variable region CDR1 of SEQ ID NO: 33; a heavy chain variable region CDR2 of SEQ ID NO: 47; a heavy chain variable region CDR3 of SEQ ID NO: 61; a light chain variable region CDR1 of SEQ ID NO: 75; a light chain variable region CDR2 of SEQ ID NO: 89; and a light chain variable region CDR3 of SEQ ID NO: 103.

In one embodiment, provided methods comprise use of antibodies wherein the antibody comprises: a heavy chain variable region CDR1 of SEQ ID NO: 34; a heavy chain variable region CDR2 of SEQ ID NO: 48; a heavy chain variable region CDR3 of SEQ ID NO: 62; a light chain variable region CDR1 of SEQ ID NO: 76; a light chain variable region CDR2 of SEQ ID NO: 90; and a light chain variable region CDR3 of SEQ ID NO: 104.

In one embodiment, provided methods comprise use of antibodies wherein the antibody comprises: a heavy chain variable region CDR1 of SEQ ID NO: 35; a heavy chain variable region CDR2 of SEQ ID NO: 49; a heavy chain variable region CDR3 of SEQ ID NO: 63; a light chain variable region CDR1 of SEQ ID NO: 77; a light chain variable region CDR2 of SEQ ID NO: 91; and a light chain variable region CDR3 of SEQ ID NO: 105.

In one embodiment, provided methods comprise use of antibodies wherein the antibody comprises: (a) the variable heavy chain sequence of SEQ ID NO: 120 and variable light chain sequence of SEQ ID NO: 106; (b) the variable heavy chain sequence of SEQ ID NO: 121 and variable light chain sequence of SEQ ID NO: 107; (c) the variable heavy chain sequence of SEQ ID NO: 122 and variable light chain sequence of SEQ ID NO: 108; (d) the variable heavy chain sequence of SEQ ID NO: 123 and variable light chain sequence of SEQ ID NO: 109; (e) the variable heavy chain sequence of SEQ ID NO: 124 and variable light chain sequence of SEQ ID NO: 110; (f) the variable heavy chain sequence of SEQ ID NO: 125 and variable light chain sequence of SEQ ID NO: 111; (g) the variable heavy chain sequence of SEQ ID NO: 126 and variable light chain sequence of SEQ ID NO: 112; (h) the variable heavy chain sequence of SEQ ID NO: 127 and variable light chain sequence of SEQ ID NO: 113; (i) the variable heavy chain sequence of SEQ ID NO: 128 and variable light chain sequence of SEQ ID NO: 114; (j) the variable heavy chain sequence of SEQ ID NO: 129 and variable light chain sequence of SEQ ID NO: 115; (k) the variable heavy chain sequence of SEQ ID NO: 130 and variable light chain sequence of SEQ ID NO: 116; (1) the variable heavy chain sequence of SEQ ID NO: 131 and variable light chain sequence of SEQ ID NO: 117; (m) the variable heavy chain sequence of SEQ ID NO: 132 and variable light chain sequence of SEQ ID NO: 118; or (n) the variable heavy chain sequence of SEQ ID NO: 133 and variable light chain sequence of SEQ ID NO: 119.

In one embodiment, provided methods comprise use of antibodies wherein the antibody comprises: (a) the heavy chain sequence of SEQ ID NO: 167 and light chain sequence of SEQ ID NO: 162; (b) the heavy chain sequence of SEQ ID NO: 168 and light chain sequence of SEQ ID NO: 163; (c) the heavy chain sequence of SEQ ID NO: 169 and light chain sequence of SEQ ID NO: 164; (d) the heavy chain sequence of SEQ ID NO: 170 and light chain sequence of SEQ ID NO: 165; (e) the heavy chain sequence of SEQ ID NO: 171 and light chain sequence of SEQ ID NO: 166; (f) the heavy chain sequence of SEQ ID NO: 177 and light chain sequence of SEQ ID NO: 172; (g) the heavy chain sequence of SEQ ID NO: 178 and light chain sequence of SEQ ID NO: 173; (h) the heavy chain sequence of SEQ ID NO: 179 and light chain sequence of SEQ ID NO: 174; (i) the heavy chain sequence of SEQ ID NO: 180 and light chain sequence of SEQ ID NO: 175; or (j) the heavy chain sequence of SEQ ID NO: 181 and light chain sequence of SEQ ID NO: 176.

In certain embodiments, methods of the invention utilize an antibody having full length heavy and light chain amino acid sequences; full length heavy and light chain nucleotide sequences, variable region heavy and light chain nucleotide sequences, or variable region heavy and light chain amino acid sequences that are homologous to the amino acid and nucleotide sequences of the antibodies described herein, and wherein the antibodies retain the desired functional properties of the anti-ActRII antibodies of the invention. For example, the invention provides use of an isolated recombinant anti-ActRII antibody (or a functional protein comprising an antigen binding portion thereof) comprising a heavy chain variable region and a light chain variable region, wherein: the heavy chain variable region comprises an amino acid sequence that is at least 85%, or at least 90% (preferably at least 95, 97 or 99%) identical to an amino acid sequence selected from the group consisting of SEQ ID NOs: 106-119; the light chain variable region comprises an amino acid sequence that is at least 85%, or at least 90% (preferably at least 95, 97 or 99%) identical to an amino acid sequence selected from the group consisting of SEQ ID NOs: 120-133; and the antibody exhibits at least one of the following functional properties: (i) it inhibits activin binding in vitro or in vivo and/or (ii) decreases an immune response.

In a further embodiment, the invention methods provide for use of an isolated recombinant anti-ActRII antibody, (or a functional protein comprising an antigen binding portion thereof) comprising a full length heavy chain and a full length light chain, wherein: the full length heavy chain comprises an amino acid sequence that is at least 85%, or at least 90% (preferably at least 95, 97 or 99%) identical to an amino acid sequence selected from the group consisting of SEQ ID NOs: 167-171 and 177-181; the full length light chain comprises an amino acid sequence that is at least 85%, or at least 90% (preferably at least 95, 97 or 99%) identical to an amino acid sequence selected from the group consisting of SEQ ID NOs: 162-166 and 172-176; and the antibody exhibits at least one of the following functional properties: (i) it inhibits activin binding in vitro or in vivo and/or (ii) decreases an immune response.

In another embodiment, the invention methods provide for use of an isolated recombinant anti-ActRII antibody (or a functional protein comprising an antigen binding portion thereof), comprising a full length heavy chain and a full length light chain, wherein: the full length heavy chain is encoded by a nucleotide sequence that is at least 85%, or at least 90% (preferably at least 95, 97 or 99%) identical to a nucleotide sequence selected from the group consisting of SEQ ID NOs: 187-191 and 197-201; the full length light chain is encoded by a nucleotide sequence that is at least 85%, or at least 90% (preferably at least 95, 97 or 99%) identical to a nucleotide sequence selected from the group consisting of SEQ ID NOs: 182-186 and 192-196; and the antibody exhibits at least one of the following functional properties: (i) it inhibits activin binding in vitro or in vivo and/or (ii) decreases an immune response.

In various embodiments, an antibody may exhibit one or more, two or more, or three of the functional properties discussed herein. The antibody can be, for example, a human antibody, a humanized antibody or a chimeric antibody. Preferably the antibody is a fully human IgG1 antibody.

In other embodiments, the V_{H} and/or V_{L} amino acid sequences may be 85%, 90%, 95%, 96%, 97%, 98% or 99% identical to the sequences set forth above. In other embodiments, the V_{H} and/or V_{L} amino acid sequences may be identical except an amino acid substitution in no more than 1, 2, 3, 4 or 5 amino acid position. An antibody having V_{H} and V_{L} regions having high (i.e. 85% or greater) identity to the V_{H} and V_{L} regions of SEQ ID NOs 120-133 and SEQ ID NOs: 106-119 respectively, can be obtained by mutagenesis (e.g. site-directed or PCR-mediated mutagenesis) of nucleic acid molecules SEQ ID NOs: 148-161 and 134-147 respectively, followed by testing of the encoded altered antibody for retained function (i.e. the functions set forth above) using the functional assays described herein.

In other embodiments, the full length heavy chain and/or full length light chain amino acid sequences may be 85%, 90%, 95%, 96%, 97%, 98% or 99% identical to the sequences set forth above. An antibody having a full length heavy chain and full length light chain having high (i.e. 85% or greater) identity to the full length heavy chains of any of SEQ ID NOs: 167-171 and 177-181 and full length light chains of any of SEQ ID NOs: 162-166 and 172-176 respectively, can be obtained by mutagenesis (e.g. site-directed or PCR-mediated mutagenesis) of nucleic acid molecules SEQ ID NOs: 187-191 and 197-201 and SEQ ID NOs: 182-186 and 192-196 respectively, followed by testing of the encoded altered antibody for retained function (i.e. the functions set forth above) using the functional assays described herein.

As used herein, the percent identity between the two sequences is a function of the number of identical positions shared by the sequences (i.e. % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

In another embodiment, the invention methods provide for use of an isolated recombinant anti-ActRII antibody (or a functional protein comprising an antigen binding portion thereof), comprising an antibody having a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the anti-ActRII antibodies of the invention. Accordingly, the invention provides an isolated recombinant anti-ActRII antibody, or a functional protein comprising an antigen binding portion thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 22-35, and conservative modifications thereof; the heavy chain variable region CDR2 amino acid sequences are selected from the group consisting of SEQ ID NOs: 36-49, and conservative modifications thereof; the heavy chain variable region CDR3 amino acid sequences are selected from the group consisting of SEQ ID NOs: 50-63, and conservative modifications thereof; the light chain variable regions CDR1 amino acid sequences are selected from the group consisting of SEQ ID NOs: 64-77, and conservative modifications thereof; the light chain variable regions CDR2 amino acid sequences are selected from the group consisting of SEQ ID NOs: 78-91, and conservative modifications thereof; the light chain variable regions of CDR3 amino acid sequences are selected from the group consisting of SEQ ID NOs: 92-105, and conservative modifications thereof. Preferably the antibody exhibits at least one of the following functional properties: (i) it inhibits activin binding in vitro or in vivo and/or (ii) decreases an immune response.

In various embodiments, the antibody may exhibit one or both of the functional properties described herein. Such antibodies can be, for example, human antibodies, humanized antibodies or chimeric antibodies.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain Families of amino acid residues having similar side chains have been defined in the art. These families include amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine, tryptophan), nonpolar side chains (e.g. alanine, valine, leucine, isoleucine, proline, phenylalanine, methionine), beta-branched side chains (e.g. threonine, valine, isoleucine) and aromatic side chains (e.g. tyrosine, phenylalanine, tryptophan, histidine). Thus, one or more amino acid residues within the CDR regions of an antibody of use in the methods of the invention can be replaced with other amino acid residues from the same side chain family, and the altered antibody can be tested for retained function using the functional assays described herein.

Activin or activin receptor antibodies and functional (e.g., binding) subsequences, can have substantially the same, greater or less relative activity for than a reference antibody. For example, an activin or activin receptor antibody can have substantially the same, greater or less relative binding affinity or avidity for activin or activin receptor than a reference antibody. Such antibodies having measurable affinity for activin or activin receptor compete for binding of the reference antibody to activin or activin receptor. Activin or activin receptor antibodies and subsequences therefore include those that do not compete with a reference antibody for binding to activin or activin receptor, and those that compete with a reference antibody for binding to activin or activin receptor, and have substantially the same, greater or less relative binding affinity or avidity for binding to activin or activin receptor as compared to a reference antibody. In particular embodiments, a activin or activin receptor antibody competes for binding of antibody denoted as M244B, M248, LS Bio LS-C195902, R&D Systems Clone 132815, R&D Systems Clone 69403, R&D Systems AF338, Novus Biologicals MM0074-7L18, GeneTex 7L18, or Ray Biotech ACVR1B

Activin or activin receptor antibodies and subsequences can have a binding affinity for binding to activin or activin receptor within about K_{d} 10⁻² M to about 10⁻¹ M, or within about K_{d} 10⁻⁶ M to about 10⁻² M. In particular embodiments, binding affinity is less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M 5×10⁻¹² M, 10⁻¹² M, 5×10⁻¹³ M, 10⁻¹³ M, 5×10⁻¹⁴ M, 10⁻¹⁴ M, 5×10⁻¹⁵ M, and 10⁻¹⁵ M.

Activin or activin receptor antibodies and subsequences can have a greater or less than 2-5, 5-10, 10-100, 100-1000 or 1000-10,000-fold binding affinity for binding to activin or activin receptor, or any numerical value or range within or encompassing such values, than a reference antibody. In one embodiment, an antibody or a functional subsequence thereof has a binding affinity within about 1-5000 fold (more or less than) of a reference antibody for binding to activin or activin receptor.

In certain embodiments, useful compositions in provided methods include an antibody that “specifically binds to ActRII polypeptide” which is intended to refer to an antibody that binds to human ActRII polypeptide with a K_{D} of a 100 nM or less, 10 nM or less, 1 nM or less. An antibody that “cross-reacts with an antigen other than ActRII” is intended to refer to an antibody that binds that antigen with a K_{D} of 10×10⁻⁹ M or less, 5×10⁻⁹ M or less, or 2×10⁻⁹ M or less. An antibody that “does not cross-react with a particular antigen” is intended to refer to an antibody that binds to that antigen, with a K_{D} of 1.5×10⁻⁸ M or greater, or a K_{D} of 5-10×10⁻⁸ M, or 1×10⁻⁷ M or greater. In certain embodiments, such antibodies that do not cross-react with the antigen exhibit essentially undetectable binding against these proteins in standard binding assays. K_{D} may be determined using a biosensor system, such as a Biacore® system.

Activin or activin receptor antibodies and subsequences can have substantially the same binding affinity for activin or activin receptor as a reference antibody. In particular embodiments, a activin or activin receptor antibody has substantially the same binding affinity or avidity for activin or activin receptor as antibody denoted as M244B, M248, LS Bio LS-C195902, R&D Systems Clone 132815, R&D Systems Clone 69403, R&D Systems AF338, Novus Biologicals MM0074-7L18, GeneTex 7L18, or Ray Biotech ACVR1B.

The term “substantially the same” when used in reference to antibody or functional subsequence binding affinity or avidity for antigen, means that the binding affinity is within 100 fold of the binding affinity of a reference antibody for activin or activin receptor or a subsequence thereof. Binding affinity can be determined by association (Ka) and dissociation (Kd) rate. Equilibrium affinity constant, K, is the ratio of Ka/Kd. Association (Ka) and dissociation (Kd) rates can be measured using surface plasmon resonance (SPR) (Rich and Myszka, Curr. Opin. Biotechnol. 11:54 (2000); Englebienne, Analyst. 123: 599 (1998)). Instrumentation and methods for real time detection and monitoring of binding rates are known and are commercially available (BiaCore 2000, Biacore AB, Upsala, Sweden; and Malmqvist, Biochem. Soc. Trans. 27:335 (1999)). Thus, for example, if binding of a reference antibody to activin or activin receptor has a Kd 10⁻⁹ M, than an antibody which has substantially the same binding affinity as the reference activin or activin receptor antibody will have a Kd from 10⁻⁷ M to 10⁻¹¹ M for binding to activin or activin receptor.

### Polypeptides

Activin or activin receptor polypeptides and subsequences thereof, and amino acid substitutions thereof, can be used to practice invention methods and uses. Such activin or activin receptor polypeptides and subsequences thereof can exhibit sequence identity to a reference activin or activin receptor polypeptide or subsequence thereof. Activin comprises two subunits selected from an Inhibin-βa polypeptide and an Inhibin-βb polypeptide. An exemplary human Inhibin-βa polypeptide sequence is set forth as:

(SEQ ID NO: 1)
MPLLWLRGFLLASCWIIVRSSPTPGSEGHSAAPDCPSCALAALPKDVPN
SQPEMVEAVKKHILNMLHLKKRPDVTQPVPKAALLNAIRKLHVGKVGEN
GYVEIEDDIGRRAEMNELMEQTSEIITFAESGTARKTLHFEISKEGSDL
SVVERAEVWLFLKVPKANRTRTKVTIRLFQQQKHPQGSLDTGEEAEEVG
LKGERSELLLSEKVVDARKSTWHVFPVSSSIQRLLDQGKSSLDVRIACE
QCQESGASLVLLGKKKKKEEEGEGKKKGGGEGGAGADEEKEQSHRPFLM
LQARQSEDHPHRRRRRGLECDGKVNICCKKQFFVSFKDIGWNDWIIAPS
GYHANYCEGECPSHIAGTSGSSLSFHSTVINHYRMRGHSPFANLKSCCV
PTKLRPMSMLYYDDGQNIIKKDIQNMIVEECGCS.

An exemplary human Inhibin-βb polypeptide sequence is set forth as:

(SEQ ID NO: 2)
MDGLPGRALGAACLLLLAAGWLGPEAWGSPTPPPTPAAPPPPPPPGSPG
GSQDTCTSCGGIRRPEELGRVDGDFLEAVKRHILSRLQMRGRPNITHAV
PKAAMVTALRKLHAGKVREDGRVEIPHLDGHASPGADGQERVSEIISFA
ETDGLASSRVRLYFFISNEGNQNLFVVQASLWLYLKLLPYVLEKGSRRK
VRVKVYFQEQGHGDRWNMVEKRVDLKRSGWHTFPLTEAIQALFERGERR
LNLDVQCDSCQELAVVPVFVDPGEESHRPFVVVQARLGDSRHRIRKRGL
ECDGRTNLCCRQQFFIDFRLIGWNDWIIAPTGYYGNYCEGSCPAYLAGV
PGSASSFHTAVVNQYRMRGLNPGTVNSCCIPTKLSTMSMLYFDDEYNIV
KRDVPNMIVEECGCA.

Activin receptor comprises two subunits selected from one of ActRIIA or ActRIIB and one of ALK2 or ALK4. An exemplary human ActRIIA polypeptide sequence is set forth as one of:

(SEQ ID NO: 3)
MGAAAKLAFAVFLISCSSGAILGRSETQECLFFNANWEKDRTNQTGVEP
CYGDKDKRRHCFATWKNISGSIEIVKQGCWLDDINCYDRTDCVEKKDSP
EVYFCCCEGNMCNEKFSYFPEMEVTQPTSNPVTPKPPYYNILLYSLVPL
MLIAGIVICAFWVYRHHKMAYPPVLVPTQDPGPPPPSPLLGLKPLQLLE
VKARGRFGCVWKAQLLNEYVAVKIFPIQDKQSWQNEYEVYSLPGMKHEN
ILQFIGAEKRGTSVDVDLWLITAFHEKGSLSDFLKANVVSWNELCHIAE
TMARGLAYLHEDIPGLKDGHKPAISHRDIKSKNVLLKNNLTACIADFGL
ALKFEAGKSAGDTHGQVGTRRYMAPEVLEGAINFQRDAFLRIDMYAMGL
VLWELASRCTAADGPVDEYMLPFEEEIGQHPSLEDMQEVVVHKKKRPVL
RDYWQKHAGMAMLCETIEECWDHDAEARLSAGCVGERITQMQRLTNIIT
TEDIVTVVTMVTNVDFPPKESSL,
(SEQ ID NO: 4)
MCNEKFSYFPEMEVTQPTSNPVTPKPPYYNILLYSLVPLMLIAGIVICA
FWVYRHHKMAYPPVLVPTQDPGPPPPSPLLGLKPLQLLEVKARGRFGCV
WKAQLLNEYVAVKIFPIQDKQSWQNEYEVYSLPGMKHENILQFIGAEKR
GTSVDVDLWLITAFHEKGSLSDFLKANVVSWNELCHIAETMARGLAYLH
EDIPGLKDGHKPAISHRDIKSKNVLLKNNLTACIADFGLALKFEAGKSA
GDTHGQVGTRRYMAPEVLEGAINFQRDAFLRIDMYAMGLVLWELASRCT
AADGPVDEYMLPFEEEIGQHPSLEDMQEVVVHKKKRPVLRDYWQKHAGM
AMLCETIEECWDHDAEARLSAGCVGERITQMQRLTNIITTEDIVTVVTM
VTNVDFPPKESSL
or
(SEQ ID NO: 5)
MGAAAKLAFAVFLISCSSGAILGRSETQECLFFNANWEKDRTNQTGVEP
CYGDKDKRRHCFATWKNISGSIEIVKQGCWLDDINCYDRTDCVEKKDSP
EVYFCCCEGNMCNEKFSYFPEMEVTQPTSNPVTPKPPYYNILLYSLVPL
MLIAGIVICAFWVYRHHKMAYPPVLVPTQDPGPPPPSPLLGLKPLQLLE
VKARGRFGCVWKAQLLNEYVAVKIFPIQDKQSWQNEYEVYSLPGMKHEN
ILQFIGAEKRGTSVDVDLWLITAFHEKGSLSDFLKANVVSWNELCHIAE
TMARGLAYLHEDIPGLKDGHKPAISHRDIKSKNVLLKNNLTACIADFGL
ALKFEAGKSAGDTHGQVGTRRYMAPEVLEGAINFQRDAFLRIDMYAMGL
VLWELASRCTAADGPVDEYMLPFEEEIGQHPSLEDMQEVVVHKKKRPVL
RDYWQKHAGMAMLCETIEECWDHDAEARLSAGCVGERITQMQRLTNIIT
TEDIVTVVTMVTNVDFPPKESSL.

An exemplary human ActRIIB polypeptide sequence is set forth as:

(SEQ ID NO: 6)
MTAPWVALALLWGSLCAGSGRGEAETRECIYYNANWELERTNQSGLERC
EGEQDKRLHCYASWRNSSGTIELVKKGCWLDDFNCYDRQECVATEENPQ
VYFCCCEGNFCNERFTHLPEAGGPEVTYEPPPTAPTLLTVLAYSLLPIG
GLSLIVLLAFWMYRHRKPPYGHVDIHEDPGPPPPSPLVGLKPLQLLEIK
ARGRFGCVWKAQLMNDFVAVKIFPLQDKQSWQSEREIFSTPGMKHENLL
QFIAAEKRGSNLEVELWLITAFHDKGSLTDYLKGNIITWNELCHVAETM
SRGLSYLHEDVPWCRGEGHKPSIAHRDFKSKNVLLKSDLTAVLADFGLA
VRFEPGKPPGDTHGQVGTRRYMAPEVLEGAINFQRDAFLRIDMYAMGLV
LWELVSRCKAADGPVDEYMLPFEEEIGQHPSLEELQEVVVHKKMRPTIK
DHWLKHPGLAQLCVTIEECWDHDAEARLSAGCVEERVSLIRRSVNGTTS
DCLVSLVTSVTNVDLPPKESSI

An exemplary ALK2 polypeptide sequence is set forth as:

(SEQ ID NO: 7)
MVDGVMILPVLIMIALPSPSMEDEKPKVNPKLYMCVCEGLSCGNEDHCE
GQQCFSSLSINDGFHVYQKGCFQVYEQGKMTCKTPPSPGQAVECCQGDW
CNRNITAQLPTKGKSFPGTQNFHLEVGLIILSVVFAVCLLACLLGVALR
KFKRRNQERLNPRDVEYGTIEGLITTNVGDSTLADLLDHSCTSGSGSGL
PFLVQRTVARQITLLECVGKGRYGEVWRGSWQGENVAVKIFSSRDEKSW
FRETELYNTVMLRHENILGFIASDMTSRHSSTQLWLITHYHEMGSLYDY
LQLTTLDTVSCLRIVLSIASGLAHLHIEIFGTQGKPAIAHRDLKSKNIL
VKKNGQCCIADLGLAVMHSQSTNQLDVGNNPRVGTKRYMAPEVLDETIQ
VDCFDSYKRVDIWAFGLVLWEVARRMVSNGIVEDYKPPFYDVVPNDPSF
EDMRKVVCVDQQRPNIPNRWFSDPTLTSLAKLMKECWYQNPSARLTALR
IKKTLTKIDNSLDKLKTDC.

An exemplary ALK4 polypeptide sequence is set forth as one of:

(SEQ ID NO: 8)
MAESAGASSFFPLVVLLLAGSGGSGPRGVQALLCACTSCLQANYTCETD
GACMVSIFNLDGMEHHVRTCIPKVELVPAGKPFYCLSSEDLRNTHCCYT
DYCNRIDLRVPSGHLKEPEHPSMWGPVELVGIIAGPVFLLFLIIIIVFL
VINYHQRVYHNRQRLDMEDPSCEMCLSKDKTLQDLVYDLSTSGSGSGLP
LFVQRTVARTIVLQEIIGKGRFGEVWRGRWRGGDVAVKIFSSREERSWF
REAEIYQTVMLRHENILGFIAADNKDNGTWTQLWLVSDYHEHGSLFDYL
NRYTVTIEGMIKLALSAASGLAHLHMEIVGTQGKPGIAHRDLKSKNILV
KKNGMCAIADLGLAVRHDAVTDTIDIAPNQRVGTKRYMAPEVLDETINM
KHFDSFKCADIYALGLVYWEIARRCNSGGVHEEYQLPYYDLVPSDPSIE
EMRKVVCDQKLRPNIPNWWQSYEALRVMGKMMRECWYANGAARLTALRI
KKTLSQLSVQEDVKI.
(SEQ ID NO: 9)
MVSIFNLDGMEHHVRTCIPKVELVPAGKPFYCLSSEDLRNTHCCYTDYC
NRIDLRVPSGHLKEPEHPSMWGPVELVGIIAGPVFLLFLIIIIVFLVIN
YHQRVYHNRQRLDMEDPSCEMCLSKDKTLQDLVYDLSTSGSGSGLPLFV
QRTVARTIVLQEIIGKGRFGEVWRGRWRGGDVAVKIFSSREERSWFREA
EIYQTVMLRHENILGFIAADNKDNGTWTQLWLVSDYHEHGSLFDYLNRY
TVTIEGMIKLALSAASGLAHLHMEIVGTQGKPGIAHRDLKSKNILVKKN
GMCAIADLGLAVRHDAVTDTIDIAPNQRVGTKRYMAPEVLDETINMKHF
DSFKCADIYALGLVYWEIARRCNSGGVHEEYQLPYYDLVPSDPSIEEMR
KVVCDQKLRPNIPNWWQSYEALRVMGKMMRECWYANGAARLTALRIKKT
LSQLSVQEDVKI
(SEQ ID NO: 10)
MAESAGASSFFPLVVLLLAGSGGSGPRGVQALLCACTSCLQANYTCETD
GACMVSIFNLDGMEHHVRTCIPKVELVPAGKPFYCLSSEDLRNTHCCYT
DYCNRIDLRVPSGHLKEPEHPSMWGPVELVGIIAGPVFLLFLIIIIVFL
VINYHQRVYHNRQRLDMEDPSCEMCLSKDKTLQDLVYDLSTSGSGSGLP
LFVQRTVARTIVLQEIIGKGRFGEVWRGRWRGGDVAVKIFSSREERSWF
REAEIYQTVMLRHENILGFIAADNKADCSFLTLPWEVVMVSAAPKLRSL
RLQYKGGRGRARFLFPLNNGTWTQLWLVSDYHEHGSLFDYLNRYTVTIE
GMIKLALSAASGLAHLHMEIVGTQGKPGIAHRDLKSKNILVKKNGMCAI
ADLGLAVRHDAVTDTIDIAPNQRVGTKRYMAPEVLDETINMKHFDSFKC
ADIYALGLVYWEIARRCNSGGVHEEYQLPYYDLVPSDPSIEEMRKVVCD
QKLRPNIPNWWQSYEALRVMGKMMRECWYANGAARLTALRIKKTLSQLS
VQEDVKI.

A non-limiting exemplary activin or activin receptor polypeptide or subsequence thereof includes or consists of a sequence at least 60% or more (e.g., 65%, 70%, 75%, 80%, 85%, 90%, 95%, etc.) identical to any one of SEQ ID NO: 1-10, wherein the activin or activin receptor polypeptide or subsequence stimulates, induces, increases or enhances an immune response, Tfh (follicular helper) cell differentiation, germinal center B cell development, an antibody response, or an immune response, such as a response induced by a vaccine.

In accordance with the invention, there are also provided activin and subsequences thereof that exhibit sequence identity to a reference activin subunit polypeptide or subsequence thereof, and which have an ability to modulate an immune response, for example, stimulates, induces, increases or enhances an immune response, Tfh (follicular helper) cell differentiation, germinal center B cell development, an antibody response, or an immune response, such as a response induced by a vaccine, in vitro or in vivo. In one embodiment, an activin or subsequence thereof includes or consists of a sequence at least 60% or more (e.g., 65%, 70%, 75%, 80%, 85%, 90%, 95%, etc.) up to 100% identical to any activin or activin receptor polypeptide or subsequence thereof (e.g., SEQ ID NOs: 1-10)

In another embodiment, activin or subsequence thereof includes or consists of a activin or subsequence thereof set forth as SEQ ID NO: 1-2, wherein the activin subunit polypeptide or subsequence thereof has one or more amino acid additions, deletions or substitutions of any of SEQ ID NO: 1-2. In particular aspects, activin subunit or subsequence is at least 80% or more, e.g., 80-85%, 85-90%, 90-95%, 95-100% identical to a activin subunit polypeptide or subsequence thereof set forth in SEQ ID NO: 1-2.

Activin comprises two subunits selected from an Inhibin-βa polypeptide and an Inhibin-βb polypeptide. An exemplary human Inhibin-βa polypeptide sequence is set forth as (SEQ ID NO: 1).

An exemplary human Inhibin-polypeptide sequence is set forth as (SEQ ID NO: 2).

The invention provides isolated activin polypeptides, including or consisting of a activin subsequence, which exhibits sequence identity to a reference activin polypeptide or subsequence thereof (e.g., SEQ ID NO: 1-2), and which has one or more functions or activities of full length activin.

Such activin subsequences can be from about 5 to 300 amino acids in length, for example, from 5 to 15, 20 to 25, 25, to 50, 50 to 100, 100 to 150, 150 to 200, or 200 to 300 amino acids in length, provided that said subsequence or portion is at least one amino acid less in length than a full-length activin sequence (e.g., SEQ ID NO: 1-2).

In particular embodiments, a subsequence of an activin sequence or subsequence inhibits, decreases or reduces Tfh (follicular helper) cell differentiation, germinal center B cell development or an antibody response, inhibits, inhibits, decreases or reduces an undesirable or aberrant immune response, inhibits, decreases or reduces an undesirable or aberrant inflammatory response or inflammation. In additional embodiments, an activin subsequence stimulates, induces or increases an immune response, or stimulates, induces or increases Tfh (follicular helper) cell differentiation, germinal center B cell development, an antibody response, or stimulates, induces or increases an immune response, such as a response induced by a vaccine (e.g., elicits, promotes or enhances an immune response against a vaccine antigen in vitro or in vivo).

The term “isolated,” when used as a modifier of a composition (e.g., activin polypeptides and subsequences thereof, etc.), means that the compositions are made by the hand of man or are separated, completely or at least in part, from their naturally occurring in vivo environment.

Generally, isolated compositions are substantially free of one or more materials with which they normally associate with in nature, for example, one or more protein, nucleic acid, lipid, carbohydrate, cell membrane. The term “isolated” does not exclude alternative physical forms of the composition, such as fusions/chimeras, multimers/oligomers, modifications (e.g., phosphorylation, glycosylation, lipidation) or derivatized forms, or forms expressed in host cells produced by the hand of man

An “isolated” composition (e.g., activin or subsequence thereof) can also be “substantially pure” or “purified” when free of most or all of the materials with which it typically associates with in nature. Thus, isolated activin or subsequence thereof, that also is substantially pure or purified does not include polypeptides or polynucleotides present among millions of other sequences, such as peptides of an peptide library or nucleic acids in a genomic or cDNA library, for example. A “substantially pure” or “purified” composition can be combined with one or more other molecules. Thus, “substantially pure” or “purified” does not exclude combinations of compositions, such as combinations of activin or subsequence thereof and adjuvants, vaccines (e.g., antigens), agents, drugs or therapies.

The term “chimeric” and grammatical variations thereof, when used in reference to a sequence, means that the amino acid sequence contains one or more portions that are derived from, obtained or isolated from, or based upon two or more different proteins. For example, a portion of the sequence may be activin or subsequence thereof, and another portion of the sequence may be from a different peptide sequence, or a non-activin polypeptide sequence.

Activin and subsequences thereof of the invention include those having at least partial sequence identity to one or more exemplary activin sequences or a subsequence thereof (e.g., SEQ ID NO: 1-2). The percent identity of such sequences can be as little as 60%, or can be greater (e.g., 65%, 70%, 75%, 80%, 85%, 90%, 95%, 96%, 97%, 98%, 99%, etc.). The percent identity can extend over the entire sequence length or a portion of the sequence. In particular aspects, the length of the sequence sharing the percent identity is 2, 3, 4, 5 or more contiguous amino acids, e.g., 6, 7, 8, 9, 10, 11, 12, 13, 14, 15, 16, 17, 18, 19, 20, etc. contiguous amino acids. In additional particular aspects, the length of the sequence sharing the percent identity is 20 or more contiguous amino acids, e.g., 20, 21, 22, 23, 24, 25, 26, 27, 28, 29, 30, 31, 32, 33, 34, 35, etc. contiguous amino acids. In further particular aspects, the length of the sequence sharing the percent identity is 35 or more contiguous amino acids, e.g., 35, 36, 37, 38, 39, 40, 41, 42, 43, 44, 45, 45, 47, 48, 49, 50, etc., contiguous amino acids. In yet further particular aspects, the length of the sequence sharing the percent identity is 50 or more contiguous amino acids, e.g., 50-55, 55-60, 60-65, 65-70, 70-75, 75-80, 80-85, 85-90, 90-95, 95-100, 100-1 10, etc. contiguous amino acids.

The term “identity” and grammatical variations thereof, mean that two or more referenced entities are the same. Thus, where two activin polypeptides or subsequences thereof are identical, they have the same amino acid sequence. The identity can be over a defined area (region or domain) of the sequence. “Areas, regions or domains” of homology or identity mean that a portion of two or more referenced entities share homology or are the same. The extent of identity between two sequences can be ascertained using a computer program and mathematical algorithm known in the art. Such algorithms that calculate percent sequence identity (homology) generally account for sequence gaps and mismatches over the comparison region or area. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)).

Programs for quantitating protein structural similarity using Delaunay-based topological mapping have also been developed (Bostick et al., Biochem Biophys Res Commun. 304:320 (2003)).

In accordance with the invention, there are provided activin subunit polypeptides and subsequences thereof that include modified and variant forms. As used herein, the terms “modify” or “variant” and grammatical variations thereof, mean that activin subunit polypeptides or subsequence thereof deviates from a reference activin sequence (e.g., SEQ ID NO: 1-2). Modified and variant activin subunit polypeptides and subsequences thereof may therefore have greater or less activity or function than a reference activin subunit polypeptide, or subsequence thereof, but at least retain partial activity or function of the reference sequence (e.g., SEQ ID NO: 1-2). Thus, activin subunit polypeptides and subsequences thereof include sequences having substantially the same, greater or less relative activity or function as SEQ ID NO: 1-2, for example, an ability to stimulate, induce or increase Tfh (follicular helper) cell differentiation, germinal center B cell development or an antibody response in vitro or in vivo, an ability to stimulate, an ability to inhibit, decrease or reduce an undesirable or aberrant immune response in vitro or in vivo, an ability to inhibit, decrease or reduce an undesirable or aberrant inflammatory response or inflammation in vitro or in vivo, an ability to stimulate, induce or increase an immune response in vitro or in vivo, or an ability to stimulate, induce or increase an immune response induced by a vaccine in vitro or in vivo (e.g., elicit, promote or enhance an immune response against a vaccine antigen in vitro or in vivo).

Non-limiting examples of modifications include one or more amino acid substitutions (e.g., 1-3, 3-5, 5-10, 10-15, 15-20, 20-25, or more residues), additions (e.g., insertions or 1-3, 3-5, 5-10, 10-15, 15-20, 20-25, or more residues) and deletions (e.g., subsequences or fragments) of a reference activin subunit polypeptide or subsequence thereof. In particular embodiments, a modified or variant sequence retains at least part of a function or an activity of unmodified sequence. Such modified forms and variants can have less than, the same, or greater, but at least a part of, a function or activity of a reference sequence, for example, as described herein.

Specific non-limiting examples of substitutions include conservative and non-conservative amino acid substitutions. A “conservative substitution” is the replacement of one amino acid by a biologically, chemically or structurally similar residue. Biologically similar means that the substitution does not destroy a biological activity. Structurally similar means that the amino acids have side chains with similar length, such as alanine, glycine and serine, or a similar size. Chemical similarity means that the residues have the same charge or are both hydrophilic or hydrophobic.

Particular examples include the substitution of one hydrophobic residue for another (e.g., isoleucine, valine, leucine or methionine), or the substitution of one polar residue for another, (e.g., substitution of arginine for lysine, glutamic for aspartic acids, or glutamine for asparagine, serine for threonine, and the like).

A modification also includes one or more D-amino acids substituted for L-amino acids (and mixtures thereof), structural and functional analogues, for example, peptidomimetics having synthetic or non-natural amino acids or amino acid analogues and derivatized forms. Accordingly, polypeptides and peptides described herein further include compounds having amino acid structural and functional analogues, for example, peptidomimetics having synthetic or non-natural amino acids or amino acid analogues, so long as the mimetic has one or more functions or activities of a native polypeptide set forth herein. Non-natural and non-amide chemical bonds, and other coupling means can also be included, for example, glutaraldehyde, N-hydoxysuccinimide esters, bifunctional maleimides, or N, N′-dicyclohexylcarbodiimide (DCC). Non-amide bonds can include, for example, ketomethylene aminomethylene, olefin, ether, thioether and the like (see, e.g., Spatola (1983) in Chemistry and Biochemistry of Amino Acids. Peptides and Proteins, Vol. 7, pp 267-357, “Peptide and Backbone Modifications,” Marcel Decker, NY).

Modifications include cyclic structures such as an end-to-end amide bond between the amino and carboxy-terminus of the molecule or intra- or inter-molecular disulfide bond.

Polypeptides, peptides and peptidomimetics can be produced and isolated using methods known in the art. Peptides can be synthesized, whole or in part, using chemical methods known in the art (see, e.g., Caruthers (1980). Nucleic Acids Res. Symp. Ser. 15; Horn (1980); and Banga, A. K., Therapeutic Peptides and Proteins, Formulation, Processing and Delivery Systems (1995) Technomic Publishing Co., Lancaster, Pa.). Peptide synthesis can be performed using various solid phase techniques (see, e.g., Roberge Science 269:202 (1995); Merrifield, Methods Enzymol. 289:3(1997)) and automated synthesis may be achieved, e.g., using the ABI 431A Peptide Synthesizer (Perkin Elmer) in accordance with the manufacturer's instructions. Peptides and peptide mimetics can also be synthesized using combinatorial methodologies. Synthetic residues and polypeptides incorporating mimetics can be synthesized using a variety of procedures and methodologies known in the art (see, e.g., Organic Syntheses Collective Volumes, Gilman, et al. (Eds) John Wiley & Sons, Inc., NY). Modified peptides can be produced by chemical modification methods (see, for example, Belousov, Nucleic Acids Res. 25:3440 (1997); Frenkel, Free Radic. Biol. Med. 19:373 (1995); and Blommers, Biochemistry 33:7886 (1994)

An addition can be the covalent or non-covalent attachment of any type of molecule to the sequence. Specific examples of additions include glycosylation, acetylation, phosphorylation, amidation, formylation, ubiquitination, and derivatization by protecting/blocking groups and any of numerous chemical modifications. Additional specific non-limiting examples of an addition include one or more additional amino acid residues. In particular embodiments, an addition is a fusion (chimeric) sequence, an amino acid sequence having one or more molecules not normally present in a reference native (wild type) sequence covalently attached to the sequence. A particular example is an amino acid sequence of another sequence to produce a chimera. Another particular example of a modified sequence having an amino acid addition is one in which a second heterologous sequence, i.e., heterologous functional domain is attached (covalent or non-covalent binding) that confers a distinct or complementary function. Heterologous functional domains are not restricted to amino acid residues. Thus, a heterologous functional domain can consist of any of a variety of different types of small or large functional moieties. Such moieties include nucleic acid, peptide, carbohydrate, lipid or small organic compounds, such as a drug (e.g., an antiviral), a metal (gold, silver), radioisotope. Thus, in other embodiments the invention provides activin subunit polypeptides and subsequences thereof and a heterologous domain, wherein the domain confers a distinct function, i.e. a heterologous functional domain.

Further non-limiting examples of additions are detectable labels. Thus, in another embodiment, the invention provides activin subunit polypeptides and subsequences thereof that are detectably labeled. Specific examples of detectable labels include fluorophores, chromophores, radioactive isotopes (e.g., S35, P32, 1125), electron-dense reagents, enzymes, ligands and receptors. Enzymes are typically detected by their activity. For example, horseradish peroxidase is usually detected by its ability to convert a substrate such as 3,3-′,5,5-′-tetramethylbenzidine (TMB) to a blue pigment, which can be quantified.

Another non-limiting example of an addition is an insertion of an amino acid within any sequence of activin subunit polypeptides and subsequences thereof (e.g., SEQ ID NO: 1-2). In a particular embodiment, an insertion is of one or more amino acid residues in an activin subunit polypeptide or subsequence thereof (e.g., SEQ ID NO: 1-2).

Modified activin subunit polypeptides and subsequences thereof also include one or more D-amino acids substituted for L-amino acids (and mixtures thereof), structural and functional analogues, for example, peptidomimetics having synthetic or non-natural amino acids or amino acid analogues and derivatized forms.

Activin subunit polypeptides and subsequences thereof including modified forms can be produced by any of a variety of standard protein purification or recombinant expression techniques. For example, an activin subunit polypeptide or subsequence thereof can be produced by standard peptide synthesis techniques, such as solid-phase synthesis. A portion of the protein may contain an amino acid sequence such as a T7 tag or polyhistidine sequence to facilitate purification of expressed or synthesized protein. The protein may be expressed in a cell and purified. The protein may be expressed as a part of a larger protein (e.g., a fusion or chimera) by recombinant methods.

Activin subunit polypeptides and subsequences thereof including modified forms can be made using recombinant DNA technology via cell expression or in vitro translation. Polypeptide sequences including modified forms can also be produced by chemical synthesis using methods known in the art, for example, an automated peptide synthesis apparatus (see, e.g., Applied Biosystems, Foster City, Calif.).

In accordance with the invention, there are provided host cells that express activin subunit polypeptides and subsequences thereof. Host cells include but are not limited to prokaryotic and eukaryotic cells such as bacteria, fungi (yeast), plant, insect, and animal (e.g., mammalian, including primate and human, CHO cells and hybridomas) cells. For example, bacteria transformed with recombinant bacteriophage nucleic acid, plasmid nucleic acid or cosmid nucleic acid expression vectors; yeast transformed with recombinant yeast expression vectors; plant cell systems infected with recombinant virus expression vectors (e.g., cauliflower mosaic virus, CaMV; tobacco mosaic virus, TMV) or transformed with recombinant plasmid expression vectors (e.g., Ti plasmid); insect cell systems infected with recombinant virus expression vectors (e.g., baculovirus); and animal cell systems infected with recombinant virus expression vectors (e.g., retroviruses, adenovirus, vaccinia virus), or transformed animal cell systems engineered for stable expression. The cells may be a primary cell isolate, cell culture (e.g., passaged, established or immortalized cell line), or part of a plurality of cells, or a tissue or organ ex vivo or in a subject (in vivo).

The term “transformed” or “transfected” when used in reference to a cell (e.g., a host cell) or organism, means a genetic change in a cell following incorporation of an exogenous molecule, for example, a protein or nucleic acid (e.g., a transgene) into the cell. Thus, a “transfected” or “transformed” cell is a cell into which, or a progeny thereof in which an exogenous molecule has been introduced by the hand of man, for example, by recombinant DNA techniques.

An activin subunit nucleic acid or protein can be stably or transiently transfected or transformed (expressed) into the host cell and progeny thereof. The cell(s) can be propagated and the introduced protein expressed, or nucleic acid transcribed. A progeny of a transfected or transformed cell may not be identical to the parent cell, since there may be mutations that occur during replication.

### Nucleic Acids

Additional activin or activin receptor inhibitors include inhibitory and antisense nucleic acid sequences. Inhibitory, antisense and RNAi nucleic acids can modulate expression of activin or activin receptor. Antisense includes single, double or triple stranded polynucleotides and peptide nucleic acids (PNAs) that bind RNA transcript or DNA (e.g., genomic DNA). For example, a single stranded nucleic acid can target activin or activin receptor transcript (e.g., miRNA). Oligonucleotides derived from the transcription initiation site of activin or activin receptor gene, e.g., between positions −10 and +10 from the start site, are another particular example. Triplex forming antisense can bind to double strand DNA thereby inhibiting transcription of the gene. “RNAi” is the use of double stranded RNA sequences for inhibiting gene expression (see, e.g., Kennerdell et al., Cell 95: 1017 (1998); and Fire et al., Nature. 391:806 (1998)). Double stranded RNA sequences from an activin or activin receptor coding region may therefore be used to inhibit or prevent activin or activin receptor expression in accordance with the methods and uses of the invention. Antisense and RNAi can be produced based upon genes encoding activin or activin receptor sequences subunits (e.g., SEQ ID NOs: 1-10), such as nucleic acid encoding mammalian and human Inhibin-βa, Inhibin-βb, ActRIIA, ACTRIIB, ALK2 or ALK4. An exemplary human Inhibin-βa mRNA sequence is set forth as:

(SEQ ID NO: 11)
AGTACAGTATAAAACTTCACAGTGCCAATACCATGAAGAGGAGCTCAGAC
AGCTCTTACCACATGATACAAGAGCCGGCTGGTGGAAGAGTGGGGACCAG
AAAGAGAATTTGCTGAAGAGGAGAAGGAAAAAAAAAACACCAAAAAAAAA
AATAAAAAAATCCACACACACAAAAAAACCTGCGCGTGAGGGGGGAGGAA
AAGCAGGGCCTTTTAAAAAGGCAATCACAACAACTTTTGCTGCCAGGATG
CCCTTGCTTTGGCTGAGAGGATTTCTGTTGGCAAGTTGCTGGATTATAGT
GAGGAGTTCCCCCACCCCAGGATCCGAGGGGCACAGCGCGGCCCCCGACT
GTCCGTCCTGTGCGCTGGCCGCCCTCCCAAAGGATGTACCCAACTCTCAG
CCAGAGATGGTGGAGGCCGTCAAGAAGCACATTTTAAACATGCTGCACTT
GAAGAAGAGACCCGATGTCACCCAGCCGGTACCCAAGGCGGCGCTTCTGA
ACGCGATCAGAAAGCTTCATGTGGGCAAAGTCGGGGAGAACGGGTATGTG
GAGATAGAGGATGACATTGGAAGGAGGGCAGAAATGAATGAACTTATGGA
GCAGACCTCGGAGATCATCACGTTTGCCGAGTCAGGAACAGCCAGGAAGA
CGCTGCACTTCGAGATTTCCAAGGAAGGCAGTGACCTGTCAGTGGTGGAG
CGTGCAGAAGTCTGGCTCTTCCTAAAAGTCCCCAAGGCCAACAGGACCAG
GACCAAAGTCACCATCCGCCTCTTCCAGCAGCAGAAGCACCCGCAGGGCA
GCTTGGACACAGGGGAAGAGGCCGAGGAAGTGGGCTTAAAGGGGGAGAGG
AGTGAACTGTTGCTCTCTGAAAAAGTAGTAGACGCTCGGAAGAGCACCTG
GCATGTCTTCCCTGTCTCCAGCAGCATCCAGCGGTTGCTGGACCAGGGCA
AGAGCTCCCTGGACGTTCGGATTGCCTGTGAGCAGTGCCAGGAGAGTGGC
GCCAGCTTGGTTCTCCTGGGCAAGAAGAAGAAGAAAGAAGAGGAGGGGGA
AGGGAAAAAGAAGGGCGGAGGTGAAGGTGGGGCAGGAGCAGATGAGGAAA
AGGAGCAGTCGCACAGACCTTTCCTCATGCTGCAGGCCCGGCAGTCTGAA
GACCACCCTCATCGCCGGCGTCGGCGGGGCTTGGAGTGTGATGGCAAGGT
CAACATCTGCTGTAAGAAACAGTTCTTTGTCAGTTTCAAGGACATCGGCT
GGAATGACTGGATCATTGCTCCCTCTGGCTATCATGCCAACTACTGCGAG
GGTGAGTGCCCGAGCCATATAGCAGGCACGTCCGGGTCCTCACTGTCCTT
CCACTCAACAGTCATCAACCACTACCGCATGCGGGGCCATAGCCCCTTTG
CCAACCTCAAATCGTGCTGTGTGCCCACCAAGCTGAGACCCATGTCCATG
TTGTACTATGATGATGGTCAAAACATCATCAAAAAGGACATTCAGAACAT
GATCGTGGAGGAGTGTGGGTGCTCATAGAGTTGCCCAGCCCAGGGGGAAA
GGGAGCAAGAGTTGTCCAGAGAAGACAGTGGCAAAATGAAGAAATTTTTA
AGGTTTCTGAGTTAACCAGAAAAATAGAAATTAAAAACAAAACAAAAAAA
AAAACAAAAAAAAACAAAAGTAAATTAAAAACAAAACCTGATGAAACAGA
TGAAGGAAGATGTGGAAAAAATCCTTAGCCAGGGCTCAGAGATGAAGCAG
TGAAAGAGACAGGAATTGGGAGGGAAAGGGAGAATGGTGTACCCTTTATT
TCTTCTGAAATCACACTGATGACATCAGTTGTTTAAACGGGGTATTGTCC
TTTCCCCCCTTGAGGTTCCCTTGTGAGCCTTGAATCAACCAATCTAGTCT
GCAGTAGTGTGGACTAGAACAACCCAAATAGCATCTAGAAAGCCATGAGT
TTGAAAGGGCCCATCACAGGCACTTTCCTACCCAATTACCCAGGTCATAA
GGTATGTCTGTGTGACACTTATCTCTGTGTATATCAGCATACACACACAC
ACACACACACACACACACACACACAGGCATTTCCACACATTACATATATA
CACATACTGGTAAAAGAACAATCGTGTGCAGGTGGTCACACTTCCTTTTT
CTGTACCACTTTTGCAACAAAACAA.

An exemplary human Inhibin-βb mRNA sequence is set forth as:

(SEQ ID NO: 12)
ACTCGGCTCGCCTCGCGGCGGGCGCCCTCGTCGCCAGCGGCGCACCATGG
ACGGGCTGCCCGGTCGGGCGCTGGGGGCCGCCTGCCTTCTGCTGCTGGCG
GCCGGCTGGCTGGGGCCTGAGGCCTGGGGCTCACCCACGCCCCCGCCGAC
GCCTGCCGCGCCGCCGCCACCCCCGCCACCCGGATCCCCGGGTGGCTCGC
AGGACACCTGTACGTCGTGCGGCGGCTTCCGGCGGCCAGAGGAGCTCGGC
CGAGTGGACGGCGACTTCCTGGAGGCGGTGAAGCGGCACATCTTGAGCCG
CCTGCAGATGCGGGGCCGGCCCAACATCACGCACGCCGTGCCTAAGGCCG
CCATGGTCACGGCCCTGCGCAAGCTGCACGCGGGCAAGGTGCGCGAGGAC
GGCCGCGTGGAGATCCCGCACCTCGACGGCCACGCCAGCCCGGGCGCCGA
CGGCCAGGAGCGCGTTTCCGAAATCATCAGCTTCGCCGAGACAGATGGCC
TCGCCTCCTCCCGGGTCCGCCTATACTTCTTCATCTCCAACGAAGGCAAC
CAGAACCTGTTTGTGGTCCAGGCCAGCCTGTGGCTTTACCTGAAACTCCT
GCCCTACGTCCTGGAGAAGGGCAGCCGGCGGAAGGTGCGGGTCAAAGTGT
ACTTCCAGGAGCAGGGCCACGGTGACAGGTGGAACATGGTGGAGAAGAGG
GTGGACCTCAAGCGCAGCGGCTGGCATACCTTCCCACTCACGGAGGCCAT
CCAGGCCTTGTTTGAGCGGGGCGAGCGGCGACTCAACCTAGACGTGCAGT
GTGACAGCTGCCAGGAGCTGGCCGTGGTGCCGGTGTTCGTGGACCCAGGC
GAAGAGTCGCACCGGCCCTTTGTGGTGGTGCAGGCTCGGCTGGGCGACAG
CAGGCACCGCATTCGCAAGCGAGGCCTGGAGTGCGATGGCCGGACCAACC
TCTGTTGCAGGCAACAGTTCTTCATTGACTTCCGCCTCATCGGCTGGAAC
GACTGGATCATAGCACCCACCGGCTACTACGGGAACTACTGTGAGGGCAG
CTGCCCAGCCTACCTGGCAGGGGTCCCCGGCTCTGCCTCCTCCTTCCACA
CGGCTGTGGTGAACCAGTACCGCATGCGGGGTCTGAACCCCGGCACGGTG
AACTCCTGCTGCATTCCCACCAAGCTGAGCACCATGTCCATGCTGTACTT
CGATGATGAGTACAACATCGTCAAGCGGGACGTGCCCAACATGATTGTGG
AGGAGTGCGGCTGCGCCTGACAGTGCAAGGCAGGGGCACGGTGGTGGGGC
ACGGAGGGCAGTCCCGGGTGGGCTTCTTCCAGCCCCCGCGGGAACGGGGG
TACACGGTGGGCTGAGTACAGTCATTCTGTTGGGCTGTGGAGATAGTGCC
AGGGTGCGGCCTGAGATATTTTTCTACAGCTTCATAGAGCAACCAGTCAA
AACCAGAGCGAGAACCCTCAACTGACATGAAATACTTTAAAATGCACACG
TAGCCACGCACAGCCAGACGCATCCTGCCACCCACACAGCAGCCTCCAGG
ATACCAGCAAATGGATGCGGTGACAAATGGCAGCTTAGCTACAAATGCCT
GTCAGTCGGAGAGAATGGGGTGAGCAGCCACCATTCCCACCAGCTGGCCC
GGCCACTCTGAATTGCGCCTTCCGAGCACACATAAAAGCACAAAGACAGA
GACGCAGAGAGAGAGAGAGAGCCACGGAGAGGAAAAGCAGATGCAGGGGT
GGGGAGCGCAGCTCGGCGGAGGCTGCGTGTGCCCCGTGGCTTTTACCAGG
CCTGCTCTGCCTGGCTCGATGTCTGCTTCTTCCCCAGCCTGGGATCCTTC
GTGCTTCAAGGCCTGGGGAGCCTGTCCTTCCATGCCCTTGTCGAGGGAAA
GAGACCCAGAAAGGACACAACCCGTCAGAGACCTGGGAGCAGGGGCAATG
ACCGTTTGACTGTTTGTGGCTTGGGCCTCTGACATGACTTATGTGTGTGT
GTGTTTTTGGGGTGGGGAGGGAGGGAGAGAAGAGGGGGCTAAATTTGATG
CTTTAACTGATCTCCAACAGTTGACAGGTCATCCTTGCCAGTTGTATAAC
TGAAAAAGGACTTTTCTACCAGGTATGACCTTTTAAGTGAAAATCTGAAT
TGTTCTAAATGGAAAGAAAAAAAGTTGCAATCTGTGCCCTTCATTGGGGA
CATTCCTCTAGGACTGGTTTGGGGACGGGTGGGAATGACCCCTAGGCAAG
GGGATGAGACCGCAGGAGGAAATGGCGGGGAGGAGGCATTCTTGAACTGC
TGAGGATGGGGGGTGTCCCCTCAGCGGAGGCCAAGGGAGGGGAGCAGCCT
AGTTGGTCTTGGAGAGATGGGGAAGGCTTTCAGCTGATTTGCAGAAGTTG
CCCATGTGGGCCCCAGCCATCAGGGCTGGCCGTGGACGTGGCCCCTGCCC
ACTCACCTGCCCGCCTGCCCGCCCGCCCGCATAGCACTTGCAGACCTGCC
TGAACGCACATGACATAGCACTTGCCGATCTGCGTGTGTCCAGAAGTGGC
CCTTGGCCGAGCGCCGAACTCGCTCGCCCTCTAGATGTCCAAGTGCCACG
TGAACTATGCAATTTAAAGGGTTGACCCACACTAGACGAAACTGGACTCG
TACGACTCTTTTTATATTTTTTATACTTGAAATGAAATCCTTTGCTTCTT
TTTTAAGCGAATGATTGCTTTTAATGTTTGCACTGATTTAGTTGCATGAT
TAGTCAGAAACTGCCATTTGAAAAAAAGTTATTTTTATAGCAGCAAAAAA
AAAAAAAAAAGAATACAGTTAAATGTATTATACATAATTTTGGAACCAAA
GAGGCCAACAGATCAGTTTTAATTTTATTAGACGGTGAGGCCATCTGAGA
TGAGGTGGACGTTCTGAGCAGTCCCTTGAGTGGCCTGCCAACGTTTCAGG
GTATGAATGGATTTTGTTTATTCGGTTTGATGTGTCTTTTCCATCCTTAC
ACACCCAGAAGGTAGAGTAAAAATGACTATGATAGAATGCAGGTGTGTAT
CCTTAAATCCTCATCTTTATGTTTATTTAATAAAGCTCCCCTTAGATTCT
GTTTCATAATAATTTAAAACCAAACAATTTTCCCATAGACTTGCTGTTAA
AGTATTGTACGTTTGTGTACAGTTTAAGAAAATAAAAGATTGAGTGCCAC
GGGAAAAAAAAAAAAAAA.

Activin receptor comprises two subunits selected from one of ActRIIA or ActRIIB and one of ALK2 or ALK4. An exemplary human ActRIIA mRNA sequence is set forth as one of:

(SEQ ID NO: 13)
AGGAGACCGAAAACGCGGCCGAGCCCGGAGCCCGGAGCTGGAGCCAGAGC
CTGGACCAGAACTTGGCCGCCGCCTGCACCGCCGCCGCCGCTGCCGCCCG
CCGCCCCTTCCCCGCGCCGCAGCCGCCTCGCCGCCACCGCCGCGAGCTCG
GCCGCCAGTGGTCCTCGGACTTTAGGTGTCTGGGTTGAAGGAGGTTTGTC
TCCGAGGAAGACCCAGGGAACTGGATATCTAGCGAGAACTTCCTCCGGAT
TCCCCGGCGCCTCGGGAAAATGGGAGCTGCTGCAAAGTTGGCGTTTGCCG
TCTTTCTTATCTCCTGTTCTTCAGGTGCTATACTTGGTAGATCAGAAACT
CAGGAGTGTCTTTTCTTTAATGCTAATTGGGAAAAAGACAGAACCAATCA
AACTGGTGTTGAACCGTGTTATGGTGACAAAGATAAACGGCGGCATTGTT
TTGCTACCTGGAAGAATATTTCTGGTTCCATTGAAATAGTGAAACAAGGT
TGTTGGCTGGATGATATCAACTGCTATGACAGGACTGATTGTGTAGAAAA
AAAAGACAGCCCTGAAGTATATTTTTGTTGCTGTGAGGGCAATATGTGTA
ATGAAAAGTTTTCTTATTTTCCGGAGATGGAAGTCACACAGCCCACTTCA
AATCCAGTTACACCTAAGCCACCCTATTACAACATCCTGCTCTATTCCTT
GGTGCCACTTATGTTAATTGCGGGGATTGTCATTTGTGCATTTTGGGTGT
ACAGGCATCACAAGATGGCCTACCCTCCTGTACTTGTTCCAACTCAAGAC
CCAGGACCACCCCCACCTTCTCCATTACTAGGTTTGAAACCACTGCAGTT
ATTAGAAGTGAAAGCAAGGGGAAGATTTGGTTGTGTCTGGAAAGCCCAGT
TGCTTAACGAATATGTGGCTGTCAAAATATTTCCAATACAGGACAAACAG
TCATGGCAAAATGAATACGAAGTCTACAGTTTGCCTGGAATGAAGCATGA
GAACATATTACAGTTCATTGGTGCAGAAAAACGAGGCACCAGTGTTGATG
TGGATCTTTGGCTGATCACAGCATTTCATGAAAAGGGTTCACTATCAGAC
TTTCTTAAGGCTAATGTGGTCTCTTGGAATGAACTGTGTCATATTGCAGA
AACCATGGCTAGAGGATTGGCATATTTACATGAGGATATACCTGGCCTAA
AAGATGGCCACAAACCTGCCATATCTCACAGGGACATCAAAAGTAAAAAT
GTGCTGTTGAAAAACAACCTGACAGCTTGCATTGCTGACTTTGGGTTGGC
CTTAAAATTTGAGGCTGGCAAGTCTGCAGGCGATACCCATGGACAGGTTG
GTACCCGGAGGTACATGGCTCCAGAGGTATTAGAGGGTGCTATAAACTTC
CAAAGGGATGCATTTTTGAGGATAGATATGTATGCCATGGGATTAGTCCT
ATGGGAACTGGCTTCTCGCTGTACTGCTGCAGATGGACCTGTAGATGAAT
ACATGTTGCCATTTGAGGAGGAAATTGGCCAGCATCCATCTCTTGAAGAC
ATGCAGGAAGTTGTTGTGCATAAAAAAAAGAGGCCTGTTTTAAGAGATTA
TTGGCAGAAACATGCTGGAATGGCAATGCTCTGTGAAACCATTGAAGAAT
GTTGGGATCACGACGCAGAAGCCAGGTTATCAGCTGGATGTGTAGGTGAA
AGAATTACCCAGATGCAGAGACTAACAAATATTATTACCACAGAGGACAT
TGTAACAGTGGTCACAATGGTGACAAATGTTGACTTTCCTCCCAAAGAAT
CTAGTCTATGATGGTTGCGCCATCTGTGCACACTAAGAAATGGGACTCTG
AACTGGAGCTGCTAAGCTAAAGAAACTGCTTACAGTTTATTTTCTGTGTA
AAATGAGTAGGATGTCTCTTGGAAATGTTAAGAAAGAAGACCCTTTGTTG
AAAAATGTTGCTCTGGGAGACTTACTGCATTGCCGACAGCACAGATGTGA
AGGACATGAGACTAAGAGAAACCTTGCAAACTCTATAAAGAAACTTTTGA
AAAAGTGTACATGAAGAATGTAGCCCTCTCCAAATCAAGGATCTTTTGGA
CCTGGCTAATGGAGTGTTTGAAAACTGACATCAGATTTCTTAATGTCTGT
CAGAAGACACTAATTCCTTAAATGAACTACTGCTATTTTTTTTAAATCAA
AAACTTTTCATTTCAGATTTTAAAAAGGGTAACTTGTTTTTATTGCATTT
GCTGTTGTTTCTATAAATGACTATTGTAATGCCAATATGACACAGCTTGT
GAATGTTTAGTGTGCTGCTGTTCTGTGTACATAAAGTCATCAAAGTGGGG
TACAGTAAAGAGGCTTCCAAGCATTACTTTAACCTCCCTCAACAAGGTAT
ACCTCAGTTCCACGGTTGCTAAATTATAAAATTGAAAACACTAACAAAAT
TTGAATAATAAATCGATCCATGTTTTGTAACAAATTCACTGTGTTATTTA
AGGAAAAAAAGGTAAGCTATGCTTAGTGCCAACAATAAGTGGCCATTCGT
AAAGCAGTGTTTTAGCATTTCTTGTGCTGGCTTGTAATGTAGGGAAAAAA
AGTGCTGTTTTTTGAAAAGATGGTGTCATTTCCCCCTTCTTCCCATGTTT
TAAAGCCCCATCTTATATCCAGTTCCCAAAATTTGCATACTTACCTAAGT
ATTTTTTTTAGGTGTGCTGTGTTTGGGGAATATTTGAAAATTTAAAGCAT
GATTTAAAATTTTTTAAAGTGAGCTGTGACACTGGAAAGCTCTTCATTTT
ATCTTTTAAAATAGAGTTTTTTCTATTTATATATGTAAAATTGTAGTGTA
TTTCTTTTCACCAAACAGTGTGTGGGACATTCTTTATCACTGTTTTAGGA
TCACCTCAGGAAGTGTCGTTACCCAGAATTCCCCACTGTCTGCTATGAGA
CTTGTAACTTTATCACTATACTTCTGCTTGGTGCCATCTTGTCAGAGTAA
TATTTGATGTCTGTGATATGTAAAGAATTATCCTAGGATAAAGATATTAA
ACTTTAAGCAGATTTCAGATGTTACTGCTTTAAAACAAATCAGGGATAAC
AAATTAAACGTATAACTTAAAATATGCAATGACATTTAGAGGTAACCAAT
GTTGATATAGGTAGCATAGCCTAGCCTCCTCCCCAAAATTGCTTTTACAA
CTAACACTGATACTAATTTAGGATAGTTCATGCCTTATCCTTGCTAAGAA
AATGGAATTGATGGTAGGCAGGTGCTAAAGTGCTTTTCAAAACAATATTA
CGTTAGAATACAATTGGATTCTTCCTCAAATTTATACAGGCCAAAAAGTA
AAACATTAATTTTCTGAATTTCCAGATTACCAATCAATTAATCAACAAAT
AGCCAGTATTATGCTGTGTATTTCTGTCAGGTCATTTTAAAATCCATGTT
AATTTTATAAAAGAATTTTTTACATGTCACTGTCAGGAGCTCACTGTGAA
TGTGTTGTCTTCAAATGGTTATTTAACCACACAGTACACTACATTTTACA
TATATGTACGTAATCTCTGGGAATAGTAAATTAATTATGTTATTTATAAA
CAATACATAGGTCAACAGACTTTAAGCAGGGAGGAAAAGAAGAGTAATAG
CGTCTGTGTGCTGCAGACCATTCAGAACTGTCACGTGTGTCCCCATGGTC
TCATTCATTGTATTCCTAGCAATTCCCTTTTCAATGTTGAGTTCACCTCT
TTATTTCACAAAGTACTTGGTCTCTCAATTTCTTGATCTGGTTTTGCTTC
CATTTAAAAACTAATCAAGAAGGGAAAATATTGAGAATGTGCATACAAGA
AAATCATTAATTTCCTGAAGATGAATTTCTACCTGTTGTGAACATTTAAC
TTTCTTTTTAAAAGTTAAACAAAAATAAACAAGGGATATTATGATGAATG
TTTGGCTTATGTGAGTACTAGAGATAAAATTTTTAAACCCAGTTATTCAC
AATATAAAATGTTTTCAAGTTAGAAAAAATTTTTAGAAATCCTGGGTATT
GTATTTAACTGTAGCTAACCAATTTTAAAACTTGTATTCTTTTGAGAACT
ATTATTAATAGAAAAACTTTTTATAAGCAGTAAAATAAGAATGTTCCAGT
GACTACCTGTCCTTATACCTAGTCTTGTTAAAACTTTCTTTTGCAGGGTA
TTTAGTGTTTGGTTTACAGTCAGTGCAGAGTGGGCAAGTTAACAGAAAGT
TTGAGCTAGAGATACTGGAAAAAAAAAAGATCAAAGAATGAGAAAAATGG
TGATCCATTTTGGGGCAAACTGAGACCCCCCAAATAACTCTTTCCTCATG
TGTATGGTGCTCCTCATGACTCGTCTTGTATTTTGCCTTTCTGATACCCA
TCAGAACTGCTGCTGCTCTAACTTATACTCTTTACCTTGCCCAGATCTCC
GCGTAAGGAATGCTTTATGATCAACTTGCCATAGGACTGATGGATTAACC
AGTGTTCGGCTTTATTTGAAGTCTATGCCCTGCACAGCTCTTGTATGTAT
TTTAGATGCTAGAAGTTTTTTTAGCATGTGATGTGTGATTCTTGTTTGAA
TTCTAGGTACCTTGTGAATTCCAGAAAAAGAGACTGTGCTTCACGATTGT
TAGTCCCATGAACTTGCACTATCTATCTTTCATGGTGATGTTTTGAAAAT
ACAATCAGGAAAAAACCCAACACCTTTGGAATTTAAAATAGAATCATATC
ATGAAATTTAAAAAGAATCTCTTCTGTTGCATTTCCTCACCCCTAAGTAA
CAGCTACATTTAAGTAAAATGCAGGTGGTAGGGGAAAAAAAACCATGGCG
AGATGGTGGTTTAGTGGAATAAACTGATTACTGGTTTTTTTGTTTTTTTT
TTTTTTTTTAAAGAAAGAAGCTTCATCACAGATACTTTCCAGTTTCTCTT
TTATACTTTTTTGAAAGATTACTTTTTAGGAACATTTGGTATGATATGCA
TAAAATTATTTATCCATTTATGGGCAAAATGATACAAGTAGCATCTTGAT
TGAACATCATTTACCTCAGATATTCAACCAGCAGTACGTTTTTTATGCAG
TCTCAACCCATATCCCATTTGTTACCTCTCAGAATATTGGTAAGCAGTTA
TTTTCGCTTTACTCTGTATTTCTTGTGTTTTGGGCACAGGTTATTGTACT
ACTGTCAAATCGTACTTGCTATTTTTTCTGCAAGTATTTAACAGAAAGCT
TAAAATCCCCATAAAACCCCACCTTGGATAAGTGATTGTTAAATATTGTA
CAAATAAAATGTATGCTATCCCCATTCCATCCCCAAGTTAAATAAAAAAA
TGAATACGGTATGAAAAAAAAAAA,
((SEQ ID NO: 14)
TGAGCGTTTTTTTTTTTTTTTTTTTTTTTTTTTGGTCTGGGCTTCCGAAT
ATGTTTTATGACGGTTGATTTTACACCAGGAGGTTTGTCTCCGAGGAAGA
CCCAGGGAACTGGATATCTAGCGAGAACTTCCTCCGGATTCCCCGGCGCC
TCGGGAAAATGGGAGCTGCTGCAAAGTTGGCGTTTGCCGTCTTTCTTATC
TCCTGTTCTTCAGGTGCTATACTTGGTAGATCAGAAACTCAGGAGTGTCT
TTTCTTTAATGCTAATTGGGAAAAAGACAGAACCAATCAAACTGGTGTTG
AACCGTGTTATGGTGACAAAGATAAACGGCGGCATTGTTTTGCTACCTGG
AAGAATATTTCTGGTTCCATTGAAATAGTGAAACAAGGTTGTTGGCTGGA
TGATATCAACTGCTATGACAGGACTGATTGTGTAGAAAAAAAAGACAGCC
CTGAAGTATATTTTTGTTGCTGTGAGGGCAATATGTGTAATGAAAAGTTT
TCTTATTTTCCGGAGATGGAAGTCACACAGCCCACTTCAAATCCAGTTAC
ACCTAAGCCACCCTATTACAACATCCTGCTCTATTCCTTGGTGCCACTTA
TGTTAATTGCGGGGATTGTCATTTGTGCATTTTGGGTGTACAGGCATCAC
AAGATGGCCTACCCTCCTGTACTTGTTCCAACTCAAGACCCAGGACCACC
CCCACCTTCTCCATTACTAGGTTTGAAACCACTGCAGTTATTAGAAGTGA
AAGCAAGGGGAAGATTTGGTTGTGTCTGGAAAGCCCAGTTGCTTAACGAA
TATGTGGCTGTCAAAATATTTCCAATACAGGACAAACAGTCATGGCAAAA
TGAATACGAAGTCTACAGTTTGCCTGGAATGAAGCATGAGAACATATTAC
AGTTCATTGGTGCAGAAAAACGAGGCACCAGTGTTGATGTGGATCTTTGG
CTGATCACAGCATTTCATGAAAAGGGTTCACTATCAGACTTTCTTAAGGC
TAATGTGGTCTCTTGGAATGAACTGTGTCATATTGCAGAAACCATGGCTA
GAGGATTGGCATATTTACATGAGGATATACCTGGCCTAAAAGATGGCCAC
AAACCTGCCATATCTCACAGGGACATCAAAAGTAAAAATGTGCTGTTGAA
AAACAACCTGACAGCTTGCATTGCTGACTTTGGGTTGGCCTTAAAATTTG
AGGCTGGCAAGTCTGCAGGCGATACCCATGGACAGGTTGGTACCCGGAGG
TACATGGCTCCAGAGGTATTAGAGGGTGCTATAAACTTCCAAAGGGATGC
ATTTTTGAGGATAGATATGTATGCCATGGGATTAGTCCTATGGGAACTGG
CTTCTCGCTGTACTGCTGCAGATGGACCTGTAGATGAATACATGTTGCCA
TTTGAGGAGGAAATTGGCCAGCATCCATCTCTTGAAGACATGCAGGAAGT
TGTTGTGCATAAAAAAAAGAGGCCTGTTTTAAGAGATTATTGGCAGAAAC
ATGCTGGAATGGCAATGCTCTGTGAAACCATTGAAGAATGTTGGGATCAC
GACGCAGAAGCCAGGTTATCAGCTGGATGTGTAGGTGAAAGAATTACCCA
GATGCAGAGACTAACAAATATTATTACCACAGAGGACATTGTAACAGTGG
TCACAATGGTGACAAATGTTGACTTTCCTCCCAAAGAATCTAGTCTATGA
TGGTTGCGCCATCTGTGCACACTAAGAAATGGGACTCTGAACTGGAGCTG
CTAAGCTAAAGAAACTGCTTACAGTTTATTTTCTGTGTAAAATGAGTAGG
ATGTCTCTTGGAAATGTTAAGAAAGAAGACCCTTTGTTGAAAAATGTTGC
TCTGGGAGACTTACTGCATTGCCGACAGCACAGATGTGAAGGACATGAGA
CTAAGAGAAACCTTGCAAACTCTATAAAGAAACTTTTGAAAAAGTGTACA
TGAAGAATGTAGCCCTCTCCAAATCAAGGATCTTTTGGACCTGGCTAATG
GAGTGTTTGAAAACTGACATCAGATTTCTTAATGTCTGTCAGAAGACACT
AATTCCTTAAATGAACTACTGCTATTTTTTTTAAATCAAAAACTTTTCAT
TTCAGATTTTAAAAAGGGTAACTTGTTTTTATTGCATTTGCTGTTGTTTC
TATAAATGACTATTGTAATGCCAATATGACACAGCTTGTGAATGTTTAGT
GTGCTGCTGTTCTGTGTACATAAAGTCATCAAAGTGGGGTACAGTAAAGA
GGCTTCCAAGCATTACTTTAACCTCCCTCAACAAGGTATACCTCAGTTCC
ACGGTTGCTAAATTATAAAATTGAAAACACTAACAAAATTTGAATAATAA
ATCGATCCATGTTTTGTAACAAATTCACTGTGTTATTTAAGGAAAAAAAG
GTAAGCTATGCTTAGTGCCAACAATAAGTGGCCATTCGTAAAGCAGTGTT
TTAGCATTTCTTGTGCTGGCTTGTAATGTAGGGAAAAAAAGTGCTGTTTT
TTGAAAAGATGGTGTCATTTCCCCCTTCTTCCCATGTTTTAAAGCCCCAT
CTTATATCCAGTTCCCAAAATTTGCATACTTACCTAAGTATTTTTTTTAG
GTGTGCTGTGTTTGGGGAATATTTGAAAATTTAAAGCATGATTTAAAATT
TTTTAAAGTGAGCTGTGACACTGGAAAGCTCTTCATTTTATCTTTTAAAA
TAGAGTTTTTTCTATTTATATATGTAAAATTGTAGTGTATTTCTTTTCAC
CAAACAGTGTGTGGGACATTCTTTATCACTGTTTTAGGATCACCTCAGGA
AGTGTCGTTACCCAGAATTCCCCACTGTCTGCTATGAGACTTGTAACTTT
ATCACTATACTTCTGCTTGGTGCCATCTTGTCAGAGTAATATTTGATGTC
TGTGATATGTAAAGAATTATCCTAGGATAAAGATATTAAACTTTAAGCAG
ATTTCAGATGTTACTGCTTTAAAACAAATCAGGGATAACAAATTAAACGT
ATAACTTAAAATATGCAATGACATTTAGAGGTAACCAATGTTGATATAGG
TAGCATAGCCTAGCCTCCTCCCCAAAATTGCTTTTACAACTAACACTGAT
ACTAATTTAGGATAGTTCATGCCTTATCCTTGCTAAGAAAATGGAATTGA
TGGTAGGCAGGTGCTAAAGTGCTTTTCAAAACAATATTACGTTAGAATAC
AATTGGATTCTTCCTCAAATTTATACAGGCCAAAAAGTAAAACATTAATT
TTCTGAATTTCCAGATTACCAATCAATTAATCAACAAATAGCCAGTATTA
TGCTGTGTATTTCTGTCAGGTCATTTTAAAATCCATGTTAATTTTATAAA
AGAATTTTTTACATGTCACTGTCAGGAGCTCACTGTGAATGTGTTGTCTT
CAAATGGTTATTTAACCACACAGTACACTACATTTTACATATATGTACGT
AATCTCTGGGAATAGTAAATTAATTATGTTATTTATAAACAATACATAGG
TCAACAGACTTTAAGCAGGGAGGAAAAGAAGAGTAATAGCGTCTGTGTGC
TGCAGACCATTCAGAACTGTCACGTGTGTCCCCATGGTCTCATTCATTGT
ATTCCTAGCAATTCCCTTTTCAATGTTGAGTTCACCTCTTTATTTCACAA
AGTACTTGGTCTCTCAATTTCTTGATCTGGTTTTGCTTCCATTTAAAAAC
TAATCAAGAAGGGAAAATATTGAGAATGTGCATACAAGAAAATCATTAAT
TTCCTGAAGATGAATTTCTACCTGTTGTGAACATTTAACTTTCTTTTTAA
AAGTTAAACAAAAATAAACAAGGGATATTATGATGAATGTTTGGCTTATG
TGAGTACTAGAGATAAAATTTTTAAACCCAGTTATTCACAATATAAAATG
TTTTCAAGTTAGAAAAAATTTTTAGAAATCCTGGGTATTGTATTTAACTG
TAGCTAACCAATTTTAAAACTTGTATTCTTTTGAGAACTATTATTAATAG
AAAAACTTTTTATAAGCAGTAAAATAAGAATGTTCCAGTGACTACCTGTC
CTTATACCTAGTCTTGTTAAAACTTTCTTTTGCAGGGTATTTAGTGTTTG
GTTTACAGTCAGTGCAGAGTGGGCAAGTTAACAGAAAGTTTGAGCTAGAG
ATACTGGAAAAAAAAAAGATCAAAGAATGAGAAAAATGGTGATCCATTTT
GGGGCAAACTGAGACCCCCCAAATAACTCTTTCCTCATGTGTATGGTGCT
CCTCATGACTCGTCTTGTATTTTGCCTTTCTGATACCCATCAGAACTGCT
GCTGCTCTAACTTATACTCTTTACCTTGCCCAGATCTCCGCGTAAGGAAT
GCTTTATGATCAACTTGCCATAGGACTGATGGATTAACCAGTGTTCGGCT
TTATTTGAAGTCTATGCCCTGCACAGCTCTTGTATGTATTTTAGATGCTA
GAAGTTTTTTTAGCATGTGATGTGTGATTCTTGTTTGAATTCTAGGTACC
TTGTGAATTCCAGAAAAAGAGACTGTGCTTCACGATTGTTAGTCCCATGA
ACTTGCACTATCTATCTTTCATGGTGATGTTTTGAAAATACAATCAGGAA
AAAACCCAACACCTTTGGAATTTAAAATAGAATCATATCATGAAATTTAA
AAAGAATCTCTTCTGTTGCATTTCCTCACCCCTAAGTAACAGCTACATTT
AAGTAAAATGCAGGTGGTAGGGGAAAAAAAACCATGGCGAGATGGTGGTT
TAGTGGAATAAACTGATTACTGGTTTTTTTGTTTTTTTTTTTTTTTTTAA
AGAAAGAAGCTTCATCACAGATACTTTCCAGTTTCTCTTTTATACTTTTT
TGAAAGATTACTTTTTAGGAACATTTGGTATGATATGCATAAAATTATTT
ATCCATTTATGGGCAAAATGATACAAGTAGCATCTTGATTGAACATCATT
TACCTCAGATATTCAACCAGCAGTACGTTTTTTATGCAGTCTCAACCCAT
ATCCCATTTGTTACCTCTCAGAATATTGGTAAGCAGTTATTTTCGCTTTA
CTCTGTATTTCTTGTGTTTTGGGCACAGGTTATTGTACTACTGTCAAATC
GTACTTGCTATTTTTTCTGCAAGTATTTAACAGAAAGCTTAAAATCCCCA
TAAAACCCCACCTTGGATAAGTGATTGTTAAATATTGTACAAATAAAATG
TATGCTATCCCCATTCCATCCCCAAGTTAAATAAAAAAATGAATACGGTA
TGAAAAAAAAAAA,
or
(SEQ ID NO: 15)
AGGAGACCGAAAACGCGGCCGAGCCCGGAGCCCGGAGCTGGAGCCAGAGC
CTGGACCAGAACTTGGCCGCCGCCTGCACCGCCGCCGCCGCTGCCGCCCG
CCGCCCCTTCCCCGCGCCGCAGCCGCCTCGCCGCCACCGCCGCGAGCTCG
GCCGCCAGTGGTCCTCGGACTTTAGGTGTCTGGGTTGAAGACAGAACCAA
TCAAACTGGTGTTGAACCGTGTTATGGTGACAAAGATAAACGGCGGCATT
GTTTTGCTACCTGGAAGAATATTTCTGGTTCCATTGAAATAGTGAAACAA
GGTTGTTGGCTGGATGATATCAACTGCTATGACAGGACTGATTGTGTAGA
AAAAAAAGACAGCCCTGAAGTATATTTTTGTTGCTGTGAGGGCAATATGT
GTAATGAAAAGTTTTCTTATTTTCCGGAGATGGAAGTCACACAGCCCACT
TCAAATCCAGTTACACCTAAGCCACCCTATTACAACATCCTGCTCTATTC
CTTGGTGCCACTTATGTTAATTGCGGGGATTGTCATTTGTGCATTTTGGG
TGTACAGGCATCACAAGATGGCCTACCCTCCTGTACTTGTTCCAACTCAA
GACCCAGGACCACCCCCACCTTCTCCATTACTAGGTTTGAAACCACTGCA
GTTATTAGAAGTGAAAGCAAGGGGAAGATTTGGTTGTGTCTGGAAAGCCC
AGTTGCTTAACGAATATGTGGCTGTCAAAATATTTCCAATACAGGACAAA
CAGTCATGGCAAAATGAATACGAAGTCTACAGTTTGCCTGGAATGAAGCA
TGAGAACATATTACAGTTCATTGGTGCAGAAAAACGAGGCACCAGTGTTG
ATGTGGATCTTTGGCTGATCACAGCATTTCATGAAAAGGGTTCACTATCA
GACTTTCTTAAGGCTAATGTGGTCTCTTGGAATGAACTGTGTCATATTGC
AGAAACCATGGCTAGAGGATTGGCATATTTACATGAGGATATACCTGGCC
TAAAAGATGGCCACAAACCTGCCATATCTCACAGGGACATCAAAAGTAAA
AATGTGCTGTTGAAAAACAACCTGACAGCTTGCATTGCTGACTTTGGGTT
GGCCTTAAAATTTGAGGCTGGCAAGTCTGCAGGCGATACCCATGGACAGG
TTGGTACCCGGAGGTACATGGCTCCAGAGGTATTAGAGGGTGCTATAAAC
TTCCAAAGGGATGCATTTTTGAGGATAGATATGTATGCCATGGGATTAGT
CCTATGGGAACTGGCTTCTCGCTGTACTGCTGCAGATGGACCTGTAGATG
AATACATGTTGCCATTTGAGGAGGAAATTGGCCAGCATCCATCTCTTGAA
GACATGCAGGAAGTTGTTGTGCATAAAAAAAAGAGGCCTGTTTTAAGAGA
TTATTGGCAGAAACATGCTGGAATGGCAATGCTCTGTGAAACCATTGAAG
AATGTTGGGATCACGACGCAGAAGCCAGGTTATCAGCTGGATGTGTAGGT
GAAAGAATTACCCAGATGCAGAGACTAACAAATATTATTACCACAGAGGA
CATTGTAACAGTGGTCACAATGGTGACAAATGTTGACTTTCCTCCCAAAG
AATCTAGTCTATGATGGTTGCGCCATCTGTGCACACTAAGAAATGGGACT
CTGAACTGGAGCTGCTAAGCTAAAGAAACTGCTTACAGTTTATTTTCTGT
GTAAAATGAGTAGGATGTCTCTTGGAAATGTTAAGAAAGAAGACCCTTTG
TTGAAAAATGTTGCTCTGGGAGACTTACTGCATTGCCGACAGCACAGATG
TGAAGGACATGAGACTAAGAGAAACCTTGCAAACTCTATAAAGAAACTTT
TGAAAAAGTGTACATGAAGAATGTAGCCCTCTCCAAATCAAGGATCTTTT
GGACCTGGCTAATGGAGTGTTTGAAAACTGACATCAGATTTCTTAATGTC
TGTCAGAAGACACTAATTCCTTAAATGAACTACTGCTATTTTTTTTAAAT
CAAAAACTTTTCATTTCAGATTTTAAAAAGGGTAACTTGTTTTTATTGCA
TTTGCTGTTGTTTCTATAAATGACTATTGTAATGCCAATATGACACAGCT
TGTGAATGTTTAGTGTGCTGCTGTTCTGTGTACATAAAGTCATCAAAGTG
GGGTACAGTAAAGAGGCTTCCAAGCATTACTTTAACCTCCCTCAACAAGG
TATACCTCAGTTCCACGGTTGCTAAATTATAAAATTGAAAACACTAACAA
AATTTGAATAATAAATCGATCCATGTTTTGTAACAAATTCACTGTGTTAT
TTAAGGAAAAAAAGGTAAGCTATGCTTAGTGCCAACAATAAGTGGCCATT
CGTAAAGCAGTGTTTTAGCATTTCTTGTGCTGGCTTGTAATGTAGGGAAA
AAAAGTGCTGTTTTTTGAAAAGATGGTGTCATTTCCCCCTTCTTCCCATG
TTTTAAAGCCCCATCTTATATCCAGTTCCCAAAATTTGCATACTTACCTA
AGTATTTTTTTTAGGTGTGCTGTGTTTGGGGAATATTTGAAAATTTAAAG
CATGATTTAAAATTTTTTAAAGTGAGCTGTGACACTGGAAAGCTCTTCAT
TTTATCTTTTAAAATAGAGTTTTTTCTATTTATATATGTAAAATTGTAGT
GTATTTCTTTTCACCAAACAGTGTGTGGGACATTCTTTATCACTGTTTTA
GGATCACCTCAGGAAGTGTCGTTACCCAGAATTCCCCACTGTCTGCTATG
AGACTTGTAACTTTATCACTATACTTCTGCTTGGTGCCATCTTGTCAGAG
TAATATTTGATGTCTGTGATATGTAAAGAATTATCCTAGGATAAAGATAT
TAAACTTTAAGCAGATTTCAGATGTTACTGCTTTAAAACAAATCAGGGAT
AACAAATTAAACGTATAACTTAAAATATGCAATGACATTTAGAGGTAACC
AATGTTGATATAGGTAGCATAGCCTAGCCTCCTCCCCAAAATTGCTTTTA
CAACTAACACTGATACTAATTTAGGATAGTTCATGCCTTATCCTTGCTAA
GAAAATGGAATTGATGGTAGGCAGGTGCTAAAGTGCTTTTCAAAACAATA
TTACGTTAGAATACAATTGGATTCTTCCTCAAATTTATACAGGCCAAAAA
GTAAAACATTAATTTTCTGAATTTCCAGATTACCAATCAATTAATCAACA
AATAGCCAGTATTATGCTGTGTATTTCTGTCAGGTCATTTTAAAATCCAT
GTTAATTTTATAAAAGAATTTTTTACATGTCACTGTCAGGAGCTCACTGT
GAATGTGTTGTCTTCAAATGGTTATTTAACCACACAGTACACTACATTTT
ACATATATGTACGTAATCTCTGGGAATAGTAAATTAATTATGTTATTTAT
AAACAATACATAGGTCAACAGACTTTAAGCAGGGAGGAAAAGAAGAGTAA
TAGCGTCTGTGTGCTGCAGACCATTCAGAACTGTCACGTGTGTCCCCATG
GTCTCATTCATTGTATTCCTAGCAATTCCCTTTTCAATGTTGAGTTCACC
TCTTTATTTCACAAAGTACTTGGTCTCTCAATTTCTTGATCTGGTTTTGC
TTCCATTTAAAAACTAATCAAGAAGGGAAAATATTGAGAATGTGCATACA
AGAAAATCATTAATTTCCTGAAGATGAATTTCTACCTGTTGTGAACATTT
AACTTTCTTTTTAAAAGTTAAACAAAAATAAACAAGGGATATTATGATGA
ATGTTTGGCTTATGTGAGTACTAGAGATAAAATTTTTAAACCCAGTTATT
CACAATATAAAATGTTTTCAAGTTAGAAAAAATTTTTAGAAATCCTGGGT
ATTGTATTTAACTGTAGCTAACCAATTTTAAAACTTGTATTCTTTTGAGA
ACTATTATTAATAGAAAAACTTTTTATAAGCAGTAAAATAAGAATGTTCC
AGTGACTACCTGTCCTTATACCTAGTCTTGTTAAAACTTTCTTTTGCAGG
GTATTTAGTGTTTGGTTTACAGTCAGTGCAGAGTGGGCAAGTTAACAGAA
AGTTTGAGCTAGAGATACTGGAAAAAAAAAAGATCAAAGAATGAGAAAAA
TGGTGATCCATTTTGGGGCAAACTGAGACCCCCCAAATAACTCTTTCCTC
ATGTGTATGGTGCTCCTCATGACTCGTCTTGTATTTTGCCTTTCTGATAC
CCATCAGAACTGCTGCTGCTCTAACTTATACTCTTTACCTTGCCCAGATC
TCCGCGTAAGGAATGCTTTATGATCAACTTGCCATAGGACTGATGGATTA
ACCAGTGTTCGGCTTTATTTGAAGTCTATGCCCTGCACAGCTCTTGTATG
TATTTTAGATGCTAGAAGTTTTTTTAGCATGTGATGTGTGATTCTTGTTT
GAATTCTAGGTACCTTGTGAATTCCAGAAAAAGAGACTGTGCTTCACGAT
TGTTAGTCCCATGAACTTGCACTATCTATCTTTCATGGTGATGTTTTGAA
AATACAATCAGGAAAAAACCCAACACCTTTGGAATTTAAAATAGAATCAT
ATCATGAAATTTAAAAAGAATCTCTTCTGTTGCATTTCCTCACCCCTAAG
TAACAGCTACATTTAAGTAAAATGCAGGTGGTAGGGGAAAAAAAACCATG
GCGAGATGGTGGTTTAGTGGAATAAACTGATTACTGGTTTTTTTGTTTTT
TTTTTTTTTTTTAAAGAAAGAAGCTTCATCACAGATACTTTCCAGTTTCT
CTTTTATACTTTTTTGAAAGATTACTTTTTAGGAACATTTGGTATGATAT
GCATAAAATTATTTATCCATTTATGGGCAAAATGATACAAGTAGCATCTT
GATTGAACATCATTTACCTCAGATATTCAACCAGCAGTACGTTTTTTATG
CAGTCTCAACCCATATCCCATTTGTTACCTCTCAGAATATTGGTAAGCAG
TTATTTTCGCTTTACTCTGTATTTCTTGTGTTTTGGGCACAGGTTATTGT
ACTACTGTCAAATCGTACTTGCTATTTTTTCTGCAAGTATTTAACAGAAA
GCTTAAAATCCCCATAAAACCCCACCTTGGATAAGTGATTGTTAAATATT
GTACAAATAAAATGTATGCTATCCCCATTCCATCCCCAAGTTAAATAAAA
AAATGAATACGGTATGAAAAAAAAAAA.

An exemplary human ActRIIB mRNA sequence is set forth as:

(SEQ ID NO: 16)
GTGCGCGGGGCGGCGCCGCGGAACATGACGGCGCCCTGGGTGGCCCTCGC
CCTCCTCTGGGGATCGCTGTGCGCCGGCTCTGGGCGTGGGGAGGCTGAGA
CACGGGAGTGCATCTACTACAACGCCAACTGGGAGCTGGAGCGCACCAAC
CAGAGCGGCCTGGAGCGCTGCGAAGGCGAGCAGGACAAGCGGCTGCACTG
CTACGCCTCCTGGCGCAACAGCTCTGGCACCATCGAGCTCGTGAAGAAGG
GCTGCTGGCTAGATGACTTCAACTGCTACGATAGGCAGGAGTGTGTGGCC
ACTGAGGAGAACCCCCAGGTGTACTTCTGCTGCTGTGAAGGCAACTTCTG
CAACGAACGCTTCACTCATTTGCCAGAGGCTGGGGGCCCGGAAGTCACGT
ACGAGCCACCCCCGACAGCCCCCACCCTGCTCACGGTGCTGGCCTACTCA
CTGCTGCCCATCGGGGGCCTTTCCCTCATCGTCCTGCTGGCCTTTTGGAT
GTACCGGCATCGCAAGCCCCCCTACGGTCATGTGGACATCCATGAGGACC
CTGGGCCTCCACCACCATCCCCTCTGGTGGGCCTGAAGCCACTGCAGCTG
CTGGAGATCAAGGCTCGGGGGCGCTTTGGCTGTGTCTGGAAGGCCCAGCT
CATGAATGACTTTGTAGCTGTCAAGATCTTCCCACTCCAGGACAAGCAGT
CGTGGCAGAGTGAACGGGAGATCTTCAGCACACCTGGCATGAAGCACGAG
AACCTGCTACAGTTCATTGCTGCCGAGAAGCGAGGCTCCAACCTCGAAGT
AGAGCTGTGGCTCATCACGGCCTTCCATGACAAGGGCTCCCTCACGGATT
ACCTCAAGGGGAACATCATCACATGGAACGAACTGTGTCATGTAGCAGAG
ACGATGTCACGAGGCCTCTCATACCTGCATGAGGATGTGCCCTGGTGCCG
TGGCGAGGGCCACAAGCCGTCTATTGCCCACAGGGACTTTAAAAGTAAGA
ATGTATTGCTGAAGAGCGACCTCACAGCCGTGCTGGCTGACTTTGGCTTG
GCTGTTCGATTTGAGCCAGGGAAACCTCCAGGGGACACCCACGGACAGGT
AGGCACGAGACGGTACATGGCTCCTGAGGTGCTCGAGGGAGCCATCAACT
TCCAGAGAGATGCCTTCCTGCGCATTGACATGTATGCCATGGGGTTGGTG
CTGTGGGAGCTTGTGTCTCGCTGCAAGGCTGCAGACGGACCCGTGGATGA
GTACATGCTGCCCTTTGAGGAAGAGATTGGCCAGCACCCTTCGTTGGAGG
AGCTGCAGGAGGTGGTGGTGCACAAGAAGATGAGGCCCACCATTAAAGAT
CACTGGTTGAAACACCCGGGCCTGGCCCAGCTTTGTGTGACCATCGAGGA
GTGCTGGGACCATGATGCAGAGGCTCGCTTGTCCGCGGGCTGTGTGGAGG
AGCGGGTGTCCCTGATTCGGAGGTCGGTCAACGGCACTACCTCGGACTGT
CTCGTTTCCCTGGTGACCTCTGTCACCAATGTGGACCTGCCCCCTAAAGA
GTCAAGCATCTAAGCCCAGGACATGAGTGTCTGTCCAGACTCAGTGGATC
TGAAGAAAAAAGGAAAAAAAGTTGTGTTTTGTTTTGGAAATCCCATAAAA
CCAACAAACACATAAAATGCAGCTGCTATTTTACCTTGACTTTTTATTAT
TATTATTATAATTATTATAATTATTATTATTAATATTATTTTTTGGATTG
GATCAGTTTTTACCAGCATATTGCTCTACTGTATCACAAACAGCGGACAC
GTCAGCAGGCGTTGAGGTGCTGAGCTGTGGATGCAGAACCAGCGCCATGC
TGAAGAGCCTCAGCCACCTCCTGTCCTTTGGGATTCGTTTTTCCCGCTTT
CTCTTTGTTTGTCGTCTCAGAATCTGTGACACAAAGAAACCCATCTCCTG
TCTTAGGAAACCTAATGCTGCAAACTCTACCTAGAGGAACCTTTGAAGAC
TGTTACATAAGAACATACCTTCCTCAGAAGAGGAGTTTCCTCTGCCCTCT
GCCCTTCTCCCCTGCCTCCCTCCCTCCCCTCCTTTTATTTTGTTTTAGTG
AGCTTAAGAAACAGCAGATGTGTCTTTCACGGATCTAACGGGTGTTGTCC
TGATCGAGAAAAAAACTGGGATGAGAATGGTTTGGACTGGAGTTGGAAGG
GGAGGACGGTACTGGGGGTAGGGTTTGGAACAGAGCTACACTGGACTCGG
GCACATTCGGAGCAGCATCCTTTAGTATGGAGGCTACTTCTCAGGTAACC
AGGAATTGAGGGGAAGGACCTTGTGGAGGCCGAGCATTAACAGCAAGAGC
GGGGTTTGGAGAAAGTCTGAGATTGGGTGCAGCCCTGACTTACCTGCTGG
CCCTGACCAGTTTCTTTTCACTAACTTGGCCTTGGGCATAGGATGAAACA
TTTTTTCTGCCCTAATTTTAAAACTAGGTGAGGGTAGAATCATCACAGGT
TAGGAATACATTCTTCATAAGACACGATGCTGTAAATACCCTTAATGGAC
GAAAAGTTGAAATACTTTTGTTTCCTCTTGGAGCAGTTCAGGGAAATGCC
CACAGGGGATTGTCCTGCACAGATAGGGCAAGAGGATTTCCTGGGTGGAG
TCTGCCAAGGCCTGCCTCGCTGGGGACCCCAGAGTCCTGCACCTCTGGTT
CCGCCCCAGGTGGTGACATTACTGTCCCCGTTCTGTGGCTCGTGGACAAG
ACTTTCTCCAGACCCCTTAAAGTGGTACATATTCTAAAAAACTGTTTTTC
TATTATGCCATAACCTTGCTCTAGTCAGTGAATGTTCCTAATGCTGCTGT
TTCAACATTTGAATTCTTTTTAATTTATGAAACATGCTAAATTTTTTTTT
TCAAACAAAACACACACATCCACATATACACATGCTTCGCTATGTGGCTT
CCAAGGTTTAAATTTTGAAAAGTAAAAGAATTAAAACTTCACGACCACAG
ATCACCTCAAACCAGAAATACCTCAGAATTTTCTACTTATGTAAGGTTTA
TTATATATTTTGTTAGTTGTGTTGTCTTGTAGTAAGTATATTTTAATGTA
AGTTGGCTTTTGTGACAAGGAAGTTTAAAAGAAATAGAGAAAAAGAAAAA
AGTTTGCATCTTCTAGGGAGTGCTACCATTTTTGTTTGATAACGCCCCCT
TGTAAATAATTGTCATCAACTGTAGGTTGGCTGTCTGGGCCAAGTCTGGG
CATTTATCAGTCTTGTTTGTGAAGGCTTTTCCTTCTGGTTTCTTTAGATC
ATTTTATTTAAAAACAGTGCATCTCTTCATCGTGAGGGTAGGCAAGGCGG
GGGCCGTGGGGAGAGGTTGACCTGGGTGAGAACTGAAGAGGCCGCCTCCT
CTTGGGTTGTTTGGAGCTTCACATGTAATTCACATGTAACATGTAACTTG
ATCGGTCAGTGTTCAGAATGACAAGTAACCCCGCTTAAACTTGGTAGAAG
GATGGCCCTTAGACCTGAATGGGGTGATTTTACTTGGGATTTAACTTCTT
CAGCAAATTAACAGCAACGTTGGAAGAGATCTGTGGCGCCTCTGTGAAGC
ACACCGTGACTCAGGCCAGTCTTTTAGTGCAGCGTGTCTGGGAGTGAAGG
GTTTTGCCCTTGCTGGTCTTGGAGTCCACAGTGTGAGGGGCACTGCACAT
GCCTGGGCATCTACCTAGTGTGCTATGTTCAGTGTCTGGGGCTTACTGCC
CCGGGGTCCTTTCCTCTGGGTGTTGGGGCACAGGGTGCTATGGGAGGCCC
ATTTGCTTCCCTCTCGGAGCTCAGTTTTTGCTTCATGGGTCAAAATGTGG
GCTGGCCAAGTGGTTACAGGAACAGGGTTTCGGTAAGCTATGTTGTCTTT
TTTTTTTTTTTTTTTTTTTTTTTTAATGGTTTGATTTTGTGCTGTGGTAT
TTTTTTTCCCTTAGAATAATTTTTAATGGCAAAACAGGCCTTACAGCAGT
TGCTTTTCTTTACCATTTATTTCTTTAAGAAGCTTTAAAATATTTATTGA
AAAGTGCCATATCTAATTTCTTTAGCTTTCGCCTCAGGCAGTGCAGGCAT
CTTTACTTTTCATCCTCAGAAGAAACAAACGACTAACAAATGTAGCAAAT
TTACTGCAGGAATAGTTAGGTCATGATACTACCTGAACACTAAACCCCAG
CCTCTTTGTTTGGTTTTAGTTCCTCTGGGTGGTTTTTCTTTTGTGTGCTG
GCTTGATTCTTGTGAGAAGTTTTGACCTGGCCAAGGGAGGGTTGAGCCAT
GGTTCTGGTGTGGGACTTTGCGGTCAAGACACAGTACAGACAGGTCAGGC
CTGCGTGCCTTTTCTCTGGGTGGCCTCCCCGTTAGGCCCACCGTACGCTC
AGCCACTATAGTGTCCCTGTGGGGCCTTGCCATCAGATTGTGTGTCAGGA
GATGGTACCTTTTTGGTGTGGCTGGGGAGGAGTGTGGTCCATGCCAGTTC
TTTGGGCTTCAGGCCACTCTTCCCCTCATGCTGTGGTGTAAAGTGCACCC
ATCAGGTGGTATATCTGGTTCTGATGGCAAGAAGAAGGTGGGGGATCTCC
TTATAGGGCATGGGTCTAGGAGCACAGATGGGCCTTTTGCCCCGGGTAAA
TGCTTGTCTGTTTGCTGTCATGTGTTCTTTGAGGAGTGAGCCATCTCGAG
CCCTGCTTTGAATTTACTGGGTCATAGAGCCTCTGCCTGTGCTCTTTTCC
ATAATGACTTCATGTGACATGCACTTTTGGTGGGCTCAGATAATTGGTTT
CTTTTTGTTTTTGACCTCAGGCTCTGTGGCAGACTGGGGAAAATGGGGCC
TGGCATCATTTTCCCTGTCAATGGGAGGGGCTGTTCCATGCAGGGTGGGA
GGGGACCAAGTTAGCAGAGAGTAGCCAAGGATCCTTGCTTCTTCCTTTCT
AGTGTGCTGTCATCCAAGCAGGCTCCTGGCTGTAGGGATGGGCCTTGGGG
AAGAATCTTCTTTGAAAGCATCTATGATAACTGAGAAGTCATCCCTAGTT
GGAGAAATCCAGTAATGAGCAGAAGGAGGAAGCAAGTGAGGACAGAGGCC
ATTGTATTACAGTGTCACGCAGAGGGCCCTCAATGATGGGGCATTGGGGA
AGGCTGTAGACATAGTCATCAGAACATCCTGGCCTGGCATAAGCTGGGTT
TTCTCCTGGGACCATTGGTCCTCAGCAGGAGTTCTTTGCATGAGTTGCTC
AGGGGCAAGGGCTGCAAGTGGGCTGTGCTTAGGAGAAAGTGACACCTGGC
AGTGAGGGAAGATGGTGAGCATTATTAGCCTTTGTTGTCCAGCATGGCCT
TCTTGTCCTGTCTGCTCTGGAGAGGAGCCTGTGGGACCAGTCCTGCCTGG
GGAGGGCATACCCACACGTGCCAGCTGATTCTGACTCTGAATACATCATG
TCCGGACTTGGGGGTGTTTCTGCAGAAAAAGGAGGTTGTTTTTCAGCCTT
GAACATCTTCAGGAGGATAGAGACTCTTGCTCACATATTCTTAGCAAAGG
GAAGGGTCTCTCATCTCCAGGCCACAGAGATAGTTCTTCCATTGCCCTAA
GAGGCTAGGCTAACCCTCTTGACATAACTTAGACAGCAAAGCACTTCATC
CTGTAGTTGGGCTCTGTCACCTTTCTCTTCAGTTGGCCACATTCTCGTTT
CCTCCATCCTGCTATGCTTTGTGTGCTCGGGCTGTGTGTGGGGTTTTTCC
CTGGTGGAAGGAAGCCCAGCTGTGTATTGAATGTCCTTCATGTGTTGTGT
GTGGCTCAGAAAGCCTGTCACTTGGCCCCTGTGCTCTGAGCCGTGAGGGT
GGGGAGGTGGCTGTTCCATTAAAGTGGGAGTATTGGATGGCCCTCTTGAA
ACTAGAATTTTGCCTTTTTTAGTATGCAGTATAAAGTTTCCAGCATCTAT
TGGTAACACAAAGATTTGCTGGTTTTTAAAATAATACAGTAAGCATAAGT
ATGTAAGTTTTTAGAATTGGTACTAGAAGTTGGACAGCTAGTTATTCTCG
AGAACTTTATTTCACTAGAAAAATATACTAATTGGAAAGCAGTTTCCAGG
AGTTAACTCAGTTTAATTTTCAGTCTCAGTTATTTTAGCCTGTTGAGTTT
TTGATGGCACACCTTTGGAGAGATGGCCACGCCTGATTCCCATTTCAGGG
GCATCAGACCATACCTTTTTAAGAAGCTCCGTGAATCTAGTCATCTACCC
TTCATCCTGGGCGAACAGCCAAAAAGAGAAGGGGACAAGGTGTCTTTTTC
TCCTTCTCACTGGGGTGACATGAATTCTTTTAGTTAATGGCTGTTTGCAA
ATTCTAAACTAATGAAATACTTAGCAGCTAACATGTTCAATCTAGTAATG
ATGAGTTTAAATCTCAATTGACAGTAATGTTTTAGATAAACAGGCCCAGT
AATTCAGTTGATGAACTGTATATCTTCTCAGTCTAGATTTGTAAATGTTT
AATGAATTCAGGGTTATAAGCATAGTTCTTTAAGTAAGATTCCAGATAGT
TGATTTGCAACCAGCAGTCTACCTATGAATGTATCCCAAACCTTTAGAAG
ATTGGAAAAGATTTTTGAAATAATGATTTAGTTTTGTAGGAAAAACACCC
CCTTGAAAATTAATTCGGTTGACCCAGTAACATTTTTTAAAACAATTGGT
GGCTCCAAAAGGCCTGCCAACAAAGAAAAGTCCAAATTATCTAGTGGGAC
ATTTTGAATGTTTTATGTTTATTTTGGGTCCACTGTAAACTTTGGTTCAA
AAAAGAATTTGAATTTAAAGAATTTACCATTATTTAAATTATTACCAAGT
TTTTACATTTTCATGATGGTATTTTCCAGGTATGAATGAAACATGACTTT
TTGATTGTGGTACTTCCTGTATCCCCTGTAGTGCCAAAACCAGTGATACT
TTATTTGCTCCTATGGCAGCTCATAGAGGTAACCGAAGTGATTTTTCCTC
AGTAATTGAAACACATATTCTCTAAATGCCAATGTGTGGTGATGGGCCCT
GCACTGCCTTCATTTCTCTAGGGCAGTGTCTTTGGATTGTCTAGGGCCTA
GGTAATTCTGAGAACTACTGTAAACCAACCACAGGGCACTAAAGCAATGT
ACACACCACTCTTTGTGTGTATGGAAGGGGTTATATAAACCTGGGCTATG
CTGGACATCTACAGAAGAGTATTACATTCACTTGCAAAGTTTACATTTTT
GAGCTCACAGTTATGAAAAATATGACCCACAAGTTTTTCAGGCAGGTGAG
GATGGGTCTTCTTGCAAATGCATGAGTTCTGTCTTGAGTCCTGGGAACTT
CTCTGTTGGTTGAGTGTGGGCTCATTCCCTGACTCTCCTAATCATGTTTG
CGTCAGAATGTTAGCATTGTAAATAAAAGAATAGGTTGTATAATAGATAC
ACAACACTTGAAACTTTACTTTAAAAAAATCGATAGTTCTACATATATAT
TTAGTTATATCACTTGACAGATTTCTTCTACACAGTGTGGAGATTGTTTT
ATACCACAGATTATTTTTATAAAGTTAGTGAATTTGAATGATTTTGTAAT
CAGAGCTAATGAGCTTTACCTTTCAAGAGAAACGTACACTGGAGCATGAG
TGGTGTGGAACTTTTACTTAGTGTTTATATGGATTCTTGTGATACACTGG
CAGACTGGAGTCAATTTGCGGGTCTTTTTTGGCCAAAACTCCACTTGTGG
TTGTGTAGGACAGTGATATTCAGCTCAGCTTCTTGTGGATTGGGAGGAGA
GAGGGCCTGCAATGTGTTTTACATTGGTGCTTCCTCCTGAGATTTCTGTT
GAACAAAGGGTTCTGAGGTCAAAAATTAGTTTGTAAGCCTTTGCCATAGG
ACATAGTCATGTGAGAGTGTTTGGGGGAACAGAAATTGTATAGGGGTGCC
TATTGGGGTGGGATGGGACTCGAATAAGATTCAGGTACAAAAACTTTGAA
ATGAGAATCTGGTGGTTTGAGTAATCCACCAGACTGAATTATCTAAGATC
ACATTATCCAGGTTGGGGGGCAGAATTACCCAGTTAAGTAATTGTTCAGA
AAAGTGGGGAGGGTGGCATGTGGATGCAGTGATCCAATTAAATGGAGAGC
TGCCAGGCACATTTTGTCCTCTCTGGTCAGTGAGAATGGTTGGGTTGGCT
CGCTGCTTCAATCTGTGGAATCAGCCAGGAGCCCAGTGAGGAAGCTCAGA
ACCCCAGTAACAGCAGAGCATCTTTCAGATAGCTCCAGAGTTTTCCTGCT
TTTCTGAGGAAGCTCAGCATCACTGCCACAATACGGAAAGTGGTCTTCAT
TTTAGCCTATTTATTTTTAGGCAGAGAGTGGATGGTTATTTGTGTGGGAC
TTTTGGTGGCGATATATAATGAATAATTAAGTTAATTTCTGGTATGCATA
ATGGCCAGTCCTGAGGCCCAGCTGAAGACCTGTCCCCCAGACCCTGCCCG
CTGGCTTCAGGCTGCTGCTTCTAGACAGAGGTGCACTGGACGGGATAGTT
TTATCAAGAGAATCCCTAATGTGTCATTTTAAACCAGCTGTGCTTTTTAT
TCATTCTGGTTGAGCGTATAGGTTTACACTTTACCCTTTTTATACTTGGA
ATAAATTTAGTTCCAGCAGATCTAGTAGCACTCCAGAAACCAACCCCATC
TGTTCCCCATAAAAAGAACATTTTCTCTGCTCTCCAGCCACGTGTCTTGG
AATGTAATTCTGTTGTGCCTTTGTTTTTATCACTCTCTTCGCCCCAAAAG
CAACTGCTGTAAGCTTTTTTCTACTTGTCTTTTCTAGTCCCCAACCTCTA
CCTTTTTCCTTTTTCCCAGCCCTAATTTCTGGATGCACTTCTGTGATCCA
GGTATTTTAAGAACCAGTTACCTCAGACCTCATGTTGAACAGTGTCGCCA
TCTGGGTCCTCTTGATACTGCAGACTTTTAACGTACACATGCAGGAACCC
TGCTGAGCGTGGGCACTTGTTTTAAAGCAAAACTCTTCCCAAGGACTGAA
GAAAGGGCTTCTGGCAAGCTCGTCATGGCATTGTGGTGGGATGGGTCTAG
AGTGTCATCTGAATGGTGCTTCCTGTGTTCCTCTTTGAATTCTGCCATTT
TCAGTATTCTTGTGTGTCTGAATAGGCAAAGCGATTTAATTGGCTGGTCT
TGCACGCAAATTAGTTCCAAAGATAAGCTCTTTGTAACACATTTCCAGTC
GCTAATGCTCAAATGTAGAACATTCCTTTAAATGGCAGGATAAAAAACCC
ACTATCCACCATAGTGCATTTTGGGAAGATGTCTGTAGCATATGTTGCTG
TGAAATTAGGCCTTGTGGGATATGGCTGTTTGTCATTTTGATGTATTTTA
AATAAATATATATATTTTTTAAAGAGCCTTTTTTACCAGTTCAAAAAGTT
TAATTAACCAGCAGTCACCGCATCTGAATTTTTGTCTCTGGGGCATAGAT
GGCAGACCAAGATTAAAAGTGGTAACTCAGCTATACGAGCATGGGCTACC
TTCCTGGGCTCTCCTGCAGTCCTGTAGACCTGCTGTTCCGCAGACCATGG
GACACAAGGTCAGTGTGTTCCCAGTGAGGGTCCCAAGTCAGTCATCTTAA
GTGTTTGTTCTCTGCCCCATTCAGTGGACTGTTGACTTCAGTCCCTGCAA
GTGCTTTAGCCCGAGTGGGGTTTTCTCAGAGCACTGCCACGAGTTAAGTG
TGTGTTTAGCCAAATAATTTCTCCGTAAGGGAAAAATGCAGTCACCCAAA
TTTTACCAACAATGACAGAGATGAGAGTAGAAAAGATTAGGCAACATCTG
AGTTTTAACTTGAAAAGTGTCCAAGTCATCATGAAAGGCCGACTGGGAGC
AAGTGATTATTAGAGATTCTTCAGGAGACCTCATCTGAAAATGTTAAGAC
TGCCAGTGAGGGAAGGAATTGTTAAAATGCCAGCGGCTTTTTTTTCCTCT
TTTTTTCTGTAATTCTGTAAAAATGCAGAGAAAGTTGAGTGGTACTTCAG
AATTGAGGGAGAGGGTTACCGCAGAGTAGAAATATATTTCTAGATTTCAG
TTCCACACCACAAATCCACAACAATGCCATTTTTCAACTGTACAAAAATC
TGCTTATGAACTGGACATGATCTTAATGGTAGTGTCAAAGGCCAAGTTTT
TCACCTGTTAATATTTTTCCACATTTGTCCTTGAATCTGAATAACTTTAT
ACAGTACTGTAAATTTAACTTACATCGAGTTTGTTGTCAATTCTTATGAA
AAGAGCTTTCTGCATGTAACACATACGGTTAAAGAACACAGCAAAGGACA
AAATTTGCAGGAACAGTTTTGGAACCAACAGAAAATGTCACCTTTTATTT
GCCATCTTATATATATCTATCAGTTTTACCAGCTACTTCTAAATTTGTAC
ATTATTTGTAAGGGAAAGAAGGAAAACCCTAAGACTTGTCTAACTTAGTG
GAGAATGTGTGTGTTGGGCTTAGGATGGATAGCTAAGTCTTATTGAGCTG
TGTTACCTAACTTGTATATAAAAATTGTAATTAAAAGTTTGGGTTCACCT
GTTTCTCACAGTTTAAAATGATGAGTAATTGCAAACTCTGGAAATGTGAC
TAGTATATGATTTAAGGCTGTAGAAGCAAGGAAGCTCTTTCAAGTGCTAA
AACTAAAGACTTCTAGTTTTTGGCTCAAATAAGTACTGTTTGTATACCAG
GATATGTGAGATGTAAATGTAGTAGGTCACTTTTCACCCTTGTAGCTATA
AAATAAAAATTTTGTAGAACAGAAATAGCTTGTACTACTGAATTAACAAA
AGTTATACTAAAGTATCATGTTTAAAAAAAATATATATATATATACAGAG
TTAAGCTTGTTGCTGTTACCCTGTCTGGATTTGAAAAGTGTGCTGATTTA
TATATATATATTACACACACACACACACACACACACACACACACACACAC
ACACACACACACACACACACACACACACACACACATACACCTAAAATGGC
CTAAAGCAGACATCCATGTAATTACAGTTGCAAAATGAAAACATTTTGGA
AAGAACATTGTATCATAGTTCATTCATTTGCAGTGGATCTTTGTTCCTTT
TTACTGTGGTAATTTTAGAAATGAGTGTCAAGTTTGAAATTAGATCTGCT
AAGTTGGGGTTTTGCTGCTTGAACTCTGCACTGGGTCCTCAAATAAACCG
ATGTGAATGTAGTTTTTTCCCCCTGTGTGAAGAAGCAGTTACACCCCAAC
AATAGGAGGAAAAATCTAGAACTATTTCAAGTTTTATCTTTTTGTATATG
AAAATAAAATAATAATAAAACAA.

An exemplary ALK2 mRNA sequence is set forth as one of:

(SEQ ID NO: 17)
GAAGAGATGTGGGCCTCTGGGGCCGCTGGATTCAGTAACTTCCGTCGGGT
TCTAGACTGGCTCGGCTCTGTCCAGTTTGTGCCAGATAGTCTCCCACCCC
CTCCCCACCCCTCCTTTCCCCTGGAGATTTGAACGCTGCTTGCATGGGAG
AAAAGCTACTTAGAGAAGAAAACGTTCCACTTAGTAACAGAAGAAAAGTC
TTGGTTAAAAAGTTGTCATGAATTTGGCTTTTGGAGAGAGGCAGCAAGCC
TGGAGCATTGGTAAGCGTCACACTGCCAAAGTGAGAGCTGCTGGAGAACT
CATAATCCCAGGAACGCCTCTTCTACTCTCCGAGTACCCCAGTGACCAGA
GTGAGAGAAGCTCTGAACGAGGGCACGCGGCTTGAAGGACTGTGGGCAGA
TGTGACCAAGAGCCTGCATTAAGTTGTACAATGGTAGATGGAGTGATGAT
TCTTCCTGTGCTTATCATGATTGCTCTCCCCTCCCCTAGTATGGAAGATG
AGAAGCCCAAGGTCAACCCCAAACTCTACATGTGTGTGTGTGAAGGTCTC
TCCTGCGGTAATGAGGACCACTGTGAAGGCCAGCAGTGCTTTTCCTCACT
GAGCATCAACGATGGCTTCCACGTCTACCAGAAAGGCTGCTTCCAGGTTT
ATGAGCAGGGAAAGATGACCTGTAAGACCCCGCCGTCCCCTGGCCAAGCC
GTGGAGTGCTGCCAAGGGGACTGGTGTAACAGGAACATCACGGCCCAGCT
GCCCACTAAAGGAAAATCCTTCCCTGGAACACAGAATTTCCACTTGGAGG
TTGGCCTCATTATTCTCTCTGTAGTGTTCGCAGTATGTCTTTTAGCCTGC
CTGCTGGGAGTTGCTCTCCGAAAATTTAAAAGGCGCAACCAAGAACGCCT
CAATCCCCGAGACGTGGAGTATGGCACTATCGAAGGGCTCATCACCACCA
ATGTTGGAGACAGCACTTTAGCAGATTTATTGGATCATTCGTGTACATCA
GGAAGTGGCTCTGGTCTTCCTTTTCTGGTACAAAGAACAGTGGCTCGCCA
GATTACACTGTTGGAGTGTGTCGGGAAAGGCAGGTATGGTGAGGTGTGGA
GGGGCAGCTGGCAAGGGGAGAATGTTGCCGTGAAGATCTTCTCCTCCCGT
GATGAGAAGTCATGGTTCAGGGAAACGGAATTGTACAACACTGTGATGCT
GAGGCATGAAAATATCTTAGGTTTCATTGCTTCAGACATGACATCAAGAC
ACTCCAGTACCCAGCTGTGGTTAATTACACATTATCATGAAATGGGATCG
TTGTACGACTATCTTCAGCTTACTACTCTGGATACAGTTAGCTGCCTTCG
AATAGTGCTGTCCATAGCTAGTGGTCTTGCACATTTGCACATAGAGATAT
TTGGGACCCAAGGGAAACCAGCCATTGCCCATCGAGATTTAAAGAGCAAA
AATATTCTGGTTAAGAAGAATGGACAGTGTTGCATAGCAGATTTGGGCCT
GGCAGTCATGCATTCCCAGAGCACCAATCAGCTTGATGTGGGGAACAATC
CCCGTGTGGGCACCAAGCGCTACATGGCCCCCGAAGTTCTAGATGAAACC
ATCCAGGTGGATTGTTTCGATTCTTATAAAAGGGTCGATATTTGGGCCTT
TGGACTTGTTTTGTGGGAAGTGGCCAGGCGGATGGTGAGCAATGGTATAG
TGGAGGATTACAAGCCACCGTTCTACGATGTGGTTCCCAATGACCCAAGT
TTTGAAGATATGAGGAAGGTAGTCTGTGTGGATCAACAAAGGCCAAACAT
ACCCAACAGATGGTTCTCAGACCCGACATTAACCTCTCTGGCCAAGCTAA
TGAAAGAATGCTGGTATCAAAATCCATCCGCAAGACTCACAGCACTGCGT
ATCAAAAAGACTTTGACCAAAATTGATAATTCCCTCGACAAATTGAAAAC
TGACTGTTGACATTTTCATAGTGTCAAGAAGGAAGATTTGACGTTGTTGT
CATTGTCCAGCTGGGACCTAATGCTGGCCTGACTGGTTGTCAGAATGGAA
TCCATCTGTCTCCCTCCCCAAATGGCTGCTTTGACAAGGCAGACGTCGTA
CCCAGCCATGTGTTGGGGAGACATCAAAACCACCCTAACCTCGCTCGATG
ACTGTGAACTGGGCATTTCACGAACTGTTCACACTGCAGAGACTAATGTT
GGACAGACACTGTTGCAAAGGTAGGGACTGGAGGAACACAGAGAAATCCT
AAAAGAGATCTGGGCATTAAGTCAGTGGCTTTGCATAGCTTTCACAAGTC
TCCTAGACACTCCCCACGGGAAACTCAAGGAGGTGGTGAATTTTTAATCA
GCAATATTGCCTGTGCTTCTCTTCTTTATTGCACTAGGAATTCTTTGCAT
TCCTTACTTGCACTGTTACTCTTAATTTTAAAGACCCAACTTGCCAAAAT
GTTGGCTGCGTACTCCACTGGTCTGTCTTTGGATAATAGGAATTCAATTT
GGCAAAACAAAATGTAATGTCAGACTTTGCTGCATTTTACACATGTGCTG
ATGTTTACAATGATGCCGAACATTAGGAATTGTTTATACACAACTTTGCA
AATTATTTATTACTTGTGCACTTAGTAGTTTTTACAAAACTGCTTTGTGC
ATATGTTAAAGCTTATTTTTATGTGGTCTTATGATTTTATTACAGAAATG
TTTTTAACACTATACTCTAAAATGGACATTTTCTTTTATTATCAGTTAAA
ATCACATTTTAAGTGCTTCACATTTGTATGTGTGTAGACTGTAACTTTTT
TTCAGTTCATATGCAGAACGTATTTAGCCATTACCCACGTGACACCACCG
AATATATTACTGATTTAGAAGCAAAGATTTCAGTAGAATTTTAGTCCTGA
ACGCTACGGGGAAAATGCATTTTCTTCAGAATTATCCATTACGTGCATTT
AAACTCTGCCAGAAAAAAATAACTATTTTGTTTTAATCTACTTTTTGTAT
TTAGTAGTTATTTGTATAAATTAAATAAACTGTTTTCAAGTCAAAAAAAA
AAAAAAAAAAAA
or
(SEQ ID NO: 18)
CTGCAGCGCCCGGCTGCCTCGCACTCCGCCTCCCCCGGCTCAGCCCCCGG
CCGCGGCGGGACCCGAGCCTGGAGCATTGGTAAGCGTCACACTGCCAAAG
TGAGAGCTGCTGGAGAACTCATAATCCCAGGAACGCCTCTTCTACTCTCC
GAGTACCCCAGTGACCAGAGTGAGAGAAGCTCTGAACGAGGGCACGCGGC
TTGAAGGACTGTGGGCAGATGTGACCAAGAGCCTGCATTAAGTTGTACAA
TGGTAGATGGAGTGATGATTCTTCCTGTGCTTATCATGATTGCTCTCCCC
TCCCCTAGTATGGAAGATGAGAAGCCCAAGGTCAACCCCAAACTCTACAT
GTGTGTGTGTGAAGGTCTCTCCTGCGGTAATGAGGACCACTGTGAAGGCC
AGCAGTGCTTTTCCTCACTGAGCATCAACGATGGCTTCCACGTCTACCAG
AAAGGCTGCTTCCAGGTTTATGAGCAGGGAAAGATGACCTGTAAGACCCC
GCCGTCCCCTGGCCAAGCCGTGGAGTGCTGCCAAGGGGACTGGTGTAACA
GGAACATCACGGCCCAGCTGCCCACTAAAGGAAAATCCTTCCCTGGAACA
CAGAATTTCCACTTGGAGGTTGGCCTCATTATTCTCTCTGTAGTGTTCGC
AGTATGTCTTTTAGCCTGCCTGCTGGGAGTTGCTCTCCGAAAATTTAAAA
GGCGCAACCAAGAACGCCTCAATCCCCGAGACGTGGAGTATGGCACTATC
GAAGGGCTCATCACCACCAATGTTGGAGACAGCACTTTAGCAGATTTATT
GGATCATTCGTGTACATCAGGAAGTGGCTCTGGTCTTCCTTTTCTGGTAC
AAAGAACAGTGGCTCGCCAGATTACACTGTTGGAGTGTGTCGGGAAAGGC
AGGTATGGTGAGGTGTGGAGGGGCAGCTGGCAAGGGGAGAATGTTGCCGT
GAAGATCTTCTCCTCCCGTGATGAGAAGTCATGGTTCAGGGAAACGGAAT
TGTACAACACTGTGATGCTGAGGCATGAAAATATCTTAGGTTTCATTGCT
TCAGACATGACATCAAGACACTCCAGTACCCAGCTGTGGTTAATTACACA
TTATCATGAAATGGGATCGTTGTACGACTATCTTCAGCTTACTACTCTGG
ATACAGTTAGCTGCCTTCGAATAGTGCTGTCCATAGCTAGTGGTCTTGCA
CATTTGCACATAGAGATATTTGGGACCCAAGGGAAACCAGCCATTGCCCA
TCGAGATTTAAAGAGCAAAAATATTCTGGTTAAGAAGAATGGACAGTGTT
GCATAGCAGATTTGGGCCTGGCAGTCATGCATTCCCAGAGCACCAATCAG
CTTGATGTGGGGAACAATCCCCGTGTGGGCACCAAGCGCTACATGGCCCC
CGAAGTTCTAGATGAAACCATCCAGGTGGATTGTTTCGATTCTTATAAAA
GGGTCGATATTTGGGCCTTTGGACTTGTTTTGTGGGAAGTGGCCAGGCGG
ATGGTGAGCAATGGTATAGTGGAGGATTACAAGCCACCGTTCTACGATGT
GGTTCCCAATGACCCAAGTTTTGAAGATATGAGGAAGGTAGTCTGTGTGG
ATCAACAAAGGCCAAACATACCCAACAGATGGTTCTCAGACCCGACATTA
ACCTCTCTGGCCAAGCTAATGAAAGAATGCTGGTATCAAAATCCATCCGC
AAGACTCACAGCACTGCGTATCAAAAAGACTTTGACCAAAATTGATAATT
CCCTCGACAAATTGAAAACTGACTGTTGACATTTTCATAGTGTCAAGAAG
GAAGATTTGACGTTGTTGTCATTGTCCAGCTGGGACCTAATGCTGGCCTG
ACTGGTTGTCAGAATGGAATCCATCTGTCTCCCTCCCCAAATGGCTGCTT
TGACAAGGCAGACGTCGTACCCAGCCATGTGTTGGGGAGACATCAAAACC
ACCCTAACCTCGCTCGATGACTGTGAACTGGGCATTTCACGAACTGTTCA
CACTGCAGAGACTAATGTTGGACAGACACTGTTGCAAAGGTAGGGACTGG
AGGAACACAGAGAAATCCTAAAAGAGATCTGGGCATTAAGTCAGTGGCTT
TGCATAGCTTTCACAAGTCTCCTAGACACTCCCCACGGGAAACTCAAGGA
GGTGGTGAATTTTTAATCAGCAATATTGCCTGTGCTTCTCTTCTTTATTG
CACTAGGAATTCTTTGCATTCCTTACTTGCACTGTTACTCTTAATTTTAA
AGACCCAACTTGCCAAAATGTTGGCTGCGTACTCCACTGGTCTGTCTTTG
GATAATAGGAATTCAATTTGGCAAAACAAAATGTAATGTCAGACTTTGCT
GCATTTTACACATGTGCTGATGTTTACAATGATGCCGAACATTAGGAATT
GTTTATACACAACTTTGCAAATTATTTATTACTTGTGCACTTAGTAGTTT
TTACAAAACTGCTTTGTGCATATGTTAAAGCTTATTTTTATGTGGTCTTA
TGATTTTATTACAGAAATGTTTTTAACACTATACTCTAAAATGGACATTT
TCTTTTATTATCAGTTAAAATCACATTTTAAGTGCTTCACATTTGTATGT
GTGTAGACTGTAACTTTTTTTCAGTTCATATGCAGAACGTATTTAGCCAT
TACCCACGTGACACCACCGAATATATTACTGATTTAGAAGCAAAGATTTC
AGTAGAATTTTAGTCCTGAACGCTACGGGGAAAATGCATTTTCTTCAGAA
TTATCCATTACGTGCATTTAAACTCTGCCAGAAAAAAATAACTATTTTGT
TTTAATCTACTTTTTGTATTTAGTAGTTATTTGTATAAATTAAATAAACT
GTTTTCAAGTCAAAAAAAAAAAAAAAAAAAA.

An exemplary ALK4 mRNA sequence is set forth as one of:

(SEQ ID NO: 19)
GGGGAGGCGCCGGGGGCGCGCGCGCGCGCGCTGGGCGCTGCTGGGCTGCG
GCGGCGGCGGCGGCGGCGGTGGTTACTATGGCGGAGTCGGCCGGAGCCTC
CTCCTTCTTCCCCCTTGTTGTCCTCCTGCTCGCCGGCAGCGGCGGGTCCG
GGCCCCGGGGGGTCCAGGCTCTGCTGTGTGCGTGCACCAGCTGCCTCCAG
GCCAACTACACGTGTGAGACAGATGGGGCCTGCATGGTTTCCATTTTCAA
TCTGGATGGGATGGAGCACCATGTGCGCACCTGCATCCCCAAAGTGGAGC
TGGTCCCTGCCGGGAAGCCCTTCTACTGCCTGAGCTCGGAGGACCTGCGC
AACACCCACTGCTGCTACACTGACTACTGCAACAGGATCGACTTGAGGGT
GCCCAGTGGTCACCTCAAGGAGCCTGAGCACCCGTCCATGTGGGGCCCGG
TGGAGCTGGTAGGCATCATCGCCGGCCCGGTGTTCCTCCTGTTCCTCATC
ATCATCATTGTTTTCCTTGTCATTAACTATCATCAGCGTGTCTATCACAA
CCGCCAGAGACTGGACATGGAAGATCCCTCATGTGAGATGTGTCTCTCCA
AAGACAAGACGCTCCAGGATCTTGTCTACGATCTCTCCACCTCAGGGTCT
GGCTCAGGGTTACCCCTCTTTGTCCAGCGCACAGTGGCCCGAACCATCGT
TTTACAAGAGATTATTGGCAAGGGTCGGTTTGGGGAAGTATGGCGGGGCC
GCTGGAGGGGTGGTGATGTGGCTGTGAAAATATTCTCTTCTCGTGAAGAA
CGGTCTTGGTTCAGGGAAGCAGAGATATACCAGACGGTCATGCTGCGCCA
TGAAAACATCCTTGGATTTATTGCTGCTGACAATAAAGATAATGGCACCT
GGACACAGCTGTGGCTTGTTTCTGACTATCATGAGCACGGGTCCCTGTTT
GATTATCTGAACCGGTACACAGTGACAATTGAGGGGATGATTAAGCTGGC
CTTGTCTGCTGCTAGTGGGCTGGCACACCTGCACATGGAGATCGTGGGCA
CCCAAGGGAAGCCTGGAATTGCTCATCGAGACTTAAAGTCAAAGAACATT
CTGGTGAAGAAAAATGGCATGTGTGCCATAGCAGACCTGGGCCTGGCTGT
CCGTCATGATGCAGTCACTGACACCATTGACATTGCCCCGAATCAGAGGG
TGGGGACCAAACGATACATGGCCCCTGAAGTACTTGATGAAACCATTAAT
ATGAAACACTTTGACTCCTTTAAATGTGCTGATATTTATGCCCTCGGGCT
TGTATATTGGGAGATTGCTCGAAGATGCAATTCTGGAGGAGTCCATGAAG
AATATCAGCTGCCATATTACGACTTAGTGCCCTCTGACCCTTCCATTGAG
GAAATGCGAAAGGTTGTATGTGATCAGAAGCTGCGTCCCAACATCCCCAA
CTGGTGGCAGAGTTATGAGGCACTGCGGGTGATGGGGAAGATGATGCGAG
AGTGTTGGTATGCCAACGGCGCAGCCCGCCTGACGGCCCTGCGCATCAAG
AAGACCCTCTCCCAGCTCAGCGTGCAGGAAGACGTGAAGATCTAACTGCT
CCCTCTCTCCACACGGAGCTCCTGGCAGCGAGAACTACGCACAGCTGCCG
CGTTGAGCGTACGATGGAGGCCTACCTCTCGTTTCTGCCCAGCCCTCTGT
GGCCAGGAGCCCTGGCCCGCAAGAGGGACAGAGCCCGGGAGAGACTCGCT
CACTCCCATGTTGGGTTTGAGACAGACACCTTTTCTATTTACCTCCTAAT
GGCATGGAGACTCTGAGAGCGAATTGTGTGGAGAACTCAGTGCCACACCT
CGAACTGGTTGTAGTGGGAAGTCCCGCGAAACCCGGTGCATCTGGCACGT
GGCCAGGAGCCATGACAGGGGCGCTTGGGAGGGGCCGGAGGAACCGAGGT
GTTGCCAGTGCTAAGCTGCCCTGAGGGTTTCCTTCGGGGACCAGCCCACA
GCACACCAAGGTGGCCCGGAAGAACCAGAAGTGCAGCCCCTCTCACAGGC
AGCTCTGAGCCGCGCTTTCCCCTCCTCCCTGGGATGGACGCTGCCGGGAG
ACTGCCAGTGGAGACGGAATCTGCCGCTTTGTCTGTCCAGCCGTGTGTGC
ATGTGCCGAGGTGCGTCCCCCGTTGTGCCTGGTTCGTGCCATGCCCTTAC
ACGTGCGTGTGAGTGTGTGTGTGTGTCTGTAGGTGCGCACTTACCTGCTT
GAGCTTTCTGTGCATGTGCAGGTCGGGGGTGTGGTCGTCATGCTGTCCGT
GCTTGCTGGTGCCTCTTTTCAGTAGTGAGCAGCATCTAGTTTCCCTGGTG
CCCTTCCCTGGAGGTCTCTCCCTCCCCCAGAGCCCCTCATGCCACAGTGG
TACTCTGTGTCTGGCAGGCTACTCTGCCCACCCCAGCATCAGCACAGCTC
TCCTCCTCCATCTCAGACTGTGGAACCAAAGCTGGCCCAGTTGTCCATGA
CAAAAGAGGCTTTTGGGCCAAAATGTGAGGGTGGTGGGTGGGATGGGCAG
GGAAGGAATCCTGGTGGAAGTCTTGGGTGTTAGTGTCAGCCATGGGAAAT
GAGCCAGCCCAAGGGCATCATCCTCAGCAGCATCGAGGAAGGGCCGAGGA
ATGTGAAGCCAGATCTCGGGACTCAGATTGGAATGTTACATCTGTCTTTC
ATCTCCCAGATCCTGGAAACAGCAGTGTATATTTTTGGTGGTGGTGGGTT
TGGGGTGGGGAAGGGAAGGGCGGGCAAGGAGTGGGGAGGGAGTCTGGGGT
GGGAGGGAGGCATCTGCATGGGTCTTCTTTTACTGGACTGTCTGATCAGG
GTGGAGGGAAGGTGAGAGGTTTGCATCCACTTCAGGAGCCCTACTGAAGG
GAACAGCCTGAGCCGAACATGTTATTTAACCTGAGTATAGTATTTAACGA
AGCCTAGAAGCACGGCTGTGGGTGGTGATTTGGTCAGCATATCTTAGGTA
TATAATAACTTTGAAGCCATAACTTTTAACTGGAGTGGTTTGATTTCTTT
TTTTAATTTTATTGGGAGGGTTTGGATTTTAACTTTTTTTAATGTTGTTA
AATATTAAGTTTTTGTAAAAGGAAAACCATCTCTGTGATTACCTCTCAAT
CTATTTGTTTTTAAAGAAATCCCTAAAAAAAAAAATTATCCAATTGAACG
CACATAGCTCAATCACACTGGAAATGTTTGTCCTTGCACCTGAGCCTGTT
CCCACTCAGCAGTGAGAGTTCCTCTTTGCCCTGAGGCTCAGTCTCTCTCG
TATTTTGTCCCCACCCCCAATTCCTTGAGTGGTTTTTGCTCTAGGGCCCT
TTCTTGCACTGTCCAGCTGGTTGTACCCTCTCCAGGCATTTATTCAACAA
ATGTGGGTGAAGTGCCTGCTGGGTGCCAGGTGCTGGGAATACATCTGTGG
ACAAGACATGCTTGGGTCCTACTCCTGGAGCACTGTAAAAAGAGCTGATT
CAAGTAAGTAGATGCCTGTTTTGAGACCAGAAGGTTTCATAATTGGTTCT
ACGACCCTTTTGAGCCTAGAATTATTGTTCTTATATAAGATCACTGAAGA
AAGAGGAACCCCCACAACCCCCTCCACAAAGAGACCAGGGGCGGGTGATG
AGACCTGGGGTTTAGAACCCCAGGTGAGACCTCAAATCACTGCATTCATT
CTGAGCCCCCTTCCTGTCCCCAGGGGAGGTGTATTGTGTATGTAGCCTTA
GAGCATCTCTGCCTCCAACCCAGCAGTTCTCTGCCAAAGCTTGTGGAGGA
GGGAGAGCCCTGTCCCTGCCCTCAGGCTCCCCAGTGCTCCTGGCCCTTCT
ATTTATTTGACTGATTATTGCTTCTTTCCTTGCATTAAAGGAGATCTTCC
CCTAACCTTTGGGCCAATTTACTGGCCACTAATTTCGTTTAAATACCATT
GTGTCATTGGGGGGACCGTCTTTACCCCTGCTGACCTCCCACCTATCCGC
CCTGCAGCAGAACCTTGGCGGTTTATAGGTAATGATGGAACTTAGACTCC
TCTTCCCAGAGTCACAAGTAGCCTCTGGGATCTGCCAACACACGTCCACT
CCCAAGCCACTAGCCCACTCCCCAGTTGGCCCTTCTGCCCTTACCCCACA
CACAGTCCAACTCTTCCACCTCTGGGGAAGATGGAGCAGGTCTTTGGGAA
GCTCCCACACCCACCTCTGCCACTCTTAACACTAAGTGAGAGTTGGGGAG
AAACTGAAGCCGTGTTTTTGGCCCCCCGAGGCTAACCCTGATCCATAGTG
CTACCTGCACCTCTGGATTCTGGATTCACAGACCAAGTCCAAGCCCGTTC
TTACGTCGCCATAAAGGCCCCCGAACGGCATTCTCGGTACTTCTGTTTGT
TTTTGTACATTTTATTAGAAAGGACTGTAAAATAGCCACTTAGACACTTT
ACCTCTTCAGTATGCAAATGTAAATAAATTGTAATATAGGAAATCTTTTG
TTTTAATATAAGAATGAGCCTGTCCAATTTCTGCTGTACATTATTAAAAG
TTTTATTCACAGAG,
(SEQ ID NO: 20)
AATGCTTCTCCTAAGCACCTCGTGTGTGTTCTTCGGCCTCACTGCTCTGT
GGCTTAGGTATCTGTGCTGTGGGGTTTGAGAAACATGGTGAAGGTGTATG
AACAGAGCTTGACATTTGTGCTCTGCTGTGTGCGTGCACCAGCTGCCTCC
AGGCCAACTACACGTGTGAGACAGATGGGGCCTGCATGGTTTCCATTTTC
AATCTGGATGGGATGGAGCACCATGTGCGCACCTGCATCCCCAAAGTGGA
GCTGGTCCCTGCCGGGAAGCCCTTCTACTGCCTGAGCTCGGAGGACCTGC
GCAACACCCACTGCTGCTACACTGACTACTGCAACAGGATCGACTTGAGG
GTGCCCAGTGGTCACCTCAAGGAGCCTGAGCACCCGTCCATGTGGGGCCC
GGTGGAGCTGGTAGGCATCATCGCCGGCCCGGTGTTCCTCCTGTTCCTCA
TCATCATCATTGTTTTCCTTGTCATTAACTATCATCAGCGTGTCTATCAC
AACCGCCAGAGACTGGACATGGAAGATCCCTCATGTGAGATGTGTCTCTC
CAAAGACAAGACGCTCCAGGATCTTGTCTACGATCTCTCCACCTCAGGGT
CTGGCTCAGGGTTACCCCTCTTTGTCCAGCGCACAGTGGCCCGAACCATC
GTTTTACAAGAGATTATTGGCAAGGGTCGGTTTGGGGAAGTATGGCGGGG
CCGCTGGAGGGGTGGTGATGTGGCTGTGAAAATATTCTCTTCTCGTGAAG
AACGGTCTTGGTTCAGGGAAGCAGAGATATACCAGACGGTCATGCTGCGC
CATGAAAACATCCTTGGATTTATTGCTGCTGACAATAAAGATAATGGCAC
CTGGACACAGCTGTGGCTTGTTTCTGACTATCATGAGCACGGGTCCCTGT
TTGATTATCTGAACCGGTACACAGTGACAATTGAGGGGATGATTAAGCTG
GCCTTGTCTGCTGCTAGTGGGCTGGCACACCTGCACATGGAGATCGTGGG
CACCCAAGGGAAGCCTGGAATTGCTCATCGAGACTTAAAGTCAAAGAACA
TTCTGGTGAAGAAAAATGGCATGTGTGCCATAGCAGACCTGGGCCTGGCT
GTCCGTCATGATGCAGTCACTGACACCATTGACATTGCCCCGAATCAGAG
GGTGGGGACCAAACGATACATGGCCCCTGAAGTACTTGATGAAACCATTA
ATATGAAACACTTTGACTCCTTTAAATGTGCTGATATTTATGCCCTCGGG
CTTGTATATTGGGAGATTGCTCGAAGATGCAATTCTGGAGGAGTCCATGA
AGAATATCAGCTGCCATATTACGACTTAGTGCCCTCTGACCCTTCCATTG
AGGAAATGCGAAAGGTTGTATGTGATCAGAAGCTGCGTCCCAACATCCCC
AACTGGTGGCAGAGTTATGAGGCACTGCGGGTGATGGGGAAGATGATGCG
AGAGTGTTGGTATGCCAACGGCGCAGCCCGCCTGACGGCCCTGCGCATCA
AGAAGACCCTCTCCCAGCTCAGCGTGCAGGAAGACGTGAAGATCTAACTG
CTCCCTCTCTCCACACGGAGCTCCTGGCAGCGAGAACTACGCACAGCTGC
CGCGTTGAGCGTACGATGGAGGCCTACCTCTCGTTTCTGCCCAGCCCTCT
GTGGCCAGGAGCCCTGGCCCGCAAGAGGGACAGAGCCCGGGAGAGACTCG
CTCACTCCCATGTTGGGTTTGAGACAGACACCTTTTCTATTTACCTCCTA
ATGGCATGGAGACTCTGAGAGCGAATTGTGTGGAGAACTCAGTGCCACAC
CTCGAACTGGTTGTAGTGGGAAGTCCCGCGAAACCCGGTGCATCTGGCAC
GTGGCCAGGAGCCATGACAGGGGCGCTTGGGAGGGGCCGGAGGAACCGAG
GTGTTGCCAGTGCTAAGCTGCCCTGAGGGTTTCCTTCGGGGACCAGCCCA
CAGCACACCAAGGTGGCCCGGAAGAACCAGAAGTGCAGCCCCTCTCACAG
GCAGCTCTGAGCCGCGCTTTCCCCTCCTCCCTGGGATGGACGCTGCCGGG
AGACTGCCAGTGGAGACGGAATCTGCCGCTTTGTCTGTCCAGCCGTGTGT
GCATGTGCCGAGGTGCGTCCCCCGTTGTGCCTGGTTCGTGCCATGCCCTT
ACACGTGCGTGTGAGTGTGTGTGTGTGTCTGTAGGTGCGCACTTACCTGC
TTGAGCTTTCTGTGCATGTGCAGGTCGGGGGTGTGGTCGTCATGCTGTCC
GTGCTTGCTGGTGCCTCTTTTCAGTAGTGAGCAGCATCTAGTTTCCCTGG
TGCCCTTCCCTGGAGGTCTCTCCCTCCCCCAGAGCCCCTCATGCCACAGT
GGTACTCTGTGTCTGGCAGGCTACTCTGCCCACCCCAGCATCAGCACAGC
TCTCCTCCTCCATCTCAGACTGTGGAACCAAAGCTGGCCCAGTTGTCCAT
GACAAAAGAGGCTTTTGGGCCAAAATGTGAGGGTGGTGGGTGGGATGGGC
AGGGAAGGAATCCTGGTGGAAGTCTTGGGTGTTAGTGTCAGCCATGGGAA
ATGAGCCAGCCCAAGGGCATCATCCTCAGCAGCATCGAGGAAGGGCCGAG
GAATGTGAAGCCAGATCTCGGGACTCAGATTGGAATGTTACATCTGTCTT
TCATCTCCCAGATCCTGGAAACAGCAGTGTATATTTTTGGTGGTGGTGGG
TTTGGGGTGGGGAAGGGAAGGGCGGGCAAGGAGTGGGGAGGGAGTCTGGG
GTGGGAGGGAGGCATCTGCATGGGTCTTCTTTTACTGGACTGTCTGATCA
GGGTGGAGGGAAGGTGAGAGGTTTGCATCCACTTCAGGAGCCCTACTGAA
GGGAACAGCCTGAGCCGAACATGTTATTTAACCTGAGTATAGTATTTAAC
GAAGCCTAGAAGCACGGCTGTGGGTGGTGATTTGGTCAGCATATCTTAGG
TATATAATAACTTTGAAGCCATAACTTTTAACTGGAGTGGTTTGATTTCT
TTTTTTAATTTTATTGGGAGGGTTTGGATTTTAACTTTTTTTAATGTTGT
TAAATATTAAGTTTTTGTAAAAGGAAAACCATCTCTGTGATTACCTCTCA
ATCTATTTGTTTTTAAAGAAATCCCTAAAAAAAAAAATTATCCAATTGAA
CGCACATAGCTCAATCACACTGGAAATGTTTGTCCTTGCACCTGAGCCTG
TTCCCACTCAGCAGTGAGAGTTCCTCTTTGCCCTGAGGCTCAGTCTCTCT
CGTATTTTGTCCCCACCCCCAATTCCTTGAGTGGTTTTTGCTCTAGGGCC
CTTTCTTGCACTGTCCAGCTGGTTGTACCCTCTCCAGGCATTTATTCAAC
AAATGTGGGTGAAGTGCCTGCTGGGTGCCAGGTGCTGGGAATACATCTGT
GGACAAGACATGCTTGGGTCCTACTCCTGGAGCACTGTAAAAAGAGCTGA
TTCAAGTAAGTAGATGCCTGTTTTGAGACCAGAAGGTTTCATAATTGGTT
CTACGACCCTTTTGAGCCTAGAATTATTGTTCTTATATAAGATCACTGAA
GAAAGAGGAACCCCCACAACCCCCTCCACAAAGAGACCAGGGGCGGGTGA
TGAGACCTGGGGTTTAGAACCCCAGGTGAGACCTCAAATCACTGCATTCA
TTCTGAGCCCCCTTCCTGTCCCCAGGGGAGGTGTATTGTGTATGTAGCCT
TAGAGCATCTCTGCCTCCAACCCAGCAGTTCTCTGCCAAAGCTTGTGGAG
GAGGGAGAGCCCTGTCCCTGCCCTCAGGCTCCCCAGTGCTCCTGGCCCTT
CTATTTATTTGACTGATTATTGCTTCTTTCCTTGCATTAAAGGAGATCTT
CCCCTAACCTTTGGGCCAATTTACTGGCCACTAATTTCGTTTAAATACCA
TTGTGTCATTGGGGGGACCGTCTTTACCCCTGCTGACCTCCCACCTATCC
GCCCTGCAGCAGAACCTTGGCGGTTTATAGGTAATGATGGAACTTAGACT
CCTCTTCCCAGAGTCACAAGTAGCCTCTGGGATCTGCCAACACACGTCCA
CTCCCAAGCCACTAGCCCACTCCCCAGTTGGCCCTTCTGCCCTTACCCCA
CACACAGTCCAACTCTTCCACCTCTGGGGAAGATGGAGCAGGTCTTTGGG
AAGCTCCCACACCCACCTCTGCCACTCTTAACACTAAGTGAGAGTTGGGG
AGAAACTGAAGCCGTGTTTTTGGCCCCCCGAGGCTAACCCTGATCCATAG
TGCTACCTGCACCTCTGGATTCTGGATTCACAGACCAAGTCCAAGCCCGT
TCTTACGTCGCCATAAAGGCCCCCGAACGGCATTCTCGGTACTTCTGTTT
GTTTTTGTACATTTTATTAGAAAGGACTGTAAAATAGCCACTTAGACACT
TTACCTCTTCAGTATGCAAATGTAAATAAATTGTAATATAGGAAATCTTT
TGTTTTAATATAAGAATGAGCCTGTCCAATTTCTGCTGTACATTATTAAA
AGTTTTATTCACAGAG
or
(SEQ ID NO: 21)
GGGGAGGCGCCGGGGGCGCGCGCGCGCGCGCTGGGCGCTGCTGGGCTGCG
GCGGCGGCGGCGGCGGCGGTGGTTACTATGGCGGAGTCGGCCGGAGCCTC
CTCCTTCTTCCCCCTTGTTGTCCTCCTGCTCGCCGGCAGCGGCGGGTCCG
GGCCCCGGGGGGTCCAGGCTCTGCTGTGTGCGTGCACCAGCTGCCTCCAG
GCCAACTACACGTGTGAGACAGATGGGGCCTGCATGGTTTCCATTTTCAA
TCTGGATGGGATGGAGCACCATGTGCGCACCTGCATCCCCAAAGTGGAGC
TGGTCCCTGCCGGGAAGCCCTTCTACTGCCTGAGCTCGGAGGACCTGCGC
AACACCCACTGCTGCTACACTGACTACTGCAACAGGATCGACTTGAGGGT
GCCCAGTGGTCACCTCAAGGAGCCTGAGCACCCGTCCATGTGGGGCCCGG
TGGAGCTGGTAGGCATCATCGCCGGCCCGGTGTTCCTCCTGTTCCTCATC
ATCATCATTGTTTTCCTTGTCATTAACTATCATCAGCGTGTCTATCACAA
CCGCCAGAGACTGGACATGGAAGATCCCTCATGTGAGATGTGTCTCTCCA
AAGACAAGACGCTCCAGGATCTTGTCTACGATCTCTCCACCTCAGGGTCT
GGCTCAGGGTTACCCCTCTTTGTCCAGCGCACAGTGGCCCGAACCATCGT
TTTACAAGAGATTATTGGCAAGGGTCGGTTTGGGGAAGTATGGCGGGGCC
GCTGGAGGGGTGGTGATGTGGCTGTGAAAATATTCTCTTCTCGTGAAGAA
CGGTCTTGGTTCAGGGAAGCAGAGATATACCAGACGGTCATGCTGCGCCA
TGAAAACATCCTTGGATTTATTGCTGCTGACAATAAAGCAGACTGCTCAT
TCCTCACATTGCCATGGGAAGTTGTAATGGTCTCTGCTGCCCCCAAGCTG
AGGAGCCTTAGACTCCAATACAAGGGAGGAAGGGGAAGAGCAAGATTTTT
ATTCCCACTGAATAATGGCACCTGGACACAGCTGTGGCTTGTTTCTGACT
ATCATGAGCACGGGTCCCTGTTTGATTATCTGAACCGGTACACAGTGACA
ATTGAGGGGATGATTAAGCTGGCCTTGTCTGCTGCTAGTGGGCTGGCACA
CCTGCACATGGAGATCGTGGGCACCCAAGGGAAGCCTGGAATTGCTCATC
GAGACTTAAAGTCAAAGAACATTCTGGTGAAGAAAAATGGCATGTGTGCC
ATAGCAGACCTGGGCCTGGCTGTCCGTCATGATGCAGTCACTGACACCAT
TGACATTGCCCCGAATCAGAGGGTGGGGACCAAACGATACATGGCCCCTG
AAGTACTTGATGAAACCATTAATATGAAACACTTTGACTCCTTTAAATGT
GCTGATATTTATGCCCTCGGGCTTGTATATTGGGAGATTGCTCGAAGATG
CAATTCTGGAGGAGTCCATGAAGAATATCAGCTGCCATATTACGACTTAG
TGCCCTCTGACCCTTCCATTGAGGAAATGCGAAAGGTTGTATGTGATCAG
AAGCTGCGTCCCAACATCCCCAACTGGTGGCAGAGTTATGAGGCACTGCG
GGTGATGGGGAAGATGATGCGAGAGTGTTGGTATGCCAACGGCGCAGCCC
GCCTGACGGCCCTGCGCATCAAGAAGACCCTCTCCCAGCTCAGCGTGCAG
GAAGACGTGAAGATCTAACTGCTCCCTCTCTCCACACGGAGCTCCTGGCA
GCGAGAACTACGCACAGCTGCCGCGTTGAGCGTACGATGGAGGCCTACCT
CTCGTTTCTGCCCAGCCCTCTGTGGCCAGGAGCCCTGGCCCGCAAGAGGG
ACAGAGCCCGGGAGAGACTCGCTCACTCCCATGTTGGGTTTGAGACAGAC
ACCTTTTCTATTTACCTCCTAATGGCATGGAGACTCTGAGAGCGAATTGT
GTGGAGAACTCAGTGCCACACCTCGAACTGGTTGTAGTGGGAAGTCCCGC
GAAACCCGGTGCATCTGGCACGTGGCCAGGAGCCATGACAGGGGCGCTTG
GGAGGGGCCGGAGGAACCGAGGTGTTGCCAGTGCTAAGCTGCCCTGAGGG
TTTCCTTCGGGGACCAGCCCACAGCACACCAAGGTGGCCCGGAAGAACCA
GAAGTGCAGCCCCTCTCACAGGCAGCTCTGAGCCGCGCTTTCCCCTCCTC
CCTGGGATGGACGCTGCCGGGAGACTGCCAGTGGAGACGGAATCTGCCGC
TTTGTCTGTCCAGCCGTGTGTGCATGTGCCGAGGTGCGTCCCCCGTTGTG
CCTGGTTCGTGCCATGCCCTTACACGTGCGTGTGAGTGTGTGTGTGTGTC
TGTAGGTGCGCACTTACCTGCTTGAGCTTTCTGTGCATGTGCAGGTCGGG
GGTGTGGTCGTCATGCTGTCCGTGCTTGCTGGTGCCTCTTTTCAGTAGTG
AGCAGCATCTAGTTTCCCTGGTGCCCTTCCCTGGAGGTCTCTCCCTCCCC
CAGAGCCCCTCATGCCACAGTGGTACTCTGTGTCTGGCAGGCTACTCTGC
CCACCCCAGCATCAGCACAGCTCTCCTCCTCCATCTCAGACTGTGGAACC
AAAGCTGGCCCAGTTGTCCATGACAAAAGAGGCTTTTGGGCCAAAATGTG
AGGGTGGTGGGTGGGATGGGCAGGGAAGGAATCCTGGTGGAAGTCTTGGG
TGTTAGTGTCAGCCATGGGAAATGAGCCAGCCCAAGGGCATCATCCTCAG
CAGCATCGAGGAAGGGCCGAGGAATGTGAAGCCAGATCTCGGGACTCAGA
TTGGAATGTTACATCTGTCTTTCATCTCCCAGATCCTGGAAACAGCAGTG
TATATTTTTGGTGGTGGTGGGTTTGGGGTGGGGAAGGGAAGGGCGGGCAA
GGAGTGGGGAGGGAGTCTGGGGTGGGAGGGAGGCATCTGCATGGGTCTTC
TTTTACTGGACTGTCTGATCAGGGTGGAGGGAAGGTGAGAGGTTTGCATC
CACTTCAGGAGCCCTACTGAAGGGAACAGCCTGAGCCGAACATGTTATTT
AACCTGAGTATAGTATTTAACGAAGCCTAGAAGCACGGCTGTGGGTGGTG
ATTTGGTCAGCATATCTTAGGTATATAATAACTTTGAAGCCATAACTTTT
AACTGGAGTGGTTTGATTTCTTTTTTTAATTTTATTGGGAGGGTTTGGAT
TTTAACTTTTTTTAATGTTGTTAAATATTAAGTTTTTGTAAAAGGAAAAC
CATCTCTGTGATTACCTCTCAATCTATTTGTTTTTAAAGAAATCCCTAAA
AAAAAAAATTATCCAATTGAACGCACATAGCTCAATCACACTGGAAATGT
TTGTCCTTGCACCTGAGCCTGTTCCCACTCAGCAGTGAGAGTTCCTCTTT
GCCCTGAGGCTCAGTCTCTCTCGTATTTTGTCCCCACCCCCAATTCCTTG
AGTGGTTTTTGCTCTAGGGCCCTTTCTTGCACTGTCCAGCTGGTTGTACC
CTCTCCAGGCATTTATTCAACAAATGTGGGTGAAGTGCCTGCTGGGTGCC
AGGTGCTGGGAATACATCTGTGGACAAGACATGCTTGGGTCCTACTCCTG
GAGCACTGTAAAAAGAGCTGATTCAAGTAAGTAGATGCCTGTTTTGAGAC
CAGAAGGTTTCATAATTGGTTCTACGACCCTTTTGAGCCTAGAATTATTG
TTCTTATATAAGATCACTGAAGAAAGAGGAACCCCCACAACCCCCTCCAC
AAAGAGACCAGGGGCGGGTGATGAGACCTGGGGTTTAGAACCCCAGGTGA
GACCTCAAATCACTGCATTCATTCTGAGCCCCCTTCCTGTCCCCAGGGGA
GGTGTATTGTGTATGTAGCCTTAGAGCATCTCTGCCTCCAACCCAGCAGT
TCTCTGCCAAAGCTTGTGGAGGAGGGAGAGCCCTGTCCCTGCCCTCAGGC
TCCCCAGTGCTCCTGGCCCTTCTATTTATTTGACTGATTATTGCTTCTTT
CCTTGCATTAAAGGAGATCTTCCCCTAACCTTTGGGCCAATTTACTGGCC
ACTAATTTCGTTTAAATACCATTGTGTCATTGGGGGGACCGTCTTTACCC
CTGCTGACCTCCCACCTATCCGCCCTGCAGCAGAACCTTGGCGGTTTATA
GGTAATGATGGAACTTAGACTCCTCTTCCCAGAGTCACAAGTAGCCTCTG
GGATCTGCCAACACACGTCCACTCCCAAGCCACTAGCCCACTCCCCAGTT
GGCCCTTCTGCCCTTACCCCACACACAGTCCAACTCTTCCACCTCTGGGG
AAGATGGAGCAGGTCTTTGGGAAGCTCCCACACCCACCTCTGCCACTCTT
AACACTAAGTGAGAGTTGGGGAGAAACTGAAGCCGTGTTTTTGGCCCCCC
GAGGCTAACCCTGATCCATAGTGCTACCTGCACCTCTGGATTCTGGATTC
ACAGACCAAGTCCAAGCCCGTTCTTACGTCGCCATAAAGGCCCCCGAACG
GCATTCTCGGTACTTCTGTTTGTTTTTGTACATTTTATTAGAAAGGACTG
TAAAATAGCCACTTAGACACTTTACCTCTTCAGTATGCAAATGTAAATAA
ATTGTAATATAGGAAATCTTTTGTTTTAATATAAGAATGAGCCTGTCCAA
TTTCTGCTGTACATTATTAAAAGTTTTATTCACAGAG.

Introduction of nucleic acid into target cells (e.g., host cells) can be carried out by methods known in the art such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.”

### Therapeutic and Prophylactic Methods

In accordance with the invention, treatment methods and uses are provided that include therapeutic and prophylactic methods and uses. Such methods and uses can, for example, stimulate, induce, increase, enhance, or inhibit, decrease or reduce, an immune response in a subject. In one embodiment, a method includes administering to a subject in need of treatment an amount of activin or subsequence thereof, or an activin receptor activator or inhibitor, to treat the subject. In another embodiment, a method includes administering to a subject an amount of an activin receptor activator or inhibitor, or an activin or a subsequence thereof, sufficient to provide the subject with an immune response that is stimulated, induced, increased or enhanced, or inhibited, decreased or reduced.

Methods are applicable to immune responses, cell mediated and/or antibody mediated (e.g., a humoral immune response). Methods are applicable to immune responses, including undesirable or aberrant immune responses, such as undesirable or aberrant inflammatory responses or inflammation. Methods are also applicable to acute or chronic immune responses, and acute or chronic undesirable or aberrant immune responses, such as acute or chronic undesirable or aberrant inflammatory responses or inflammation.

As used herein, an “undesirable immune response” or “aberrant immune response” refers to any immune response, activity or function that is greater or less than desired or physiologically normal. An undesirable immune response, function or activity can be a normal response, function or activity. Thus, normal immune responses so long as they are undesirable, even if not considered abnormal, are included within the meaning of these terms. An undesirable immune response, function or activity can also be an abnormal response, function or activity. An abnormal (aberrant) immune response, function or activity deviates from normal. Undesirable and aberrant immune responses can be humoral, cell-mediated or a combination thereof, either chronic or acute.

One non-limiting example of an undesirable or aberrant immune response is where the immune response is hyper-responsive, such as in the case of an autoimmune disorder or disease. Another example of an undesirable or aberrant immune response is where an immune response leads to acute or chronic inflammatory response or inflammation in any tissue or organ, such as an allergy. Yet another example of an undesirable or aberrant immune response is where an immune response leads to destruction of cells, tissue or organ, such as a bone marrow transplant, as in graft vs. host disease (GVHD). Still another example of an undesirable or aberrant immune response is where the immune response is hypo-responsive, such as where response to an antigen is less than desired, e.g., tolerance has occurred.

The terms “immune disorder” and “immune disease” mean, an immune function or activity, that is greater than (e.g., autoimmunity) or less than (e.g., immunodeficiency) desired, and which is characterized by different physiological symptoms or abnormalities, depending upon the disorder or disease. Particular non-limiting examples of immune disorders and diseases to which the invention applies include autoimmune disorders. Autoimmune disorders are generally characterized as an undesirable or aberrant increased or inappropriate response, activity or function of the immune system. Disorders and diseases that can be treated in accordance with the invention include, but are not limited to, disorders and disease that cause cell or tissue/organ damage in the subject.

As used herein, the term “autoimmune disease” encompasses a condition, disorder, dysfunction or disease that results when the immune system attacks the body's own cells, tissues or organs. In particular, the term “autoimmune disease” includes a disorder wherein the immune system of a mammal mounts a humoral or cellular immune response to the mammal's own cells, tissues or organs. It is understood that more than one type of cell, tissue or organ may be attacked in the course of autoimmune disease.

Specific non-limiting examples of autoimmune diseases include rheumatoid arthritis, juvenile rheumatoid arthritis, osteoarthritis, psoriatic arthritis, diabetes mellitus, multiple sclerosis, encephalomyelitis, myasthenia gravis, systemic lupus erythematous (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, asthma, allergic asthma, cutaneous lupus erythematous, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, Vitiligo, autoimmune hemolytic anemia, autoimmune thrombocytopenic purpura, pernicious anemia, Guillain-Barre syndrome, acute rheumatic fever, sympathetic ophthalmia, Goodpasture's syndrome, systemic necrotizing vasculitis, antiphospholipid syndrome and allergies.

An undesirable or aberrant inflammatory response or inflammation may cause, directly or indirectly, cell, tissue or organ damage, either to multiple cells, tissues or organs, or specifically to a single cell type, tissue type or organ. Exemplary tissues and organs that can exhibit damage include epidermal or mucosal tissue, gut, bowel, pancreas, thymus, liver, kidney, spleen, skin, or a skeletal joint (e.g., knee, ankle, hip, shoulder, wrist, finger, toe, or elbow). Treatment in accordance with the invention can result in reducing, inhibiting or preventing progression or worsening of tissue damage. Such treatments can in turn lead to regeneration or restoration of a damaged organ or tissue, e.g., skin, mucosum, liver.

In one embodiment, provided is a method of use of an anti ActRII antibody or antigen binding fragment thereof selected from

(i) an antibody or antigen binding fragment thereof that binds activin receptor, wherein said antibody or antigen binding fragment comprises a heavy chain variable region CDR1 comprising an amino acid sequence selected from the group consisting of SEQ ID NOs: 22-35; a heavy chain variable region CDR2 comprising an amino acid sequence selected from the group consisting of SEQ ID NOs: 36-49; a heavy chain variable region CDR3 comprising an amino acid sequence selected from the group consisting of SEQ ID NOs: 50-63; a light chain variable region CDR1 comprising an amino acid sequence selected from the group consisting of SEQ ID NOs: 64-77; a light chain variable region CDR2 comprising an amino acid sequence selected from the group consisting of SEQ ID NOs: 78-91; and a light chain variable region CDR3 comprising an amino acid sequence selected from the group consisting of SEQ ID NOs: 92-105;
(ii) an antibody or antigen binding fragment thereof that binds activing receptor, wherein said antibody or antigen binding fragment comprises:
(a) a heavy chain variable region CDR1 of SEQ ID NO: 22; a heavy chain variable region CDR2 of SEQ ID NO: 36; a heavy chain variable region CDR3 of SEQ ID NO: 50; a light chain variable region CDR1 of SEQ ID NO: 64; a light chain variable region CDR2 of SEQ ID NO: 78; and a light chain variable region CDR3 of SEQ ID NO: 92,
(b) a heavy chain variable region CDR1 of SEQ ID NO: 23 a heavy chain variable region CDR2 of SEQ ID NO: 37; a heavy chain variable region CDR3 of SEQ ID NO: 51; a light chain variable region CDR1 of SEQ ID NO: 65; a light chain variable region CDR2 of SEQ ID NO: 79; and a light chain variable region CDR3 of SEQ ID NO: 93,
(c) a heavy chain variable region CDR1 of SEQ ID NO: 24; a heavy chain variable region CDR2 of SEQ ID NO: 38; a heavy chain variable region CDR3 of SEQ ID NO: 52; a light chain variable region CDR1 of SEQ ID NO: 66; a light chain variable region CDR2 of SEQ ID NO: 80; and a light chain variable region CDR3 of SEQ ID NO: 94,
(d) a heavy chain variable region CDR1 of SEQ ID NO: 25; a heavy chain variable region CDR2 of SEQ ID NO: 39; a heavy chain variable region CDR3 of SEQ ID NO: 53; a light chain variable region CDR1 of SEQ ID NO: 67; a light chain variable region CDR2 of SEQ ID NO: 81; and a light chain variable region CDR3 of SEQ ID NO: 95,
(e) a heavy chain variable region CDR1 of SEQ ID NO: 26; a heavy chain variable region CDR2 of SEQ ID NO: 40; a heavy chain variable region CDR3 of SEQ ID NO: 54; a light chain variable region CDR1 of SEQ ID NO: 68; a light chain variable region CDR2 of SEQ ID NO: 82; and a light chain variable region CDR3 of SEQ ID NO: 96,
(f) a heavy chain variable region CDR1 of SEQ ID NO: 27; a heavy chain variable region CDR2 of SEQ ID NO: 41; a heavy chain variable region CDR3 of SEQ ID NO: 55; a light chain variable region CDR1 of SEQ ID NO: 69; a light chain variable region CDR2 of SEQ ID NO: 62; and a light chain variable region CDR3 of SEQ ID NO: 97,
(g) a heavy chain variable region CDR1 of SEQ ID NO: 28; a heavy chain variable region CDR2 of SEQ ID NO: 42; a heavy chain variable region CDR3 of SEQ ID NO: 56; a light chain variable region CDR1 of SEQ ID NO: 70; a light chain variable region CDR2 of SEQ ID NO: 84; and a light chain variable region CDR3 of SEQ ID NO: 98,
(h) a heavy chain variable region CDR1 of SEQ ID NO: 29; a heavy chain variable region CDR2 of SEQ ID NO: 43; a heavy chain variable region CDR3 of SEQ ID NO: 57; a light chain variable region CDR1 of SEQ ID NO: 71 a light chain variable region CDR2 of SEQ ID NO: 85; and a light chain variable region CDR3 of SEQ ID NO: 99,
(i) a heavy chain variable region CDR1 of SEQ ID NO: 30; a heavy chain variable region CDR2 of SEQ ID NO: 44; a heavy chain variable region CDR3 of SEQ ID NO: 58; a light chain variable region CDR1 of SEQ ID NO: 72; a light chain variable region CDR2 of SEQ ID NO: 86; and a light chain variable region CDR3 of SEQ ID NO: 100,
(j) a heavy chain variable region CDR1 of SEQ ID NO: 31; a heavy chain variable region CDR2 of SEQ ID NO: 45; a heavy chain variable region CDR3 of SEQ ID NO: 59; a light chain variable region CDR1 of SEQ ID NO: 73; a light chain variable region CDR2 of SEQ ID NO: 87; and a light chain variable region CDR3 of SEQ ID NO: 101,
(k) a heavy chain variable region CDR1 of SEQ ID NO: 32; a heavy chain variable region CDR2 of SEQ ID NO: 46; a heavy chain variable region CDR3 of SEQ ID NO: 60; a light chain variable region CDR1 of SEQ ID NO: 74; a light chain variable region CDR2 of SEQ ID NO: 88; and a light chain variable region CDR3 of SEQ ID NO: 102,
(l) a heavy chain variable region CDR1 of SEQ ID NO: 33; a heavy chain variable region CDR2 of SEQ ID NO: 47; a heavy chain variable region CDR3 of SEQ ID NO: 61; a light chain variable region CDR1 of SEQ ID NO: 75; a light chain variable region CDR2 of SEQ ID NO: 89; and a light chain variable region CDR3 of SEQ ID NO: 103,
(m) a heavy chain variable region CDR1 of SEQ ID NO: 34; a heavy chain variable region CDR2 of SEQ ID NO: 48; a heavy chain variable region CDR3 of SEQ ID NO: 62; a light chain variable region CDR1 of SEQ ID NO: 76; a light chain variable region CDR2 of SEQ ID NO: 90; and a light chain variable region CDR3 of SEQ ID NO: 104, or
(n) a heavy chain variable region CDR1 of SEQ ID NO: 35; a heavy chain variable region CDR2 of SEQ ID NO: 49; a heavy chain variable region CDR3 of SEQ ID NO: 63; a light chain variable region CDR1 of SEQ ID NO: 77; a light chain variable region CDR2 of SEQ ID NO: 91; and a light chain variable region CDR3 of SEQ ID NO: 105;
   (iii) an antibody or antigen binding fragment thereof that binds activing receptor, wherein said antibody or antigen binding fragment comprises a VH polypeptide sequence having at least 95% sequence identity to at least one of SEQ ID NOs: 120-133 and a VL polypeptide sequence having at least 95% sequence identity to at least one of SEQ ID NOs: 106-119;
   (iv) an antibody or antigen binding fragment thereof that binds activing receptor, wherein said antibody or antigen binding fragment comprises a full length heavy chain amino acid sequence having at least 95% sequence identity to at least one sequence selected from the group consisting of SEQ ID NOs:167-171 and 177-181 and a full length light chain amino acid sequence having at least 95% sequence identity to at least one sequence selected from the group consisting of SEQ ID NOs:162-166 and 172-176;
   (v) an antibody or antigen binding fragment thereof that binds activing receptor, wherein said antibody or antigen binding fragment comprises
(a) the variable heavy chain sequence of SEQ ID NO: 120 and variable light chain sequence of SEQ ID NO: 106;
(b) the variable heavy chain sequence of SEQ ID NO: 121 and variable light chain sequence of SEQ ID NO: 107;
(c) the variable heavy chain sequence of SEQ ID NO: 122 and variable light chain sequence of SEQ ID NO: 108;
(d) the variable heavy chain sequence of SEQ ID NO: 123 and variable light chain sequence of SEQ ID NO: 109;
(e) the variable heavy chain sequence of SEQ ID NO: 124 and variable light chain sequence of SEQ ID NO: 110;
(f) the variable heavy chain sequence of SEQ ID NO: 125 and variable light chain sequence of SEQ ID NO: 111;
(g) the variable heavy chain sequence of SEQ ID NO: 126 and variable light chain sequence of SEQ ID NO: 112;
(h) the variable heavy chain sequence of SEQ ID NO: 127 and variable light chain sequence of SEQ ID NO: 113;
(i) the variable heavy chain sequence of SEQ ID NO: 128 and variable light chain sequence of SEQ ID NO: 114;
(j) the variable heavy chain sequence of SEQ ID NO: 129 and variable light chain sequence of SEQ ID NO: 115;
(k) the variable heavy chain sequence of SEQ ID NO: 130 and variable light chain sequence of SEQ ID NO: 116;
(l) the variable heavy chain sequence of SEQ ID NO: 131 and variable light chain sequence of SEQ ID NO: 117;
(m) the variable heavy chain sequence of SEQ ID NO: 132 and variable light chain sequence of SEQ ID NO: 118; or
(n) the variable heavy chain sequence of SEQ ID NO: 133 and variable light chain sequence of SEQ ID NO: 119; or
   (vi) an antibody or antigen binding fragment thereof that binds activing receptor, wherein said antigen binding fragment comprises
(a) the heavy chain sequence of SEQ ID NO: 167 and light chain sequence of SEQ ID NO: 162;
(b) the heavy chain sequence of SEQ ID NO: 168 and light chain sequence of SEQ ID NO: 163;
(c) the heavy chain sequence of SEQ ID NO: 169 and light chain sequence of SEQ ID NO: 164;
(d) the heavy chain sequence of SEQ ID NO: 170 and light chain sequence of SEQ ID NO: 165;
(e) the heavy chain sequence of SEQ ID NO: 171 and light chain sequence of SEQ ID NO: 166;
(f) the heavy chain sequence of SEQ ID NO: 177 and light chain sequence of SEQ ID NO: 172;
(g) the heavy chain sequence of SEQ ID NO: 178 and light chain sequence of SEQ ID NO: 173;
(h) the heavy chain sequence of SEQ ID NO: 179 and light chain sequence of SEQ ID NO: 174;
(i) the heavy chain sequence of SEQ ID NO: 180 and light chain sequence of SEQ ID NO: 175; or
(j) the heavy chain sequence of SEQ ID NO: 181 and light chain sequence of SEQ ID NO: 176;
   or a pharmaceutical composition comprising such antibody or antigen binding fragment in the manufacture of a medicament for the treatment of an undesirable or aberrant immune response, inflammatory response or inflammation.

In a particular embodiment, provided is an anti ActRII antibody or antigen binding fragment thereof selected from:

(i) an antibody or antigen binding fragment thereof that binds activin receptor, wherein said antibody or antigen binding fragment comprises a heavy chain variable region CDR1 comprising an amino acid sequence selected from the group consisting of SEQ ID NOs: 22-35; a heavy chain variable region CDR2 comprising an amino acid sequence selected from the group consisting of SEQ ID NOs: 36-49; a heavy chain variable region CDR3 comprising an amino acid sequence selected from the group consisting of SEQ ID NOs: 50-63; a light chain variable region CDR1 comprising an amino acid sequence selected from the group consisting of SEQ ID NOs: 64-77; a light chain variable region CDR2 comprising an amino acid sequence selected from the group consisting of SEQ ID NOs: 78-91; and a light chain variable region CDR3 comprising an amino acid sequence selected from the group consisting of SEQ ID NOs: 92-105;
(ii) an antibody or antigen binding fragment thereof that binds activing receptor, wherein said antibody or antigen binding fragment comprises:
(a) a heavy chain variable region CDR1 of SEQ ID NO: 22; a heavy chain variable region CDR2 of SEQ ID NO: 36; a heavy chain variable region CDR3 of SEQ ID NO: 50; a light chain variable region CDR1 of SEQ ID NO: 64; a light chain variable region CDR2 of SEQ ID NO: 78; and a light chain variable region CDR3 of SEQ ID NO: 92,
(b) a heavy chain variable region CDR1 of SEQ ID NO: 23 a heavy chain variable region CDR2 of SEQ ID NO: 37; a heavy chain variable region CDR3 of SEQ ID NO: 51; a light chain variable region CDR1 of SEQ ID NO: 65; a light chain variable region CDR2 of SEQ ID NO: 79; and a light chain variable region CDR3 of SEQ ID NO: 93,
(c) a heavy chain variable region CDR1 of SEQ ID NO: 24; a heavy chain variable region CDR2 of SEQ ID NO: 38; a heavy chain variable region CDR3 of SEQ ID NO: 52; a light chain variable region CDR1 of SEQ ID NO: 66; a light chain variable region CDR2 of SEQ ID NO: 80; and a light chain variable region CDR3 of SEQ ID NO: 94,
(d) a heavy chain variable region CDR1 of SEQ ID NO: 25; a heavy chain variable region CDR2 of SEQ ID NO: 39; a heavy chain variable region CDR3 of SEQ ID NO: 53; a light chain variable region CDR1 of SEQ ID NO: 67; a light chain variable region CDR2 of SEQ ID NO: 81; and a light chain variable region CDR3 of SEQ ID NO: 95,
(e) a heavy chain variable region CDR1 of SEQ ID NO: 26; a heavy chain variable region CDR2 of SEQ ID NO: 40; a heavy chain variable region CDR3 of SEQ ID NO: 54; a light chain variable region CDR1 of SEQ ID NO: 68; a light chain variable region CDR2 of SEQ ID NO: 82; and a light chain variable region CDR3 of SEQ ID NO: 96,
(f) a heavy chain variable region CDR1 of SEQ ID NO: 27; a heavy chain variable region CDR2 of SEQ ID NO: 41; a heavy chain variable region CDR3 of SEQ ID NO: 55; a light chain variable region CDR1 of SEQ ID NO: 69; a light chain variable region CDR2 of SEQ ID NO: 62; and a light chain variable region CDR3 of SEQ ID NO: 97,
(g) a heavy chain variable region CDR1 of SEQ ID NO: 28; a heavy chain variable region CDR2 of SEQ ID NO: 42; a heavy chain variable region CDR3 of SEQ ID NO: 56; a light chain variable region CDR1 of SEQ ID NO: 70; a light chain variable region CDR2 of SEQ ID NO: 84; and a light chain variable region CDR3 of SEQ ID NO: 98,
(h) a heavy chain variable region CDR1 of SEQ ID NO: 29; a heavy chain variable region CDR2 of SEQ ID NO: 43; a heavy chain variable region CDR3 of SEQ ID NO: 57; a light chain variable region CDR1 of SEQ ID NO: 71 a light chain variable region CDR2 of SEQ ID NO: 85; and a light chain variable region CDR3 of SEQ ID NO: 99,
(i) a heavy chain variable region CDR1 of SEQ ID NO: 30; a heavy chain variable region CDR2 of SEQ ID NO: 44; a heavy chain variable region CDR3 of SEQ ID NO: 58; a light chain variable region CDR1 of SEQ ID NO: 72; a light chain variable region CDR2 of SEQ ID NO: 86; and a light chain variable region CDR3 of SEQ ID NO: 100,
(j) a heavy chain variable region CDR1 of SEQ ID NO: 31; a heavy chain variable region CDR2 of SEQ ID NO: 45; a heavy chain variable region CDR3 of SEQ ID NO: 59; a light chain variable region CDR1 of SEQ ID NO: 73; a light chain variable region CDR2 of SEQ ID NO: 87; and a light chain variable region CDR3 of SEQ ID NO: 101,
(k) a heavy chain variable region CDR1 of SEQ ID NO: 32; a heavy chain variable region CDR2 of SEQ ID NO: 46; a heavy chain variable region CDR3 of SEQ ID NO: 60; a light chain variable region CDR1 of SEQ ID NO: 74; a light chain variable region CDR2 of SEQ ID NO: 88; and a light chain variable region CDR3 of SEQ ID NO: 102,
(l) a heavy chain variable region CDR1 of SEQ ID NO: 33; a heavy chain variable region CDR2 of SEQ ID NO: 47; a heavy chain variable region CDR3 of SEQ ID NO: 61; a light chain variable region CDR1 of SEQ ID NO: 75; a light chain variable region CDR2 of SEQ ID NO: 89; and a light chain variable region CDR3 of SEQ ID NO: 103,
(m) a heavy chain variable region CDR1 of SEQ ID NO: 34; a heavy chain variable region CDR2 of SEQ ID NO: 48; a heavy chain variable region CDR3 of SEQ ID NO: 62; a light chain variable region CDR1 of SEQ ID NO: 76; a light chain variable region CDR2 of SEQ ID NO: 90; and a light chain variable region CDR3 of SEQ ID NO: 104, or
(n) a heavy chain variable region CDR1 of SEQ ID NO: 35; a heavy chain variable region CDR2 of SEQ ID NO: 49; a heavy chain variable region CDR3 of SEQ ID NO: 63; a light chain variable region CDR1 of SEQ ID NO: 77; a light chain variable region CDR2 of SEQ ID NO: 91; and a light chain variable region CDR3 of SEQ ID NO: 105;
   (iii) an antibody or antigen binding fragment thereof that binds activing receptor, wherein said antibody or antigen binding fragment comprises a VH polypeptide sequence having at least 95% sequence identity to at least one of SEQ ID NOs: 120-133 and a VL polypeptide sequence having at least 95% sequence identity to at least one of SEQ ID NOs: 106-119;
(iv) an antibody or antigen binding fragment thereof that binds activing receptor, wherein said antibody or antigen binding fragment comprises a full length heavy chain amino acid sequence having at least 95% sequence identity to at least one sequence selected from the group consisting of SEQ ID NOs:167-171 and 177-181 and a full length light chain amino acid sequence having at least 95% sequence identity to at least one sequence selected from the group consisting of SEQ ID NOs:162-166 and 172-176;
   (v) an antibody or antigen binding fragment thereof that binds activing receptor, wherein said antibody or antigen binding fragment comprises
(a) the variable heavy chain sequence of SEQ ID NO: 120 and variable light chain sequence of SEQ ID NO: 106;
(b) the variable heavy chain sequence of SEQ ID NO: 121 and variable light chain sequence of SEQ ID NO: 107;
(c) the variable heavy chain sequence of SEQ ID NO: 122 and variable light chain sequence of SEQ ID NO: 108;
(d) the variable heavy chain sequence of SEQ ID NO: 123 and variable light chain sequence of SEQ ID NO: 109;
(e) the variable heavy chain sequence of SEQ ID NO: 124 and variable light chain sequence of SEQ ID NO: 110;
(f) the variable heavy chain sequence of SEQ ID NO: 125 and variable light chain sequence of SEQ ID NO: 111;
(g) the variable heavy chain sequence of SEQ ID NO: 126 and variable light chain sequence of SEQ ID NO: 112;
(h) the variable heavy chain sequence of SEQ ID NO: 127 and variable light chain sequence of SEQ ID NO: 113;
(i) the variable heavy chain sequence of SEQ ID NO: 128 and variable light chain sequence of SEQ ID NO: 114;
(j) the variable heavy chain sequence of SEQ ID NO: 129 and variable light chain sequence of SEQ ID NO: 115;
(k) the variable heavy chain sequence of SEQ ID NO: 130 and variable light chain sequence of SEQ ID NO: 116;
(l) the variable heavy chain sequence of SEQ ID NO: 131 and variable light chain sequence of SEQ ID NO: 117;
(m) the variable heavy chain sequence of SEQ ID NO: 132 and variable light chain sequence of SEQ ID NO: 118; or
(n) the variable heavy chain sequence of SEQ ID NO: 133 and variable light chain sequence of SEQ ID NO: 119; or
   (vi) an antibody or antigen binding fragment thereof that binds activing receptor, wherein said antigen binding fragment comprises
(a) the heavy chain sequence of SEQ ID NO: 167 and light chain sequence of SEQ ID NO: 162;
(b) the heavy chain sequence of SEQ ID NO: 168 and light chain sequence of SEQ ID NO: 163;
(c) the heavy chain sequence of SEQ ID NO: 169 and light chain sequence of SEQ ID NO: 164;
(d) the heavy chain sequence of SEQ ID NO: 170 and light chain sequence of SEQ ID NO: 165;
(e) the heavy chain sequence of SEQ ID NO: 171 and light chain sequence of SEQ ID NO: 166;
(f) the heavy chain sequence of SEQ ID NO: 177 and light chain sequence of SEQ ID NO: 172;
(g) the heavy chain sequence of SEQ ID NO: 178 and light chain sequence of SEQ ID NO: 173;
(h) the heavy chain sequence of SEQ ID NO: 179 and light chain sequence of SEQ ID NO: 174;
(i) the heavy chain sequence of SEQ ID NO: 180 and light chain sequence of SEQ ID NO: 175; or
(j) the heavy chain sequence of SEQ ID NO: 181 and light chain sequence of SEQ ID NO: 176;
or a pharmaceutical composition comprising such antibody or antigen binding fragment for use in the treatment of an undesirable or aberrant immune response, inflammatory response or inflammation.

Methods and uses of the invention include administering activin or subsequence thereof, or a activin receptor activator, in order to increase, stimulate enhance or promote an immune response in general. Such methods and uses can be used for treatment of chronic or acute immunosuppression or an immunocompromised subject, or an immunodeficiency.

Non-limiting examples of immunosuppression and immunodeficiency treatable in accordance with the invention include severe combined immunodeficiency (SCID) such as recombinase activating gene (RAG 1/2) deficiency, adenosine deaminase (ADA) deficiency, interleukin receptor γ chain deficiency, Janus-associated kinase 3 (JAK 3) deficiency and reticular dysgenesis; primary T cell immunodeficiency such as DiGeorge syndrome, Nude syndrome, T cell receptor deficiency, MHC class II deficiency, TAP-2 deficiency (MHC class I deficiency), ZAP70 tyrosine kinase deficiency and purine nucleotide phosphorylase (PNP) deficiency; predominantly antibody deficiencies such as X-linked agammaglobulinemia (Bruton's tyrosine kinase deficiency); autosomal recessive agammaglobulinemia such as Mu heavy chain deficiency; surrogate light chain (γ 5/14.1) deficiency; Hyper-IgM syndrome either X-linked (CD40 ligand deficiency) and others; Ig heavy chain gene deletion; IgA deficiency; deficiency of IgG subclasses (with or without IgA deficiency); common variable immunodeficiency (CVID); antibody deficiency with normal immunoglobulins; transient hypogammaglobulinemia of infancy; interferon γ receptor (IFNGR1, IFNGR2) deficiency; interleukin 1 and interleukin 12 receptor deficiency; immunodeficiency with thymoma; Wiskott-Aldrich syndrome (WAS protein deficiency); ataxia telangiectasia (ATM deficiency); X-linked lymphoproliferative syndrome (SH2D1 A/SAP deficiency); and hyper IgE syndrome). Exemplary immunodeficiencies also include disorders associated with or secondary to another disease (e.g., chromosomal instability or defective repair such as Bloom syndrome, Xeroderma pigmentosum, Fanconi anemia, ICF syndrome, Nijmegen breakage syndrome and Seckel syndrome; chromosomal defects such as Down syndrome (Trisomy 21), Turner syndrome and Deletions or rings of chromosome 18 (18p- and 18q-); skeletal abnormalities such as short-limbed skeletal dysplasia (short-limbed dwarfism) and cartilage-hair hypoplasia (metaphyseal chondroplasia); immunodeficiency associated with generalized growth retardation such as Schimke immuno-osseous dysplasia, Dubowitz syndrome, Kyphomelic dysplasia with SCID, Mulibrey's nannism, Growth retardation, facial anomalies and immunodeficiency and Progeria (Hutchinson-Gilford syndrome); immunodeficiency with dermatologic defects such as ectrodactyly-ectodermal dysplasia-clefting syndrome, immunodeficiency with absent thumbs, anosmia and ichthyosis, partial albinism, Dyskeratosis congenita, Netherton syndrome, Anhidrotic ectodermal dysplasia, Papillon-Lefevre syndrome and congenital ichthyosis; hereditary metabolic defects such as acrodermatitis enteropathica, transcobalamin 2 deficiency, type 1 hereditary orotic aciduria, intractable diarrhea, abnormal facies, trichorrhexis and immunodeficiency, methylmalonic acidemia, biotin dependent carboxylase deficiency, mannosidosis, glycogen storage disease, type 1b, Chediak-Higashi syndrome; hypercatabolism of immunoglobulin such as familial hypercatabolism, intestinal lymphangiectasia; chronic muco-cutaneous candidiasis; hereditary or congenital hyposplenia or asplenia; and Ivermark syndrome. Methods and uses of the invention include administering activin or subsequence thereof, or a activin receptor activator, in order to increase, stimulate enhance or promote a response elicited by vaccination or immunization. Such methods and uses can be used in particular with any pathogen infection to which a vaccination or immunization may be desired.

Pathogens include, without limitation, bacteria, virus, fungi and parasites.

Non-limiting examples of a bacterial pathogen include Bordetella, Borellia, Brucella, Burkholderia, Campylobacter, Chlamydia, Cosotridia, Heliobacter, Legionella, Listeria, Mycobacterium, Mycoplasma, Neisseria, Pseudomonas, Rickettsia, Staphlyococcus, Streptococcus, Salmonella, Shigella, Treponema, Vibrio, and Yersenia.

Non-limiting examples of viral pathogens include a poxvirus, herpesvirus, hepatitis virus, immunodeficiency virus, flavivirus, papilloma virus (PV), polyoma virus, rhabdovirus, a myxovirus, an arenavirus, a coronavirus, adenovirus, reovirus, picornavirus, togavirus, bunyavirus, parvovirus and retrovirus.

Non-limiting examples of poxvirus include vaccinia virus, Molluscum contagiosum, variola major or variola minor smallpox virus, cow pox, camel pox, sheep pox, and monkey pox.

Non-limiting examples of herpesvirus include alpha-herpesvirus, beta-herpesvirus, gamma-herpesvirus, Epstein Bar Virus (EBV), Cytomegalovirus (CMV), varicella zoster virus (VZV/HHV-3), and human herpes virus 1, 2, 4, 5, 6, 7, and 8 (HHV-8, Kaposi's sarcoma-associated virus).

Non-limiting examples of hepatitis virus include hepatitis A, B, C, D, E and G.

Non-limiting examples of immunodeficiency virus (HIV) include human HIV, such as HIV-1, HIV-2 and HIV-3.

Non-limiting examples of flavivirus include Yellow Fever virus, Dengue virus, Japanese Encephalitis and West Nile viruses.

Non-limiting examples of papilloma virus include a human papilloma virus (HPV), such as HPV strain 1, 6, 11, 16, 18, 30, 31, 42, 43, 44, 45, 51, 52, and 54.

Non-limiting examples of polyoma virus include BK virus (BKV) and JC virus (JCV).

Non-limiting examples of rhabdovirus include rabies virus and vesiculovirus.

Non-limiting examples of myxovirus include paramyxovirus and orthomyoxovirus. Non-limiting examples of paramyxovirus include measles, mumps, pneumovirus and respiratory syncytial virus (RSV).

Non-limiting examples of orthomyoxovirus include influenza virus, such as influenza A, influenza B and influenza C.

Non-limiting examples of arenavirus include lymphocytic choriomeningitis virus (LCMV), Junin virus, Lassa virus, Guanarito virus, Sabia virus and Machupo virus.

Non-limiting examples of coronavirus include a virus that causes a common cold, and severe acute respiratory syndrome (SARS).

Non-limiting examples of adenovirus include viral infections of bronchii, lung, stomach, intestine (gastroenteritis), eye (conjunctivitis), bladder (cystitis) and skin.

Non-limiting examples of reovirus include a rotavirus, cypovirus and orbivirus.

Non-limiting examples of picornavirus include a rhinovirus, apthovirus, hepatovirus, enterovirus and cardiovirus. Rhinovirus can cause the common cold.

Non-limiting examples of togavirus include alphavirus, sindbus virus, and rubellavirus.

Non-limiting examples of bunyavirus include hantavirus, phlebovirus and nairovirus.

Non-limiting examples of retrovirus include an alpha, beta, delta, gamma, epsilon, lentivirus, spumavirus and human T-cell leukemia virus.

Non-limiting examples of lentivirus include an immunodeficiency virus, such as immunodeficiency virus (e.g., a bovine, porcine, equine, canine, feline or primate virus).

Non-limiting examples of human T-cell leukemia viruses include human T-cell leukemia virus 1 and 2 (HTLV-1 and HTLV-2).

Non-limiting examples of a fungal pathogen include yeasts and molds. More particular examples include Candida, Aspergillus, Cryptococcus, Histoplasma, Pneumocysti and Stachybotrys.

Non-limiting examples of a parasite pathogen include protozoa. More particular examples include Plasmodium, which causes malaria, Leishmania, Toxoplasma and Trypanosoma.

Pathogens and pathogen antigens, useful in accordance with the invention methods include any pathogen or pathogen antigen, or live or attenuated or weakened pathogen, suitable as a vaccine or immunizing agent, which typically can or is likely to provide protection against the pathogen.

Non-limiting examples include live pathogen, a pathogen antigen, pathogen extract, heat or ultraviolet light inactivated or attenuated or weakened pathogen.

Methods and uses of the invention include administering activin or subsequence thereof, or a activin receptor activator, in order to increase, stimulate enhance or promote a response elicited by vaccination or immunization. Such methods and uses can be used in particular with any cancer or tumor to which a vaccination, immunization or immunotherapy may be desired.

Non-limiting examples of cancer include Acute lymphoblastic leukemia (ALL); Acute myeloid leukemia; Adrenocortical carcinoma; AIDS-related cancers; AIDS-related lymphoma; Anal cancer; Appendix cancer; Astrocytoma; childhood cerebellar or cerebral; Basal-cell carcinoma; Bile duct cancer; extrahepatic (see Cholangiocarcinoma);

Bladder cancer; Bone tumor; Osteosarcoma/Malignant fibrous histiocytoma; Brainstem glioma; Brain cancer; Brain tumor; cerebellar astrocytoma; Brain tumor; cerebral astrocytoma/malignant glioma; Brain tumor; ependymoma; Brain tumor; medulloblastoma; Brain tumor; supratentorial primitive neuroectodermal tumors; Brain tumor; visual pathway and hypothalamic glioma; Breast cancer; Bronchial adenomas/carcinoids; Burkitt's lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer; Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liposarcoma; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Macroglobulinemia, Waldenström; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sézary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma; Squamous neck cancer with occult primary, metastatic; Stomach cancer; Supratentorial primitive neuroectodermal tumor, childhood; T-Cell lymphoma, cutaneous; Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenström macroglobulinemia or Wilms tumor (kidney cancer), childhood.

Therapeutic and prophylactic methods of treating a subject with an activin receptor activator or inhibitor, or activin or a subsequence thereof, include, for example, treatment of a subject having or at risk of having an immune disorder, such as an undesirable or aberrant immune response, inflammatory response, or inflammation. Such methods can treat the immune disorder, thereby providing the subject with a benefit.

In methods and uses of the invention, any activin receptor activator or inhibitor, or activin or subsequence thereof, can be administered or used. Non-limiting examples include an activin or activin receptor antibody or subsequence thereof, or activin or subsequence thereof (e.g., of SEQ ID NO: 1-2), or an amino acid insertion, addition or substitution thereof.

In particular methods and embodiments, treatment methods will modulate an immune response. Such modulation can stimulate, induce, increase or enhance, or inhibit, decrease or reduce, as needed. For example, proliferation, survival, differentiation, or activity of immune responses or cells (e.g., T cells, B cells, macrophages, neutrophils, dendritic cells, etc.), can be modulated.

Methods of the invention include treatment methods, which result in any therapeutic or beneficial effect. In various methods embodiments, an immune response is stimulated, induced, increased or enhanced, or inhibited, decreased or reduced. Methods of the invention further include inhibiting, decreasing or reducing one or more adverse (e.g., physical) symptoms, disorders, illnesses, diseases or complications caused by or associated with the immune response, such as (e.g., swelling, fever, rash, headache, infiltration of tissue or organs with immune cells, muscle or joint pain, nausea, vomiting, loss of appetite, etc.).

A therapeutic or beneficial effect of treatment is therefore any objective or subjective measurable or detectable improvement or benefit provided to a particular subject. A therapeutic or beneficial effect can but need not be complete ablation of all or any particular adverse symptom, disorder, illness, disease or complication caused by or associated with an immune response in a subject. Thus, a satisfactory clinical endpoint is achieved when there is an incremental improvement or a partial reduction in an adverse symptom, disorder, illness, disease or complication caused by or associated with an immune response, or an inhibition, decrease, reduction, suppression, prevention, limit or control of worsening or progression of one or more adverse symptoms, disorders, illnesses, diseases or complications caused by or associated with the immune response, over a short or long duration (hours, days, weeks, months, etc.).

Accordingly, methods of the invention that include treatment of an inflammatory response or inflammation include reducing, inhibiting or preventing occurrence, progression, severity, frequency or duration of a symptom or characteristic of an inflammatory response or inflammation. At the whole body, regional or local level, an inflammatory response or inflammation is generally characterized by swelling, pain, headache, fever, nausea, skeletal joint stiffness or lack of mobility, rash, redness or other discoloration. At the cellular level, an inflammatory response or inflammation is characterized by one or more of cell infiltration of the region, production of antibodies (e.g., autoantibodies), production of cytokines, lymphokines, chemokines, interferons and interleukins, cell growth and maturation factors (e.g., differentiation factors), cell proliferation, cell differentiation, cell accumulation or migration and cell, tissue or organ damage. Thus, treatment will reduce, inhibit or prevent occurrence, progression, severity, frequency or duration of any one or more of such symptoms or characteristics of an inflammatory response or inflammation.

A therapeutic or beneficial effect also includes reducing or eliminating the need, dosage frequency or amount of a second active such as another drug or other agent (e.g., small molecule, protein) used for treating a subject having or at risk of having an undesirable or aberrant immune response. For example, reducing an amount of an adjunct therapy, for example, a reduction or decrease of a treatment for an undesirable or aberrant immune response, inflammatory response or inflammation is considered a beneficial effect. In addition, reducing or decreasing an amount of a pathogen (live or attenuated or weakened) or pathogen antigen used for vaccination or immunization of a subject to provide protection to the subject is considered a beneficial effect.

Methods and compositions of the invention also include increasing, stimulating, promoting, enhancing, augmenting or inducing an immune response in a subject. In one embodiment, a method includes administering to a subject an amount of an activin receptor activator, or activin or subsequence thereof sufficient to increase, stimulate, promote, enhance, augment or induce an immune response in the subject. In another embodiment, a method includes administering to a subject an amount of an activin receptor activator, or activin or subsequence thereof, and administering pathogen (live or attenuated or weakened) or pathogen antigen sufficient to increase, stimulate, promote, enhance, augment or induce anti-pathogen immune response in the subject.

Methods and compositions of the invention include administration of an activin receptor inhibitor (e.g., Follistatin, Follistatin-like 3, Inhibin, Betaglycan, Cripto, BAMBI) activin or activin receptor antibody or inhibitory nucleic acid sequence, such as antisense RNA), or activin or subsequence thereof, to a subject prior to an undesirable or aberrant immune response, inflammatory response or inflammation, administration prior to, substantially contemporaneously with or after a subject has been afflicted with an undesirable or aberrant immune response, inflammatory response or inflammation, and administration prior to, substantially contemporaneously with or after development of one or more adverse symptoms caused by or associated with an undesirable or aberrant immune response, inflammatory response or inflammation. A subject with an undesirable or aberrant immune response, inflammatory response or inflammation, may have a chronic or acute undesirable or aberrant immune response, inflammatory response or inflammation, for example, a period of days, months, or years.

Methods and compositions of the invention also include administration of an activin receptor activator or activin or subsequence thereof to a subject prior to, substantially contemporaneously with or following a pathogen infection, or an adverse symptom, disorder, illness or disease caused by or associated with a pathogen infection or pathology. A subject infected with a pathogen may have a chronic or acute infection, for example, an infection for a period of days, months, or years.

Invention compositions (e.g., activin subunit polypeptides or subsequences thereof) and methods can be combined with any compound, agent, drug, treatment or other therapeutic regimen or protocol having a desired therapeutic, beneficial, additive, synergistic or complementary activity or effect.

Exemplary combination compositions and treatments include second actives, such as anti-immune response, inflammatory response or inflammation compounds, agents and drugs, as well as agents that assist, promote, stimulate or enhance an immune response where desired. Such drugs, agents, treatments and therapies can be administered or performed prior to, substantially contemporaneously with or following any other method of the invention, for example, a therapeutic method of treating a subject for an undesirable or aberrant immune response, inflammatory response or inflammation, or a method of vaccination or immunization.

An activin receptor activator or inhibitor, or activin or subsequence thereof, can be administered as a combination composition, or administered separately, such as concurrently or in series or sequentially (prior to or following) administering a second active, to a subject. The invention therefore provides combinations in which a method of the invention is used in a combination with any compound, agent, drug, therapeutic regimen, treatment protocol, process, remedy or composition, such as an anti-immune response, inflammatory response or inflammation, or an immune response stimulating, increasing, enhancing or augmenting protocol, such as pathogen vaccination or immunization set forth herein or known to one of skill in the art. The compound, agent, drug, therapeutic regimen, treatment protocol, process, remedy or composition can be administered or performed prior to, substantially contemporaneously with or following administration of an activin receptor activator or inhibitor, or activin or subsequence or thereof, to a subject. Specific non-limiting examples of combination embodiments therefore include the foregoing or other compound, agent, drug, therapeutic regimen, treatment protocol, process, remedy or composition.

Combination methods embodiments include, for example, second actives such as anti inflammatory or anti-inflammation agents and drugs and immune stimulating agents and drugs. Combination methods embodiments also include, for example, second actives such as anti-pathogen drugs (e.g., protease inhibitors, reverse transcriptase inhibitors, virus fusion inhibitors and virus entry inhibitors, antibodies to pathogen, live or attenuated or weakened pathogen, or a nucleic acid encoding all or a portion (e.g., an epitope) of any protein or proteinaceous pathogen antigen) immune stimulating agents and drugs, etc.

Particular non-limiting examples of anti-inflammatory, anti-inflammation and anti-autoimmune disease agents include immunosuppressive agents such as corticosteroids (steroid receptor agonists) including budesonide, prednisone, flunisolide, flunisolide hydrofluoroalkane, estrogen, progesterone, dexamethasone and loteprednol; beta-agonists (e.g., short or long-acting) such as bambuterol, formoterol, salmeterol, albuterol; anticholinergics such as ipratropium bromide, oxitropium bromide, cromolyn and calcium-channel blocking agents; antihistamines such as terfenadine, astemizole, hydroxyzine, tripelennamine, cetirizine, desloratadine, mizolastine, fexofenadine, olopatadine hydrochloride, norastemizole, levocetirizine, levocabastine, azelastine, ebastine and loratadine; antileukotrienes (e.g., anti-cysteinyl leukotrienes (CysLTs)) such as oxatomide, montelukast, zafirlukast and zileuton; phosphodiesterase inhibitors (e.g., PDE4 subtype) such as ibudilast, cilomilast, BAY 19-8004, theophylline (e.g., sustained-release) and other xanthine derivatives (e.g., doxofylline); thromboxane antagonists such as seratrodast, ozagrel hydrochloride and ramatroban; prostaglandin antagonists such as COX-1 and COX-2 inhibitors (e.g., celecoxib and rofecoxib), aspirin; potassium channel openers; and methotrexate (anti-metabolite), mycophenolate mofetil (purine biosynthesis inhibitor), and hydroxychloroquine (anti-malarial).

Additional specific examples of anti-inflammatory and anti-inflammation agents include antibodies, receptors or receptor ligands, such as anti-IgE (e.g., rhuMAb-E25 omalizumab), -IgA and -IgG antibodies; antibodies and soluble receptors against cytokines such as IL-1, IL-4, IL-5, IL-6, IL-9, IL-13, IL-16 and IL-21 or growth factors such as granulocyte/macrophage colony-stimulating factor; cytokines such as IL-10; mucolytics (depolymerize polymers of mucin or DNA/actin, or increase cough clearance) such as ambroxol and N-acetylcysteine; expectorants; and allergens (allergen immunotherapy).

Methods of the invention also include, among other things, methods that result in a reduced need or use of another compound, agent, drug, therapeutic regimen, treatment protocol, process, or remedy. For example, for an undesirable or aberrant immune response, inflammatory response or inflammation, a method of the invention has a therapeutic benefit if in a given subject a less frequent or reduced dose or elimination of another anti-inflammatory or anti-inflammation compound, agent, drug, therapeutic regimen, treatment protocol, process, or remedy. For example, for vaccination or immunization, a method of the invention has a therapeutic benefit if in a given subject a less frequent or reduced dose or elimination of a vaccine. Thus, in accordance with the invention, methods of reducing need or use of another treatment or therapy are provided.

In invention methods in which there is a desired outcome, such as a therapeutic or prophylactic method that provides a benefit from treatment, vaccination or immunization with an activin receptor activator or inhibitor, or activin or subsequence thereof can be administered in a sufficient or effective amount. As used herein, a “sufficient amount” or “effective amount” or an “amount sufficient” or an “amount effective” refers to an amount that provides, in single or multiple doses, alone or in combination with one or more other compounds, treatments, therapeutic regimens or agents (e.g., a drug), a long term or a short term detectable or measurable improvement in a given subject or any objective or subjective benefit to a given subject of any degree or for any time period or duration (e.g., for minutes, hours, days, months, years, or cured).

An amount sufficient or an amount effective can but need not be provided in a single administration and can but need not be achieved by an activin receptor activator or inhibitor, or activin or subsequence thereof, alone, in a combination composition or method that includes a second active. In addition, an amount sufficient or an amount effective need not be sufficient or effective if given in single or multiple doses without a second or additional administration or dosage, since additional doses, amounts or duration above and beyond such doses, or additional antigens, compounds, drugs, agents, treatment or therapeutic regimens may be included in order to provide a given subject with a detectable or measurable improvement or benefit to the subject.

An amount sufficient or an amount effective need not be therapeutically or prophylactically effective in each and every subject treated, nor a majority of subjects treated in a given group or population. An amount sufficient or an amount effective means sufficiency or effectiveness in a particular subject, not a group of subjects or the general population. As is typical for such methods, different subjects will exhibit varied responses to treatment.

In the case of an undesirable or aberrant immune response, treatment methods include reducing or increasing numbers or an activity of immune cells (e.g., lymphocytes, T cells, antigen presenting cells, B cells, etc.) towards physiologically normal baseline levels. Similarly, a reduction or decrease of circulating antibodies (e.g., auto-antibodies) towards normal is considered a successful treatment outcome.

Additional examples of a therapeutic benefit for an undesirable or aberrant immune response, immune disorder or immune disease is an improvement in a histopathological change caused by or associated with the immune response, disorder or disease. For example, preventing further or reducing skeletal joint infiltration or tissue destruction, or pancreas, thymus, kidney, liver, spleen, epidermal (skin) or mucosal tissue, gut or bowel infiltration or tissue destruction.

The term “subject” refers to an animal, typically a mammalian animal (mammal), such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Subjects include animal disease models, for example, mouse and other animal models of inflammation, undesirable and aberrant immune responses, inflammatory and autoimmune diseases and others known to those of skill in the art (e.g., CIA, BXSB, EAE and SC murine models).

Subjects appropriate for treatment include those having or at risk of having an undesirable or aberrant immune response, inflammatory response or inflammation, or vaccination or immunization, or a pathogen infection. Target subjects therefore include subjects that have an undesirable or aberrant immune response, inflammatory response or inflammation, or have been or are in need of vaccination or immunization, or have been exposed to or contacted with a pathogen, regardless of the type, timing or degree of onset, progression, severity, frequency, duration of the symptoms.

The invention methods are therefore applicable to treating a subject who is at risk of undesirable or aberrant immune response, inflammatory response or inflammation, or vaccination or immunization, or a pathogen infection, but may not have a symptom of an undesirable or aberrant immune response, inflammatory response or inflammation, or a pathogen infection, or have been exposed to or contacted with the pathogen. Prophylactic methods are therefore included. Such subjects are considered in need of treatment due to being at risk.

Target subjects need not be at increased risk but may be from the general population in which it is desired to inhibit, decrease or reduce an undesirable or aberrant immune response, inflammatory response or inflammation, or vaccination or immunization, or a pathogen infection. For example, a child such as an infant or toddler in which it is desired to vaccinate or immunize against a pathogen can be administered an Actvin or activin receptor activator or inhibitor, or activin or subsequence thereof, alone or in combination with a pathogen antigen. In another non-limiting example, a subject that is not specifically at risk of an undesirable or aberrant immune response, inflammatory response or inflammation, or vaccination or immunization, or pathogen infection, exposure to or contact, but nevertheless desires protection from an undesirable or aberrant immune response, inflammatory response or inflammation, or a pathogen infection or pathology, can be administered an activin receptor activator or inhibitor, or activin or subsequence thereof. Such subjects are also considered in need of treatment.

Target subjects also include subjects having or at risk of having immunosuppression or are immunocompromised or have or are at risk of an immunodeficiency. Specific non-limiting examples of such subjects have or at risk of having an immunodeficiency, such as that caused by chemotherapy or radiotherapy (ionizing or chemical) or immune-suppressive therapy following a transplant (e.g., organ or tissue such as heart, liver, lung, bone marrow, etc.). Additional non-limiting examples include subjects having or at risk of having a graft vs. host disease, e.g., a subject that is a candidate for a transplant or a subject undergoing or having received a transplant.

At risk subjects appropriate for treatment also include subjects exposed to environments in which subjects are at risk of a pathogen infection. Subjects appropriate for treatment therefore include human subjects exposed to pathogens.

“Prophylaxis” and grammatical variations thereof mean a method in which contact, administration or in vivo delivery to a subject is prior to an undesirable or aberrant immune response, inflammatory response or inflammation, or vaccination or immunization, or exposure to or contact with a pathogen. Administration or in vivo delivery to a subject can be performed prior to development of an adverse symptom, condition, complication, etc. caused by or associated with undesirable or aberrant immune response, inflammatory response or inflammation, or vaccination or immunization, or pathogen infection. In such case, a method can eliminate, prevent, inhibit, suppress, limit, decrease or reduce the probability of an undesirable or aberrant immune response, inflammatory response or inflammation, or a pathogen infection or susceptibility.

Administration of an activin receptor activator or inhibitor, or activin or subsequence thereof, for treatment of an undesirable or aberrant immune response, inflammatory response or inflammation, vaccination or immunization, or a pathogen infection can be at any time. Methods of the invention may be practiced by any mode of administration or delivery, or by any route, via systemic, regional and local administration or delivery. For example, an activin receptor activator or inhibitor, or a activin or subsequence, may be administered systemically, regionally or locally, intravenously, orally (e.g., ingestion or inhalation), intramuscularly, intraperitoneally, intradermally, subcutaneously, intracavity, intracranially, transdermally (topical), parenterally, e.g. transmucosally or rectally. Exemplary administration and delivery routes include intravenous (i.v.), intraperitoneal (i.p.), intrartenal, intramuscular, parenteral, subcutaneous, intra-pleural, topical, dermal, intradermal, transdermal, transmucosal, intra-cranial, intra-spinal, rectal, oral (alimentary), mucosal, inhalation, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, intralymphatic.

An activin receptor activator or inhibitor, or activin or subsequence thereof, can be administered as a combination (e.g., with a second active), or separately concurrently or in sequence (sequentially or serially) in accordance with the methods as a single or multiple dose e.g., one or more times hourly, daily, weekly, monthly or annually or between about 1 to 10 weeks, or for as long as appropriate, for example, to achieve a desired effect or activity. Thus, a method can be practiced one or more times (e.g., 1-10, 1-5 or 1-3 times) an hour, day, week, month, or year. The skilled artisan will know when it is appropriate to delay or discontinue administration. A non-limiting dosage schedule is 1-7 times per week, for 1, 2, 3, 4, 5, 6, 7, 8, 9, 10, 15, 20 or more weeks, and any numerical value or range or value within such ranges.

Doses can be based upon current existing protocols, empirically determined, using animal disease models or optionally in human clinical trials. Initial study doses can be based upon animal studies set forth herein, for a mouse, which weighs about 30 grams, and the amount of an activin receptor activator or inhibitor, or activin or subsequence thereof administered that is determined to be effective. Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. The dose can be adjusted according to the mass of a subject, and will generally be in a range from about 1-10 ug/kg, 10-25 ug/kg, 25-50 ug/kg, 50-100 ug/kg, 100-500 ug/kg, 500-1,000 ug/kg, 1-5 mg/kg, 5-10 mg/kg, 10-20 mg/kg, 20-50 mg/kg, 50-100 mg/kg, 100-250 mg/kg, 250-500 mg/kg, or more, two, three, four, or more times per hour, day, week, month or annually. A typical range will be from about 0.3 mg/kg to about 50 mg/kg, 0-25 mg/kg, or 1.0-10 mg/kg, or any numerical value or range or value within such ranges.

Doses can vary and depend upon whether the treatment is prophylactic or therapeutic, the type, onset, progression, severity, frequency, duration, or probability of the undesirable or aberrant immune response, inflammatory response or inflammation, vaccination or immunization, or pathogen infection to which treatment is directed, the clinical endpoint desired, previous or simultaneous treatments, the general health, age, gender, race or immunological competency of the subject and other factors that will be appreciated by the skilled artisan. The skilled artisan will appreciate the factors that may influence the dosage and timing required to provide an amount sufficient for providing a therapeutic or prophylactic benefit.

Typically, for therapeutic treatment, an activin receptor activator or inhibitor, or activin or subsequence thereof will be administered as soon as practical, typically within 1-2, 2-4, 4-12, 12-24 or 24-72 hours after a subject has a symptom or is exposed to or contacted with a pathogen, or within 1-2, 2-4, 4-12, 12-24 or 24-48 hours after onset or development of one or more adverse symptoms, conditions, pathologies, complications, etc., associated with or caused by. For prophylactic treatment in connection with vaccination or immunization, an activin receptor activator or inhibitor, or activin or subsequence thereof can be administered for a duration of 0-4 weeks, e.g., 2-3 weeks, prior to exposure to, contact or infection with pathogen, or at least within 1-2, 2-4, 4-12, 12-24, 24-48 or 48-72 hours prior to exposure to, contact or infection with pathogen. For an acute or chronic undesirable or aberrant immune response, inflammatory response or inflammation, or vaccination or immunization, or a pathogen infection, an activin receptor activator or inhibitor, or activin or subsequence thereof, is administered at any appropriate time.

The dose amount, number, frequency or duration may be proportionally increased or reduced, as indicated by the status of the subject. The dose amount, number, frequency or duration may also be proportionally increased or reduced, as indicated by any adverse side effects, complications or other risk factors of the treatment or therapy.

Activin Receptor activators, inhibitors, and activin and subsequences thereof can be incorporated into pharmaceutical compositions, e.g., a pharmaceutically acceptable carrier or excipient. Such pharmaceutical compositions are useful for, among other things, administration to a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration. Thus, pharmaceutical compositions include carriers, diluents, or excipients suitable for administration by various routes. Exemplary routes of administration for contact or in vivo delivery which a composition can optionally be formulated include inhalation, respiration, intranasal, intubation, intrapulmonary instillation, oral, buccal, intrapulmonary, intradermal, topical, dermal, parenteral, sublingual, subcutaneous, intravascular, intrathecal, intraarticular, intracavity, transdermal, iontophoretic, intraocular, opthalmic, optical, intravenous (i.v.), intramuscular, intraglandular, intraorgan, intralymphatic.

Formulations suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils.

Carriers which may be used include Vaseline, lanolin, polyethylene glycols, alcohols, transdermal enhancers, and combinations thereof.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

An antimicrobial agent or compound directly or indirectly inhibits, reduces, delays, halts, eliminates, arrests, suppresses or prevents contamination by or growth, infectivity, replication, proliferation, reproduction, of a pathogenic or non-pathogenic microbial organism. Classes of antimicrobials include, antibacterial, antiviral, antifungal and antiparasitics. Antimicrobials include agents and compounds that kill or destroy (-cidal) or inhibit (-static) contamination by or growth, infectivity, replication, proliferation, reproduction of the microbial organism.

Exemplary antibacterials (antibiotics) include penicillins (e.g., penicillin G, ampicillin, methicillin, oxacillin, and amoxicillin), cephalosporins (e.g., cefadroxil, ceforanid, cefotaxime, and ceftriaxone), tetracyclines (e.g., doxycycline, chlortetracycline, minocycline, and tetracycline), aminoglycosides (e.g., amikacin, gentamycin, kanamycin, neomycin, streptomycin, netilmicin, paromomycin and tobramycin), macrolides (e.g., azithromycin, clarithromycin, and erythromycin), fluoroquinolones (e.g., ciprofloxacin, lomefloxacin, and norfloxacin), and other antibiotics including chloramphenicol, clindamycin, cycloserine, isoniazid, rifampin, vancomycin, aztreonam, clavulanic acid, imipenem, polymyxin, bacitracin, amphotericin and nystatin.

Particular non-limiting classes of anti-virals include reverse transcriptase inhibitors; protease inhibitors; thymidine kinase inhibitors; sugar or glycoprotein synthesis inhibitors; structural protein synthesis inhibitors; nucleoside analogues; and viral maturation inhibitors. Specific non-limiting examples of anti-virals include nevirapine, delavirdine, efavirenz, saquinavir, ritonavir, indinavir, nelfinavir, amprenavir, zidovudine (AZT), stavudine (d4T), larnivudine (3TC), didanosine (DDI), zalcitabine (ddC), abacavir, acyclovir, penciclovir, ribavirin, valacyclovir, ganciclovir, 1,-D-ribofuranosyl-1,2,4-triazole-3 carboxamide, 9->2-hydroxy-ethoxy methylguanine, adamantanamine, 5-iodo-2′-deoxyuridine, trifluorothymidine, interferon and adenine arabinoside.

Pharmaceutical formulations and delivery systems appropriate for the compositions and methods of the invention are known in the art (see, e.g., Remington: The Science and Practice of Pharmacy (2003) 20th ed., Mack Publishing Co., Easton, Pa.; Remington's Pharmaceutical Sciences (1990) 18 h ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al, Drug Delivery Systems (1980), R. L. Juliano, ed., Oxford, N.Y., pp. 253-315).

An activin receptor activator or inhibitor, or activin or subsequence thereof, along with any adjunct agent, compound drug, composition, whether active or inactive, etc., can be packaged in unit dosage form (capsules, tablets, troches, cachets, lozenges) for ease of administration and uniformity of dosage. A “unit dosage form” as used herein refers to physically discrete units suited as unitary dosages for the subject to be treated; each unit containing a predetermined quantity of active ingredient optionally in association with a pharmaceutical carrier (excipient, diluent, vehicle or filling agent) which, when administered in one or more doses, is calculated to produce a desired effect (e.g., prophylactic or therapeutic effect). Unit dosage forms also include, for example, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Individual unit dosage forms can be included in multi-dose kits or containers. Pharmaceutical formulations can be packaged in single or multiple unit dosage form for ease of administration and uniformity of dosage.

The invention provides kits that include activin receptor activators, inhibitors, activin and subsequences thereof, optionally with a second active, and pharmaceutical formulations thereof, packaged into suitable packaging material. A kit typically includes a label or packaging insert including a description of the components or instructions for use in vitro, in vivo, or ex vivo, of the components therein. A kit can contain a collection of such components, e.g., activin or subsequence thereof and optionally a second active, such as another compound, agent, drug or composition.

The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits of the invention can include labels or inserts. Labels or inserts can include information identifying manufacturer, lot numbers, manufacturer location and date, expiration dates. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer information, lot numbers, manufacturer location and date.

Labels or inserts can include information on a condition, disorder or disease (e.g., viral infection, vaccination or immunization) for which a kit component may be used. Labels or inserts can include instructions for the clinician or subject for using one or more of the kit components in a method, or treatment protocol or therapeutic regimen. Instructions can include dosage amounts, frequency or duration, and instructions for practicing any of the methods, treatment protocols or prophylactic or therapeutic regimes described herein. Exemplary instructions include, instructions for administering an activin receptor activator or inhibitor, or activin or subsequence thereof.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., compound structures) are an example of a genus of equivalent or similar features.

As used herein, the singular forms “a”, “and,” and “the” include plural referents unless the context clearly indicates otherwise. Thus, for example, reference to “a first, second, third, fourth, fifth, etc. predictor gene” or a “positive or negative predictor gene” includes a plurality of such first, second, third, fourth, fifth, etc., genes, or a plurality of positive and/or negative predictor genes.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

Reference to a number with more (greater) or less than includes any number greater or less than the reference number, respectively. Thus, for example, a reference to less than 30,000, includes 29,999, 29,998, 29,997, etc. all the way down to the number one (1); and less than 20,000, includes 19,999, 19,998, 19,997, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as a percentage range, 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 1-5 fold therefore includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

Reference to a series of ranges includes ranges which combine the values of the boundaries of different ranges within the series. Thus, to illustrate reference to a series of ranges of 2-72 hours, 2-48 hours, 4-24 hours, 4-18 hours and 6-12 hours, includes ranges of 2-6 hours, 2, 2 hours, 2-18 hours, 2-24 hours, etc., and 4-27 hours, 4-48 hours, 4-6 hours, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

Materials and Methods

Human Samples

Leukapheresis sample from a healthy donor was obtained from AllCells, Inc., after a prescreening of multiple donors. Fresh whole blood samples from healthy donors were obtained from the La Jolla Institute for Allergy and Immunology (LJI) in-house normal blood donor program (NBDP). Informed consent was obtained from all donors. Fresh human tonsils were obtained from the National Disease Resource Interchange. Tonsil preparation was previously described. Peripheral blood mononuclear cells (PBMCs) were isolated from both leukapheresis and whole blood samples by density-gradient centrifugation using Histopaque-1077 (Sigma-Aldrich).

Flow Cytometry and Cell Sorting

For surface staining, primary-staining panels used for phenotypic analysis or for cell sorting known in the art were used. For the intranuclear staining: cells were treated with the FoxP3 Fixation/Permeabilization kit (eBioscience) and stained in Permeabilization buffer (eBioscience). For cytokine staining: day 3 or day 5 in vitro differentiated cells (specified in the main text or in figure legends) cells were stimulated for 5 hours at 37° C. with Phorbol 12-myristate 13-acetate (PMA, 25 ng/ml) and Ionomycin (1 μg/ml) in the presence of Brefeldin A (5 μg/ml). Cells were fixed with PBS 2% FBS, 1% PFA and permeabilized with PBS 2% FBS, 0.5% Saponin (from quillaja bark, Sigma-Aldrich). Samples were analyzed on a LSR Fortessa Cell Analyzer (BD, Bioscience). Flow cytometry data were analyzed with FlowJo (Tree Star). For cell sorting, cells were sorted using sorted using a FACS Aria (BD, Bioscience).

Screening of “Secretomics” Recombinant Proteins

Naïve CD4⁺ T cells were isolated from leukapheresis-derived mononuclear cells via CliniMACS negative selection (Miltenyi Biotec). The “secretomics” collection was previously described¹⁹. An expanded secretomics set (Phasell), composed of 2688 unique proteins (total 1772 genes) was tested. Naïve CD4⁺ T cells (1.25×10⁴ cells/well) were cultured with Secretomics proteins in the presence of Dynabeads Human T-Activator CD3/CD28 (0.5 μl/well, Life Technologies), recombinant human IL-7 (4 ng/ml, Peprotech), and anti-human-TGFβ blocking mAb (100 μg/ml, clone 1D11.16.8, BioXcell) in RPMI medium (Cellgro), supplemented with 10% fetal bovine serum (Omega Scientific), GlutaMAX (Gibco) and Penicillin/Streptomycin (Gibco). Induction of CXCR5 and PD-1 was assessed after 5 days of in vitro culture using automated FACS acquisition via HTS on LSR Fortessa (BD Biosciences). For each 384 well plate, mean and standard deviation of PD-1⁺CXCR5⁺ cell frequency induced by each individual recombinant proteins was determined and used to calculate the Z score. Z scores of replicate plates were analyzed independently.

In Vitro Differentiation

Unless differently specified, naïve CD4⁺ T cells were enriched from PBMCs by magnetic bead negative selection with the Naïve CD4⁺ T Cell Isolation Kit (Stem Cell Technologies). Purity (CD4⁺CD45RA⁺) was 90% or higher. Cells (7.5×10⁴ cells/well) were activated by Dynabeads Human T-Activator CD3/CD28 (2 μl/well, Life Technologies) and cultured with recombinant human/mouse/rat activin A (50 ng/ml or 100 ng/ml), recombinant human IL-12 (5 ng/ml), recombinant human TGFβ (1 ng/ml), recombinant human IL-23 (10 ng/ml) or recombinant human IL-35 (10 ng/ml) in the presence of and recombinant human IL-7 (4 ng/ml) in AIM-V medium (Life Technologies). All cytokines were from R&D System and Peprotech. The 14 kDa mature human beta A chain of human activin A shares 100% amino acid sequence identity with bovine, feline, mouse, porcine, and rat beta A. Phenotype was quantified after 3 or 5 days, as specified in the main text of Figure legends. Similar results were obtained when using Treg-depleted FACS sorted naïve CD4 T cells, in which cells were sorted from total PBMCs by flow cytometry as CD19⁻CD8⁻CD14⁻CD16⁻ CD4⁺CD45RO⁻CD25⁻ cells. For experiments quantifying supernatant CXCL13 concentration, cells were activated by plate-bound anti-human CD3 (5 μg/ml, BD) and ICOS-L (5 μg/ml, recombinant human B7-H2, R&D). CXCL13 concentrations were determined via human CXCL13 DuoSet ELISA (R&D) on supernatants harvested after 5 days of in vitro culture. For blocking experiments, cells were cultured with anti-activin A blocking mAb (5 μg/ml, clone 69403, R&D), anti-TGFβ mAb (100 μg/ml, clone 1D11.16.8, BioXcell), or equivalent concentration of mouse IgG1 isotype mAb (BioXcell). For blockade of IL-2, cells were cultured with anti-IL-2 blocking mAb (10 μg/ml, clone 5334, R&D) or mouse IgG1 isotype mAb (BioXcell). For SMAD2/3 inhibition experiments, cells were cultured with different concentrations of SB 431542 (Sigma-Aldrich), Galunisertib (LY2157299, Selleck Chemicals) or vehicle (DMSO) for 5 days in AIM-V supplemented with 2% heat-inactivated FBS or in RPMI-1640 (Corning) supplemented with 10% heat-inactivated FBS, 1% GlutaMax, 1% PenStrep.

Confocal Microscopy

Tonsils were fixed for 4 hours in freshly diluted 4% paraformaldehyde in PBS (Electron Microscopy Sciences, Hatfield, Pa. EMS Diasum), then washed 3 times in PBS and equilibrated in 30% w/v sucrose solution in PBS for 24 hours. Tissues were briefly washed in PBS, frozen in OCT and stored at −80° C. Cryostat sections were cut at 12 μm thickness, and air-dried for 30 minutes. Sections were rehydrated for 10 minutes in PBS and non-specific binding sites were saturated with 5% normal donkey serum (Jackson Immunoresearch) in the presence of 0.3% Triton X-100 in PBS for 1 hour. Tissues were incubated with rabbit polyclonal anti-INHBA Ab (HPA020031, Sigma-Aldrich), mouse anti-CD3 FITC (UCHT1, Tonbo Biosciences) and mouse anti-Bcl6 Alexa Fluor 647 (K112-91, BD Biosciences) overnight at 4° C. Following washing, sections were reacted with donkey anti-rabbit Alexa Fluor 568 and goat anti-FITC Alexa Fluor 488 secondary antibodies (Invitrogen) for 1 hour at room temperature. Slides were counterstained with 10 μg/ml Hoechst for 10 minutes, washed and mounted in Prolong Gold antifade reagent (Invitrogen) with a cover slip and examined with AxioScan Z1 slide scanner equipped in 20×/0.8 NA air objective using appropriate filter sets. High resolution imaging of selected regions was done on a FluoView FV10i confocal microscope (Olympus) using 60×/1.35 NA oil objective. To reduce blurriness epifluorescence images were processed with an extended depth of field algorithm (ZEN2, Zeiss). To improve feature visibility images were processed by contrast stretching using identical procedures (ZEN2 and Olympus FluoView software).

T-B Co-Culture

Naïve CD4⁺ T cells were differentiated with activin A and/or IL-12. At day 3, live CD4⁺ T cells were sorted by FACS. Fresh autologous B cells were enriched from PBMCs by CD19 magnetic-bead positive selection (Miltenyi Biotec). Memory B cells were then FACS sorted as CD3⁻CD14⁻CD4⁻CD19⁺IgD⁻CD27⁺CD38⁻ cells and cultured (4×10⁵ cells/well) with the in vitro differentiated T cells (2.5×10³ cells/well) in the presence of staphylococcal enterotoxin B (SEB, 0.25 ng/ml, Toxin Technology) in AIM-V medium. B cell counts and plasmablast frequencies were measured after 7 days by FACS. Ig concentrations in the supernatants were determined by ELISA.

Mouse CD4 T Cell Differentiation

Naïve CD4⁺ T cells were isolated from spleen using the Naïve CD4⁺ T cell isolation kit (Stem Cell). Purity was 92% or higher. Naïve CD4⁺ T cells (2×10⁵ cells/well) were cultured for 3 days with recombinant human/mouse/rat activin A (50 ng/ml, R&D Systems), recombinant mouse IL-12 (10 ng/ml, Peprotech), and/or recombinant mouse IL-6 (20 ng/ml, Peprotech) in the presence of plate-bound anti-mouse-CD3 mAb (8 μg/ml, clone 145-2C11, BioXcell) and anti-mouse-CD28 (8 μg/ml, clone 37.51, BioXcell), in RPMI medium, supplemented with 10% fetal bovine serum, GlutaMAX, penicillin/streptomycin, and 2.5 μM β-mercaptoethanol. After 3 days, cells were removed from stimuli and further cultured for 2 additional days in IL-2 (50 U/mL,) and the same cytokine combination used at day 0. Phenotype was quantified by FACS at day 5 of the in vitro culture.

Non-Human Primate (NHP) Differentiation

CD4⁺ T cells from rhesus macaque PBMCs were enriched using the Non-Human Primate CD4⁺ T Cell Isolation Kit (Miltenyi Biotec). Naïve CD4⁺ T cells were isolated by FACS sorting as CD4⁺CD45RA⁺CD95⁻CD28⁺CCR7⁺ cells. Naïve CD4⁺ T cells (7.5×10⁴ cells/well) were activated by plate-bound anti-human-CD3 and anti-human-CD28 (both at 5 μg/ml, BD) and cultured with recombinant human/mouse/rat activin A (50 ng/ml) and/or recombinant human IL-12 (5 ng/ml) for 5 days. Phenotype was assessed by flow cytometry.

RNA Sequencing

Differentiation of naïve CD4⁺ T cells with activin A and/or IL-12 was described above. Naïve CD4 T cells from 4 or more donors were cultured with the following conditions: beads only (beads), IL-12, activin A (100 ng/ml), activin A (100 ng/ml)+IL-12, TGFβ, TGFβ+IL-12. After 3 days of in vitro culture, live CD4⁺ T cells were sorted by FACS. Total RNA was purified using miRNAeasy Mini kit (Qiagen). Standard quality control steps were included to determine total RNA quality using Agilent Bioanalyzer (RNA integrity number (RIN)>8.5; Agilent RNA 6000 Nano Kit, USA) and quantity using a nanoliter spectrophotometer (Nanodrop, Thermofisher, USA). For every sample, 500 ng of purified total RNA was prepared into mRNA libraries, according to manufacturer's instructions, using the Truseq Stranded mRNA Library Prep Kit (Illumina, RS-122-2103). The resulting libraries were deep sequenced, using the Illumina HiSeq2500 system in rapid run mode, to obtain between 6 to 7 millions of 50-bp length single-end reads per library.

Analysis of RNA-Seq Data

The single-end reads that passed Illumina filters were filtered for reads aligning to tRNA, rRNA, adapter sequences, and spike-in controls. The reads were then aligned to UCSC hg19 reference genome using TopHat⁵⁵(v 1.4.1). DUST scores were calculated with PRINSEQ Lite⁵⁶ (v 0.20.3) and low-complexity reads (DUST >4) were removed from the BAM files. The alignment results were parsed via the SAMtools⁵⁷ to generate SAM files. Read counts to each genomic feature were obtained with the htseq-count program⁵⁸ (v 0.6.0) using the “union” option. After removing absent features (zero counts in all samples), the raw counts were converted to RPKM value followed by quantile normalization via R library ‘aroma.light’ ⁵⁹. Then the quantile normalized RPKM values were filtered by setting a cutoff value of 1 and analyzed with the Multiplot module in GenePattern suite (http://www.broadinstitute.org/cancer/software/genepattern/) to generate the Volcano, Heatmap and Expression plots. Meanwhile the filtered raw counts were imported to R/Bioconductor package DESeq2⁶⁰ (v 3.1) to normalize counts and identify differentially expressed genes among conditions.

Gene Set Enrichment Analysis (GSEA)

Gene Set Enrichment Analysis (v 2.2.0; http://www.broadinstitute.org/gsea/index.jsp) was used to evaluate if a pre-defined tonsil GC-Tfh gene set showed statistically significant enrichment between two phenotypes when cells are given a particular stimulus. The tonsil GC Tfh gene set was generated from previously published microarray data³⁸ and defined by genes with more than two-fold higher expression in GC-Tfh cells (CD4⁺CD45RO⁺PD-1^{hi}CXCR5^{hi}) compared to non-Tfh cells (CD4⁺CD45RO⁺PD-1⁻CXCR5⁻). For RNA-seq profiles used as the query list, genes were ranked from the most up-regulated to the most down-regulated in cells stimulated with beads versus activin A+IL-12, IL-12 versus activin A+IL-12, and activin A versus activin A+IL-12 on the basis of the DESeq2 analysis results. The signal-to-noise metric was used for ranking the genes in the RNA-seq expression datasets and 10⁵ cycles of permutations on the phenotype labels were performed to determine the normalized enrichment score (NES).

Quantitative Real-Time PCR

RNA was isolated by QIAGEN RNeasy spin columns and reverse-transcribed into cDNA using Superscript II Reverse Transcriptase (Invitrogen). Quantitative real-time PCR of ACTB, LIF, and PRDM1 was performed using the following primers: ACTB forward, 5′-ACCTTCTACAATGAGCTGCG-3′, ACTB reverse, 5′-CCTGGATAGCAACGTACATGG-3′; LIF forward, 5′-ATACGCCACCCATGTCAC-3′, LIF reverse, 5′-CCACATAGCTTGTCCAGGTTG-3′; and PRDM1 forward, 5′-TGTGGTATTGTCGGGACTTTG-3′, PRDM1 reverse 5′-CTTTGGGACATTCTTTGGGC-CTG-3′. Real-time PCR was set up with Applied Biosystem SYBR Green Master Mix.

Analysis of Phosphorylated SMAD2/3

Fresh PBMCs and tonsil mononuclear cells were serum-starved over night in AIM-V medium. The following day, the cells (10̂⁶ cells/condition) were stimulated for 10, 30, 60, 120 or 180 minutes with activin A (400 ng/ml)+vehicle (DMSO), activin A+SB 431542 (10 μM, Sigma-Aldrich), Galunisertib (10 μg/ml, Selleck Chemicals) or vehicle only in AIM-V medium. Following stimulation, the cells were fixed in BD Phosflow™ Fix Buffer I (BD Biosciences), then permeabilized using BD Phosflow™ Perm Buffer III (BD Biosciences) following manufacture's instruction.

Statistical Analysis

All the statistical analyses, unless differently specified, were done with two-tailed Wilcoxon matched-pairs signed ranked test, which does not assume Gaussian distribution of the data, but it allows the direct comparison of populations within donors. Independent experiments were repeated several times with controls. Prism 6.0 (GraphPad) was used for all analysis.

Anti-Activin a Type II Receptor (ACVR2) Blocking mAb

Naïve CD4⁺ T cells were enriched from PBMCs by magnetic bead negative selection with the Naïve CD4⁺ T Cell Isolation Kit (Stem Cell Technologies). Purity (CD4⁺CD45RA⁺) was 90% or higher. Cells (7.5×10⁴ cells/well) were activated by Dynabeads Human T-Activator CD3/CD28 (2 μl/well, Life Technologies) and cultured with recombinant human/mouse/rat activin A (50 ng/ml) or recombinant human IL-12 (5 ng/ml) in the presence of and recombinant human IL-7 (4 ng/ml) in AIM-V medium (Life Technologies). Phenotype was quantified after 5 days, by flow cytometry. For blocking experiments, cells were cultured with pan anti-activin A Type II receptor (ACVR2) blocking mAb (100 μg/ml, clone MOR8806, Novartis, described previously in International Publication No. WO2010125003, published Nov. 4, 2010, the contents of which are incorporated herein by reference.) or equivalent concentration of isotype mAb (Novartis).

### Example 1: Summary of Initial Experiments

T follicular helper (Tfh) cells are CD4 T cells specialized in helping B cell responses. Tfh cells are characterized by the expression of signature surface molecules, including CXCR5 and PD-1. CXCR5 is chemokine receptor involved in the location of Tfh cells at the border with B cell follicle, while PD-1 is an inhibitory receptor controlling the expansion of Tfh cells. An unbiased high throughput human Tfh cell differentiation screen was conducted using recombinant proteins. Overall, over 3000 unique human proteins were tested for their ability to induce expression of the Tfh signature markers CXCR5 and PD-1 in human naive CD4 T cells upon activation. The screen identified TGFβ as regulator T cell differentiation and unexpectedly, the cytokine activin A emerged from the screen as a potent regulator of the human Tfh gene program.

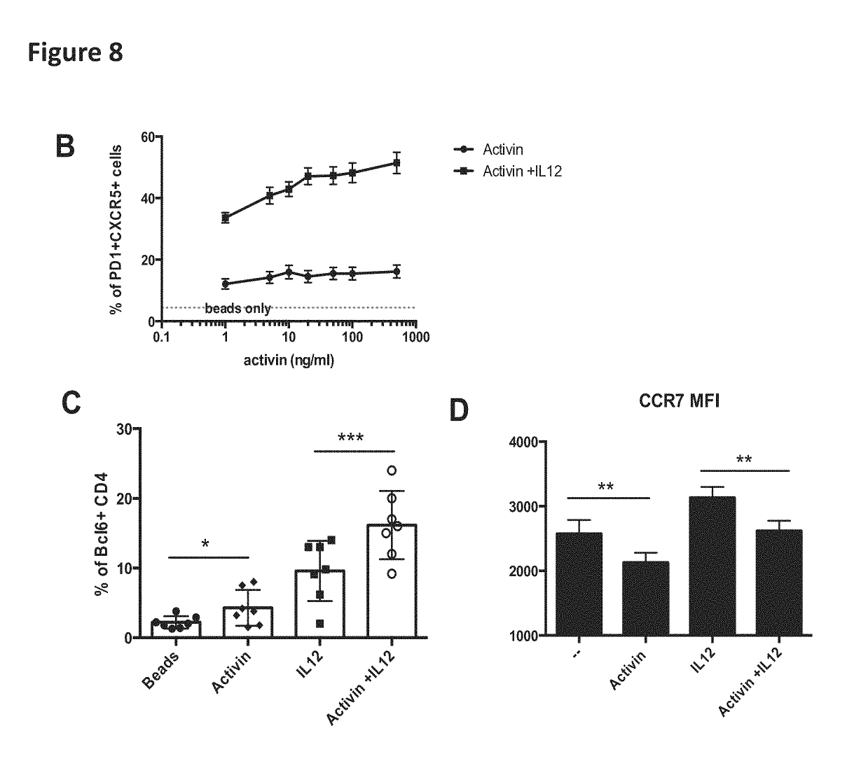

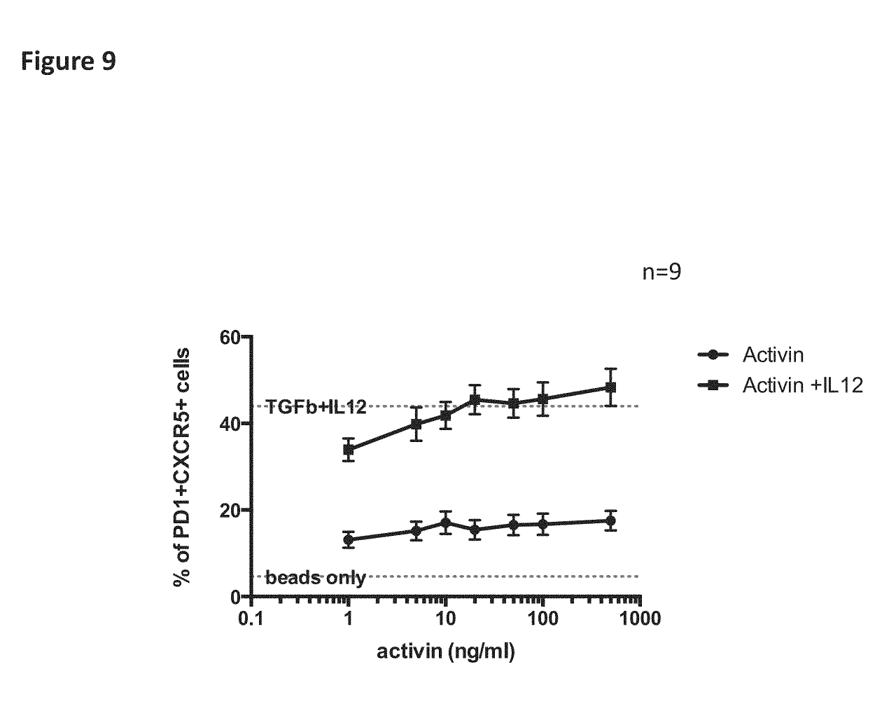

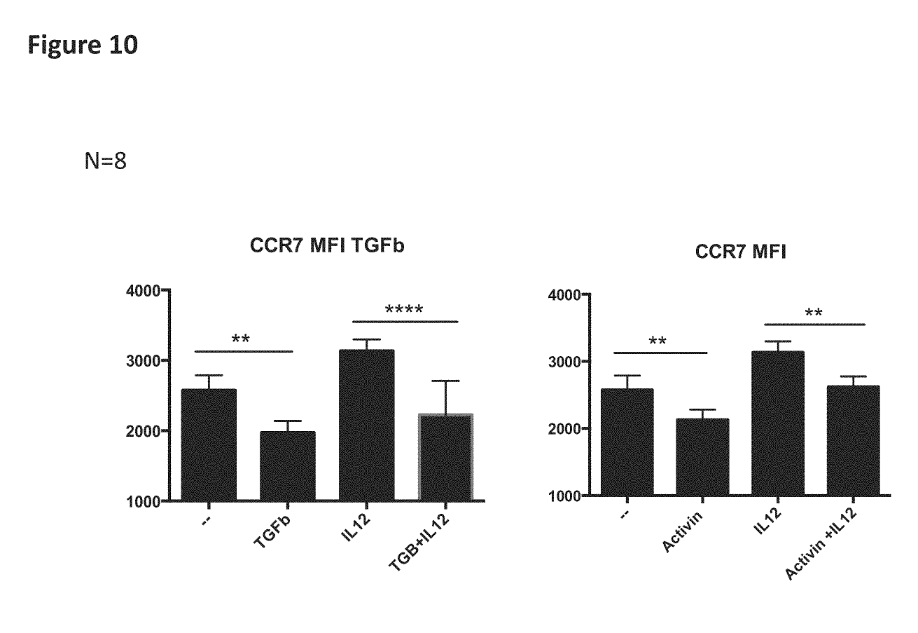

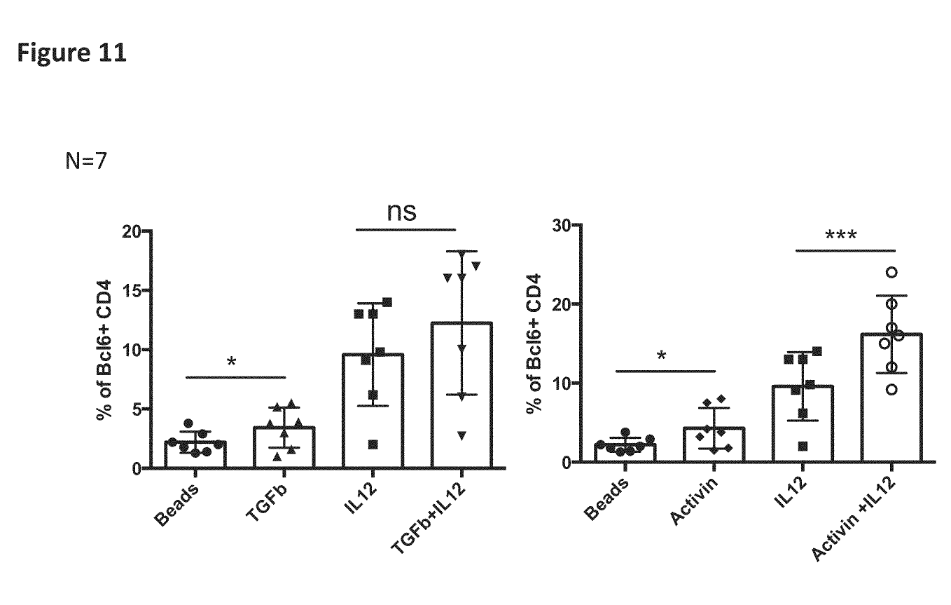

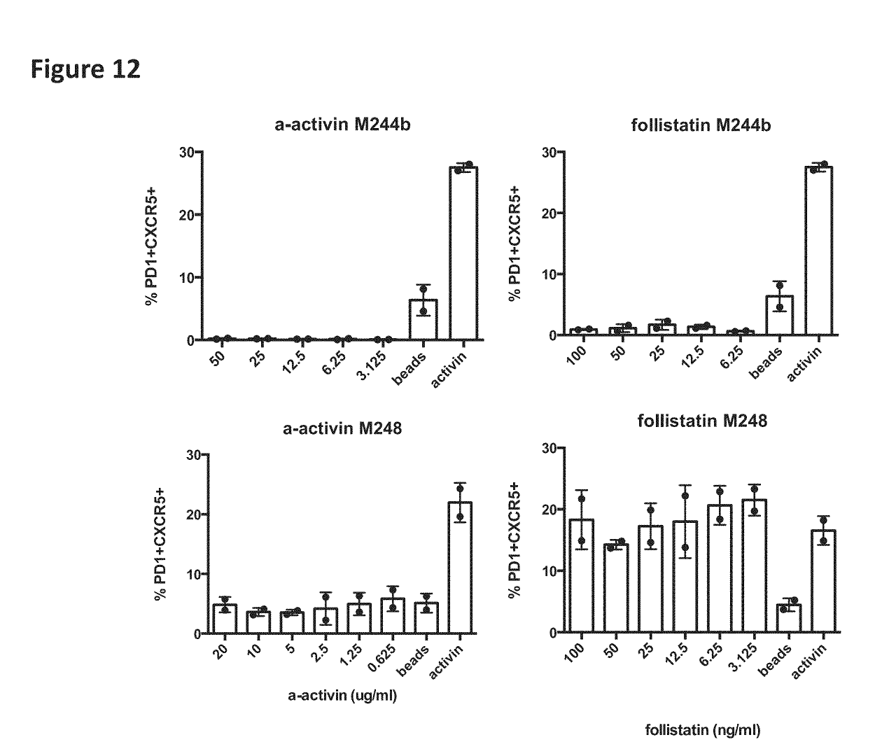

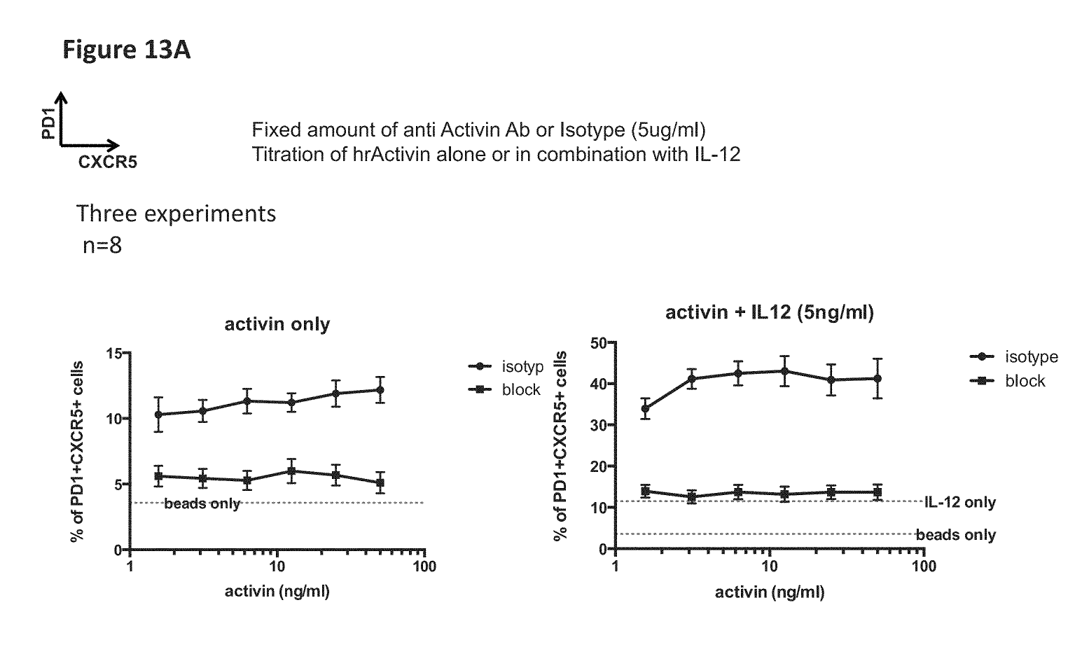

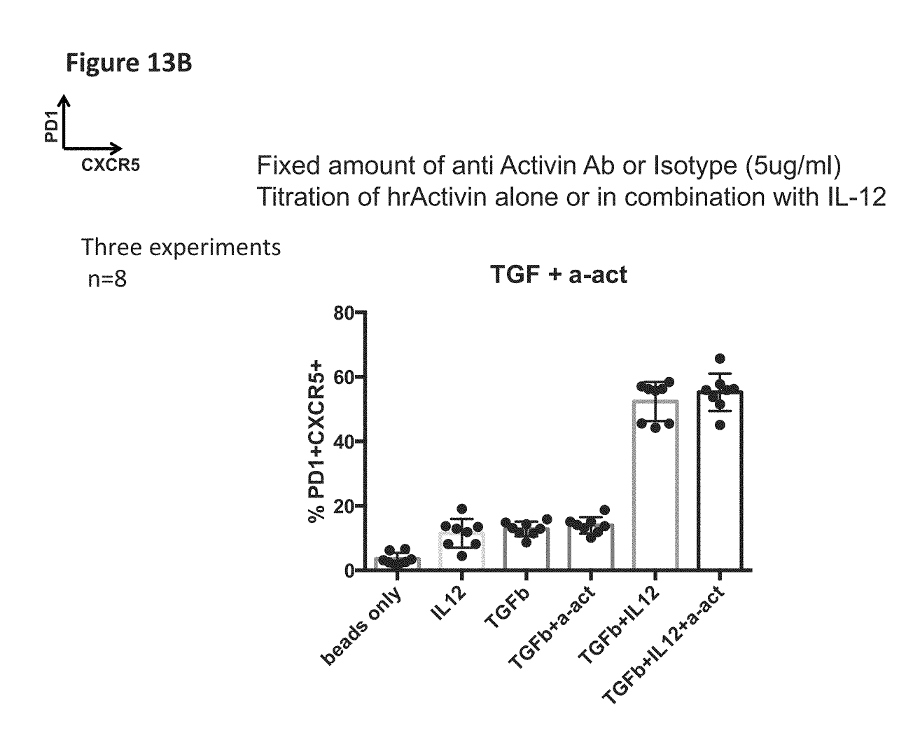

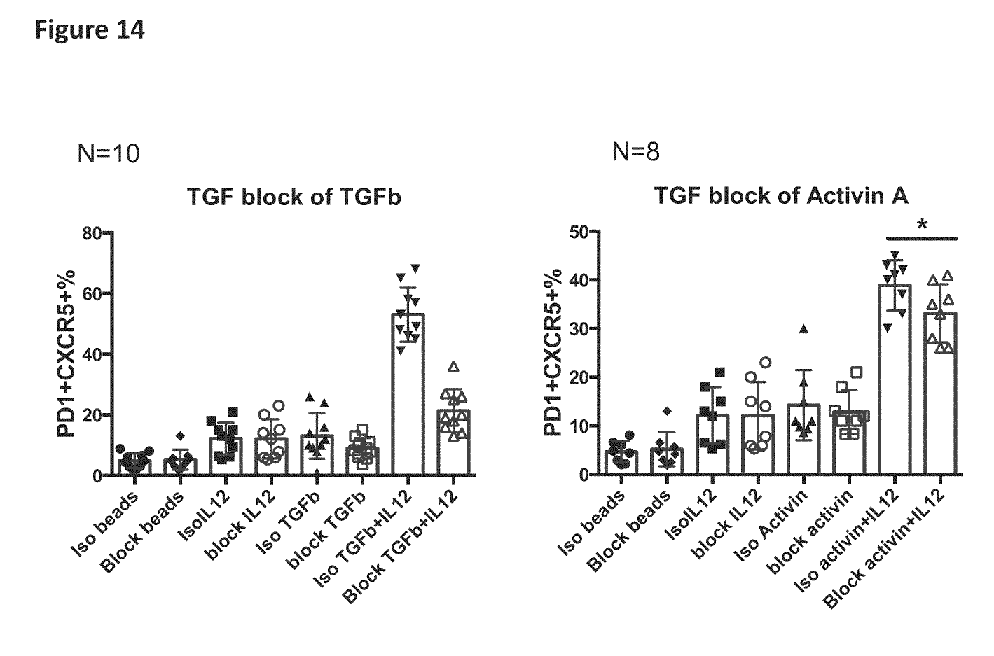

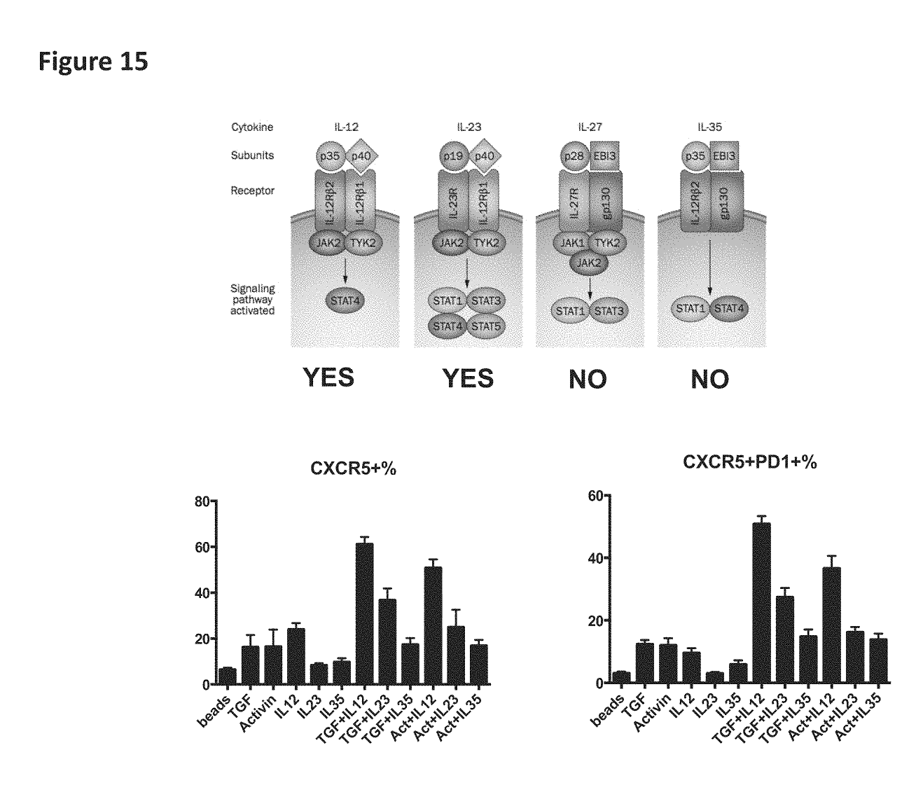

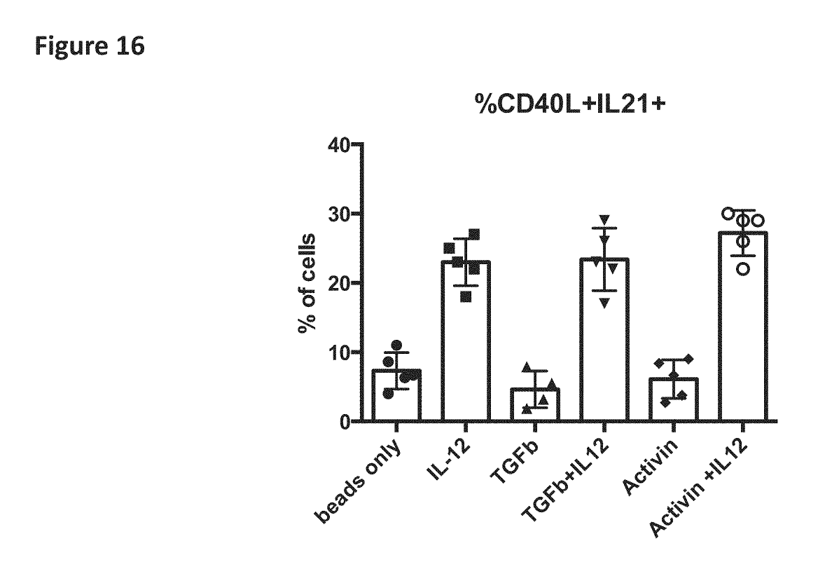

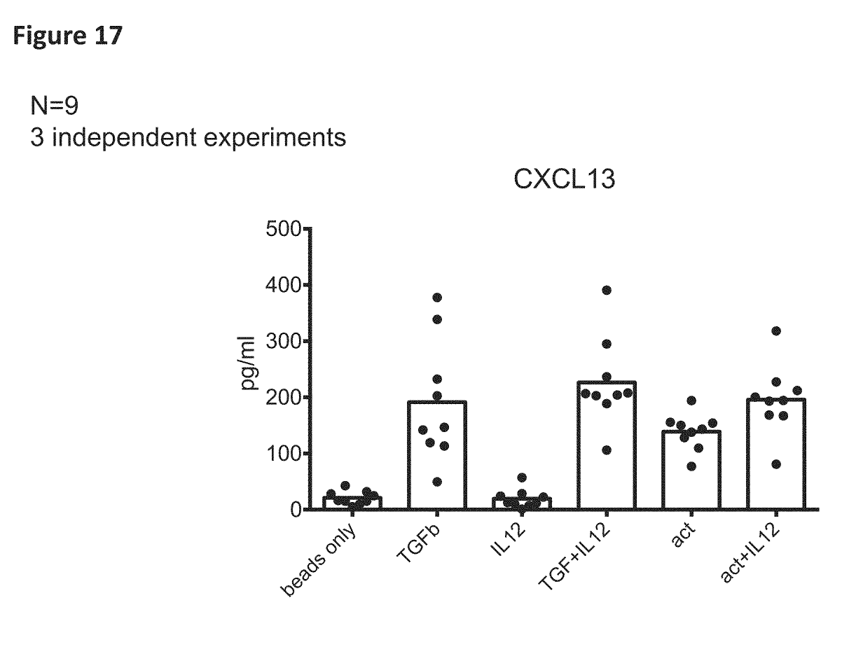

To test the effect of activin A on the differentiation of Tfh cells, human naïve CD4 T cells were isolated from PBMCs and stimulated in vitro with anti CD3/CD28 coated beads and activin A, with or without IL-12, a cytokine previously associated with Tfh cell differentiation. It was found that activin A alone was able to drive a massive PD-1 induction and some CXCR5 expression. Moreover, in combination with IL-12, activin A caused a dramatic increase in the coexpression of PD-1 and CXCR5, a feature of bona fide Tfh cells (FIG. 8B).

Bcl6 is a transcription factor playing a crucial role in the development of Tfh cells. Importantly, it was observed that activin A induced a significant expression of Bcl6, particularly when combined with IL-12 (FIG. 8C). It has been shown that Bcl6 controls the down regulation of chemokine receptor CCR7, which is important for the localization of Tfh cells at the B cell follicle border in lymphoid tissues. Consistent with its ability to induce Bcl6 expression, it was found that activin A presence resulted in a decreased CCR7 expression (FIG. 8D). Therefore, activin A regulates human Tfh differentiation.

Titration of recombinant activin A, with or without recombinant IL-12, and its effect on the induction of FoxP3⁺CXCR5⁺ cells was measured by flow cytometry. The effect of recombinant TGFβ with or without recombinant IL-12 was also measured. TGFβ but not activin A was found to induce high FoxP3 expression.

Titration of recombinant activin A, with or without recombinant TGFβ and its effect on the induction of FoxP3⁺CXCR5⁺ cells was measured by flow cytometry. It was found that TGFβ and activin A synergize in inducing FoxP3 upregulation.

Titration of recombinant activin A, with or without recombinant TGFβ and its effect on the induction of PD1⁺CXCR5⁺ cells was measured by flow cytometry. It was found that there was no synergy between TGFβ and activin A.

### Example 2: A high throughput screen revealed activin A as a novel regulator of human CD4 T cell CXCR5 and PD-1 expression

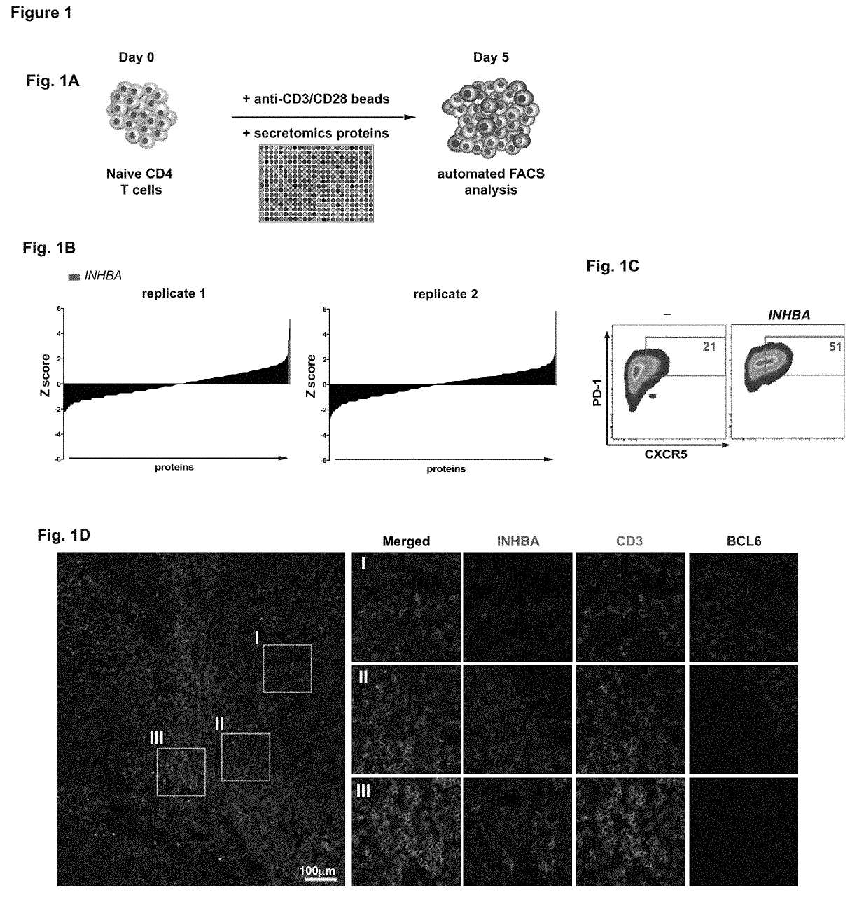

To discover novel regulators of human Tfh cell differentiation, we performed an unbiased high throughput screen of a human extracellular proteome library consisting of over 2000 human proteins predicted or known to be cytokines, chemokines, morphogens, costimulatory receptors, or single pass transmembrane molecules¹⁹. Each unique protein in this proteome, or “secretomics”, library was produced as a secreted recombinant molecule and tested for its capacity to modulate the differentiation of activated naïve CD4 T cells into Tfh cells in vitro. The Tfh screen experimental design and workflow is illustrated in FIG. 1A. Briefly, purified human naïve CD4 T cells were stimulated by anti-CD3/CD28 beads in the presence of the human secretome library arrayed on 384 well plates. Each recombinant protein was tested in duplicate. Following 5 days, expression of the Tfh signature markers CXCR5 and PD-1 were measured on the cells by flow cytometry, in an automated fashion. The primary screen revealed multiple recombinant proteins that behaved either as an inducer or inhibitor of CXCR5 and PD-1 expression (FIG. 1B). The product of the Inhibin beta A gene (INHBA) emerged as the most potent inducer of CXCR5 and PD-1 (FIG. 1B and S1B). The primary screen results were next confirmed, showing that the product of INHBA from the secretomics collection was responsible for CXCR5 and PD-1 induction (FIG. 1C).

The product of INHBA forms the cytokine activin A as a homodimer. Activin A belongs to the family of activins/inhibins, a group of 5 distinct dimeric cytokines resulting from the combination of 3 different monomers: inhibin beta A, beta B, and alpha^{20,21}. Activin A is a pleiotropic cytokine involved in multiple vital biological processes²²⁻²⁶. Activin A was the first cytokine discovered to sustain human pluripotent stem cells²⁷. Additionally, activin A has complex activities on human stem cells in combination with other factors²⁸⁻³⁰. However, the role of activin A in the immune system is not fully understood, with only a small number of studies providing evidence for involvement of activin A in T cell biology^{26,31-34}.

It was then asked whether activin A is present in sites where Tfh cell differentiation physiologically occurs. To this aim, human tonsils were stained, a tissue enriched in Tfh cells and GCs, for INHBA. Importantly, INHBA was found in T cell zones, including the T-B borders, of all the tonsils analyzed (FIG. 1D and S1C), and the majority of INHBA production was in CD3⁻ cells. Therefore, the in vivo localization of INHBA subunit is compatible with an early role of activin A in human Tfh differentiation.

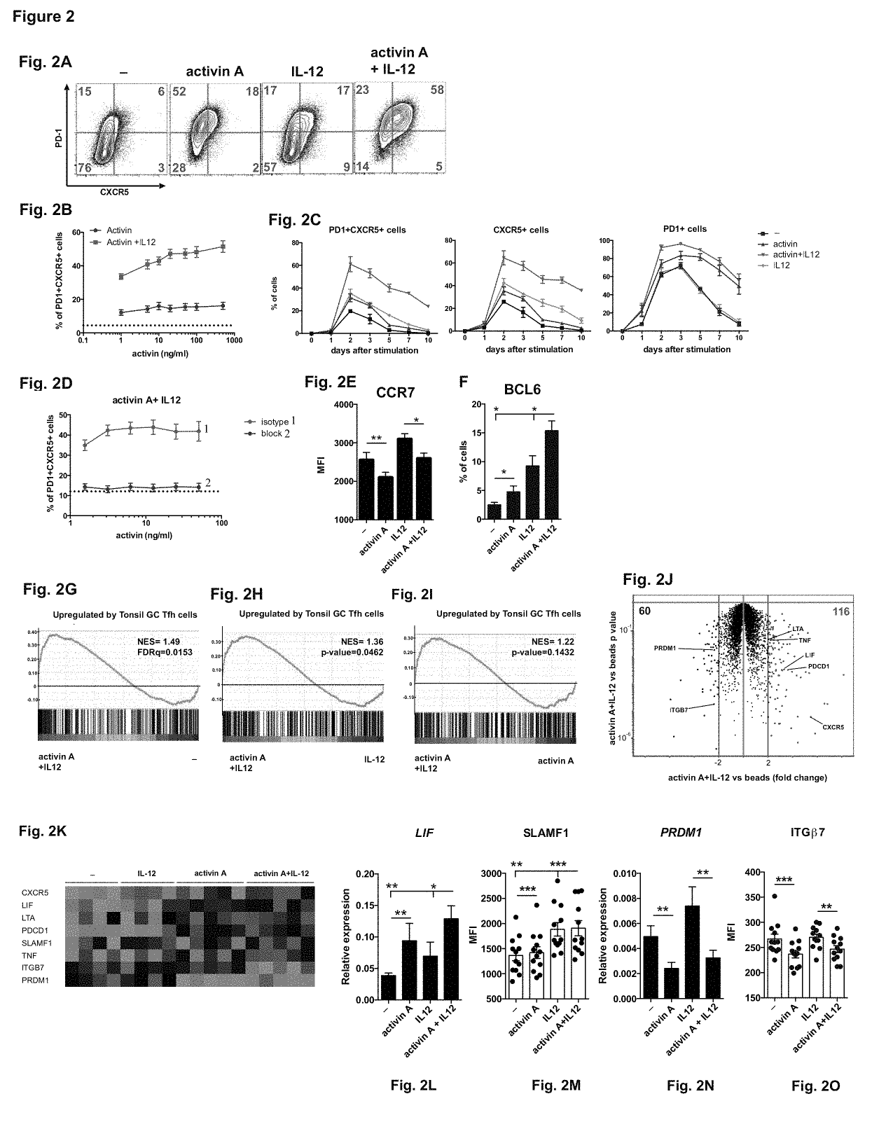

The role of activin A in in vitro Tfh differentiation was then independently confirmed by testing activin A from multiple commercial vendors (data not shown), and validated using primary naïve CD4 T cells from numerous human donors (FIG. 2A-C). Furthermore, many uncharacterized proteins are present in serum used to supplement T cell cultures. Thus, serum-free medium was used as a critical control to rule out possible indirect or synergistic effects of undefined serum components. In stringent serum free conditions, activin A induces both PD-1 and CXCR5 expression on activated naïve CD4 T cells in a dose dependent fashion (FIG. 2A-C and S2A), demonstrating a direct effect of activin A on human Tfh differentiation. Overall, these data indicate that a high throughput screen of human proteins enables the identification of factors that can function as early regulators of human Tfh differentiation, with activin A identified as the top hit in the screen.

### Example 3: Activin A Synergizes with IL-12

IL-12 is the strongest identified stimulus driving IL-21 production by human CD4 T cells¹⁷. IL-12 can also modulate the induction of CXCR5 expression³⁵ (FIG. 2A). Therefore, it was asked whether IL-12 might act in synergy with activin A. Activin A regulation of CXCR5 was enhanced by IL-12 (FIG. 2A-C), indicating the existence of a synergy between these two cytokines in promoting CXCR5 expression. Two additional cytokines of the IL-12 cytokine family, IL-23 and IL-35, were evaluated for their ability to synergize with activin A. While IL-23 had a modest but detectable synergistic activity with activin A, IL-35 had no relevant effect on CXCR5 and PD-1 expression in serum-free conditions. Expression of CXCR5 and PD-1 was retained by cells stimulated with activin A in combination with IL-12 (FIG. 2C). These data point to IL-12 as an enhancer of activin A induction of CXCR5 and PD-1.

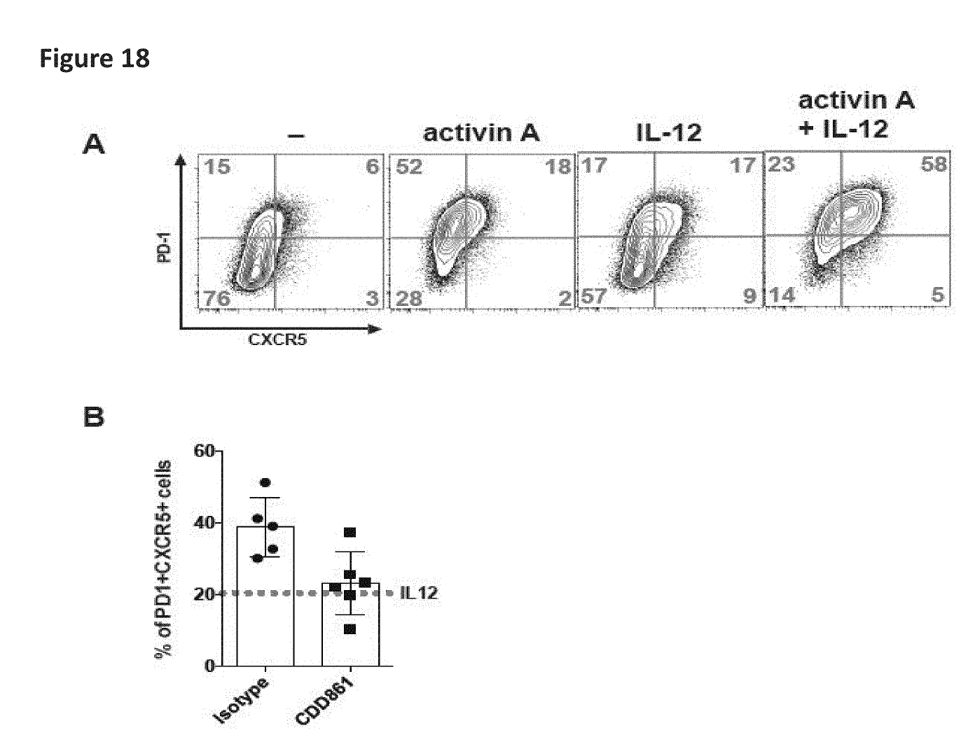

Treatment of activated CD4 T cells with an anti-activin A neutralizing monoclonal antibody (mAb) abolished activin A-mediated differentiation of PD-1⁺CXCR5⁺ cells (FIG. 2D and FIG. 18B). Thus, activin A acts directly on activated human CD4 T cells and regulates differentiation of PD-1⁺CXCR5⁺ cells in a specific manner

### Example 4: Activin A Regulates Human Tfh Differentiation Associated Genes

The finding that activin A, combined with IL-12, induced the expression of multiple Tfh canonical receptors on in vitro activated naïve CD4 T cells prompted investigation of whether additional molecules important for Tfh biology were under the control of activin A or activin A/IL-12 synergy. Activin A was sufficient to decrease CCR7 expression on activated CD4 T cells (FIG. 2E). Moreover, a fraction of CD4 T cells differentiated with activin A+IL-12 increased expression of BCL6 (FIG. 2F and S4). Next, the global transcriptomic profile of the in vitro differentiated Tfh-like cells was ascertained by RNA sequencing (RNA-seq) gene expression analysis. A comparison of gene expression profiles of in vitro cultured CD4 T cells and bona fide human GC Tfh cells revealed that cells differentiated in presence of activin A or activin A+IL-12 were imprinted with bona fide GC Tfh signature genes (FIG. 2G-I). Activin A contributed more than IL-12 to driving expression of bona fide GC Tfh associated genes (FIG. 2H-I). Activin A+IL-12 resulted in a selective upregulation of 116 genes versus cells differentiated with beads only (FIG. 2J). Examination of the 116 upregulated genes confirmed the presence of numerous genes of interest, including SLAMF1, LIF, LTA, and TNF (FIG. 2K), in addition to CXCR5, and PDCD1 (PD-1). Interestingly, activin A emerged as the major factor regulating several Tfh-signature genes such as LIF, LTA, PDCD1, and TNF genes (FIG. 2K). Differential LIF and SLAMF1 expression were confirmed (FIG. 2L-M).

In addition to the upregulated genes, a set of 60 genes was suppressed by activin A+IL-12 (FIG. 2J and table S2). Activin A caused significant downregulation of PRDM1 (FIGS. 2K and 2N). The transcription factor BLIMP1 (PRDM1) is a potent and well-established BCL6 antagonist that is expressed at low levels in bona fide Tfh cells⁸. ITGβ7 was an additional target of interest downregulated in the presence of activin A (FIG. 20). ITGβ7 is an integrin downregulated on human GC Tfh cells. Altogether, gene expression analysis of CD4 T cells cultured in the presence of activin A indicated that activin A is sufficient to regulate the expression of multiple molecules important for Tfh cell biology, including genes involved in migration, differentiation and proliferation.

### Example 5: Activin A Contributes to the Generation of Functionally Competent Tfh-Like Cells

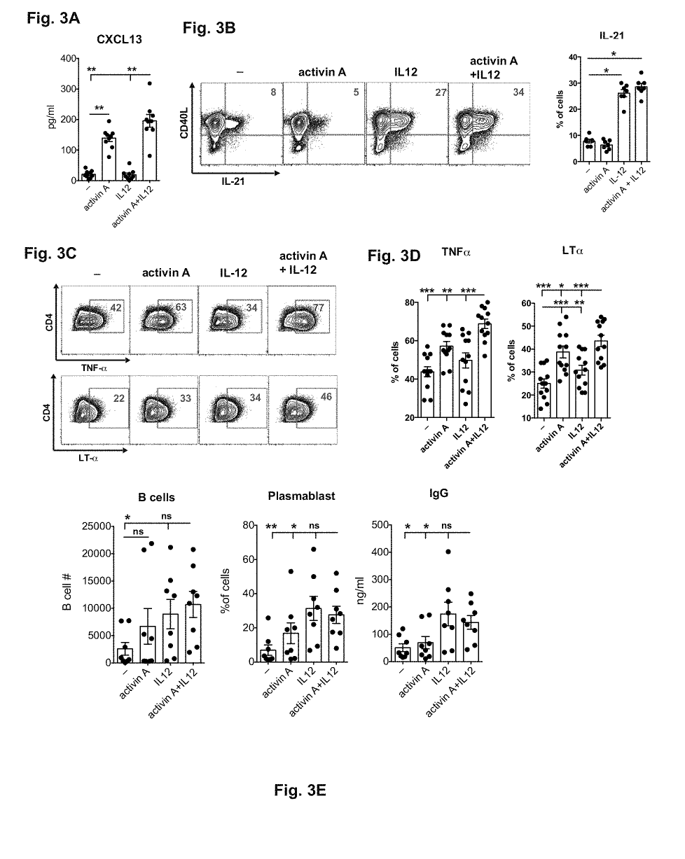

Tfh cells are defined by their specialized function as B cell helpers. To investigate whether activin A can modulate Tfh cell function, the production of Tfh canonical lymphokines by the in vitro differentiated cells was evaluated. First, the ability of the in vitro differentiated cells to secrete CXCL13, the chemokine ligand of CXCR5, was tested. CXCL13 is one of the defining factors secreted by human Tfh cells, in that it is constitutively produced in large quantities by human GC Tfh cells and selectively expressed by circulating memory Tfh cells upon restimulation³⁶⁻³⁸ In addition to its chemoattractant properties, CXCL13 also exhibits cytokine-type activity on B lymphocytes³⁹. Here, it was found that activin A selectively induces CXCL13 expression by CD4 T cells in vitro (FIG. 3A).

IL-21 is a cytokine highly produced by GC Tfh cells and circulating memory Tfh cells, and is a potent mediator of GC B cell survival and plasma cell differentiation^{2,38,40} It was observed that CD4 T cells cultured in vitro with IL-12 were capable of producing IL-21 after a short re-stimulation with PMA/Ionomycin (FIG. 3B). Moreover, cells cultured with activin A+IL-12 retained full IL-21 production potential (FIG. 3B).

The RNA-seq data from CD4 T cells cultured in the presence of activin A indicated that expression of two cytokine encoding genes that are also highly expressed by human GC Tfh cells: TNF and LTA (FIG. 2K). Subsequent experiments confirmed a superior ability of CD4 T cells cultured with activin A to express TNFα and LTα proteins upon restimulation (FIG. 3C-D).

Finally, the capacity of activin A differentiated cells to provide help to B cells was evaluated. CD4 T cells differentiated with activin A+IL-12 were functionally competent B cell helpers, capable of supporting B cell proliferation and survival, plasmablast differentiation, and IgG production (FIG. 3E). Overall, the data indicate that activin A, in combination with IL-12, promotes the generation of Tfh-like cells that express high levels of CXCR5 and PD-1 and display Tfh functional properties, including B cell helper activity and production of CXCL13, IL-21, TNFα and LTα.

### Example 6: Activin a and TGFβ have a Similar Capacity to Drive Human Tfh Cell Differentiation, but Act Independently of Each Other

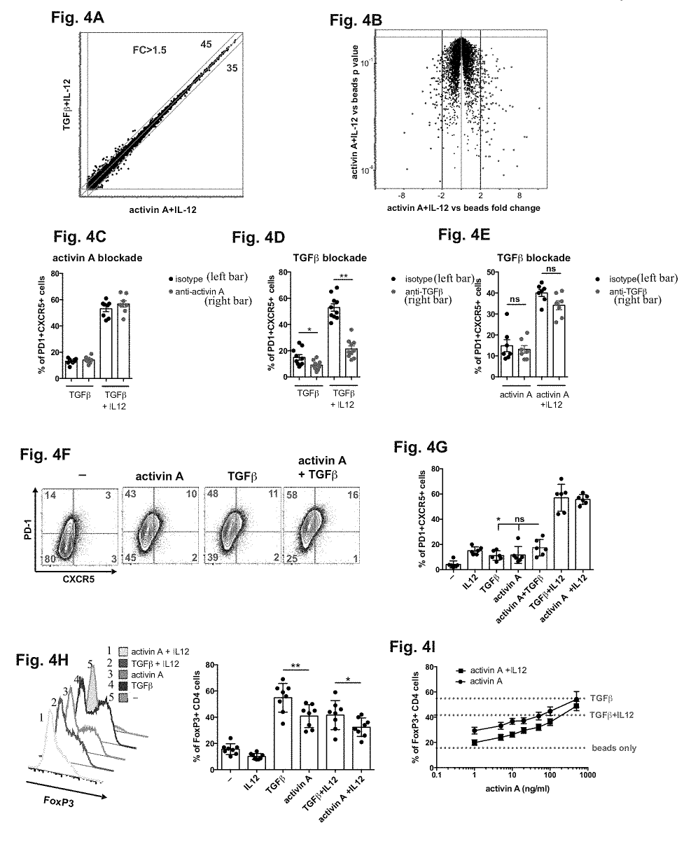

TGFβ is a pleotropic cytokine involved in the differentiation of multiple CD4 T cell subsets in a context dependent fashion⁴¹. A recent study showed that TGFβ, in conjunction with IL-12 and other cytokines, played a role in human Tfh differentiation¹⁵. An intriguing connection links activin A and TGFβ: while activin A and TGFβ use independent receptors, both cytokines can trigger the SMAD2/3 signaling pathway downstream of their respective receptors⁴¹⁻⁴⁴. In stringent conditions using serum free media, TGFβ can synergize with IL-12 to induce CXCR5 and PD-1 expression. TGFβ and activin A displayed a similar capacity to regulate the differentiation of PD-1⁺CXCR5⁺ cells when combined with IL-12. To gain insight into the similarity between activin A and TGFβ in the regulation of human CD4 T cells, the trascriptomes of cells cultured in vitro with activin A+IL-12 versus TGFβ+IL-12 was compared. This comparison revealed a high degree of similarity (FIG. 4A-B), with only 35 genes differentially upregulated by activin A+IL-12 and 45 genes differentially upregulated by TGFβ+IL-12 (FIG. 4A). Similarly, “Volcano” plot analysis indicated that the majority of the genes that were selectively upregulated or downregulated by activin A+IL-12 compare to no exogenous cytokines, followed the same trend in TGFβ+IL-12 samples (FIG. 4B). Because TGFβ mirrored activin A in driving human Tfh differentiation, we next explored the possibility that activin A accounted for the TGFβ-induced phenotype in vitro. This is possible because activated CD4 T cells are capable of secreting activin A following TCR stimulation³⁴. However, activin A blockade did not alter the frequency of PD-1⁺CXCR5⁺ cells induced by TGFβ (FIG. 4C), indicating that activin A is not responsible for TGFβ-induced Tfh cell differentiation. Conversely, anti-TGFβ mAbs did not have any relevant effect on activin A mediated Tfh differentiation, indicating that TGFβ is not responsible for activin A-induced Tfh cell differentiation (FIG. 4D-E). Next, it was evaluated if these two cytokines could induce PD-1 and CXCR5 expression in an additive or synergistic fashion. However, neither PD-1 nor CXCR5 expression changed when activin A and TGFβ were combined in vitro (FIG. 4F-G). Interestingly, despite driving very similar gene programming, activin A and TGFβ significantly differed in their ability to induce FoxP3 expression, with activin A being less potent than TGFβ in driving the generation of FoxP3⁺ cells (FIG. 4H-I). Differential induction of FoxP3 may be important in vivo, since FoxP3 is the lineage defining transcription factor of regulatory T (Treg) cells. Thus, TGFβ may preferentially bias cells toward a Treg phenotype and activin A may bias cells toward a Tfh phenotype. In summary, activin A and TGFβ do not account for each other's ability to regulate Tfh cell biology, and TGFβ preferentially induces FoxP3.

### Example 7: Activin a and IL-2 Directly Compete in Tfh Differentiation

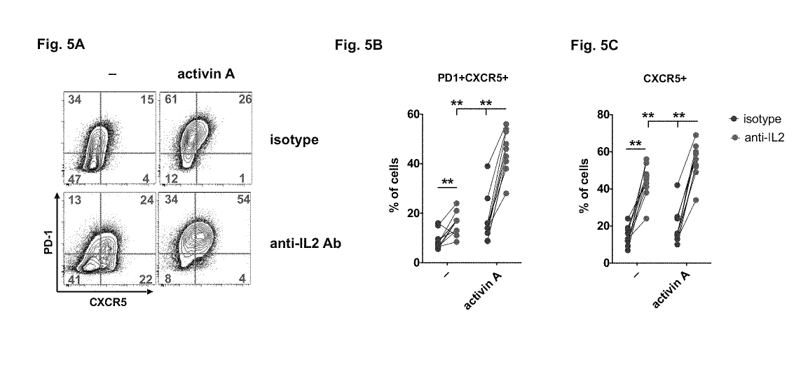

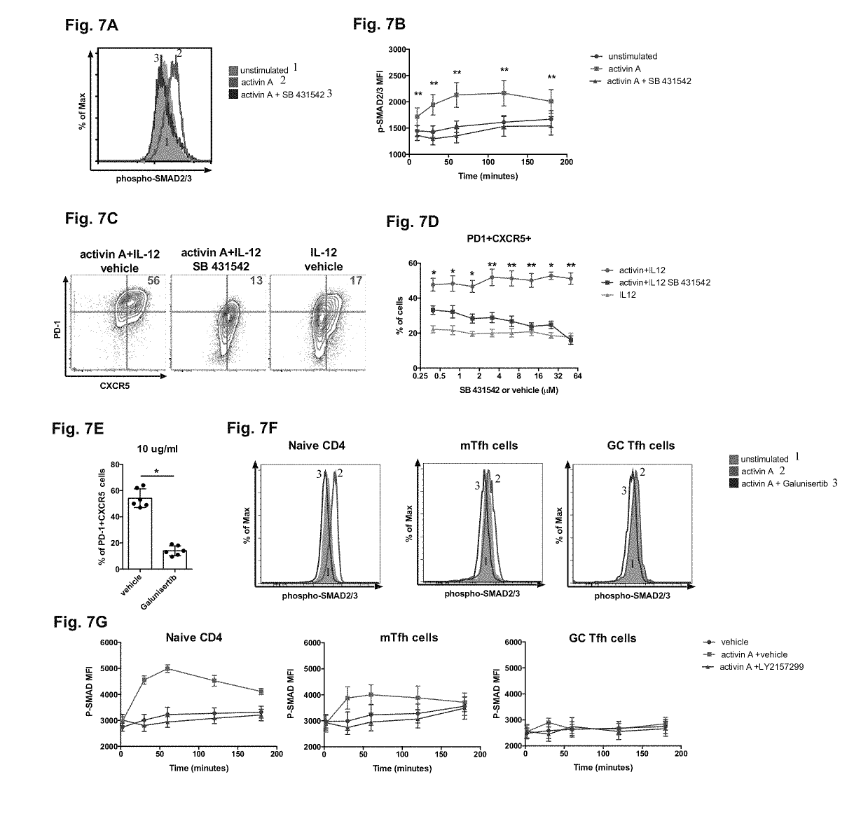

While activin A-stimulated cells express less PRDM1 (Blimp1) (FIG. 2N), IL-2 potently induces Blimp1 in mouse CD4 T cells^{8,45-47} Blimp1 is one of the strongest negative regulators of the Tfh gene program, both through its ability to antagonize BCL6 and directly suppress CXCR5 expression. Thus, it was hypothesized that activin A and IL-2 signaling may antagonize each other in the regulation of BLIMP1 expression and human Tfh differentiation. This was tested by evaluating the effect of IL-2 blockade on activin A differentiated CD4 T cells. In the presence of activin A alone, differentiation of PD-1⁺CXCR5⁺ cells was dramatically increased when IL-2 was neutralized (FIG. 5A-C). Thus, human IL-2 normally counteracts activin A signaling and impairs the human Tfh gene program. IL-2 blockade also modestly potentiated the expression of PD-1 and CXCR5 in cells cultured with activin A+IL-12 (FIG. 7S). Thus, IL-2 is a potent inhibitor of human Tfh differentiation, and activin A can directly induce human Tfh associated gene expression in the absence of any additional cytokines.

### Example 8: Evolutionary Divergence in the Regulation of Tfh Differentiation by Activin A

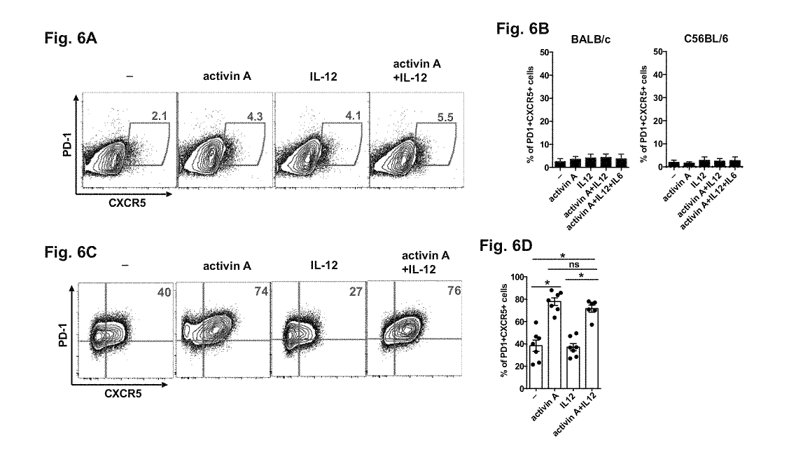

To determine if the role of activin A in Tfh cell differentiation was conserved in mice, murine naïve CD4 T cells were activated in the presence of activin A alone or combined with IL-12. Strikingly, activin A did not have any relevant effect on PD-1 and CXCR5 expression by mouse CD4 T cells (FIG. 6A-B). This was true for both C57BL/6 and BALB/c mouse strains (FIG. 6A-B). Even the presence of IL-6 did not result in activin A mediated promotion of in vitro mouse Tfh differentiation (FIG. 6B).

The lack of any measurable effect of activin A on Tfh associated gene expression by mouse CD4 T cells prompted investigation of whether the role of activin A in Tfh cell differentiation was unique to humans. Thus, the capacity of activin A to drive in vitro Tfh differentiation by a species in a different taxonomial family, macaca mulatta (rhesus macaques) of the non-human primate family Cercopithecidae was evaluated. Potent induction of PD-1⁺CXCR5⁺ cells was observed by activated m. mulatta naïve CD4 T cells in response to activin A (FIG. 6C-D). These data demonstrate that the Tfh-like differentiation mediated by activin A in vitro is not preserved in mice, but is conserved between non-primate species and humans. This suggests the existence of a possible evolutionary divergence in activin A-mediated Tfh cell differentiation.

### Example 9: Activin A-Induced Tfh Differentiation is Mediated by a SMAD2/3 Signaling Pathway

SMAD2/3 is the canonical signaling pathway activated downstream of activin A binding to type IIA and/or IIB receptors and type I receptor ALK4^{43,44}. Phosphorylation of SMAD2/3 in naïve human CD4 T cells in response to activin A was observed (FIG. 7A-B). No relevant induction of SMAD-independent pathways downstream of activin A was observed at the time points analyzed. Therefore, it was hypothesized that SMAD2/3 activation is a central signaling pathway in activin A mediated induction of Tfh associated gene expression. In line with this hypothesis, the usage of a pharmacological inhibitor of ALK4, SB 431542, known to suppress SMAD2/3 activation but not the SMAD-independent pathways⁴⁸, caused a strong reduction of activin A-mediated SMAD2/3 phosphorylation (FIG. 7A-B). Furthermore, it inhibited the differentiation of PD-1⁺CXCR5⁺ cells in activin A containing culture conditions (FIG. 7C-D). To rule out the possibility of non-specific Tfh differentiation suppression by ALK inhibitors, the effect of Galunisertib⁴⁹, an alternative ALK4 inhibitor currently in clinical trials, was tested for its capacity to dampen activin A-mediated Tfh differentiation. Galunisertib mediated a potent suppression of PD-1⁺CXCR5⁺ cell generation in response to activin A (FIG. 7E and S7C continued). Finally, it was found that naïve CD4 T cells from tonsils, similarly to naïve CD4 T cells from blood, quickly phosphorylated SMAD2/3 when cultured with activin A, and the phosphorylation was abolished by Galunisertib (FIGS. 7F and G). Interestingly, PD-1^{int}CXCR5^{int} Tfh cells, a heterogeneous population of early Tfh and post-GC Tfh cells located at the T-B border (referred here to as marginal Tfh, mTfh) shown a moderate but specific P-SMAD induction in response to activin A, whereas fully mature GC Tfh cells did not. Altogether, these data point to SMAD2/3 as an important signaling pathway downstream activin A in the early regulation of human Tfh cell biology.

### Example 10: Effect of Anti-Activin a Type II Receptor (ACVR2) Blocking mAb

Activin A belongs to the family of activins/inhibins, a group of 5 distinct dimeric cytokines resulting from the combination of 3 different monomers: inhibin beta A, beta B, and alpha (Gold and Risbridger, 2012; Thompson et al., 2004). Activin A is a pleiotropic cytokine involved in multiple vital biological processes (Aleman-Muench and Soldevila, 2012; Lotinun et al., 2012; Munz et al., 2001; Muttukrishna et al., 2004; Phillips et al., 2009). However, activin A's role in the immune system is understudied, and a small number of studies provide evidence for involvement of activin A in T cell biology (Aleman-Muench and Soldevila, 2012; Huber et al., 2009; Jones et al., 2012; Ogawa et al., 2006; Semitekolou et al., 2009).

The data provided herein shows that activin A is responsible for induction of the Tfh signature markers CXCR5 and PD-1 on numerous human donors, and its activity is enhanced by IL12 (FIG. 18A).

Activin A primarily binds and signals through the type I receptor ALK4, in complex with the activin A receptor type IIA (ACVR2A) or IIB (ACVR2B). A monoclonal antibody against human Type II activin A receptors, capable of blocking activin A binding to ACVR2A and ACVR2B (“anti-ACVR2 pan Ab”), resulted in a severe decrease of PD-1 and CXCR5 induction driven by activin A (FIG. 18 B). Thus, activin A acts directly on activated human CD4 T cells and regulates differentiation of PD-1⁺CXCR5⁺ cells in a Type II activin A receptor-specific manner

### Example 11: Discussion of Results

Tfh cells are crucial regulators of most humoral responses, and at least two biomedical fields could benefit from Tfh cell manipulation in vivo: vaccinations and autoimmune therapies. In vaccination, generating more Tfh cells could promote larger GC B cell responses and increased production of long-lived plasma cells and memory B cells. Therefore, it is highly desirable to foster Tfh cells in vivo from a vaccine design perspective, when the generation of a protective vaccine relies on the production of neutralizing Abs¹. In contrast, dysregulated Ab responses may lead to auto-Ab mediated autoimmune diseases⁷. Direct studies of bona fide Tfh cells in autoimmune disease are often not possible because of unavailability of lymphoid organs. However, memory blood Tfh cells, that we and others have described as the circulating counterpart of bona fide Tfh cells^{37,38,40,50} or recently activated blood Tfh cells^{38,50} have been found to be elevated in patients affected by rheumatoid arthritis, systemic lupus erythematous and juvenile dermatomyositis⁵¹. Furthermore, the abundance of Tfh-related cells in blood correlated with disease score and/or serum auto-Ab levels⁵¹. In this context, hampering Tfh in a targeted fashion may represent a promising therapeutic approach.

Activin a Signals Foster the Early Expression of Tfh Associated Molecules Involved in Localization and Function

Herein, it is shown that the signals delivered by the cytokine activin A convert activated human naïve CD4 T cells into Tfh-like cells. Early regulators of Tfh cell differentiation that are capable of imprinting the Tfh gene program are secreted by DCs or other myeloid APCs in the T cell area. In line with an early role of activin A in educating the initial stages of Tfh differentiation, DCs and other APCs have been shown to produce activin A following stimulation²⁶. Moreover, B cells and activated T cells are also capable of producing activin A²⁶. Consistent with this hypothesis, evidence is provided for the expression of activin A subunit INHBA in tonsillar T cell areas and it is shown that, in these areas, INHBA is mainly produced by non-T cells. In addition, a gradient in the capacity of tonsillar CD4 T cells to phosphorylate SMAD2/3 following activin A exposure was found, with naïve CD4 T cells having the highest and GC Tfh cells the lowest capacity to sense and respond to activin A. Overall, these data indicate that activin A may behave as early regulator of human Tfh cell differentiation in vivo.

Proper localization is a key feature of Tfh biology. Activin A acts by dampening CCR7 while fostering CXCR5 expression. In addition, activin A also suppresses the expression of the gut homing receptor ITGβ7, thus adding another level of control in the Tfh localization process. Besides regulating localization, activin A also modulates the expression of Tfh functional regulators. Activin A regulates the production of CXCL13, a chemokine with cytokine-like activity highly expressed by bona fide tonsillar Tfh cells. CXCL13 likely amplifies GC reactions by recruiting B cells and newly generated Tfh cells to B cell follicles and by enhancing BCR-mediated B cell activation via CXCR5 binding. TNFα, which was also highly induced by activin A, is a cytokine produced by GC Tfh cells that can act as co-stimulatory signal to mediate B cell activation and Ig production⁵². Moreover, activin A also enhanced CD4 T cell expression of LIF and LTα, two cytokines highly expressed by bona fide GC Tfh cells. The roles of LIF and LTα in B cell help have not formally addressed. However, B cell hyperplasia and polyclonal hypergammaglobulemia is found in mice overexpressing LIF⁵³, suggesting a role for LIF in regulating B cell responses. Disruption of lymphoid organ architecture caused by genetic depletion of LTα does not currently allow for interpretation of any T cell intrinsic role of LTα in the generation of affinity matured memory B cells and long-lived plasma cells. Thus, additional studies are required to address the role of LTα in B cell help. In T-B co-cultures, the B cell helper activity of the in vitro differentiated Tfh cells was driven entirely by IL-12. Although this assay is the best in vitro proxy to test the B cell helper activity of T cells, it has major limitations in that it mostly relies on CD40L (which is equally expressed by all activated cells), and IL-21, which is IL-12 dependent¹⁷. Therefore, additional Tfh features regulated by activin A (with or without IL-12) that are vital for B cell helper function in vivo, such as location (CXCR5, CCR7), the recruitment of additional Tfh and B cells (CXCL13), and the formation of stable contacts with cognate B cells (SLAM), are not emphasized in the in vitro help assay.

Relationships Between Activin a and Other Cytokines in Human Tfh Differentiation

Activin A alone was sufficient for the regulation of many Tfh signature molecules. Nevertheless, the integration of activin A and IL-12 mediated signal was required to acquire a more complete Tfh-like phenotype, which also included high expression of IL-21. This finding demonstrates that activin A is at the center of a cytokine network orchestrating human Tfh differentiation. The cytokine network controlling Tfh differentiation includes not only agonistic players such as IL-12, but also antagonistic signals. It is shown that IL-2 is an example of an antagonistic signal, as indicated by the remarkable enhancement of activin A-mediated CXCR5 induction when IL-2 is specifically blocked in vitro. The enhancement of activin A mediated CXCR5 induction under low IL-2 conditions relies at least partially on the capacity of activin A and IL-2 to exert opposing effects on the expression of BLIMP1, which can directly modulate CXCR5 and BCL6 expression. Thus, IL-2 blockade reduces BLIMP1 levels and poises the activated CD4 T cells to become Tfh-like cells, while the simultaneous presence of activin A further suppresses BLIMP1 and amplifies Tfh differentiation.

TGFβ likely has similar interactions with IL-12 and IL-2 in Tfh programming. However, while activin A induced similar levels of CXCR5 and PD-1 expression compared to TGFβ, activin A induced less FoxP3 expression. Although activated human T cells can express FoxP3 in the absence of suppressive activity⁵⁴, it is still considered controversial as to whether the acquisition of FoxP3 in vitro by human CD4 T cells confers them with the capacity to suppress immune responses. Nevertheless, the lower FoxP3 induction by activin A appears to be a favorable event when the ultimate goal is the induction induce human Tfh cells in vitro.

Evolutionary Divergence in the Regulation of Tfh Differentiation

Mouse models represent important tools to investigate the biological relevance of molecular pathways involved in the regulation of Tfh cell differentiation and B cell responses in vivo. However, a significant limitation in the usage of rodent models results from the existence of evolutionary divergence in signaling pathways shaping Tfh cell biology. Fully mature mouse and human Tfh cells express a virtually identical set of signature molecules, including CXCR5, PD-1, BCL6, CD200, BTLA, TIGIT, SAP, IL-21 and IL-4, while lacking expression of CCR7, PSGL-1 and BLIMPE This phenotypical similarity indicates that the majority of the target genes of the Tfh differentiation program have been conserved through evolution. Nevertheless, a growing body of evidence suggested that the mechanisms regulating this gene expression pattern are not the same in mice and humans and there are signaling pathways that are crucial for the regulation of human, but not mouse Tfh differentiation, such as IL-12 and TGFβ. No relevant CXCR5 and PD-1 induction was found when mouse naïve CD4 T cells were stimulated with activin A in vitro. By contrast, activin A was a potent CXCR5 and PD-1 inducer for both human and non-human primate activated naïve CD4 T cells. This data demonstrates the existence of a potential evolutionary divergence in the role that activin A plays in Tfh cell biology. Nevertheless, the inability of activin A to induce CXCR5 and PD-1 by mouse CD4 T cells may alternatively reflect an unknown inhibitor or cofactor. Thus far, a reproducible approach to generate robust PD-1⁺CXCR5⁺ cells in vitro starting from mouse naïve CD4 T cells is still missing. Thus, a possible interpretation is that, in addition to TCR activation and costimulatory signals, activin A needs the presence of additional pathways in vitro in order to drive mouse Tfh cell differentiation.

### Example 12: Additional Antibody Sequences for Sequence Listing

| SEQ ID NO: 22 | |
|---|---|
| Gly Tyr Thr Phe Thr Ser Ser Tyr Ile Asn | |
| 1 5 10 | |
| SEQ ID NO: 23 | |
| Gly Tyr Thr Phe Thr Ser Ser Tyr Ile Asn | |
| 1 5 10 | |
| SEQ ID NO: 24 | |
| Gly Tyr Thr Phe Thr Ser Ser Tyr Ile Asn | |
| 1 5 10 | |
| SEQ ID NO: 25 | |
| Gly Tyr Thr Phe Thr Ser Ser Tyr Ile Asn | |
| 1 5 10 | |
| SEQ ID NO: 26 | |
| Gly Tyr Thr Phe Thr Ser Ser Tyr Ile Asn | |
| 1 5 10 | |
| SEQ ID NO: 27 | |
| Gly Tyr Thr Phe Thr Ser Ser Tyr Ile Asn | |
| 1 5 10 | |
| SEQ ID NO: 28 | |
| Gly Tyr Thr Phe Thr Ser Ser Tyr Ile Asn | |
| 1 5 10 | |
| SEQ ID NO: 29 | |
| Gly Tyr Thr Phe Thr Ser Ser Tyr Ile Asn | |
| 1 5 10 | |
| SEQ ID NO: 30 | |
| Gly Tyr Thr Phe Thr Ser Ser Tyr Ile Asn | |
| 1 5 10 | |
| SEQ ID NO: 31 | |
| Gly Tyr Thr Phe Thr Ser Ser Tyr Ile Asn | |
| 1 5 10 | |
| SEQ ID NO: 32 | |
| Gly Tyr Thr Phe Thr Ser Ser Tyr Ile Asn | |
| 1 5 10 | |
| SEQ ID NO: 33 | |
| Gly Tyr Thr Phe Thr Ser Ser Tyr Ile Asn | |
| 1 5 10 | |
| SEQ ID NO: 34 | |
| Gly Tyr Thr Phe Thr Ser Ser Tyr Ile Asn | |
| 1 5 10 | |
| SEQ ID NO: 35 | |
| Gly Tyr Thr Phe Thr Ser Ser Tyr Ile Asn | |
| 1 5 10 | |
| SEQ ID NO: 36 | |
| Thr Ile Asn Pro Val Ser Gly Asn Thr Ser Tyr Ala Gln Lys Phe Gln | |
| 1 5 10 15 | |
| Gly | |
| SEQ ID NO: 37 | |
| Thr Ile Asn Pro Val Ser Gly Asn Thr Ser Tyr Ala Gln Lys Phe Gln | |
| 1 5 10 15 | |
| Gly | |
| SEQ ID NO: 38 | |
| Thr Ile Asn Pro Val Ser Gly Asn Thr Ser Tyr Ala Gln Lys Phe Gln | |
| 1 5 10 15 | |
| Gly | |
| SEQ ID NO: 39 | |
| Thr Ile Asn Pro Val Ser Gly Asn Thr Ser Tyr Ala Gln Lys Phe Gln | |
| 1 5 10 15 | |
| Gly | |
| SEQ ID NO: 40 | |
| Met Ile Asn Ala Pro Ile Gly Thr Thr Arg Tyr Ala Gln Lys Phe Gln | |
| 1 5 10 15 | |
| Gly | |
| SEQ ID NO: 41 | |
| Gln Ile Asn Ala Ala Ser Gly Met Thr Arg Tyr Ala Gln Lys Phe Gln | |
| 1 5 10 15 | |
| Gly | |
| SEQ ID NO: 42 | |
| Met Ile Asn Ala Pro Ile Gly Thr Thr Arg Tyr Ala Gln Lys Phe Gln | |
| 1 5 10 15 | |
| Gly | |
| SEQ ID NO: 43 | |
| Thr Ile Asn Pro Val Ser Gly Asn Thr Arg Tyr Ala Gln Lys Phe Gln | |
| 1 5 10 15 | |
| Gly | |
| SEQ ID NO: 44 | |
| Thr Ile Asn Pro Val Ser Gly Ser Thr Ser Tyr Ala Gln Lys Phe Gln | |
| 1 5 10 15 | |
| Gly | |
| SEQ ID NO: 45 | |
| Gln Ile Asn Ala Ala Ser Gly Met Thr Arg Tyr Ala Gln Lys Phe Gln | |
| 1 5 10 15 | |
| Gly | |
| SEQ ID NO: 46 | |
| Asn Ile Asn Ala Ala Ala Gly Ile Thr Leu Tyr Ala Gln Lys Phe Gln | |
| 1 5 10 15 | |
| Gly | |
| SEQ ID NO: 47 | |
| Thr Ile Asn Pro Pro Thr Gly Gly Thr Tyr Tyr Ala Gln Lys Phe Gln | |
| 1 5 10 15 | |
| Gly | |
| SEQ ID NO: 48 | |
| Gly Ile Asn Pro Pro Ala Gly Thr Thr Ser Tyr Ala Gln Lys Phe Gln | |
| 1 5 10 15 | |
| Gly | |
| SEQ ID NO: 49 | |
| Asn Ile Asn Pro Ala Thr Gly His Ala Asp Tyr Ala Gln Lys Phe Gln | |
| 1 5 10 15 | |
| Gly | |
| SEQ ID NO: 50 | |
| Gly Gly Trp Phe Asp Tyr | |
| 1 5 | |
| SEQ ID NO: 51 | |
| Gly Gly Trp Phe Asp Tyr | |
| 1 5 | |
| SEQ ID NO: 52 | |
| Gly Gly Trp Phe Asp Tyr | |
| 1 5 | |
| SEQ ID NO: 53 | |
| Gly Gly Trp Phe Asp Tyr | |
| 1 5 | |
| SEQ ID NO: 54 | |
| Gly Gly Trp Phe Asp Tyr | |
| 1 5 | |
| SEQ ID NO: 55 | |
| Gly Gly Trp Phe Asp Tyr | |
| 1 5 | |
| SEQ ID NO: 56 | |
| Gly Gly Trp Phe Asp Tyr | |
| 1 5 | |
| SEQ ID NO: 57 | |
| Gly Gly Trp Phe Asp Tyr | |
| 1 5 | |
| SEQ ID NO: 58 | |
| Gly Gly Trp Phe Asp Tyr | |
| 1 5 | |
| SEQ ID NO: 59 | |
| Gly Gly Trp Phe Asp Tyr | |
| 1 5 | |
| SEQ ID NO: 60 | |
| Gly Gly Trp Phe Asp Tyr | |
| 1 5 | |
| SEQ ID NO: 61 | |
| Gly Gly Trp Phe Asp Tyr | |
| 1 5 | |
| SEQ ID NO: 62 | |
| Gly Gly Trp Phe Asp Tyr | |
| 1 5 | |
| SEQ ID NO: 63 | |
| Gly Gly Trp Phe Asp Tyr | |
| 1 5 | |
| SEQ ID NO: 64 | |
| Thr Gly Thr Ser Ser Asp Val Gly Ser Tyr Asn Tyr Val Asn | |
| 1 5 10 | |
| SEQ ID NO: 65 | |
| Thr Gly Thr Ser Ser Asp Val Gly Ser Tyr Asn Tyr Val Asn | |
| 1 5 10 | |
| SEQ ID NO: 66 | |
| Thr Gly Thr Ser Ser Asp Val Gly Ser Tyr Asn Tyr Val Asn | |
| 1 5 10 | |
| SEQ ID NO: 67 | |
| Thr Gly Thr Ser Ser Asp Val Gly Ser Tyr Asn Tyr Val Asn | |
| 1 5 10 | |
| SEQ ID NO: 68 | |
| Thr Gly Thr Ser Ser Asp Val Gly Ser Tyr Asn Tyr Val Asn | |
| 1 5 10 | |
| SEQ ID NO: 69 | |
| Thr Gly Thr Ser Ser Asp Val Gly Ser Tyr Asn Tyr Val Asn | |
| 1 5 10 | |
| SEQ ID NO: 70 | |
| Thr Gly Thr Ser Ser Asp Val Gly Ser Tyr Asn Tyr Val Asn | |
| 1 5 10 | |
| SEQ ID NO: 71 | |
| Thr Gly Thr Ser Ser Asp Val Gly Ser Tyr Asn Tyr Val Asn | |
| 1 5 10 | |
| SEQ ID NO: 72 | |
| Thr Gly Thr Ser Ser Asp Val Gly Ser Tyr Asn Tyr Val Asn | |
| 1 5 10 | |
| SEQ ID NO: 73 | |
| Thr Gly Thr Ser Ser Asp Val Gly Ser Tyr Asn Tyr Val Asn | |
| 1 5 10 | |
| SEQ ID NO: 74 | |
| Thr Gly Thr Ser Ser Asp Val Gly Ser Tyr Asn Tyr Val Asn | |
| 1 5 10 | |
| SEQ ID NO: 75 | |
| Thr Gly Thr Ser Ser Asp Val Gly Ser Tyr Asn Tyr Val Asn | |
| 1 5 10 | |
| SEQ ID NO: 76 | |
| Thr Gly Thr Ser Ser Asp Val Gly Ser Tyr Asn Tyr Val Asn | |
| 1 5 10 | |
| SEQ ID NO: 77 | |
| Thr Gly Thr Ser Ser Asp Val Gly Ser Tyr Asn Tyr Val Asn | |
| 1 5 10 | |
| SEQ ID NO: 78 | |
| Leu Met Ile Tyr Gly Val Ser Lys Arg Pro Ser | |
| 1 5 10 | |
| SEQ ID NO: 79 | |
| Leu Met Ile Tyr Gly Val Ser Lys Arg Pro Ser | |
| 1 5 10 | |
| SEQ ID NO: 80 | |
| Leu Met Ile Tyr Gly Val Ser Lys Arg Pro Ser | |
| 1 5 10 | |
| SEQ ID NO: 81 | |
| Leu Met Ile Tyr Gly Val Ser Lys Arg Pro Ser | |
| 1 5 10 | |
| SEQ ID NO: 82 | |
| Leu Met Ile Tyr Gly Val Ser Lys Arg Pro Ser | |
| 1 5 10 | |
| SEQ ID NO: 83 | |
| Leu Met Ile Tyr Gly Val Ser Lys Arg Pro Ser | |
| 1 5 10 | |
| SEQ ID NO: 84 | |
| Leu Met Ile Tyr Gly Val Ser Lys Arg Pro Ser | |
| 1 5 10 | |
| SEQ ID NO: 85 | |
| Leu Met Ile Tyr Gly Val Ser Lys Arg Pro Ser | |
| 1 5 10 | |
| SEQ ID NO: 86 | |
| Leu Met Ile Tyr Gly Val Ser Lys Arg Pro Ser | |
| 1 5 10 | |
| SEQ ID NO: 87 | |
| Leu Met Ile Tyr Gly Val Ser Lys Arg Pro Ser | |
| 1 5 10 | |
| SEQ ID NO: 88 | |
| Leu Met Ile Tyr Gly Val Ser Lys Arg Pro Ser | |
| 1 5 10 | |
| SEQ ID NO: 89 | |
| Leu Met Ile Tyr Gly Val Ser Lys Arg Pro Ser | |
| 1 5 10 | |
| SEQ ID NO: 90 | |
| Leu Met Ile Tyr Gly Val Ser Lys Arg Pro Ser | |
| 1 5 10 | |
| SEQ ID NO: 91 | |
| Leu Met Ile Tyr Gly Val Ser Lys Arg Pro Ser | |
| 1 5 10 | |
| SEQ ID NO: 92 | |
| Gln Ala Trp Thr Ser Lys Met Ala Gly | |
| 1 5 | |
| SEQ ID NO: 93 | |
| Ser Ser Tyr Thr Arg Met Gly His Pro | |
| 1 5 | |
| SEQ ID NO: 94 | |
| Ala Thr Tyr Gly Lys Gly Val Thr Pro Pro | |
| 1 5 10 | |
| SEQ ID NO: 95 | |
| Gly Thr Phe Ala Gly Gly Ser Tyr Tyr Gly | |
| 1 5 10 | |
| SEQ ID NO: 96 | |
| Gln Ala Trp Thr Ser Lys Met Ala Gly | |
| 1 5 | |
| SEQ ID NO: 97 | |
| Gln Ala Trp Thr Ser Lys Met Ala Gly | |
| 1 5 | |
| SEQ ID NO: 98 | |
| Gly Thr Phe Ala Gly Gly Ser Tyr Tyr Gly | |
| 1 5 10 | |
| SEQ ID NO: 99 | |
| Gly Thr Phe Ala Gly Gly Ser Tyr Tyr Gly | |
| 1 5 10 | |
| SEQ ID NO: 100 | |
| Gly Thr Phe Ala Gly Gly Ser Tyr Tyr Gly | |
| 1 5 10 | |
| SEQ ID NO: 101 | |
| Gly Thr Phe Ala Gly Gly Ser Tyr Tyr Gly | |
| 1 5 10 | |
| SEQ ID NO: 102 | |
| Gly Thr Phe Ala Gly Gly Ser Tyr Tyr Gly | |
| 1 5 10 | |
| SEQ ID NO: 103 | |
| Gly Thr Phe Ala Gly Gly Ser Tyr Tyr Gly | |
| 1 5 10 | |
| SEQ ID NO: 104 | |
| Gly Thr Phe Ala Gly Gly Ser Tyr Tyr Gly | |
| 1 5 10 | |
| SEQ ID NO: 105 | |
| Gly Thr Phe Ala Gly Gly Ser Tyr Tyr Gly | |
| 1 5 10 | |
| SEQ ID NO: 106 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gln Ala Trp Thr Ser Lys | |
| 85 90 95 | |
| Met Ala Gly Val Phe Gly Gly Gly Thr Lys Leu Thr Val Leu Gly Gln | |
| 100 105 110 | |
| SEQ ID NO: 107 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Ser Ser Tyr Thr Arg Met | |
| 85 90 95 | |
| Gly His Pro Val Phe Gly Gly Gly Thr Lys Leu Thr Val Leu Gly Gln | |
| 100 105 110 | |
| SEQ ID NO: 108 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Ala Thr Tyr Gly Lys Gly | |
| 85 90 95 | |
| Val Thr Pro Pro Val Phe Gly Gly Gly Thr Lys Leu Thr Val Leu Gly | |
| 100 105 110 | |
| Gln | |
| SEQ ID NO: 109 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 110 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gln Ala Trp Thr Ser Lys | |
| 85 90 95 | |
| Met Ala Gly Val Phe Gly Gly Gly Thr Lys Leu Thr Val Leu Gly Gln | |
| 100 105 110 | |
| SEQ ID NO: 111 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gln Ala Trp Thr Ser Lys | |
| 85 90 95 | |
| Met Ala Gly Val Phe Gly Gly Gly Thr Lys Leu Thr Val Leu Gly Gln | |
| 100 105 110 | |
| SEQ ID NO: 112 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 113 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 114 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 115 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 116 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 117 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 118 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 119 | |
| Asp Ile Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln | |
| SEQ ID NO: 120 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Val Ser Gly Asn Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 121 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Val Ser Gly Asn Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 122 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Val Ser Gly Asn Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 123 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Val Ser Gly Asn Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 124 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Met Ile Asn Ala Pro Ile Gly Thr Thr Arg Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 125 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Gln Ile Asn Ala Ala Ser Gly Met Thr Arg Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 126 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Met Ile Asn Ala Pro Ile Gly Thr Thr Arg Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 127 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Val Ser Gly Asn Thr Arg Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 128 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Val Ser Gly Ser Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 129 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Gln Ile Asn Ala Ala Ser Gly Met Thr Arg Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 130 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Asn Ile Asn Ala Ala Ala Gly Ile Thr Leu Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 131 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Pro Thr Gly Gly Thr Tyr Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 132 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Gly Ile Asn Pro Pro Ala Gly Thr Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 133 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Asn Ile Asn Pro Ala Thr Gly His Ala Asp Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Ser Leu Arg Ser Glu Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser | |
| 115 | |
| SEQ ID NO: 134 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc caggcttgga cttctaagat ggctggtgtg | 300 |
| tttggcggcg gcacgaagtt aaccgttctt ggccag | 336 |
| SEQ ID NO: 135 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc tcttcttata ctcgtatggg tcatcctgtg | 300 |
| tttggcggcg gcacgaagtt aaccgttctt ggccag | 336 |
| SEQ ID NO: 136 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc gctacttatg gtaagggtgt tactcctcct | 300 |
| gtgtttggcg gcggcacgaa gttaaccgtt cttggccag | 339 |
| SEQ ID NO: 137 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 138 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc caggcttgga cttctaagat ggctggtgtg | 300 |
| tttggcggcg gcacgaagtt aaccgttctt ggccag | 336 |
| SEQ ID NO: 139 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc caggcttgga cttctaagat ggctggtgtg | 300 |
| tttggcggcg gcacgaagtt aaccgttctt ggccag | 336 |
| SEQ ID NO: 140 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 141 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 142 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 143 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 144 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 145 | |
| gatatcgcac tgacccagcc agcttcagtg agcggctcac caggtcagag cattaccatc | 60 |
| tcgtgtactg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 146 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 147 | |
| gatatcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtt cttggccag | 339 |
| SEQ ID NO: 148 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcact atcaatccgg tttctggcaa tacgtcttac | 180 |
| gcgcagaagt ttcagggccg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 149 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcact atcaatccgg tttctggcaa tacgtcttac | 180 |
| gcgcagaagt ttcagggccg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 150 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcact atcaatccgg tttctggcaa tacgtcttac | 180 |
| gcgcagaagt ttcagggccg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 151 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcact atcaatccgg tttctggcaa tacgtcttac | 180 |
| gcgcagaagt ttcagggccg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 152 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcatg attaatgctc ctattggtac tactcgttat | 180 |
| gctcagaagt ttcagggtcg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 153 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggccag attaatgctg cttctggtat gactcgttat | 180 |
| gctcagaagt ttcagggtcg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 154 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcatg attaatgctc ctattggtac tactcgttat | 180 |
| gctcagaagt ttcagggtcg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 155 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcact atcaatccgg tttctggcaa tacgcgttac | 180 |
| gcgcagaagt ttcagggccg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 156 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcact atcaatccgg tttctggctc tacgtcttac | 180 |
| gcgcagaagt ttcagggccg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 157 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggccag attaatgctg cttctggtat gactcgttat | 180 |
| gctcagaagt ttcagggtcg ggtcaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 158 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcaat attaatgctg ctgctggtat tactctttat | 180 |
| gctcagaagt ttcagggtcg ggtcaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 159 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcact attaatcctc ctactggagg tacttattat | 180 |
| gctcagaagt ttcagggtcg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 160 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcggt attaatcctc ctgctggtac tacttcttat | 180 |
| gctcagaagt ttcagggtcg ggtcaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 161 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcaat attaatcctg ctactggtca tgctgattat | 180 |
| gctcagaagt ttcagggtcg ggtgaccatg acccgtgata ccagcattag caccgcgtat | 240 |
| atggaactga gcagcctgcg tagcgaagat acggccgtgt attattgcgc gcgtggtggt | 300 |
| tggtttgatt attggggcca aggcaccctg gtgacggtta gctca | 345 |
| SEQ ID NO: 162 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 163 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 164 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 165 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 166 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 167 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Val Ser Gly Ser Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Ser Ser Lys Ser Thr Ser Gly Gly Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Ser Leu Gly | |
| 180 185 190 | |
| Thr Gln Thr Tyr Ile Cys Asn Val Asn His Lys Pro Ser Asn Thr Lys | |
| 195 200 205 | |
| Val Asp Lys Arg Val Glu Pro Lys Ser Cys Asp Lys Thr His Thr Cys | |
| 210 215 220 | |
| Pro Pro Cys Pro Ala Pro Glu Ala Ala Gly Gly Pro Ser Val Phe Leu | |
| 225 230 235 240 | |
| Phe Pro Pro Lys Pro Lys Asp Thr Leu Met Ile Ser Arg Thr Pro Glu | |
| 245 250 255 | |
| Val Thr Cys Val Val Val Asp Val Ser His Glu Asp Pro Glu Val Lys | |
| 260 265 270 | |
| Phe Asn Trp Tyr Val Asp Gly Val Glu Val His Asn Ala Lys Thr Lys | |
| 275 280 285 | |
| Pro Arg Glu Glu Gln Tyr Asn Ser Thr Tyr Arg Val Val Ser Val Leu | |
| 290 295 300 | |
| Thr Val Leu His Gln Asp Trp Leu Asn Gly Lys Glu Tyr Lys Cys Lys | |
| 305 310 315 320 | |
| Val Ser Asn Lys Ala Leu Pro Ala Pro Ile Glu Lys Thr Ile Ser Lys | |
| 325 330 335 | |
| Ala Lys Gly Gln Pro Arg Glu Pro Gln Val Tyr Thr Leu Pro Pro Ser | |
| 340 345 350 | |
| Arg Glu Glu Met Thr Lys Asn Gln Val Ser Leu Thr Cys Leu Val Lys | |
| 355 360 365 | |
| Gly Phe Tyr Pro Ser Asp Ile Ala Val Glu Trp Glu Ser Asn Gly Gln | |
| 370 375 380 | |
| Pro Glu Asn Asn Tyr Lys Thr Thr Pro Pro Val Leu Asp Ser Asp Gly | |
| 385 390 395 400 | |
| Ser Phe Phe Leu Tyr Ser Lys Leu Thr Val Asp Lys Ser Arg Trp Gln | |
| 405 410 415 | |
| Gln Gly Asn Val Phe Ser Cys Ser Val Met His Glu Ala Leu His Asn | |
| 420 425 430 | |
| His Tyr Thr Gln Lys Ser Leu Ser Leu Ser Pro Gly Lys | |
| 435 440 445 | |
| SEQ ID NO: 168 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Gln Ile Asn Ala Ala Ser Gly Met Thr Arg Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Ser Ser Lys Ser Thr Ser Gly Gly Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Ser Leu Gly | |
| 180 185 190 | |
| Thr Gln Thr Tyr Ile Cys Asn Val Asn His Lys Pro Ser Asn Thr Lys | |
| 195 200 205 | |
| Val Asp Lys Arg Val Glu Pro Lys Ser Cys Asp Lys Thr His Thr Cys | |
| 210 215 220 | |
| Pro Pro Cys Pro Ala Pro Glu Ala Ala Gly Gly Pro Ser Val Phe Leu | |
| 225 230 235 240 | |
| Phe Pro Pro Lys Pro Lys Asp Thr Leu Met Ile Ser Arg Thr Pro Glu | |
| 245 250 255 | |
| Val Thr Cys Val Val Val Asp Val Ser His Glu Asp Pro Glu Val Lys | |
| 260 265 270 | |
| Phe Asn Trp Tyr Val Asp Gly Val Glu Val His Asn Ala Lys Thr Lys | |
| 275 280 285 | |
| Pro Arg Glu Glu Gln Tyr Asn Ser Thr Tyr Arg Val Val Ser Val Leu | |
| 290 295 300 | |
| Thr Val Leu His Gln Asp Trp Leu Asn Gly Lys Glu Tyr Lys Cys Lys | |
| 305 310 315 320 | |
| Val Ser Asn Lys Ala Leu Pro Ala Pro Ile Glu Lys Thr Ile Ser Lys | |
| 325 330 335 | |
| Ala Lys Gly Gln Pro Arg Glu Pro Gln Val Tyr Thr Leu Pro Pro Ser | |
| 340 345 350 | |
| Arg Glu Glu Met Thr Lys Asn Gln Val Ser Leu Thr Cys Leu Val Lys | |
| 355 360 365 | |
| Gly Phe Tyr Pro Ser Asp Ile Ala Val Glu Trp Glu Ser Asn Gly Gln | |
| 370 375 380 | |
| Pro Glu Asn Asn Tyr Lys Thr Thr Pro Pro Val Leu Asp Ser Asp Gly | |
| 385 390 395 400 | |
| Ser Phe Phe Leu Tyr Ser Lys Leu Thr Val Asp Lys Ser Arg Trp Gln | |
| 405 410 415 | |
| Gln Gly Asn Val Phe Ser Cys Ser Val Met His Glu Ala Leu His Asn | |
| 420 425 430 | |
| His Tyr Thr Gln Lys Ser Leu Ser Leu Ser Pro Gly Lys | |
| 435 440 445 | |
| SEQ ID NO: 169 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Asn Ile Asn Ala Ala Ala Gly Ile Thr Leu Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Ser Ser Lys Ser Thr Ser Gly Gly Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Ser Leu Gly | |
| 180 185 190 | |
| Thr Gln Thr Tyr Ile Cys Asn Val Asn His Lys Pro Ser Asn Thr Lys | |
| 195 200 205 | |
| Val Asp Lys Arg Val Glu Pro Lys Ser Cys Asp Lys Thr His Thr Cys | |
| 210 215 220 | |
| Pro Pro Cys Pro Ala Pro Glu Ala Ala Gly Gly Pro Ser Val Phe Leu | |
| 225 230 235 240 | |
| Phe Pro Pro Lys Pro Lys Asp Thr Leu Met Ile Ser Arg Thr Pro Glu | |
| 245 250 255 | |
| Val Thr Cys Val Val Val Asp Val Ser His Glu Asp Pro Glu Val Lys | |
| 260 265 270 | |
| Phe Asn Trp Tyr Val Asp Gly Val Glu Val His Asn Ala Lys Thr Lys | |
| 275 280 285 | |
| Pro Arg Glu Glu Gln Tyr Asn Ser Thr Tyr Arg Val Val Ser Val Leu | |
| 290 295 300 | |
| Thr Val Leu His Gln Asp Trp Leu Asn Gly Lys Glu Tyr Lys Cys Lys | |
| 305 310 315 320 | |
| Val Ser Asn Lys Ala Leu Pro Ala Pro Ile Glu Lys Thr Ile Ser Lys | |
| 325 330 335 | |
| Ala Lys Gly Gln Pro Arg Glu Pro Gln Val Tyr Thr Leu Pro Pro Ser | |
| 340 345 350 | |
| Arg Glu Glu Met Thr Lys Asn Gln Val Ser Leu Thr Cys Leu Val Lys | |
| 355 360 365 | |
| Gly Phe Tyr Pro Ser Asp Ile Ala Val Glu Trp Glu Ser Asn Gly Gln | |
| 370 375 380 | |
| Pro Glu Asn Asn Tyr Lys Thr Thr Pro Pro Val Leu Asp Ser Asp Gly | |
| 385 390 395 400 | |
| Ser Phe Phe Leu Tyr Ser Lys Leu Thr Val Asp Lys Ser Arg Trp Gln | |
| 405 410 415 | |
| Gln Gly Asn Val Phe Ser Cys Ser Val Met His Glu Ala Leu His Asn | |
| 420 425 430 | |
| His Tyr Thr Gln Lys Ser Leu Ser Leu Ser Pro Gly Lys | |
| 435 440 445 | |
| SEQ ID NO: 170 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Gly Ile Asn Pro Pro Ala Gly Thr Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Ser Ser Lys Ser Thr Ser Gly Gly Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Ser Leu Gly | |
| 180 185 190 | |
| Thr Gln Thr Tyr Ile Cys Asn Val Asn His Lys Pro Ser Asn Thr Lys | |
| 195 200 205 | |
| Val Asp Lys Arg Val Glu Pro Lys Ser Cys Asp Lys Thr His Thr Cys | |
| 210 215 220 | |
| Pro Pro Cys Pro Ala Pro Glu Ala Ala Gly Gly Pro Ser Val Phe Leu | |
| 225 230 235 240 | |
| Phe Pro Pro Lys Pro Lys Asp Thr Leu Met Ile Ser Arg Thr Pro Glu | |
| 245 250 255 | |
| Val Thr Cys Val Val Val Asp Val Ser His Glu Asp Pro Glu Val Lys | |
| 260 265 270 | |
| Phe Asn Trp Tyr Val Asp Gly Val Glu Val His Asn Ala Lys Thr Lys | |
| 275 280 285 | |
| Pro Arg Glu Glu Gln Tyr Asn Ser Thr Tyr Arg Val Val Ser Val Leu | |
| 290 295 300 | |
| Thr Val Leu His Gln Asp Trp Leu Asn Gly Lys Glu Tyr Lys Cys Lys | |
| 305 310 315 320 | |
| Val Ser Asn Lys Ala Leu Pro Ala Pro Ile Glu Lys Thr Ile Ser Lys | |
| 325 330 335 | |
| Ala Lys Gly Gln Pro Arg Glu Pro Gln Val Tyr Thr Leu Pro Pro Ser | |
| 340 345 350 | |
| Arg Glu Glu Met Thr Lys Asn Gln Val Ser Leu Thr Cys Leu Val Lys | |
| 355 360 365 | |
| Gly Phe Tyr Pro Ser Asp Ile Ala Val Glu Trp Glu Ser Asn Gly Gln | |
| 370 375 380 | |
| Pro Glu Asn Asn Tyr Lys Thr Thr Pro Pro Val Leu Asp Ser Asp Gly | |
| 385 390 395 400 | |
| Ser Phe Phe Leu Tyr Ser Lys Leu Thr Val Asp Lys Ser Arg Trp Gln | |
| 405 410 415 | |
| Gln Gly Asn Val Phe Ser Cys Ser Val Met His Glu Ala Leu His Asn | |
| 420 425 430 | |
| His Tyr Thr Gln Lys Ser Leu Ser Leu Ser Pro Gly Lys | |
| 435 440 445 | |
| SEQ ID NO: 171 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Asn Ile Asn Pro Ala Thr Gly His Ala Asp Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Ser Ser Lys Ser Thr Ser Gly Gly Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Ser Leu Gly | |
| 180 185 190 | |
| Thr Gln Thr Tyr Ile Cys Asn Val Asn His Lys Pro Ser Asn Thr Lys | |
| 195 200 205 | |
| Val Asp Lys Arg Val Glu Pro Lys Ser Cys Asp Lys Thr His Thr Cys | |
| 210 215 220 | |
| Pro Pro Cys Pro Ala Pro Glu Ala Ala Gly Gly Pro Ser Val Phe Leu | |
| 225 230 235 240 | |
| Phe Pro Pro Lys Pro Lys Asp Thr Leu Met Ile Ser Arg Thr Pro Glu | |
| 245 250 255 | |
| Val Thr Cys Val Val Val Asp Val Ser His Glu Asp Pro Glu Val Lys | |
| 260 265 270 | |
| Phe Asn Trp Tyr Val Asp Gly Val Glu Val His Asn Ala Lys Thr Lys | |
| 275 280 285 | |
| Pro Arg Glu Glu Gln Tyr Asn Ser Thr Tyr Arg Val Val Ser Val Leu | |
| 290 295 300 | |
| Thr Val Leu His Gln Asp Trp Leu Asn Gly Lys Glu Tyr Lys Cys Lys | |
| 305 310 315 320 | |
| Val Ser Asn Lys Ala Leu Pro Ala Pro Ile Glu Lys Thr Ile Ser Lys | |
| 325 330 335 | |
| Ala Lys Gly Gln Pro Arg Glu Pro Gln Val Tyr Thr Leu Pro Pro Ser | |
| 340 345 350 | |
| Arg Glu Glu Met Thr Lys Asn Gln Val Ser Leu Thr Cys Leu Val Lys | |
| 355 360 365 | |
| Gly Phe Tyr Pro Ser Asp Ile Ala Val Glu Trp Glu Ser Asn Gly Gln | |
| 370 375 380 | |
| Pro Glu Asn Asn Tyr Lys Thr Thr Pro Pro Val Leu Asp Ser Asp Gly | |
| 385 390 395 400 | |
| Ser Phe Phe Leu Tyr Ser Lys Leu Thr Val Asp Lys Ser Arg Trp Gln | |
| 405 410 415 | |
| Gln Gly Asn Val Phe Ser Cys Ser Val Met His Glu Ala Leu His Asn | |
| 420 425 430 | |
| His Tyr Thr Gln Lys Ser Leu Ser Leu Ser Pro Gly Lys | |
| 435 440 445 | |
| SEQ ID NO: 172 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 173 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 174 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 175 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 176 | |
| Gln Ser Ala Leu Thr Gln Pro Ala Ser Val Ser Gly Ser Pro Gly Gln | |
| 1 5 10 15 | |
| Ser Ile Thr Ile Ser Cys Thr Gly Thr Ser Ser Asp Val Gly Ser Tyr | |
| 20 25 30 | |
| Asn Tyr Val Asn Trp Tyr Gln Gln His Pro Gly Lys Ala Pro Lys Leu | |
| 35 40 45 | |
| Met Ile Tyr Gly Val Ser Lys Arg Pro Ser Gly Val Ser Asn Arg Phe | |
| 50 55 60 | |
| Ser Gly Ser Lys Ser Gly Asn Thr Ala Ser Leu Thr Ile Ser Gly Leu | |
| 65 70 75 80 | |
| Gln Ala Glu Asp Glu Ala Asp Tyr Tyr Cys Gly Thr Phe Ala Gly Gly | |
| 85 90 95 | |
| Ser Tyr Tyr Gly Val Phe Gly Gly Gly Thr Lys Leu Thr Val Leu Gly | |
| 100 105 110 | |
| Gln Pro Lys Ala Ala Pro Ser Val Thr Leu Phe Pro Pro Ser Ser Glu | |
| 115 120 125 | |
| Glu Leu Gln Ala Asn Lys Ala Thr Leu Val Cys Leu Ile Ser Asp Phe | |
| 130 135 140 | |
| Tyr Pro Gly Ala Val Thr Val Ala Trp Lys Ala Asp Ser Ser Pro Val | |
| 145 150 155 160 | |
| Lys Ala Gly Val Glu Thr Thr Thr Pro Ser Lys Gln Ser Asn Asn Lys | |
| 165 170 175 | |
| Tyr Ala Ala Ser Ser Tyr Leu Ser Leu Thr Pro Glu Gln Trp Lys Ser | |
| 180 185 190 | |
| His Arg Ser Tyr Ser Cys Gln Val Thr His Glu Gly Ser Thr Val Glu | |
| 195 200 205 | |
| Lys Thr Val Ala Pro Thr Glu Cys Ser | |
| 210 215 | |
| SEQ ID NO: 177 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Thr Ile Asn Pro Val Ser Gly Ser Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Cys Ser Arg Ser Thr Ser Glu Ser Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Asn Phe Gly | |
| 180 185 190 | |
| Thr Gln Thr Tyr Thr Cys Asn Val Asp His Lys Pro Ser Asn Thr Lys | |
| 195 200 205 | |
| Val Asp Lys Thr Val Glu Arg Lys Cys Cys Val Glu Cys Pro Pro Cys | |
| 210 215 220 | |
| Pro Ala Pro Pro Val Ala Gly Pro Ser Val Phe Leu Phe Pro Pro Lys | |
| 225 230 235 240 | |
| Pro Lys Asp Thr Leu Met Ile Ser Arg Thr Pro Glu Val Thr Cys Val | |
| 245 250 255 | |
| Val Val Asp Val Ser His Glu Asp Pro Glu Val Gln Phe Asn Trp Tyr | |
| 260 265 270 | |
| Val Asp Gly Val Glu Val His Asn Ala Lys Thr Lys Pro Arg Glu Glu | |
| 275 280 285 | |
| Gln Phe Asn Ser Thr Phe Arg Val Val Ser Val Leu Thr Val Val His | |
| 290 295 300 | |
| Gln Asp Trp Leu Asn Gly Lys Glu Tyr Lys Cys Lys Val Ser Asn Lys | |
| 305 310 315 320 | |
| Gly Leu Pro Ala Pro Ile Glu Lys Thr Ile Ser Lys Thr Lys Gly Gln | |
| 325 330 335 | |
| Pro Arg Glu Pro Gln Val Tyr Thr Leu Pro Pro Ser Arg Glu Glu Met | |
| 340 345 350 | |
| Thr Lys Asn Gln Val Ser Leu Thr Cys Leu Val Lys Gly Phe Tyr Pro | |
| 355 360 365 | |
| Ser Asp Ile Ala Val Glu Trp Glu Ser Asn Gly Gln Pro Glu Asn Asn | |
| 370 375 380 | |
| Tyr Lys Thr Thr Pro Pro Met Leu Asp Ser Asp Gly Ser Phe Phe Leu | |
| 385 390 395 400 | |
| Tyr Ser Lys Leu Thr Val Asp Lys Ser Arg Trp Gln Gln Gly Asn Val | |
| 405 410 415 | |
| Phe Ser Cys Ser Val Met His Glu Ala Leu His Asn His Tyr Thr Gln | |
| 420 425 430 | |
| Lys Ser Leu Ser Leu Ser Pro Gly Lys | |
| 435 440 | |
| SEQ ID NO: 178 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Gln Ile Asn Ala Ala Ser Gly Met Thr Arg Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Cys Ser Arg Ser Thr Ser Glu Ser Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Asn Phe Gly | |
| 180 185 190 | |
| Thr Gln Thr Tyr Thr Cys Asn Val Asp His Lys Pro Ser Asn Thr Lys | |
| 195 200 205 | |
| Val Asp Lys Thr Val Glu Arg Lys Cys Cys Val Glu Cys Pro Pro Cys | |
| 210 215 220 | |
| Pro Ala Pro Pro Val Ala Gly Pro Ser Val Phe Leu Phe Pro Pro Lys | |
| 225 230 235 240 | |
| Pro Lys Asp Thr Leu Met Ile Ser Arg Thr Pro Glu Val Thr Cys Val | |
| 245 250 255 | |
| Val Val Asp Val Ser His Glu Asp Pro Glu Val Gln Phe Asn Trp Tyr | |
| 260 265 270 | |
| Val Asp Gly Val Glu Val His Asn Ala Lys Thr Lys Pro Arg Glu Glu | |
| 275 280 285 | |
| Gln Phe Asn Ser Thr Phe Arg Val Val Ser Val Leu Thr Val Val His | |
| 290 295 300 | |
| Gln Asp Trp Leu Asn Gly Lys Glu Tyr Lys Cys Lys Val Ser Asn Lys | |
| 305 310 315 320 | |
| Gly Leu Pro Ala Pro Ile Glu Lys Thr Ile Ser Lys Thr Lys Gly Gln | |
| 325 330 335 | |
| Pro Arg Glu Pro Gln Val Tyr Thr Leu Pro Pro Ser Arg Glu Glu Met | |
| 340 345 350 | |
| Thr Lys Asn Gln Val Ser Leu Thr Cys Leu Val Lys Gly Phe Tyr Pro | |
| 355 360 365 | |
| Ser Asp Ile Ala Val Glu Trp Glu Ser Asn Gly Gln Pro Glu Asn Asn | |
| 370 375 380 | |
| Tyr Lys Thr Thr Pro Pro Met Leu Asp Ser Asp Gly Ser Phe Phe Leu | |
| 385 390 395 400 | |
| Tyr Ser Lys Leu Thr Val Asp Lys Ser Arg Trp Gln Gln Gly Asn Val | |
| 405 410 415 | |
| Phe Ser Cys Ser Val Met His Glu Ala Leu His Asn His Tyr Thr Gln | |
| 420 425 430 | |
| Lys Ser Leu Ser Leu Ser Pro Gly Lys | |
| 435 440 | |
| SEQ ID NO: 179 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Asn Ile Asn Ala Ala Ala Gly Ile Thr Leu Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Cys Ser Arg Ser Thr Ser Glu Ser Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Asn Phe Gly | |
| 180 185 190 | |
| Thr Gln Thr Tyr Thr Cys Asn Val Asp His Lys Pro Ser Asn Thr Lys | |
| 195 200 205 | |
| Val Asp Lys Thr Val Glu Arg Lys Cys Cys Val Glu Cys Pro Pro Cys | |
| 210 215 220 | |
| Pro Ala Pro Pro Val Ala Gly Pro Ser Val Phe Leu Phe Pro Pro Lys | |
| 225 230 235 240 | |
| Pro Lys Asp Thr Leu Met Ile Ser Arg Thr Pro Glu Val Thr Cys Val | |
| 245 250 255 | |
| Val Val Asp Val Ser His Glu Asp Pro Glu Val Gln Phe Asn Trp Tyr | |
| 260 265 270 | |
| Val Asp Gly Val Glu Val His Asn Ala Lys Thr Lys Pro Arg Glu Glu | |
| 275 280 285 | |
| Gln Phe Asn Ser Thr Phe Arg Val Val Ser Val Leu Thr Val Val His | |
| 290 295 300 | |
| Gln Asp Trp Leu Asn Gly Lys Glu Tyr Lys Cys Lys Val Ser Asn Lys | |
| 305 310 315 320 | |
| Gly Leu Pro Ala Pro Ile Glu Lys Thr Ile Ser Lys Thr Lys Gly Gln | |
| 325 330 335 | |
| Pro Arg Glu Pro Gln Val Tyr Thr Leu Pro Pro Ser Arg Glu Glu Met | |
| 340 345 350 | |
| Thr Lys Asn Gln Val Ser Leu Thr Cys Leu Val Lys Gly Phe Tyr Pro | |
| 355 360 365 | |
| Ser Asp Ile Ala Val Glu Trp Glu Ser Asn Gly Gln Pro Glu Asn Asn | |
| 370 375 380 | |
| Tyr Lys Thr Thr Pro Pro Met Leu Asp Ser Asp Gly Ser Phe Phe Leu | |
| 385 390 395 400 | |
| Tyr Ser Lys Leu Thr Val Asp Lys Ser Arg Trp Gln Gln Gly Asn Val | |
| 405 410 415 | |
| Phe Ser Cys Ser Val Met His Glu Ala Leu His Asn His Tyr Thr Gln | |
| 420 425 430 | |
| Lys Ser Leu Ser Leu Ser Pro Gly Lys | |
| 435 440 | |
| SEQ ID NO: 180 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Gly Ile Asn Pro Pro Ala Gly Thr Thr Ser Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Cys Ser Arg Ser Thr Ser Glu Ser Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Asn Phe Gly | |
| 180 185 190 | |
| Thr Gln Thr Tyr Thr Cys Asn Val Asp His Lys Pro Ser Asn Thr Lys | |
| 195 200 205 | |
| Val Asp Lys Thr Val Glu Arg Lys Cys Cys Val Glu Cys Pro Pro Cys | |
| 210 215 220 | |
| Pro Ala Pro Pro Val Ala Gly Pro Ser Val Phe Leu Phe Pro Pro Lys | |
| 225 230 235 240 | |
| Pro Lys Asp Thr Leu Met Ile Ser Arg Thr Pro Glu Val Thr Cys Val | |
| 245 250 255 | |
| Val Val Asp Val Ser His Glu Asp Pro Glu Val Gln Phe Asn Trp Tyr | |
| 260 265 270 | |
| Val Asp Gly Val Glu Val His Asn Ala Lys Thr Lys Pro Arg Glu Glu | |
| 275 280 285 | |
| Gln Phe Asn Ser Thr Phe Arg Val Val Ser Val Leu Thr Val Val His | |
| 290 295 300 | |
| Gln Asp Trp Leu Asn Gly Lys Glu Tyr Lys Cys Lys Val Ser Asn Lys | |
| 305 310 315 320 | |
| Gly Leu Pro Ala Pro Ile Glu Lys Thr Ile Ser Lys Thr Lys Gly Gln | |
| 325 330 335 | |
| Pro Arg Glu Pro Gln Val Tyr Thr Leu Pro Pro Ser Arg Glu Glu Met | |
| 340 345 350 | |
| Thr Lys Asn Gln Val Ser Leu Thr Cys Leu Val Lys Gly Phe Tyr Pro | |
| 355 360 365 | |
| Ser Asp Ile Ala Val Glu Trp Glu Ser Asn Gly Gln Pro Glu Asn Asn | |
| 370 375 380 | |
| Tyr Lys Thr Thr Pro Pro Met Leu Asp Ser Asp Gly Ser Phe Phe Leu | |
| 385 390 395 400 | |
| Tyr Ser Lys Leu Thr Val Asp Lys Ser Arg Trp Gln Gln Gly Asn Val | |
| 405 410 415 | |
| Phe Ser Cys Ser Val Met His Glu Ala Leu His Asn His Tyr Thr Gln | |
| 420 425 430 | |
| Lys Ser Leu Ser Leu Ser Pro Gly Lys | |
| 435 440 | |
| SEQ ID NO: 181 | |
| Gln Val Gln Leu Val Gln Ser Gly Ala Glu Val Lys Lys Pro Gly Ala | |
| 1 5 10 15 | |
| Ser Val Lys Val Ser Cys Lys Ala Ser Gly Tyr Thr Phe Thr Ser Ser | |
| 20 25 30 | |
| Tyr Ile Asn Trp Val Arg Gln Ala Pro Gly Gln Gly Leu Glu Trp Met | |
| 35 40 45 | |
| Gly Asn Ile Asn Pro Ala Thr Gly His Ala Asp Tyr Ala Gln Lys Phe | |
| 50 55 60 | |
| Gln Gly Arg Val Thr Met Thr Arg Asp Thr Ser Ile Ser Thr Ala Tyr | |
| 65 70 75 80 | |
| Met Glu Leu Ser Arg Leu Arg Ser Asp Asp Thr Ala Val Tyr Tyr Cys | |
| 85 90 95 | |
| Ala Arg Gly Gly Trp Phe Asp Tyr Trp Gly Gln Gly Thr Leu Val Thr | |
| 100 105 110 | |
| Val Ser Ser Ala Ser Thr Lys Gly Pro Ser Val Phe Pro Leu Ala Pro | |
| 115 120 125 | |
| Cys Ser Arg Ser Thr Ser Glu Ser Thr Ala Ala Leu Gly Cys Leu Val | |
| 130 135 140 | |
| Lys Asp Tyr Phe Pro Glu Pro Val Thr Val Ser Trp Asn Ser Gly Ala | |
| 145 150 155 160 | |
| Leu Thr Ser Gly Val His Thr Phe Pro Ala Val Leu Gln Ser Ser Gly | |
| 165 170 175 | |
| Leu Tyr Ser Leu Ser Ser Val Val Thr Val Pro Ser Ser Asn Phe Gly | |
| 180 185 190 | |
| Thr Gln Thr Tyr Thr Cys Asn Val Asp His Lys Pro Ser Asn Thr Lys | |
| 195 200 205 | |
| Val Asp Lys Thr Val Glu Arg Lys Cys Cys Val Glu Cys Pro Pro Cys | |
| 210 215 220 | |
| Pro Ala Pro Pro Val Ala Gly Pro Ser Val Phe Leu Phe Pro Pro Lys | |
| 225 230 235 240 | |
| Pro Lys Asp Thr Leu Met Ile Ser Arg Thr Pro Glu Val Thr Cys Val | |
| 245 250 255 | |
| Val Val Asp Val Ser His Glu Asp Pro Glu Val Gln Phe Asn Trp Tyr | |
| 260 265 270 | |
| Val Asp Gly Val Glu Val His Asn Ala Lys Thr Lys Pro Arg Glu Glu | |
| 275 280 285 | |
| Gln Phe Asn Ser Thr Phe Arg Val Val Ser Val Leu Thr Val Val His | |
| 290 295 300 | |
| Gln Asp Trp Leu Asn Gly Lys Glu Tyr Lys Cys Lys Val Ser Asn Lys | |
| 305 310 315 320 | |
| Gly Leu Pro Ala Pro Ile Glu Lys Thr Ile Ser Lys Thr Lys Gly Gln | |
| 325 330 335 | |
| Pro Arg Glu Pro Gln Val Tyr Thr Leu Pro Pro Ser Arg Glu Glu Met | |
| 340 345 350 | |
| Thr Lys Asn Gln Val Ser Leu Thr Cys Leu Val Lys Gly Phe Tyr Pro | |
| 355 360 365 | |
| Ser Asp Ile Ala Val Glu Trp Glu Ser Asn Gly Gln Pro Glu Asn Asn | |
| 370 375 380 | |
| Tyr Lys Thr Thr Pro Pro Met Leu Asp Ser Asp Gly Ser Phe Phe Leu | |
| 385 390 395 400 | |
| Tyr Ser Lys Leu Thr Val Asp Lys Ser Arg Trp Gln Gln Gly Asn Val | |
| 405 410 415 | |
| Phe Ser Cys Ser Val Met His Glu Ala Leu His Asn His Tyr Thr Gln | |
| 420 425 430 | |
| Lys Ser Leu Ser Leu Ser Pro Gly Lys | |
| 435 440 | |
| SEQ ID NO: 182 | |
| cagagcgccc tgacccagcc cgccagcgtg tccggcagcc caggccagtc tatcacaatc | 60 |
| agctgcaccg gcacctccag cgacgtgggc agctacaact acgtgaactg gtatcagcag | 120 |
| caccccggca aggcccccaa gctgatgatc tacggcgtga gcaagaggcc cagcggcgtg | 180 |
| tccaacaggt tcagcggcag caagagcggc aacaccgcca gcctgacaat cagtgggctg | 240 |
| caggctgagg acgaggccga ctactactgc ggcacctttg ccggcggatc atactacggc | 300 |
| gtgttcggcg gagggaccaa gctgaccgtg ctgggccagc ctaaggctgc ccccagcgtg | 360 |
| accctgttcc cccccagcag cgaggagctg caggccaaca aggccaccct ggtgtgcctg | 420 |
| atcagcgact tctacccagg cgccgtgacc gtggcctgga aggccgacag cagccccgtg | 480 |
| aaggccggcg tggagaccac cacccccagc aagcagagca acaacaagta cgccgccagc | 540 |
| agctacctga gcctgacccc cgagcagtgg aagagccaca ggtcctacag ctgccaggtg | 600 |
| acccacgagg gcagcaccgt ggaaaagacc gtggccccaa ccgagtgcag c | 651 |
| SEQ ID NO: 183 | |
| cagagcgccc tgacccagcc cgccagcgtg tccggcagcc caggccagtc tatcacaatc | 60 |
| agctgcaccg gcacctccag cgacgtgggc agctacaact acgtgaactg gtatcagcag | 120 |
| caccccggca aggcccccaa gctgatgatc tacggcgtga gcaagaggcc cagcggcgtg | 180 |
| tccaacaggt tcagcggcag caagagcggc aacaccgcca gcctgacaat cagtgggctg | 240 |
| caggctgagg acgaggccga ctactactgc ggcacctttg ccggcggatc atactacggc | 300 |
| gtgttcggcg gagggaccaa gctgaccgtg ctgggccagc ctaaggctgc ccccagcgtg | 360 |
| accctgttcc cccccagcag cgaggagctg caggccaaca aggccaccct ggtgtgcctg | 420 |
| atcagcgact tctacccagg cgccgtgacc gtggcctgga aggccgacag cagccccgtg | 480 |
| aaggccggcg tggagaccac cacccccagc aagcagagca acaacaagta cgccgccagc | 540 |
| agctacctga gcctgacccc cgagcagtgg aagagccaca ggtcctacag ctgccaggtg | 600 |
| acccacgagg gcagcaccgt ggaaaagacc gtggccccaa ccgagtgcag c | 651 |
| SEQ ID NO: 184 | |
| cagagcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtc ctaggtcagc ccaaggctgc cccctcggtc | 360 |
| actctgttcc cgccctcctc tgaggagctt caagccaaca aggccacact ggtgtgtctc | 420 |
| ataagtgact tctacccggg agccgtgaca gtggcctgga aggcagatag cagccccgtc | 480 |
| aaggcgggag tggagaccac cacaccctcc aaacaaagca acaacaagta cgcggccagc | 540 |
| agctatctga gcctgacgcc tgagcagtgg aagtcccaca gaagctacag ctgccaggtc | 600 |
| acgcatgaag ggagcaccgt ggagaagaca gtggccccta cagaatgttc a | 651 |
| SEQ ID NO: 185 | |
| cagagcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtc ctaggtcagc ccaaggctgc cccctcggtc | 360 |
| actctgttcc cgccctcctc tgaggagctt caagccaaca aggccacact ggtgtgtctc | 420 |
| ataagtgact tctacccggg agccgtgaca gtggcctgga aggcagatag cagccccgtc | 480 |
| aaggcgggag tggagaccac cacaccctcc aaacaaagca acaacaagta cgcggccagc | 540 |
| agctatctga gcctgacgcc tgagcagtgg aagtcccaca gaagctacag ctgccaggtc | 600 |
| acgcatgaag ggagcaccgt ggagaagaca gtggccccta cagaatgttc a | 651 |
| SEQ ID NO: 186 | |
| cagagcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtc ctaggtcagc ccaaggctgc cccctcggtc | 360 |
| actctgttcc cgccctcctc tgaggagctt caagccaaca aggccacact ggtgtgtctc | 420 |
| ataagtgact tctacccggg agccgtgaca gtggcctgga aggcagatag cagccccgtc | 480 |
| aaggcgggag tggagaccac cacaccctcc aaacaaagca acaacaagta cgcggccagc | 540 |
| agctatctga gcctgacgcc tgagcagtgg aagtcccaca gaagctacag ctgccaggtc | 600 |
| acgcatgaag ggagcaccgt ggagaagaca gtggccccta cagaatgttc a | 651 |
| SEQ ID NO: 187 | |
| caggtgcagc tggtgcagag cggagctgag gtgaagaagc caggcgccag cgtcaaggtg | 60 |
| tcctgcaagg ccagcggcta caccttcacc agcagctaca tcaactgggt ccgccaggct | 120 |
| cctgggcagg gactggagtg gatgggcacc atcaaccccg tgtccggcag caccagctac | 180 |
| gcccagaagt tccagggcag agtcaccatg accagggaca ccagcatcag caccgcctac | 240 |
| atggagctgt ccaggctgag aagcgacgac accgccgtgt actactgcgc caggggcggc | 300 |
| tggttcgact actggggcca gggcaccctg gtgaccgtgt cctcagctag caccaagggc | 360 |
| cccagcgtgt tccccctggc ccccagcagc aagagcacct ccggcggcac agccgccctg | 420 |
| ggctgcctgg tgaaggacta cttccccgag cccgtgaccg tgtcctggaa cagcggagcc | 480 |
| ctgaccagcg gcgtgcacac cttccccgcc gtgctgcaga gcagcggcct gtacagcctg | 540 |
| tccagcgtgg tgacagtgcc cagcagcagc ctgggcaccc agacctacat ctgcaacgtg | 600 |
| aaccacaagc ccagcaacac caaggtggac aagagagtgg agcccaagag ctgcgacaag | 660 |
| acccacacct gccccccctg cccagccccc gaagctgcag gcggcccttc cgtgttcctg | 720 |
| ttccccccca agcccaagga caccctgatg atcagcagga cccccgaggt gacctgcgtg | 780 |
| gtggtggacg tgagccacga ggacccagag gtgaagttca actggtacgt ggacggcgtg | 840 |
| gaggtgcaca acgccaagac caagcccaga gaggagcagt acaacagcac ctacagggtg | 900 |
| gtgtccgtgc tgaccgtgct gcaccaggac tggctgaacg gcaaagaata caagtgcaag | 960 |
| gtctccaaca aggccctgcc tgcccccatc gaaaagacca tcagcaaggc caagggccag | 1020 |
| ccacgggagc cccaggtgta caccctgccc ccttctcggg aggagatgac caagaaccag | 1080 |
| gtgtccctga cctgtctggt gaagggcttc taccccagcg acatcgccgt ggagtgggag | 1140 |
| agcaacggcc agcccgagaa caactacaag accacccccc cagtgctgga cagcgacggc | 1200 |
| agcttcttcc tgtacagcaa gctgaccgtg gacaagagca ggtggcagca gggcaacgtg | 1260 |
| ttcagctgca gcgtgatgca cgaggccctg cacaaccact acacccagaa gagcctgagc | 1320 |
| ctgtcacccg gcaag | 1335 |
| SEQ ID NO: 188 | |
| caggtgcagc tggtgcagag cggagctgag gtgaagaagc caggcgccag cgtcaaggtg | 60 |
| tcctgcaagg ccagcggcta caccttcacc agcagctaca tcaactgggt gcgccaggct | 120 |
| ccagggcagg gactggagtg gatgggccag atcaacgccg ccagcggcat gaccagatac | 180 |
| gcccagaagt tccagggcag agtcacaatg accagggaca cctctatcag caccgcctac | 240 |
| atggagctgt ccaggctgag aagcgacgac accgccgtgt actactgcgc caggggcggc | 300 |
| tggttcgact actggggcca gggcaccctg gtgaccgtgt cctcagctag caccaagggc | 360 |
| cccagcgtgt tccccctggc ccccagcagc aagagcacct ccggcggcac agccgccctg | 420 |
| ggctgcctgg tgaaggacta cttccccgag cccgtgaccg tgtcctggaa cagcggagcc | 480 |
| ctgaccagcg gcgtgcacac cttccccgcc gtgctgcaga gcagcggcct gtacagcctg | 540 |
| tccagcgtgg tgacagtgcc cagcagcagc ctgggcaccc agacctacat ctgcaacgtg | 600 |
| aaccacaagc ccagcaacac caaggtggac aagagagtgg agcccaagag ctgcgacaag | 660 |
| acccacacct gccccccctg cccagccccc gaagctgcag gcggcccttc cgtgttcctg | 720 |
| ttccccccca agcccaagga caccctgatg atcagcagga cccccgaggt gacctgcgtg | 780 |
| gtggtggacg tgagccacga ggacccagag gtgaagttca actggtacgt ggacggcgtg | 840 |
| gaggtgcaca acgccaagac caagcccaga gaggagcagt acaacagcac ctacagggtg | 900 |
| gtgtccgtgc tgaccgtgct gcaccaggac tggctgaacg gcaaagaata caagtgcaag | 960 |
| gtctccaaca aggccctgcc tgcccccatc gaaaagacca tcagcaaggc caagggccag | 1020 |
| ccacgggagc cccaggtgta caccctgccc ccttctcggg aggagatgac caagaaccag | 1080 |
| gtgtccctga cctgtctggt gaagggcttc taccccagcg acatcgccgt ggagtgggag | 1140 |
| agcaacggcc agcccgagaa caactacaag accacccccc cagtgctgga cagcgacggc | 1200 |
| agcttcttcc tgtacagcaa gctgaccgtg gacaagagca ggtggcagca gggcaacgtg | 1260 |
| ttcagctgca gcgtgatgca cgaggccctg cacaaccact acacccagaa gagcctgagc | 1320 |
| ctgtcacccg gcaag | 1335 |
| SEQ ID NO: 189 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcaat attaatgctg ctgctggtat tactctttat | 180 |
| gctcagaagt ttcagggtcg ggtcaccatg acccgtgata ccagcattag caccgcgtat | 240 |
| atggaactga gccgcctgcg tagcgatgat acggccgtgt attattgcgc gcgtggtggt | 300 |
| tggtttgatt attggggcca aggcaccctg gtgacggtta gctcagcctc caccaagggt | 360 |
| ccatcggtct tccccctggc accctcctcc aagagcacct ctgggggcac agcggccctg | 420 |
| ggctgcctgg tcaaggacta cttccccgaa ccggtgacgg tgtcgtggaa ctcaggcgcc | 480 |
| ctgaccagcg gcgtgcacac cttcccggct gtcctacagt cctcaggact ctactccctc | 540 |
| agcagcgtgg tgaccgtgcc ctccagcagc ttgggcaccc agacctacat ctgcaacgtg | 600 |
| aatcacaagc ccagcaacac caaggtggac aagagagttg agcccaaatc ttgtgacaaa | 660 |
| actcacacat gcccaccgtg cccagcacct gaagcagcgg ggggaccgtc agtcttcctc | 720 |
| ttccccccaa aacccaagga caccctcatg atctcccgga cccctgaggt cacatgcgtg | 780 |
| gtggtggacg tgagccacga agaccctgag gtcaagttca actggtacgt ggacggcgtg | 840 |
| gaggtgcata atgccaagac aaagccgcgg gaggagcagt acaacagcac gtaccgggtg | 900 |
| gtcagcgtcc tcaccgtcct gcaccaggac tggctgaatg gcaaggagta caagtgcaag | 960 |
| gtctccaaca aagccctccc agcccccatc gagaaaacca tctccaaagc caaagggcag | 1020 |
| ccccgagaac cacaggtgta caccctgccc ccatcccggg aggagatgac caagaaccag | 1080 |
| gtcagcctga cctgcctggt caaaggcttc tatcccagcg acatcgccgt ggagtgggag | 1140 |
| agcaatgggc agccggagaa caactacaag accacgcctc ccgtgctgga ctccgacggc | 1200 |
| tccttcttcc tctacagcaa gctcaccgtg gacaagagca ggtggcagca ggggaacgtc | 1260 |
| ttctcatgct ccgtgatgca tgaggctctg cacaaccact acacgcagaa gagcctctcc | 1320 |
| ctgtctccgg gtaaa | 1335 |
| SEQ ID NO: 190 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcggt attaatcctc ctgctggtac tacttcttat | 180 |
| gctcagaagt ttcagggtcg ggtcaccatg acccgtgata ccagcattag caccgcgtat | 240 |
| atggaactga gccgcctgcg tagcgatgat acggccgtgt attattgcgc gcgtggtggt | 300 |
| tggtttgatt attggggcca aggcaccctg gtgacggtta gctcagcctc caccaagggt | 360 |
| ccatcggtct tccccctggc accctcctcc aagagcacct ctgggggcac agcggccctg | 420 |
| ggctgcctgg tcaaggacta cttccccgaa ccggtgacgg tgtcgtggaa ctcaggcgcc | 480 |
| ctgaccagcg gcgtgcacac cttcccggct gtcctacagt cctcaggact ctactccctc | 540 |
| agcagcgtgg tgaccgtgcc ctccagcagc ttgggcaccc agacctacat ctgcaacgtg | 600 |
| aatcacaagc ccagcaacac caaggtggac aagagagttg agcccaaatc ttgtgacaaa | 660 |
| actcacacat gcccaccgtg cccagcacct gaagcagcgg ggggaccgtc agtcttcctc | 720 |
| ttccccccaa aacccaagga caccctcatg atctcccgga cccctgaggt cacatgcgtg | 780 |
| gtggtggacg tgagccacga agaccctgag gtcaagttca actggtacgt ggacggcgtg | 840 |
| gaggtgcata atgccaagac aaagccgcgg gaggagcagt acaacagcac gtaccgggtg | 900 |
| gtcagcgtcc tcaccgtcct gcaccaggac tggctgaatg gcaaggagta caagtgcaag | 960 |
| gtctccaaca aagccctccc agcccccatc gagaaaacca tctccaaagc caaagggcag | 1020 |
| ccccgagaac cacaggtgta caccctgccc ccatcccggg aggagatgac caagaaccag | 1080 |
| gtcagcctga cctgcctggt caaaggcttc tatcccagcg acatcgccgt ggagtgggag | 1140 |
| agcaatgggc agccggagaa caactacaag accacgcctc ccgtgctgga ctccgacggc | 1200 |
| tccttcttcc tctacagcaa gctcaccgtg gacaagagca ggtggcagca ggggaacgtc | 1260 |
| ttctcatgct ccgtgatgca tgaggctctg cacaaccact acacgcagaa gagcctctcc | 1320 |
| ctgtctccgg gtaaa | 1335 |
| SEQ ID NO: 191 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcaat attaatcctg ctactggtca tgctgattat | 180 |
| gctcagaagt ttcagggtcg ggtgaccatg acccgtgata ccagcattag caccgcgtat | 240 |
| atggaactga gccgcctgcg tagcgatgat acggccgtgt attattgcgc gcgtggtggt | 300 |
| tggtttgatt attggggcca aggcaccctg gtgacggtta gctcagcctc caccaagggt | 360 |
| ccatcggtct tccccctggc accctcctcc aagagcacct ctgggggcac agcggccctg | 420 |
| ggctgcctgg tcaaggacta cttccccgaa ccggtgacgg tgtcgtggaa ctcaggcgcc | 480 |
| ctgaccagcg gcgtgcacac cttcccggct gtcctacagt cctcaggact ctactccctc | 540 |
| agcagcgtgg tgaccgtgcc ctccagcagc ttgggcaccc agacctacat ctgcaacgtg | 600 |
| aatcacaagc ccagcaacac caaggtggac aagagagttg agcccaaatc ttgtgacaaa | 660 |
| actcacacat gcccaccgtg cccagcacct gaagcagcgg ggggaccgtc agtcttcctc | 720 |
| ttccccccaa aacccaagga caccctcatg atctcccgga cccctgaggt cacatgcgtg | 780 |
| gtggtggacg tgagccacga agaccctgag gtcaagttca actggtacgt ggacggcgtg | 840 |
| gaggtgcata atgccaagac aaagccgcgg gaggagcagt acaacagcac gtaccgggtg | 900 |
| gtcagcgtcc tcaccgtcct gcaccaggac tggctgaatg gcaaggagta caagtgcaag | 960 |
| gtctccaaca aagccctccc agcccccatc gagaaaacca tctccaaagc caaagggcag | 1020 |
| ccccgagaac cacaggtgta caccctgccc ccatcccggg aggagatgac caagaaccag | 1080 |
| gtcagcctga cctgcctggt caaaggcttc tatcccagcg acatcgccgt ggagtgggag | 1140 |
| agcaatgggc agccggagaa caactacaag accacgcctc ccgtgctgga ctccgacggc | 1200 |
| tccttcttcc tctacagcaa gctcaccgtg gacaagagca ggtggcagca ggggaacgtc | 1260 |
| ttctcatgct ccgtgatgca tgaggctctg cacaaccact acacgcagaa gagcctctcc | 1320 |
| ctgtctccgg gtaaa | 1335 |
| SEQ ID NO: 192 | |
| cagagcgccc tgacccagcc cgccagcgtg tccggcagcc caggccagtc tatcacaatc | 60 |
| agctgcaccg gcacctccag cgacgtgggc agctacaact acgtgaactg gtatcagcag | 120 |
| caccccggca aggcccccaa gctgatgatc tacggcgtga gcaagaggcc cagcggcgtg | 180 |
| tccaacaggt tcagcggcag caagagcggc aacaccgcca gcctgacaat cagtgggctg | 240 |
| caggctgagg acgaggccga ctactactgc ggcacctttg ccggcggatc atactacggc | 300 |
| gtgttcggcg gagggaccaa gctgaccgtg ctgggccagc ctaaggctgc ccccagcgtg | 360 |
| accctgttcc cccccagcag cgaggagctg caggccaaca aggccaccct ggtgtgcctg | 420 |
| atcagcgact tctacccagg cgccgtgacc gtggcctgga aggccgacag cagccccgtg | 480 |
| aaggccggcg tggagaccac cacccccagc aagcagagca acaacaagta cgccgccagc | 540 |
| agctacctga gcctgacccc cgagcagtgg aagagccaca ggtcctacag ctgccaggtg | 600 |
| acccacgagg gcagcaccgt ggaaaagacc gtggccccaa ccgagtgcag c | 651 |
| SEQ ID NO: 193 | |
| cagagcgccc tgacccagcc cgccagcgtg tccggcagcc caggccagtc tatcacaatc | 60 |
| agctgcaccg gcacctccag cgacgtgggc agctacaact acgtgaactg gtatcagcag | 120 |
| caccccggca aggcccccaa gctgatgatc tacggcgtga gcaagaggcc cagcggcgtg | 180 |
| tccaacaggt tcagcggcag caagagcggc aacaccgcca gcctgacaat cagtgggctg | 240 |
| caggctgagg acgaggccga ctactactgc ggcacctttg ccggcggatc atactacggc | 300 |
| gtgttcggcg gagggaccaa gctgaccgtg ctgggccagc ctaaggctgc ccccagcgtg | 360 |
| accctgttcc cccccagcag cgaggagctg caggccaaca aggccaccct ggtgtgcctg | 420 |
| atcagcgact tctacccagg cgccgtgacc gtggcctgga aggccgacag cagccccgtg | 480 |
| aaggccggcg tggagaccac cacccccagc aagcagagca acaacaagta cgccgccagc | 540 |
| agctacctga gcctgacccc cgagcagtgg aagagccaca ggtcctacag ctgccaggtg | 600 |
| acccacgagg gcagcaccgt ggaaaagacc gtggccccaa ccgagtgcag c | 651 |
| SEQ ID NO: 194 | |
| cagagcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtc ctaggtcagc ccaaggctgc cccctcggtc | 360 |
| actctgttcc cgccctcctc tgaggagctt caagccaaca aggccacact ggtgtgtctc | 420 |
| ataagtgact tctacccggg agccgtgaca gtggcctgga aggcagatag cagccccgtc | 480 |
| aaggcgggag tggagaccac cacaccctcc aaacaaagca acaacaagta cgcggccagc | 540 |
| agctatctga gcctgacgcc tgagcagtgg aagtcccaca gaagctacag ctgccaggtc | 600 |
| acgcatgaag ggagcaccgt ggagaagaca gtggccccta cagaatgttc a | 651 |
| SEQ ID NO: 195 | |
| cagagcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtc ctaggtcagc ccaaggctgc cccctcggtc | 360 |
| actctgttcc cgccctcctc tgaggagctt caagccaaca aggccacact ggtgtgtctc | 420 |
| ataagtgact tctacccggg agccgtgaca gtggcctgga aggcagatag cagccccgtc | 480 |
| aaggcgggag tggagaccac cacaccctcc aaacaaagca acaacaagta cgcggccagc | 540 |
| agctatctga gcctgacgcc tgagcagtgg aagtcccaca gaagctacag ctgccaggtc | 600 |
| acgcatgaag ggagcaccgt ggagaagaca gtggccccta cagaatgttc a | 651 |
| SEQ ID NO: 196 | |
| cagagcgcac tgacccagcc agcttcagtg agcggctcac caggtcagag cattaccatc | 60 |
| tcgtgtacgg gtactagcag cgatgttggt tcttataatt atgtgaattg gtaccagcag | 120 |
| catcccggga aggcgccgaa acttatgatt tatggtgttt ctaagcgtcc ctcaggcgtg | 180 |
| agcaaccgtt ttagcggatc caaaagcggc aacaccgcga gcctgaccat tagcggcctg | 240 |
| caagcggaag acgaagcgga ttattattgc ggtacttttg ctggtggttc ttattatggt | 300 |
| gtgtttggcg gcggcacgaa gttaaccgtc ctaggtcagc ccaaggctgc cccctcggtc | 360 |
| actctgttcc cgccctcctc tgaggagctt caagccaaca aggccacact ggtgtgtctc | 420 |
| ataagtgact tctacccggg agccgtgaca gtggcctgga aggcagatag cagccccgtc | 480 |
| aaggcgggag tggagaccac cacaccctcc aaacaaagca acaacaagta cgcggccagc | 540 |
| agctatctga gcctgacgcc tgagcagtgg aagtcccaca gaagctacag ctgccaggtc | 600 |
| acgcatgaag ggagcaccgt ggagaagaca gtggccccta cagaatgttc a | 651 |
| SEQ ID NO: 197 | |
| caggtgcagc tggtgcagag cggagctgag gtgaagaagc caggcgccag cgtcaaggtg | 60 |
| tcctgcaagg ccagcggcta caccttcacc agcagctaca tcaactgggt ccgccaggct | 120 |
| cctgggcagg gactggagtg gatgggcacc atcaaccccg tgtccggcag caccagctac | 180 |
| gcccagaagt tccagggcag agtcaccatg accagggaca ccagcatcag caccgcctac | 240 |
| atggagctgt ccaggctgag aagcgacgac accgccgtgt actactgcgc caggggcggc | 300 |
| tggttcgact actggggcca gggcaccctg gtgaccgtgt cctcagctag caccaagggc | 360 |
| cccagcgtgt tccccctggc cccctgcagc agaagcacca gcgagagcac agccgccctg | 420 |
| ggctgcctgg tgaaggacta cttccccgag ccagtgaccg tgtcctggaa cagcggagcc | 480 |
| ctgaccagcg gcgtgcacac cttccccgcc gtgctgcaga gcagcggcct gtacagcctg | 540 |
| tccagcgtgg tgaccgtgcc cagcagcaac ttcggcaccc agacctacac ctgcaacgtg | 600 |
| gaccacaagc ccagcaacac caaggtggac aagaccgtgg agaggaagtg ctgcgtggag | 660 |
| tgccccccct gcccagcccc cccagtggcc ggaccctccg tgttcctgtt cccccccaag | 720 |
| cccaaggaca ccctgatgat cagcaggacc cccgaggtga cctgcgtggt ggtggacgtg | 780 |
| agccacgagg acccagaggt gcagttcaac tggtacgtgg acggcgtgga ggtgcacaac | 840 |
| gccaagacca agcccagaga ggaacagttt aacagcacct tcagggtggt gtccgtgctg | 900 |
| accgtggtgc accaggactg gctgaacggc aaagagtaca agtgcaaggt ctccaacaag | 960 |
| ggcctgccag cccccatcga gaaaaccatc agcaagacca agggccagcc acgggagccc | 1020 |
| caggtgtaca ccctgccccc cagccgggag gaaatgacca agaaccaggt gtccctgacc | 1080 |
| tgtctggtga agggcttcta ccccagcgac atcgccgtgg agtgggagag caacggccag | 1140 |
| cccgagaaca actacaagac cacccccccc atgctggaca gcgacggcag cttcttcctg | 1200 |
| tacagcaagc tgacagtgga caagagcagg tggcagcagg gcaacgtgtt cagctgcagc | 1260 |
| gtgatgcacg aggccctgca caaccactac acccagaaga gcctgagcct gtcccccggc | 1320 |
| aag | 1323 |
| SEQ ID NO: 198 | |
| caggtgcagc tggtgcagag cggagctgag gtgaagaagc caggcgccag cgtcaaggtg | 60 |
| tcctgcaagg ccagcggcta caccttcacc agcagctaca tcaactgggt gcgccaggct | 120 |
| ccagggcagg gactggagtg gatgggccag atcaacgccg ccagcggcat gaccagatac | 180 |
| gcccagaagt tccagggcag agtcacaatg accagggaca cctctatcag caccgcctac | 240 |
| atggagctgt ccaggctgag aagcgacgac accgccgtgt actactgcgc caggggcggc | 300 |
| tggttcgact actggggcca gggcaccctg gtgaccgtgt cctcagctag caccaagggc | 360 |
| cccagcgtgt tccccctggc cccctgcagc agaagcacca gcgagagcac agccgccctg | 420 |
| ggctgcctgg tgaaggacta cttccccgag ccagtgaccg tgtcctggaa cagcggagcc | 480 |
| ctgaccagcg gcgtgcacac cttccccgcc gtgctgcaga gcagcggcct gtacagcctg | 540 |
| tccagcgtgg tgaccgtgcc cagcagcaac ttcggcaccc agacctacac ctgcaacgtg | 600 |
| gaccacaagc ccagcaacac caaggtggac aagaccgtgg agaggaagtg ctgcgtggag | 660 |
| tgccccccct gcccagcccc cccagtggcc ggaccctccg tgttcctgtt cccccccaag | 720 |
| cccaaggaca ccctgatgat cagcaggacc cccgaggtga cctgcgtggt ggtggacgtg | 780 |
| agccacgagg acccagaggt gcagttcaac tggtacgtgg acggcgtgga ggtgcacaac | 840 |
| gccaagacca agcccagaga ggaacagttt aacagcacct tcagggtggt gtccgtgctg | 900 |
| accgtggtgc accaggactg gctgaacggc aaagagtaca agtgcaaggt ctccaacaag | 960 |
| ggcctgccag cccccatcga gaaaaccatc agcaagacca agggccagcc acgggagccc | 1020 |
| caggtgtaca ccctgccccc cagccgggag gaaatgacca agaaccaggt gtccctgacc | 1080 |
| tgtctggtga agggcttcta ccccagcgac atcgccgtgg agtgggagag caacggccag | 1140 |
| cccgagaaca actacaagac cacccccccc atgctggaca gcgacggcag cttcttcctg | 1200 |
| tacagcaagc tgacagtgga caagagcagg tggcagcagg gcaacgtgtt cagctgcagc | 1260 |
| gtgatgcacg aggccctgca caaccactac acccagaaga gcctgagcct gtcccccggc | 1320 |
| aag | 1323 |
| SEQ ID NO: 199 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcaat attaatgctg ctgctggtat tactctttat | 180 |
| gctcagaagt ttcagggtcg ggtcaccatg acccgtgata ccagcattag caccgcgtat | 240 |
| atggaactga gccgcctgcg tagcgatgat acggccgtgt attattgcgc gcgtggtggt | 300 |
| tggtttgatt attggggcca aggcaccctg gtgacggtta gctcagcttc caccaagggc | 360 |
| cccagcgtgt tccccctggc cccctgcagc agaagcacca gcgagagcac agccgccctg | 420 |
| ggctgcctgg tgaaggacta cttccccgag cccgtgaccg tgagctggaa cagcggagcc | 480 |
| ctgaccagcg gcgtgcacac cttccccgcc gtgctgcaga gcagcggcct gtacagcctg | 540 |
| agcagcgtgg tgaccgtgcc cagcagcaac ttcggcaccc agacctacac ctgcaacgtg | 600 |
| gaccacaagc ccagcaacac caaggtggac aagaccgtgg agcggaagtg ctgcgtggag | 660 |
| tgccccccct gccctgcccc tcctgtggcc ggaccctccg tgttcctgtt cccccccaag | 720 |
| cccaaggaca ccctgatgat cagccggacc cccgaggtga cctgcgtggt ggtggacgtg | 780 |
| agccacgagg accccgaggt gcagttcaac tggtacgtgg acggcgtgga ggtgcacaac | 840 |
| gccaagacca agccccggga ggaacagttc aacagcacct tccgggtggt gtccgtgctg | 900 |
| accgtggtgc accaggactg gctgaacggc aaagaataca agtgcaaggt gtccaacaag | 960 |
| ggcctgcctg cccccatcga gaaaaccatc agcaagacaa agggccagcc cagggaaccc | 1020 |
| caggtgtaca ccctgccccc cagccgggag gaaatgacca agaaccaggt gtccctgacc | 1080 |
| tgtctggtga agggcttcta ccccagcgac atcgccgtgg agtgggagag caacggccag | 1140 |
| cccgagaaca actacaagac cacccccccc atgctggaca gcgacggcag cttcttcctg | 1200 |
| tacagcaagc tgacagtgga caagagccgg tggcagcagg gcaacgtgtt cagctgcagc | 1260 |
| gtgatgcacg aggccctgca caaccactac acccagaaga gcctgagcct gtcccccggc | 1320 |
| aaa | 1323 |
| SEQ ID NO: 200 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcggt attaatcctc ctgctggtac tacttcttat | 180 |
| gctcagaagt ttcagggtcg ggtcaccatg acccgtgata ccagcattag caccgcgtat | 240 |
| atggaactga gccgcctgcg tagcgatgat acggccgtgt attattgcgc gcgtggtggt | 300 |
| tggtttgatt attggggcca aggcaccctg gtgacggtta gctcagcttc caccaagggc | 360 |
| cccagcgtgt tccccctggc cccctgcagc agaagcacca gcgagagcac agccgccctg | 420 |
| ggctgcctgg tgaaggacta cttccccgag cccgtgaccg tgagctggaa cagcggagcc | 480 |
| ctgaccagcg gcgtgcacac cttccccgcc gtgctgcaga gcagcggcct gtacagcctg | 540 |
| agcagcgtgg tgaccgtgcc cagcagcaac ttcggcaccc agacctacac ctgcaacgtg | 600 |
| gaccacaagc ccagcaacac caaggtggac aagaccgtgg agcggaagtg ctgcgtggag | 660 |
| tgccccccct gccctgcccc tcctgtggcc ggaccctccg tgttcctgtt cccccccaag | 720 |
| cccaaggaca ccctgatgat cagccggacc cccgaggtga cctgcgtggt ggtggacgtg | 780 |
| agccacgagg accccgaggt gcagttcaac tggtacgtgg acggcgtgga ggtgcacaac | 840 |
| gccaagacca agccccggga ggaacagttc aacagcacct tccgggtggt gtccgtgctg | 900 |
| accgtggtgc accaggactg gctgaacggc aaagaataca agtgcaaggt gtccaacaag | 960 |
| ggcctgcctg cccccatcga gaaaaccatc agcaagacaa agggccagcc cagggaaccc | 1020 |
| caggtgtaca ccctgccccc cagccgggag gaaatgacca agaaccaggt gtccctgacc | 1080 |
| tgtctggtga agggcttcta ccccagcgac atcgccgtgg agtgggagag caacggccag | 1140 |
| cccgagaaca actacaagac cacccccccc atgctggaca gcgacggcag cttcttcctg | 1200 |
| tacagcaagc tgacagtgga caagagccgg tggcagcagg gcaacgtgtt cagctgcagc | 1260 |
| gtgatgcacg aggccctgca caaccactac acccagaaga gcctgagcct gtcccccggc | 1320 |
| aaa | 1323 |
| SEQ ID NO: 201 | |
| caggtgcaat tggttcagag cggcgcggaa gtgaaaaaac cgggcgcgag cgtgaaagtg | 60 |
| agctgcaaag cctccggata tacctttact tcttcttata ttaattgggt ccgccaagcc | 120 |
| cctgggcagg gtctcgagtg gatgggcaat attaatcctg ctactggtca tgctgattat | 180 |
| gctcagaagt ttcagggtcg ggtgaccatg acccgtgata ccagcattag caccgcgtat | 240 |
| atggaactga gccgcctgcg tagcgatgat acggccgtgt attattgcgc gcgtggtggt | 300 |
| tggtttgatt attggggcca aggcaccctg gtgacggtta gctcagcttc caccaagggc | 360 |
| cccagcgtgt tccccctggc cccctgcagc agaagcacca gcgagagcac agccgccctg | 420 |
| ggctgcctgg tgaaggacta cttccccgag cccgtgaccg tgagctggaa cagcggagcc | 480 |
| ctgaccagcg gcgtgcacac cttccccgcc gtgctgcaga gcagcggcct gtacagcctg | 540 |
| agcagcgtgg tgaccgtgcc cagcagcaac ttcggcaccc agacctacac ctgcaacgtg | 600 |
| gaccacaagc ccagcaacac caaggtggac aagaccgtgg agcggaagtg ctgcgtggag | 660 |
| tgccccccct gccctgcccc tcctgtggcc ggaccctccg tgttcctgtt cccccccaag | 720 |
| cccaaggaca ccctgatgat cagccggacc cccgaggtga cctgcgtggt ggtggacgtg | 780 |
| agccacgagg accccgaggt gcagttcaac tggtacgtgg acggcgtgga ggtgcacaac | 840 |
| gccaagacca agccccggga ggaacagttc aacagcacct tccgggtggt gtccgtgctg | 900 |
| accgtggtgc accaggactg gctgaacggc aaagaataca agtgcaaggt gtccaacaag | 960 |
| ggcctgcctg cccccatcga gaaaaccatc agcaagacaa agggccagcc cagggaaccc | 1020 |
| caggtgtaca ccctgccccc cagccgggag gaaatgacca agaaccaggt gtccctgacc | 1080 |
| tgtctggtga agggcttcta ccccagcgac atcgccgtgg agtgggagag caacggccag | 1140 |
| cccgagaaca actacaagac cacccccccc atgctggaca gcgacggcag cttcttcctg | 1200 |
| tacagcaagc tgacagtgga caagagccgg tggcagcagg gcaacgtgtt cagctgcagc | 1260 |
| gtgatgcacg aggccctgca caaccactac acccagaaga gcctgagcct gtcccccggc | 1320 |
| aaa | 1323 |

### REFERENCES

1. Crotty, S. T Follicular Helper Cell Differentiation, Function, and Roles in Disease. Immunity 41, 529-542 (2014).
2. Crotty, S. Follicular helper CD4 T cells (TFH). Annu Rev Immunol 29, 621-663 (2011).
3. Victora, G. D. & Nussenzweig, M. C. Germinal centers. Annu Rev Immunol 30, 429-457 (2012).
4. Crotty, S. A brief history of T cell help to B cells. Nat. Rev. Immunol. 15, 185-189 (2015).
5. Gitlin, A. D., Shulman, Z. & Nussenzweig, M. C. Clonal selection in the germinal centre by regulated proliferation and hypermutation. Nature 509, 637-640 (2014).
6. Vinuesa, C. G. & Cyster, J. G. How T cells earn the follicular rite of passage. Immunity 35, 671-680 (2011).
7. Ueno, H., Banchereau, J. & Vinuesa, C. G. Pathophysiology of T follicular helper cells in humans and mice. Nat Immunol 16, 142-152 (2015).
8. Johnston, R. J. et al. Bcl6 and Blimp-1 are reciprocal and antagonistic regulators of T follicular helper cell differentiation. Science 325, 1006-1010 (2009).
9. Nurieva, R. I. et al. Bcl6 mediates the development of T follicular helper cells. Science 325, 1001-1005 (2009).
10. Yu, D. et al. The transcriptional repressor Bcl-6 directs T follicular helper cell lineage commitment. Immunity 31, 457-468 (2009).
11. Suto, A. et al. Development and characterization of IL-21-producing CD4+ T cells. J Exp Med 205, 1369-1379 (2008).
12. Nurieva, R. I. et al. Generation of T follicular helper cells is mediated by interleukin-21 but independent of T helper 1,2, or 17 cell lineages. Immunity 29, 138-149 (2008).
13. Eto, D. et al. IL-21 and IL-6 are critical for different aspects of B cell immunity and redundantly induce optimal follicular helper CD4 T cell (Tfh) differentiation. PLoS ONE 6, e17739 (2011).
14. Choi, Y. S., Eto, D., Yang, J. A., Lao, C. & Crotty, S. Cutting edge: STAT1 is required for IL-6-mediated Bcl6 induction for early follicular helper cell differentiation. J Immunol 190, 3049-3053 (2013).
15. Schmitt, N. et al. The cytokine TGF-β co-opts signaling via STAT3-STAT4 to promote the differentiation of human TFH cells. Nat Immunol (2014). doi:10.1038/ni.2947
16. Ma, C. S. et al. Early commitment of naïve human CD4(+) T cells to the T follicular helper (T(FH)) cell lineage is induced by IL-12. Immunol Cell Biol 87, 590-600 (2009).
17. Schmitt, N. et al. Human dendritic cells induce the differentiation of interleukin-21-producing T follicular helper-like cells through interleukin-12. Immunity 31, 158-169 (2009).
18. Nakayamada, S. et al. Early Th1 cell differentiation is marked by a Tfh cell-like transition. Immunity 35, 919-931 (2011).
19. Gonzalez, R. et al. Screening the mammalian extracellular proteome for regulators of embryonic human stem cell pluripotency. Proc Natl Acad Sci USA 107, 3552-3557 (2010).
20. Thompson, T. B., Cook, R. W., Chapman, S. C., Jardetzky, T. S. & Woodruff, T. K. Beta A versus beta B: is it merely a matter of expression? Mol. Cell. Endocrinol. 225, 9-17 (2004).
21. Gold, E. & Risbridger, G. Activins and activin antagonists in the prostate and prostate cancer. Mol. Cell. Endocrinol. 359, 107-112 (2012).
22. Muttukrishna, S., Tannetta, D., Groome, N. & Sargent, I. Activin and follistatin in female reproduction. Mol. Cell. Endocrinol. 225, 45-56 (2004).
23. Munz, B. et al. The roles of activins in repair processes of the skin and the brain. Mol. Cell. Endocrinol. 180, 169-177 (2001).
24. Phillips, D. J., de Kretser, D. M. & Hedger, M. P. Activin and related proteins in inflammation: not just interested bystanders. Cytokine Growth Factor Rev. 20, 153-164 (2009).
25. Lotinun, S., Pearsall, R. S., Home, W. C. & Baron, R. Activin receptor signaling: a potential therapeutic target for osteoporosis. Curr Mol Pharmacol 5, 195-204 (2012).
26. Aleman-Muench, G. R. & Soldevila, G. When versatility matters: activins/inhibins as key regulators of immunity. Immunol Cell Biol 90, 137-148 (2012).
27. Dalton, S. Signaling networks in human pluripotent stem cells. Curr. Opin. Cell Biol. 25, 241-246 (2013).
28. Beattie, G. M. et al. Activin A maintains pluripotency of human embryonic stem cells in the absence of feeder layers. Stem Cells 23, 489-495 (2005).
29. Vallier, L., Alexander, M. & Pedersen, R. A. Activin/Nodal and FGF pathways cooperate to maintain pluripotency of human embryonic stem cells. J. Cell. Sci. 118, 4495-4509 (2005).
30. Sulzbacher, S., Schroeder, I. S., Truong, T. T. & Wobus, A. M. Activin A-induced differentiation of embryonic stem cells into endoderm and pancreatic progenitors-the influence of differentiation factors and culture conditions. Stem Cell Rev 5, 159-173 (2009).
31. Jones, C. P., Gregory, L. G., Causton, B., Campbell, G. A. & Lloyd, C. M. Activin A and TGF-β promote T(H)9 cell-mediated pulmonary allergic pathology. J. Allergy Clin. Immunol. 129, 1000-10.e3 (2012).
32. Semitekolou, M. et al. Activin-A induces regulatory T cells that suppress T helper cell immune responses and protect from allergic airway disease. J Exp Med 206, 1769-1785 (2009).
33. Huber, S. et al. Activin a promotes the TGF-beta-induced conversion of CD4+CD25-T cells into Foxp3+ induced regulatory T cells. J Immunol 182, 4633-4640 (2009).
34. Ogawa, K., Funaba, M., Chen, Y. & Tsujimoto, M. Activin A functions as a Th2 cytokine in the promotion of the alternative activation of macrophages. J Immunol 177, 6787-6794 (2006).
35. Schmitt, N. et al. IL-12 receptor (31 deficiency alters in vivo T follicular helper cell response in humans. Blood 121, 3375-3385 (2013).
36. Kroenke, M. A. et al. Bcl6 and Maf Cooperate To Instruct Human Follicular Helper CD4 T Cell Differentiation. J Immunol 188, 3734-3744 (2012).
37. Chevalier, N. et al. CXCR5 Expressing Human Central Memory CD4 T Cells and Their Relevance for Humoral Immune Responses. J Immunol 186, 5556-5568 (2011).
38. Locci, M. et al. Human circulating PD-⁺1CXCR3⁻CXCR5⁺ memory Tfh cells are highly functional and correlate with broadly neutralizing HIV antibody responses. Immunity 39, 758-769 (2013).
39. Sáez de Guinoa, J., Barrio, L., Mellado, M. & Carrasco, Y. R. CXCL13/CXCR5 signaling enhances BCR-triggered B-cell activation by shaping cell dynamics. Blood 118, 1560-1569 (2011).
40. Morita, R. et al. Human blood CXCR5(+)CD4(+) T cells are counterparts of T follicular cells and contain specific subsets that differentially support antibody secretion. Immunity 34, 108-121 (2011).
41. Travis, M. A. & Sheppard, D. TGF-β Activation and Function in Immunity. Annu Rev Immunol (2013). doi:10.1146/annurev-immunol-032713-120257
42. Tsuchida, K. et al. Activin isoforms signal through type I receptor serine/threonine kinase ALK7. Mol. Cell. Endocrinol. 220, 59-65 (2004).
43. Abe, Y., Minegishi, T. & Leung, P. C. K. Activin receptor signaling. Growth Factors 22, 105-110 (2004).
44. Tsuchida, K. et al. Activin signaling as an emerging target for therapeutic interventions. Cell Commun. Signal 7, 15 (2009).
45. Johnston, R. J., Choi, Y. S., Diamond, J. A., Yang, J. A. & Crotty, S. STAT5 is a potent negative regulator of TFH cell differentiation. J Exp Med 209, 243-250 (2012).
46. Ballesteros-Tato, A. et al. Interleukin-2 inhibits germinal center formation by limiting T follicular helper cell differentiation. Immunity 36, 847-856 (2012).
47. Oestreich, K. J., Mohn, S. E. & Weinmann, A. S. Molecular mechanisms that control the expression and activity of Bcl-6 in TH1 cells to regulate flexibility with a TFH-like gene profile. Nat Immunol 13, 405-411 (2012).
48. Inman, G. J. et al. SB-431542 is a potent and specific inhibitor of transforming growth factor-beta superfamily type I activin receptor-like kinase (ALK) receptors ALK4, ALK5, and ALK7. Mol. Pharmacol. 62, 65-74 (2002).
49. Herbertz, S. et al. Clinical development of galunisertib (LY2157299 monohydrate), a small molecule inhibitor of transforming growth factor-beta signaling pathway. Drug Des Devel Ther 9, 4479-4499 (2015).
50. Bentebibel, S.-E. et al. Induction of ICOS+CXCR3+CXCR5+ TH Cells Correlates with Antibody Responses to Influenza Vaccination. Sci Transl Med 5, 176ra32-176ra32 (2013).
51. Ma, C. S. & Deenick, E. K. Human T follicular helper (Tfh) cells and disease. Immunol Cell Biol 92, 64-71 (2014).
52. Moens, L. & Tangye, S. G. Cytokine-Mediated Regulation of Plasma Cell Generation: IL-21 Takes Center Stage. Front Immunol 5, 65 (2014).
53. Shen, M. M. et al. Expression of LIF in transgenic mice results in altered thymic epithelium and apparent interconversion of thymic and lymph node morphologies. EMBO J 13, 1375-1385 (1994).
54. Tran, D. Q., Ramsey, H. & Shevach, E. M. Induction of FOXP3 expression in naive human CD4+FOXP3 T cells by T-cell receptor stimulation is transforming growth factor-beta dependent but does not confer a regulatory phenotype. Blood 110, 2983-2990 (2007).
55. Trapnell, C., Pachter, L. & Salzberg, S. L. TopHat: discovering splice junctions with RNA-Seq. Bioinformatics 25, 1105-1111 (2009).
56. Schmieder, R. & Edwards, R. Quality control and preprocessing of metagenomic datasets. Bioinformatics 27, 863-864 (2011).
57. Li, H. et al. The Sequence Alignment/Map format and SAMtools. Bioinformatics 25, 2078-2079 (2009).
58. Anders, S., Pyl, P. T. & Huber, W. HTSeq—a Python framework to work with high-throughput sequencing data. Bioinformatics 31, 166-169 (2015).
59. Bengtsson, H., Ray, A., Spellman, P. & Speed, T. P. A single-sample method for normalizing and combining full-resolution copy numbers from multiple platforms, labs and analysis methods. Bioinformatics 25, 861-867 (2009).
60. Love, M. I., Huber, W. & Anders, S. Moderated estimation of fold change and dispersion for RNA-seq data with DESeq2. Genome Biol. 15, 550 (2014).

1.-15. (canceled)

16. A method for inhibiting, decreasing or reducing Tfh (follicular helper) cell differentiation, germinal center B cell development or an antibody response, comprising administering an inhibitor of activin or activin receptor to a subject in an amount effective to inhibit, decrease or reduce Tfh (follicular helper) cell differentiation, germinal center B cell development or an antibody response in the subject.

17. A method for inhibiting or reducing an undesirable or aberrant immune response, comprising administering an inhibitor of activin or activin receptor to a subject in an amount effective to inhibit or reduce an undesirable or aberrant immune response in the subject.

18. A method for inhibiting or reducing an undesirable or aberrant inflammatory response or inflammation, comprising administering an inhibitor of activin or activin receptor to a subject in an amount effective to inhibit or reduce an undesirable or aberrant inflammatory response or inflammation in the subject.

19. The method of claim 16, wherein the inhibitor binds to activin or activin Receptor.

20. The method of claim 16, wherein the inhibitor comprises Follistatin.

21. The method of claim 16, wherein the inhibitor comprises an antibody or an antigen binding fragment thereof that binds to activin or activin receptor, or an antisense nucleic acid sequence of activin or activin receptor.

22. The method of claim 21, wherein the antibody or fragment thereof comprises an Fab, Fab′, F(ab′) 2, Fv, Fd, single-chain Fv (scFv), disulfide-linked Fvs (sdFv), V, VH, trispecific (Fab), bispecific (Fab2), diabody ((VL-VH)2 or (V-V)2), triabody (trivalent), tetrabody (tetravalent), minibody ((scFv-CH3)2), bispecific single-chain Fv (Bis-scFv), IgGdeltaCH2, scFv-Fc or (scFv)2-Fc fragment.

23. The method of claim 21, wherein the antibody is human or humanized.

24. The method of claim 21, wherein the antibody comprises one or more of M244B, M248, LS Bio LS-C195902, R&D Systems Clone 132815, R&D Systems Clone 69403, R&D Systems AF338, Novus Biologicals MM0074-7L18, GeneTex 7L18, or Ray Biotech ACVR1B.

25.-38. (canceled)

39. The method of claim 16, wherein production or one or more antibodies is reduced, decreased, inhibited or suppressed.

40. The method of claim 16, wherein the subject is a mammal.

41. (canceled)

42. The method of claim 16, wherein the activin inhibitor binds to a mammalian activin or activin receptor.

43. (canceled)

44. The method of claim 16, wherein the activin comprises a dimer, comprising two polypeptides each selected from inhibin-βA or inhibin-βB.

45. The method of claim 16, wherein the activin is mammalian.

46. The method of claim 45, wherein the activin is human.

47. The method of claim 16, wherein the activin comprises all or a portion of a human activin sequence.

48. The method of claim 16, wherein the activin receptor comprises one of ActRIIA or ActRIIB

49.-60. (canceled)

61. The method of claim 16, wherein the inhibitor of Activin or Activin Receptor is an anti ActRII antibody or antigen binding fragment.

62. The method of claim 61, wherein the anti ActRII antibody or antigen binding fragment is selected from:
(i) an antibody or antigen binding fragment thereof that binds activin receptor, wherein said antibody or antigen binding fragment comprises a heavy chain variable region CDR1 comprising an amino acid sequence selected from the group consisting of SEQ ID NOs: 22-35; a heavy chain variable region CDR2 comprising an amino acid sequence selected from the group consisting of SEQ ID NOs: 36-49; a heavy chain variable region CDR3 comprising an amino acid sequence selected from the group consisting of SEQ ID NOs: 50-63; a light chain variable region CDR1 comprising an amino acid sequence selected from the group consisting of SEQ ID NOs: 64-77; a light chain variable region CDR2 comprising an amino acid sequence selected from the group consisting of SEQ ID NOs: 78-91; and a light chain variable region CDR3 comprising an amino acid sequence selected from the group consisting of SEQ ID NOs: 92-105;
(ii) an antibody or antigen binding fragment thereof that binds activin receptor, wherein said antibody or antigen binding fragment comprises:
(a) a heavy chain variable region CDR1 of SEQ ID NO: 22; a heavy chain variable region CDR2 of SEQ ID NO: 36; a heavy chain variable region CDR3 of SEQ ID NO: 50; a light chain variable region CDR1 of SEQ ID NO: 64; a light chain variable region CDR2 of SEQ ID NO: 78; and a light chain variable region CDR3 of SEQ ID NO: 92,
(b) a heavy chain variable region CDR1 of SEQ ID NO: 23 a heavy chain variable region CDR2 of SEQ ID NO: 37; a heavy chain variable region CDR3 of SEQ ID NO: 51; a light chain variable region CDR1 of SEQ ID NO: 65; a light chain variable region CDR2 of SEQ ID NO: 79; and a light chain variable region CDR3 of SEQ ID NO: 93,
(c) a heavy chain variable region CDR1 of SEQ ID NO: 24; a heavy chain variable region CDR2 of SEQ ID NO: 38; a heavy chain variable region CDR3 of SEQ ID NO: 52; a light chain variable region CDR1 of SEQ ID NO: 66; a light chain variable region CDR2 of SEQ ID NO: 80; and a light chain variable region CDR3 of SEQ ID NO: 94,
(d) a heavy chain variable region CDR1 of SEQ ID NO: 25; a heavy chain variable region CDR2 of SEQ ID NO: 39; a heavy chain variable region CDR3 of SEQ ID NO: 53; a light chain variable region CDR1 of SEQ ID NO: 67; a light chain variable region CDR2 of SEQ ID NO: 81; and a light chain variable region CDR3 of SEQ ID NO: 95,
(e) a heavy chain variable region CDR1 of SEQ ID NO: 26; a heavy chain variable region CDR2 of SEQ ID NO: 40; a heavy chain variable region CDR3 of SEQ ID NO: 54; a light chain variable region CDR1 of SEQ ID NO: 68; a light chain variable region CDR2 of SEQ ID NO: 82; and a light chain variable region CDR3 of SEQ ID NO: 96,
(f) a heavy chain variable region CDR1 of SEQ ID NO: 27; a heavy chain variable region CDR2 of SEQ ID NO: 41; a heavy chain variable region CDR3 of SEQ ID NO: 55; a light chain variable region CDR1 of SEQ ID NO: 69; a light chain variable region CDR2 of SEQ ID NO: 62; and a light chain variable region CDR3 of SEQ ID NO: 97,
(g) a heavy chain variable region CDR1 of SEQ ID NO: 28; a heavy chain variable region CDR2 of SEQ ID NO: 42; a heavy chain variable region CDR3 of SEQ ID NO: 56; a light chain variable region CDR1 of SEQ ID NO: 70; a light chain variable region CDR2 of SEQ ID NO: 84; and a light chain variable region CDR3 of SEQ ID NO: 98,
(h) a heavy chain variable region CDR1 of SEQ ID NO: 29; a heavy chain variable region CDR2 of SEQ ID NO: 43; a heavy chain variable region CDR3 of SEQ ID NO: 57; a light chain variable region CDR1 of SEQ ID NO: 71 a light chain variable region CDR2 of SEQ ID NO: 85; and a light chain variable region CDR3 of SEQ ID NO: 99,
(i) a heavy chain variable region CDR1 of SEQ ID NO: 30; a heavy chain variable region CDR2 of SEQ ID NO: 44; a heavy chain variable region CDR3 of SEQ ID NO: 58; a light chain variable region CDR1 of SEQ ID NO: 72; a light chain variable region CDR2 of SEQ ID NO: 86; and a light chain variable region CDR3 of SEQ ID NO: 100,
(j) a heavy chain variable region CDR1 of SEQ ID NO: 31; a heavy chain variable region CDR2 of SEQ ID NO: 45; a heavy chain variable region CDR3 of SEQ ID NO: 59; a light chain variable region CDR1 of SEQ ID NO: 73; a light chain variable region CDR2 of SEQ ID NO: 87; and a light chain variable region CDR3 of SEQ ID NO: 101,
(k) a heavy chain variable region CDR1 of SEQ ID NO: 32; a heavy chain variable region CDR2 of SEQ ID NO: 46; a heavy chain variable region CDR3 of SEQ ID NO: 60; a light chain variable region CDR1 of SEQ ID NO: 74; a light chain variable region CDR2 of SEQ ID NO: 88; and a light chain variable region CDR3 of SEQ ID NO: 102,
(l) a heavy chain variable region CDR1 of SEQ ID NO: 33; a heavy chain variable region CDR2 of SEQ ID NO: 47; a heavy chain variable region CDR3 of SEQ ID NO: 61; a light chain variable region CDR1 of SEQ ID NO: 75; a light chain variable region CDR2 of SEQ ID NO: 89; and a light chain variable region CDR3 of SEQ ID NO: 103,
(m) a heavy chain variable region CDR1 of SEQ ID NO: 34; a heavy chain variable region CDR2 of SEQ ID NO: 48; a heavy chain variable region CDR3 of SEQ ID NO: 62; a light chain variable region CDR1 of SEQ ID NO: 76; a light chain variable region CDR2 of SEQ ID NO: 90; and a light chain variable region CDR3 of SEQ ID NO: 104, or
(n) a heavy chain variable region CDR1 of SEQ ID NO: 35; a heavy chain variable region CDR2 of SEQ ID NO: 49; a heavy chain variable region CDR3 of SEQ ID NO: 63; a light chain variable region CDR1 of SEQ ID NO: 77; a light chain variable region CDR2 of SEQ ID NO: 91; and a light chain variable region CDR3 of SEQ ID NO: 105;
(iii) an antibody or antigen binding fragment thereof that binds activin receptor, wherein said antibody or antigen binding fragment comprises a VH polypeptide sequence having at least 95% sequence identity to at least one of SEQ ID NOs: 120-133 and a VL polypeptide sequence having at least 95% sequence identity to at least one of SEQ ID NOs: 106-119;
(iv) an antibody or antigen binding fragment thereof that binds activin receptor, wherein said antibody or antigen binding fragment comprises a full length heavy chain amino acid sequence having at least 95% sequence identity to at least one sequence selected from the group consisting of SEQ ID NOs:167-171 and 177-181 and a full length light chain amino acid sequence having at least 95% sequence identity to at least one sequence selected from the group consisting of SEQ ID NOs:162-166 and 172-176;
(v) an antibody or antigen binding fragment thereof that binds activin receptor, wherein said antibody or antigen binding fragment comprises
(a) the variable heavy chain sequence of SEQ ID NO: 120 and variable light chain sequence of SEQ ID NO: 106;
(b) the variable heavy chain sequence of SEQ ID NO: 121 and variable light chain sequence of SEQ ID NO: 107;
(c) the variable heavy chain sequence of SEQ ID NO: 122 and variable light chain sequence of SEQ ID NO: 108;
(d) the variable heavy chain sequence of SEQ ID NO: 123 and variable light chain sequence of SEQ ID NO: 109;
(e) the variable heavy chain sequence of SEQ ID NO: 124 and variable light chain sequence of SEQ ID NO: 110;
(f) the variable heavy chain sequence of SEQ ID NO: 125 and variable light chain sequence of SEQ ID NO: 111;
(g) the variable heavy chain sequence of SEQ ID NO: 126 and variable light chain sequence of SEQ ID NO: 112;
(h) the variable heavy chain sequence of SEQ ID NO: 127 and variable light chain sequence of SEQ ID NO: 113;
(i) the variable heavy chain sequence of SEQ ID NO: 128 and variable light chain sequence of SEQ ID NO: 114;
(j) the variable heavy chain sequence of SEQ ID NO: 129 and variable light chain sequence of SEQ ID NO: 115;
(k) the variable heavy chain sequence of SEQ ID NO: 130 and variable light chain sequence of SEQ ID NO: 116;
(l) the variable heavy chain sequence of SEQ ID NO: 131 and variable light chain sequence of SEQ ID NO: 117;
(m) the variable heavy chain sequence of SEQ ID NO: 132 and variable light chain sequence of SEQ ID NO: 118; or
(n) the variable heavy chain sequence of SEQ ID NO: 133 and variable light chain sequence of SEQ ID NO: 119; or
(vi) an antibody or antigen binding fragment thereof that binds activin receptor, wherein said antigen binding fragment comprises
(a) the heavy chain sequence of SEQ ID NO: 167 and light chain sequence of SEQ ID NO: 162;
(b) the heavy chain sequence of SEQ ID NO: 168 and light chain sequence of SEQ ID NO: 163;
(c) the heavy chain sequence of SEQ ID NO: 169 and light chain sequence of SEQ ID NO: 164;
(d) the heavy chain sequence of SEQ ID NO: 170 and light chain sequence of SEQ ID NO: 165;
(e) the heavy chain sequence of SEQ ID NO: 171 and light chain sequence of SEQ ID NO: 166;
(f) the heavy chain sequence of SEQ ID NO: 177 and light chain sequence of SEQ ID NO: 172;
(g) the heavy chain sequence of SEQ ID NO: 178 and light chain sequence of SEQ ID NO: 173;
(h) the heavy chain sequence of SEQ ID NO: 179 and light chain sequence of SEQ ID NO: 174;
(i) the heavy chain sequence of SEQ ID NO: 180 and light chain sequence of SEQ ID NO: 175; or
(j) the heavy chain sequence of SEQ ID NO: 181 and light chain sequence of SEQ ID NO: 176;
or a pharmaceutical composition comprising such antibody or antigen binding fragment for use in the treatment of an undesirable or aberrant immune response, inflammatory response or inflammation.

63. The method of claim 62, wherein the undesirable or aberrant immune response, inflammatory response or inflammation is an autoimmune disease, rheumatoid arthritis, juvenile rheumatoid arthritis, osteoarthritis, psoriatic arthritis, diabetes mellitus, multiple sclerosis, encephalomyelitis, myasthenia gravis, systemic lupus erythematosus (SLE), autoimmune thyroiditis, atopic dermatitis, eczematous dermatitis, psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, Hashimoto's thyroiditis, autoimmune polyglandular syndrome, insulin-dependent diabetes mellitus, insulin-resistant diabetes mellitus, immune-mediated infertility, autoimmune Addison's disease, pemphigus vulgaris, pemphigus foliaceus, dermatitis herpetiformis, autoimmune alopecia, Vitiligo, autoimmune hemolytic anemia, autoimmune thrombocytopenic purpura, pernicious anemia, Guillain-Barre syndrome, acute rheumatic fever, sympathetic ophthalmia, Goodpasture's syndrome, systemic necrotizing vasculitis, antiphospholipid syndrome or an allergy.
